(19) United States
(12) Patent Application Publication
(10) Pub. No.: US 2002/0123095 A1
(43) Pub. Date: Sep. 5, 2002

(54) Estrogen receptor alpha variants and methods of detection thereof

(75) Inventors: Francis Kalush, Rockville, MD (US); Michael J. Cassel, San Leandro, CA (US); Stuart Soo-In Hwang, San Carlos, CA (US); Emily S. Winn-Deen, Potomac, MD (US)

Correspondence Address: CELERA GENOMICS CORP., ATTN: ROBERT A. MILLMAN, PATENT DIRECTOR, 45 WEST GUDE DRIVE, C2-4#20, ROCKVILLE, MD, 20850, US

(73) Assignee: PE CORPORATION (NY), Norwalk, CT (US)

(21) Appl. No.: 09/933,267

(22) Filed: Aug. 21, 2001

Related U.S. Application Data
(63) Continuation-in-part of application No. 09/826,314, filed on Apr. 5, 2001.
Application No. 09/826,314 is a continuation-in-part of application No. 09/804,076, filed on Mar. 13, 2001.
Application No. 09/804,076 is a continuation-in-part of application No. 09/768,184, filed on Jan. 24, 2001.
Application No. 09/768,184 is a continuation-in-part of application No. 09/692,414, filed on Oct. 20, 2000.
(60) Provisional application No. 60/183,756, filed on Feb. 22, 2000.
(60) Provisional application No. 60/160,626, filed on Oct. 20, 1999.

(51) Int. Cl. C07K 14/705; C07H 21/04; C07K 16/28; C12P 21/02; C12N 5/06

(52) U.S. Cl. 435/69.1; 435/325; 435/320.1; 530/350; 536/23.5; 530/388.22

(57) ABSTRACT

The present invention is based on sequencing genomic DNA from human chromosome 6 and cDNAs to define the genomic structure of estrogen receptor alpha genes and novel polymorphism/haplotypes in the estrogen receptor gene/protein. Such polymorphism/haplotypes can lead to a variety of disorders that are mediated/modulated by a variant estrogen receptor, such as a susceptibility to cancer, osteoporosis, cardiovascular disorder, etc. Based on this sequencing approach, the present invention provides genomic nucleotide sequences, cDNA sequences, amino acid sequences and sequence polymorphism/haplotypes in the ESR-alpha genes, methods of detecting these sequences/polymorphism/haplotypes in a sample, methods of determining a risk of having or developing a disorder mediated by a variant estrogen receptor and methods of screening for compounds used to treat disorders mediated by a variant estrogen receptor.

### RELATED APPLICATIONS

The present application claims priority to applications U.S. Ser. Nos. 60/160,626, filed Oct. 20, 1999 (Atty. Docket CL000119-PROV); 60/183,756, filed Feb. 22, 2000 (Atty. Docket CL000258-PROV); 09/692,414, filed Oct. 20, 2000 (Atty. Docket CL000258); 09/768,184, filed Jan. 24, 2001 (Atty. Docket CL000258CIP); 09/804,076, filed Mar. 13, 2001 (Atty. Docket CL000258CI2); and 09/826,314, filed Apr. 5, 2001 (Atty. Docket CL000258CI3).

### FIELD OF THE INVENTION

The present invention is in the field of disease detection and therapy. The present invention specifically provides the identification of previously unknown nucleic acid/amino acid polymorphisms within the estrogen receptor alpha gene (ESR-alpha) and the genomic sequence of this gene for use in the development of diagnostics and therapies for diseases and disorders mediated/modulated by the estrogen receptor.

### BACKGROUND OF THE INVENTION

Estrogen Receptor

The human estrogen receptor alpha belongs to the nuclear hormone receptor family. Nuclear hormone receptors are a family of hormone-activated transcription factors that can initiate or enhance the transcription of genes containing specific hormone response elements.

The ER protein consists of 595 amino acids with a molecular weight of 66 kDa, 8 transcribed exons, with six different functional domains. Two of those domains are highly conserved in the primary sequence of members of the nuclear hormone receptor superfamily. One of the domains, the DNA binding domain (DBD), contains two zinc fingers that mediate receptor binding to hormone response elements in the promoters of hormone-responsive genes. In the C-terminal region, the hormone-binding domain (HBD) contains two regions of sequence homology with other hormone receptors and gives hormone specificity and selectivity. The human ER-alpha gene is located in chromosome 6q.25.1.

Estrogen receptors, like other steroid receptors, are transcription factors that are activated upon binding to steroids (estradiol) or steroid analogs such as tamoxifen. Upon activation the receptors dimerize to form homodimers or heterodimers that bind to estrogen receptor elements (EREs) located in the promoter region of estrogen-activated genes and coordinate transcription by interacting with host co-activators.

Role of Estrogen in Cardiovascular Disease

Heart disease is the leading cause of mortality in women, a fact that is under appreciated by both women and physicians. One in 9 women aged 45-65 have some form of cardiovascular disease and the number increases to 1 in 3 after age 65. Each year, 240,000 U.S. women die from heart disease, and nearly 90,000 die of stroke. Moreover, approximately 44% die within one year of suffering a heart attack, compared with 26% of men (Warren M P and Kulak J Clin Obs Gyn 1998 41(4):976-987).

Estrogens exert a wide range of physiological effects on a large variety of cell types. For example, they regulate cell growth and apoptosis and a myriad of functions related to reproduction. There are two types of estrogen receptors, alpha and beta. Blood vessels and bone contain beta receptors, the liver has alpha receptors, and both alpha and beta receptors are found in the central nervous system. The interaction of these different receptor sites influences the biological effects of estrogen and selective estrogen receptor modulators (SERMs), such as raloxifene. The binding patterns dictate whether an estrogen or a SERM acts as an estrogen agonist or an antagonist (Mendelsohn M E and Karas R H New Engl J Med 1999, 340(23):1801-1811 ;Grese T A and Dodge J A Curr Pharm Design 1998, 4:71-92). Tissue-specific relationships exist between SERMs and the receptor binding sites. Estrogens also increase high-density lipoprotein cholesterol levels, decrease low-density lipoprotein cholesterol, and decrease plasminogen-activating inhibitor levels (Meisler J G Jour Women's Health 1999, 8(1):51-57). All estrogens require cellular receptors for their expression. In general, estrogen receptors are ligand-inducible transcription factors, which regulate the expression of target genes after hormone binding (Faustini-Fustini et al. Eur J Endocrin 1999, 140:111-129). Estrogen may also have important effects on the vascular wall. Estradiol and progesterone receptors have been identified in arterial endothelial and smooth muscle cells (Campisi D et al. Int J Tiss React 1987, IX(5):393-398). Estrogens act on the wall of the artery to relax vascular smooth muscle and to decrease vascular resistance. The mechanism appears to be through stimulation of endothelial-derived relaxing factors and an endogenous nitrate (Warren M P and Kulak J Clin Obs Gyn 1998 41(4):976-987). The relaxation induced by 17B-estradiol may play an important role in the regulation of coronary tone, which reduces the risk of coronary disease in postmenopausal women. The production of nitric oxide is mediated by the estrogen receptor, because when the receptor is blocked by an antiestrogen agent, nitric oxide is suppressed.

Several studies have shown that estrogen therapy reduces the risk of heart disease by up to 50% (most recently reviewed by Mendelsohn M E and Karas R H New Engl J Med 1999, 340(23):1801-1811;Rich-Edwards J W N Engl J Med, 1995, 332:1758-1765; Gerhard M, Ganz P, Circulation, 1995, 92:5-8; Grodstein F, et al N Engl J Med 1997, 336:1769-75; Chasen-Taber L and Stampfer M J Ann of Int Med, 1998, 128:467-477; Warren M P and Kulak J Clin Obstet Gyn 1998, 41(4):976-987). Loss of estrogen may be one of the most important factors in the development of cardiovascular disease in women.

While there is no direct evidence that estrogen prevents atherogenesis, considerable epidemiologic evidence exists that suggests that estrogens may have some benefit in reducing cardiovascular disease: (1) In all age groups, women have a lower incidence of cardiovascular disease than do men; (2) women who undergo a premature surgical menopause and do not take estrogens are twice as likely to have cardiovascular disease are age-matched premenopausal controls; (3) postmenopausal women who use estrogens have a significantly lower incidence of cardiovascular disease compared with those who do not; and (4) women with coronary artery disease detected by angiography have a higher survival rate if they are estrogen users.

In recent years, reports of favorable effects of estrogen therapy on cardiovascular morbidity and mortality have led to enthusiasm for widespread use of estrogens by postmenopausal women (Meinertz T Herz 1997, 22: 151-157). Guidelines for estrogen therapy issued by the American College of Physicians include the statement “Women who have coronary heart disease are likely to benefit from hormone therapy.”

More than 30 prospective studies and 13 case controlled studies have examined the effect of estrogen replacement therapy on cardiovascular incidence or prevalence and all cause mortality (Stampfer M J et al. New Engl J Med 1991, 325:756-62; Grady D et al. Ann Intern Med 1992, 117:1016-37). The majority of these studies showed lower morbidity and mortality from coronary heart disease among users of postmenopausal estrogens than among non-users. Specifically, they have shown that coronary artery disease in estrogen takers is approximately 50% that in women who do not take estrogen. Overall, the bulk of the evidence strongly supports a protective effect of estrogens yielding a relative risk of 0.56 (95% confidence interval 0.50-0.61). However, a “healthy woman selection bias” is present in these studies and potentially may confound these results (estrogen takers have better weight control, exercise more, and smoke less than women who are not prescribed estrogen). Moreover, other biases such as estrogen takers tend to have higher education, higher income, etc., are confounding these epidemiologic studies (Abrams J Clin Cardiol 1998, 21:218-222).

Since the earlier observational trials were not randomized, it is believed by many that as much as 25% of this 50% reduction in risk is due to these various methodological biases (Barrett-Conner E and Grady D 1998, Ann Rev Public Health 19:55-72). Recently, 2 meta-analyses estimated the reduction in coronary heart disease associated with estrogen use to be in the range of 35 to 44%, respectively (Grodstein F and Stampfer M J Prog Cardiol Dis 1995, 38: 199-210; Barrett-Conner E and Grady D 1998, Annu Rev Public Health 19:55-72). Recent studies are exploring the issue of opposed vs unopposed estrogen, because of a documented increased risk for uterine cancer in women with an intact uterus who are taking estrogen alone. The new lines of evidence are suggesting that women taking estrogen plus a progestin (usually a medroxyprogesterone acetate) do not receive an equivalent benefit from the cardioprotective effects compared to women taking estrogen alone (Hulley S et al 1998 JAMA 280:605-613; Abrams J Clin Cardiol 1998, 21:218-222).

The loss of estrogen at menopause is associated with a 6% decline in HDL cholesterol levels and a 5% rise in LDL cholesterol levels, which may explain the higher cardiovascular disease rate among postmenopausal women compared with premenopausal women. The lower incidence of cardiovascular disease among postmenopausal women who take estrogen may be explained in part by the resultant 15% to 19% decrease in LDL cholesterol levels and the 16% to 18% increase in HDL cholesterol levels (JAMA 1995, 273:199-208). The PEPI (Postmenopausal Estrogen/Progestin Intervention, a randomized, double-blind placebo-controlled trial, showed that HDL cholesterol levels rose significantly more in women assigned to estrogen alone than in women assigned the combined estrogen (JAMA 1995, 273:199-208). Recent non-human primates studies substantiate these findings (Clarkson T B Lab An Sci 1998, 48(6):569-72). Statistical modeling of the effect of estrogen on lipid profiles indicates that 25-50% of the apparent cardioprotection due to estrogen is mediated by favorable changes in HDL-cholesterol (Bush T L et al. 1987 Circulation 75:1102-9; Gruchow H W et al. 1988 Am Heart J 115:954-63).

Estrogen replacement therapy is not without risk. For years, studies have shown a 3-4-fold increased risk of venous thromboembolism (VTE) in users of oral contraceptives compared to non-users (Weiss G Am J Obstet Gynecol 1999 180:S295-301). One study has shown that intrinsic coagulation factors play a significant role in oral contraceptive-associated VTE (Vandenbroucke J P et al. Lancet 1994 344:1453-7; Rosing J et al. Br J Haematol 1997, 97:233-238). The Factor V Leiden mutation increases risk of VTE 5-10 fold in non users, but 30-fold in third-generation oral contraceptive users. Combined estrogens appear to induce resistance to the body's natural anticoagulation system (APC). Heterozygotes for the Factor V Leiden mutation who take oral contraceptives develop APC resistance as high as that seen in women who are homozygous.

Estrogens increase the risk of endometrial carcinoma approximately 6-fold, an effect that is eliminated, for the most part, by the addition of progestins (Barrett-Conner E and Grady D 1998, Ann Rev Public Health 19:55-72). Controversy continues over whether estrogen replacement increases the risk of breast cancer, but some studies indicate risk is elevated by as much as 30%. (Greendale G A et al. Lancet 1999, 353:571-80).

A number of prospective randomized studies designed to definitely establish whether estrogen replacement therapy reduces the risk of cardiovascular disease in women and whether it increases the risk of breast cancer, are underway. One recently completed trial (HERS—Heart and Estrogen/progestin Replacement Study) compared continuous combined estrogen plus medroxyprogesterone acetate to placebo in 2700 women with pre-existing coronary disease (Hully S et al. 1998 JAMA 280(7):605-13). Compared to controls, the intervention group had significantly more heart disease events in year one of the trial, but significantly fewer events in years 4 and 5 of the trial. Moreover, a significant increase in the rate of thromboembolic events occurred in the early years of the study in women taking hormones. Based on these results, hormone replacement therapy is not recommended for secondary prevention of heart disease.

Two other large, ongoing clinical trials on primary prevention of cardiovascular disease using estrogens are underway. The Women's Health Initiative, due to be completed in 2005 and a U.K. study called WIS-DOM, due to be completed in 2010, should shed new light on the protective effects of estrogen on cardiovascular disease (Meisler J G Jour Women's Health 1999, 8(1):51-5).

In summary, ongoing research suggests that estrogen replacement therapy, particularly involving recently formulated designer estrogens or SERMs, may have beneficial effects on the cardiovascular system as well as bone, without the untoward effects on breast and endometrial tissue. Caution still needs to be observed, nonetheless. Women who take estrogens are, on average, better educated, healthier, have higher incomes and have better access to health care. These differences rather than the estrogens may account for much of the lower risk of heart disease.

For postmenopausal women without frank disease, estrogen replacement therapy appears to have a beneficial effect when one considers the magnitude, consistency, and biological plausibility of the data. For women with pre-existing disease, questions remain as to the safety and efficacy of exogenous estrogens as protective agents against cardiovascular disease.

Estrogen and autoimmune diseases

A. Systemic Lupus Erythematosus

There is a widely held view that estrogens play a role in Systemic lupus erythematosus because:

1. Women of child bearing age are nine times more likely to develop systemic lupus erythematosus than men. Prior to pubescence the rate is three fold higher in females, while post menopausal women have an equal chance of developing SLE as aged matched males. Many studies have been done that show that the reason for the differences in the sexes is probably estrogen related (Lahita R. G., 1986: Springer Seminars in Immunopathology 9, 305-314; Krammer, G. M. and Tsokos, G. C., 1998 Clinical Immunology and Immunopathology 89: 192-195; Rider at al., 1998 Clinical Immunology and Immunopathology 89: 171-180).

Clues to the role of estrogens in SLE came from studies that concluded that oral contraceptives adversely affected the morbidity of this illness (Buton, J. P., 1996 Ann. Med. Interne, 147:259-264; Julkunen, 1991: Scan. J. Rheumatol. 20:427-433).

2. Patients with Klinefelter syndrome (XXY), have been reported with SLE (Stem et al., 1977: Arthritis and Rheumatism 20:18-22).

3. Patients with SLE have anti-estrogen antibodies (Feldman, 1987: Biochem. Biophys. Acta, 145:1342-1348: Bucala et al., 1987: Clin. Exp. Immunol. 67:167-175)

In the past, oral contraceptives have been shown to cause flare ups of SLE, their use was discouraged in women with SLE, while the current thinking is that the lower dose birth control pills are safe for SLE patients (Julkunen H A Scand J Rheumatol 1991;20(6):427-33). As well hormone replacement therapy is considered safe for SLE patients (Mok et al., Scand J Rheumatol 1998;27(5):342-6: Kreidstein et al., 1997, J Rheumatol 1997 November;24(11):2149-52)

4. The estrogen antagonist tamoxofin seems to improve the course of the disease (Sthoeger, 1997, Ann N Y Acad Sci 1997 Apr 5;815:367-8: Sthoeger, 1994, J Rheumatol 1994 Dec;21(12):2231-8).

B. Estrogen, Rheumatoid Arthritis (RA) and osteoarthritis

The literature surrounding the involvement of estrogens in Rheumatoid arthritis is less clear than with osteoarthritis. Epidemiological studies suggests that RA is influenced by female sex hormones, by one study states that the use of oral contraceptives may postpone the onset of RA, but that estrogens alone no not alleviate the symptoms of RA (Bijlsma Am J Reprod Immunol 1992 Oct-Dec;28(3-4):231-4). Adjuvant oestrogen treatment does increase bone mineral density in postmenopausal women with RA, and may protect against osteophoresis which is often a complication of RA (van den Brink: Ann Rheum Dis 1993 Apr;52(4):302-5). While the study mentioned above indicated that estrogens did not alleviate RA symptoms, another study concluded that adjuvant estrogen therapy did not even improve the symptoms. One polymorphism has been reported in the estrogen receptor that seems to be associated with the age of onset of RA (Ushiyama Ann Rheum Dis January 1999; 58(1):7-10)

Osteoarthritis on the other hand is less prevalent in postmenopausal women who take estrogen replacement therapy (ERT) (Felson Curr Opin Rheumatol 1998 May; 10(3):269-72) suggesting that ERT may be beneficial in preventing osteoarthritis.

C. Estrogen and Osteoporosis

Osteoporosis is a metabolic bone disorder that leads to bone fragility and subsequent risk of fracture. Treatment for postmenopausal women with osteoporesis includes hormone replacement, in particular estrogen. Estrogen has shown to reduce the incidence of bone loss and fractures (Weiss et al., N Engl J Med 1980 Nov 20;303(21):1195-8 :Paganini-Hill et al., Ann Intern Med 1981 Jul;95(1):28-31: Ettinger et al., Ann Intern Med 1985 Mar;102(3):319-24)

Further, polymorphisms in the estrogen receptor have been associated with bone loss in both humans and mice.(Kobayashi J Bone Miner Res 1996 Mar;11(3):306-11: Kurabayashi Am J Obstet Gynecol 1999 May; 180(5):1115-20; Deng Hum Genet 1998 Nov; 103(5):576-85)

Estrogens and Cognitive function

Compared with men, women are at greater risk of developing Alzheimer's disease. Several studies show that women who take estrogen after menopause have a lower incidence of Alzheimer's disease. Among women with Alzheimer's, those taking estrogen suffer less severe symptoms and slower mental deterioration. The duration of estrogen use also seems to be important in reducing risk. Women with a history of long-term use (more than 10 years) had the lowest risk. But even women who took estrogen for a short time also benefited.

Estrogen and breast cancer

The major risk factors for the development of breast cancer are sex, age, family history of breast cancer, age of menarche, age at first full-term pregnancy, and age of menopause. All of these factors, with the exception of family history, have been shown to be directly associated with lifetime exposure to estrogen, increased hormone exposure being associated with increased risk of developing breast cancer. The increased cancer risk is believed to be caused by an estrogen receptor-mediated proliferative response in cells of the mammary epithelium.

Tamoxifen, an estrogen receptor antagonist, has been shown to be an effective agent for both the prevention and treatment of breast cancer. Using immunohistochemical methods, it is possible to classify breast tumors as being estrogen receptor positive or negative, depending upon the amount of estrogen receptor protein expressed in the tissue. Estrogen receptor positive tumors are more likely to respond to treatment with tamoxifen than estrogen receptor negative tumors. Pre-menopausal women are more likely to develop estrogen receptor negative breast cancers than are post-menopausal women.

Mutations altering the structure and function of the estrogen receptor have been described in primary breast tumors or breast cancer cell lines. It is not clear however whether these changes are primary (and involved in the processes leading to carcinogenesis) or secondary (and a consequence of genetic instability in cancer tissues). In addition to these somatic mutations, some studies have pointed to a possible association between inherited DNA sequence changes and the development of breast cancer, but these studies are also controversial.

Further evidence for the role of estrogen receptors in breast cancer comes from the recent finding that the gene BRCA1, which when inherited in a mutant form predisposes to the development of breast cancer, inhibits estrogen receptor signaling.

Estrogens and endometrial cancer

Carcinoma of the endometrium is the most common pelvic malignancy in women, however because in approximately 75% of cases it is confined to the body of the uterus at the time of diagnosis, it can usually be cured by hysterectomy. Unopposed exposure of endometrial cells to estrogens dramatically increases the chance of developing this form of uterine cancer and it is for this reason that hormone replacement therapy consisting solely of estrogen should not be given to women with intact uteri. Cyclical or continuous co-administration of progesterone serves to prevent excessive proliferation of endometrial cells, reducing the risk of endometrial cancer in post-menopausal women receiving estrogen as part of hormone replacement therapy regimens.

The majority of cases of endometrial cancers express estrogen receptor and, in general, estrogen responsive tumors have a favorable prognosis. Acquired (somatic) mutations have been described in up to 8.5% of cases, however the role of these mutations in the development and progression of endometrial cancer is uncertain at present.

Although it remains somewhat controversial, studies suggest that use of tamoxifen may increase the chance of developing endometrial cancer. This may be because, in addition to its role in estrogen receptor blockade, tamoxifen has partial receptor agonist activity and results in low-grade induction of estrogen responsive genes that induce endometrial proliferation.

Given the involvement of the estrogen receptor in mediating/modulating various disorders, it is critical to identify sequence polymorphisms in the estrogen receptor and to correlate these with disease states, therapeutic effectiveness and the like. The present invention advances the art by providing a variety of previously unidentified polymorphisms in the ESR-alpha protein.

SNPs

The genomes of all organisms undergo spontaneous mutation in the course of their continuing evolution, generating variant forms of progenitor sequences (Gusella, Ann. Rev. Biochem. 55, 831-854 (1986)). The variant form may confer an evolutionary advantage or disadvantage relative to a progenitor form or may be neutral. In some instances, a variant form confers a lethal disadvantage and is not transmitted to subsequent generations of the organism. In other instances, a variant form confers an evolutionary advantage to the species and is eventually incorporated into the DNA of many or most members of the species and effectively becomes the progenitor form. Additionally, the effect of a variant form may be both beneficial and detrimental, depending on the circumstances. For example, a heterozygous sickle cell mutation confers resistance to malaria, but a homozygous sickle cell mutation is usually lethal. In many instances, both progenitor and variant form(s) survive and co-exist in a species population. The coexistence of multiple forms of a sequence gives rise to polymorphisms, such as SNPs.

The reference allelic form is arbitrarily designated and may be, for example, the most abundant form in a population, or the first allelic form to be identified, and other allelic forms are designated as alternative, variant or polymorphic alleles. The allelic form occurring most frequently in a selected population is sometimes referred to as the “wild type” form.

Approximately 90% of all polymorphisms in the human genome are single nucleotide polymorphisms (SNPs). SNPs are single base pair positions in DNA at which different alleles, or alternative nucleotides, exist in some population. The SNP position, or SNP site, is usually preceded by and followed by highly conserved sequences of the allele (e.g., sequences that vary in less than 1/100 or 1/1000 members of the populations). An individual may be homozygous or heterozygous for an allele at each SNP position. As defined by the present invention, the least frequent allele at a SNP position can have any frequency that is less than the frequency of the more frequent allele, including a frequency of less than 1% in a population. A SNP can, in some instances, be referred to as a “cSNP” to denote that the nucleotide sequence containing the SNP is an amino acid coding sequence.

A SNP may arise due to a substitution of one nucleotide for another at the polymorphic site. Substitutions can be transitions or transversions. A transition is the replacement of one purine nucleotide by another purine nucleotide, or one pyrimidine by another pyrimidine. A transversion is the replacement of a purine by a pyrimidine, or vice versa. A SNP may also be a single base insertion/deletion variant (referred to as “indels”). A substitution that changes a codon coding for one amino acid to a codon coding for a different amino acid is referred to as a non-synonymous codon change, or missense mutation. A synonymous codon change, or silent mutation, is one that does not result in a change of amino acid due to the degeneracy of the genetic code. A nonsense mutation is a type of non-synonymous codon change that results in the formation of a stop codon, thereby leading to premature termination of a polypeptide chain and a defective protein.

SNPs, in principle, can be bi-, tri-, or tetra-allelic. However, tri- and tetra-allelic polymorphisms are extremely rare, almost to the point of non-existence (Brookes, Gene 234 (1999) 177-186). For this reason, SNPs are often referred to as “bi-allelic markers”, or “di-allelic markers”.

Causative SNPs are those SNPs that produce alterations in gene expression or in the expression or function of a gene product, and therefore are most predictive of a possible clinical phenotype. One such class includes SNPs falling within regions of genes encoding a polypeptide product, i.e. cSNPs. These SNPs may result in an alteration of the amino acid sequence of the polypeptide product (i.e., non-synonymous codon changes) and give rise to the expression of a defective or other variant protein. Furthermore, in the case of nonsense mutations, a SNP may lead to premature termination of a polypeptide product. Such variant products can result in a pathological condition, e.g., genetic disease. Examples of genes in which a polymorphism within a coding sequence gives rise to genetic disease include sickle cell anemia and cystic fibrosis. Causative SNPs do not necessarily have to occur in coding regions; causative SNPs can occur in any region that can ultimately affect the expression and/or activity of the protein encoded by the nucleic acid. Such gene areas include those involved in transcription, such as SNPs in promoter regions, in gene areas involved in transcript processing, such as SNPs at intron-exon boundaries that may cause defective splicing, or SNPs in mRNA processing signal sequences such as polyadenylation signal regions. For example, a SNP may inhibit splicing of an intron and result in mRNA containing a premature stop codon, leading to a defective protein. Consequently, SNPs in regulatory regions can have substantial phenotypic impact.

Some SNPs that are not causative SNPs nevertheless are in close association with, and therefore segregate with, a disease-causing sequence. In this situation, the presence of the SNP correlates with the presence of, or susceptibility to, the disease. These SNPs are invaluable for diagnostics and disease susceptibility screening.

Clinical trials have shown that patient response to treatment with pharmaceuticals is often heterogeneous. Thus there is a need for improved approaches to pharmaceutical agent design and therapy. SNPs can be used to help identify patients most suited to therapy with particular pharmaceutical agents (this is often termed “pharmacogenomics”). Pharmacogenomics can also be used in pharmaceutical research to assist the drug selection process. (Linder et al. (1997), Clinical Chemistry, 43, 254; Marshall (1997), Nature Biotechnology, 15, 1249; International Patent Application WO 97/40462, Spectra Biomedical; and Schafer et al. (1998), Nature Biotechnology, 16, 3.).

Population Studies

Population Genetics is the study of how Mendel's laws and other genetic principles apply to entire populations. Such a study is essential to a proper understanding of evolution because, fundamentally, evolution is the result of progressive change in the genetic composition of a population. Population genetics thus seeks to understand and to predict the effects of such genetic phenomena as segregation, recombination, and mutation; at the same time, population genetics must take into account such ecological and evolutionary factors as population size, patterns of mating, geographic distribution of individuals, migration and natural selection.

Ideally, one would wish to know how to describe the types and frequencies of genes in a population, to explain how the population's genetic composition came to be the way it is, and to predict how the population would change as a result of natural selection or as a result of artificial selection.

In order to explain many of those issues it is important to understand the existing relation between loci denominated: Linkage.

Linkage is the coinheritance of two or more nonallelic genes because their loci are in close proximity on the same chromosome, such that after meiosis they remain associated more often than the 50% expected for unlinked genes. During meiosis, there is a physical crossing over, it is clear that during the production of germ cells there is a physical exchange of maternal and paternal genetic contributions between individual chromatids. This exchange necessarily separates genes in chromosomal regions that were contiguous in each parent and, by mixing them with retained linear order, results in “recombinants”. The process of forming recombinants through meiotic crossing-over is an essential feature in the reassortment of genetic traits and is central to understanding the transmission of genes.

Recombination generally occurs between large segments of DNA. This means that contiguous stretches of DNA and genes are likely to be moved together. Conversely, regions of the DNA that are far apart on a given chromosome are likely to become separated during the process of crossing-over.

It is possible to use molecular markers to clarify the recombination events that take place during meiosis. Some markers as (CA)n repeats of different lengths are dispersed throughout human DNA and there is little selective pressure in their lengths are used as position markers and regional identifying characters along chromosomes. Those markers can be used to distinguish paternally derived from maternally derived gene regions.

Other markers are Single Nucleotide Polymorphism (SNP), those are biallelic markers, also used to analyzed the transmission of those markers to offspring.

The pattern of a set of markers along a chromosome is referred to as a “Haplotype”. Therefore sets of alleles on the same small chromosomal segment tend to be transmitted as a block through a pedigree. By analyzing the haplotypes in a series of offspring of parents whose haplotypes are known, it is possible to establish which parental segment of which chromosome was transmitted to which child. When not broken up by recombinations, haplotypes can be treated for mapping purposes as alleles at a single highly polymorphic locus.

The existance of a preferential occurrence of a disease gene in association with specific alleles of linked markers is called “Linkage Disequilibrium”(LD). This sort of disequilibrium generally implies that most of the disease chromosomes carry the same mutation and the markers being tested are quite close to the disease gene. For example, there is considerable linkage disequilibrium across the entire HLA locus. The A3 allele is in LD with the B7 and B14 alleles, and as a result B7 and B14 are also highly associated with hemochromatosis. Thus, HLA typing alone can significantly alter the estimate of risk for hemochromatosis, even if other family members are not available for formal linkage analysis. As a result, using a combination of several markers surrounding the presumptive location of the gene, a haplotype can be determined for affected and unaffected family members.

SNP-Based Association Analysis and Linkage Disequilibrium Mapping

SNPs are useful in association studies for identifying particular SNPs, or other polymorphisms, associated with pathological conditions, such as breast cancer. Association studies may be conducted within the general population and are not limited to studies performed on related individuals in affected families (linkage studies). An association study using SNPs involves determining the frequency of the SNP allele in many patients with the disorder of interest, such as breast cancer, as well as controls of similar age and race. The appropriate selection of patients and controls is critical to the success of SNP association studies. Therefore, a pool of individuals with well-characterized phenotypes is extremely desirable. For example, blood pressure and heart rate can be correlated with SNP patterns in hypertensive individuals in whom these physiological parameters are known in order to find associations between particular SNP genotypes and known phenotypes. Significant associations between particular SNPs or SNP haplotypes and phenotypic characteristics can be determined by standard statistical methods. Association analysis can either be direct or LD based. In direct association analysis, causative SNPs are tested that are candidates for the pathogenic sequence itself.

In LD based SNP association analysis, random SNPs are tested over a large genomic region, possibly the entire genome, in order to find a SNP in LD with the true pathogenic sequence or pathogenic SNP. For this approach, high density SNP maps are required in order for random SNPs to be located close enough to an unknown pathogenic locus to be in linkage disequilibrium with that locus in order to detect an association. SNPs tend to occur with great frequency and are spaced uniformly throughout the genome. The frequency and uniformity of SNPs means that there is a greater probability, compared with other types of polymorphisms such as tandem repeat polymorphisms, that a SNP will be found in close proximity to a genetic locus of interest. SNPs are also mutationally more stable than tandem repeat polymorphisms, such as VNTRs. LD-based association studies are capable of finding a disease susceptibility gene without any a priori assumptions about what or where the gene is.

Currently, however, it is not feasible to do SNP association studies over the entire human genome, therefore candidate genes associated with breast cancer are targeted for SNP identification and association analysis. The candidate gene approach uses a priori knowledge of disease pathogenesis to identify genes that are hypothesized to directly influence development of the disease. The candidate gene approach may focus on a gene that is directly targeted by a drug used to treat the disorder. To discover SNPs associated with an increased susceptibility to breast cancer, candidate genes can be selected from systems physiologically implicated in the disease pathway. SNPs found in these genes are then tested for statistical association with disease in individuals who have the disease compared with appropriate controls. The candidate gene approach has the advantages of drastically reducing the number of candidate SNPs, and the number of individuals, that need to be typed, compared with LD-based association studies of random SNPs over large areas of, or complete, genomes. Furthermore, in the candidate gene approach, no assumptions are made about the extent of LD over any particular area of the genome.

Combined with the use of a high density map of appropriately spaced, sufficiently informative SNP markers, association studies, including linkage disequilibrium-based genome wide association studies, will enable the identification of most genes involved in complex disorders, such as breast cancer. This will enhance the selection of candidate genes most likely to contain causative SNPs associated with a particular disease. All of the SNPs disclosed by the present invention can be employed as part of genome-wide association studies or as part of candidate gene association studies.

The present invention advances the state of the art and provides commercially useful embodiments by providing previously unidentified SNPs in the estrogen receptor genes.

### SUMMARY OF THE INVENTION

The present invention is based on sequencing genomic DNA from human chromosome 6 and cDNAs to define the genomic structure of estrogen receptor alpha genes, novel polymorphisms in the estrogen receptor gene/protein and previously unknown haplotypes. Such polymorphisms/haplotypes can lead to a variety of disorders that are mediated/modulated by a variant estrogen receptor, such as a susceptibility to cancer, osteoporosis, cardiovascular disorders, etc. Based on this sequencing approach, the present invention provides genomic nucleotide sequences, cDNA sequences, amino acid sequences, sequence polymorphisms in the ESR-alpha gene, haplotypes of these polymorphisms, methods of detecting these sequences/polymorphisms in a sample, methods of determining a risk of having or developing a disorder mediated by a variant estrogen receptor and methods of screening for compounds used to treat disorders mediated by a variant estrogen receptor.

### DESCRIPTION OF THE FIGURES

FIG. 1. Complete genomic sequence of the estrogen receptor alpha gene.

FIG. 2. Sequence polymorphisms found in the ESR-alpha genomic DNA (nucleotide position is based on the sequence provided in FIG. 1.)

(a) SNPs in Liverpool clinical tissue samples.

(b) SNPs in Coriell Diversity panels.

(c) SNPs in Liverpool Control Population

(d) PCR primers.

(e) Sequencing primers.

FIG. 3. Amino acid sequence of the estrogen receptor alpha protein.

FIG. 4. Estrogen Receptor Haplotypes (See Haplotype Section).

(a) Liverpool samples from 48 patients, and each patient had a tumor and blood sample typed. Coriell samples were control.

(b) The non-singleton haplotype data fitted to a neighbor-joining tree (L is Liverpool sample).

FIG. 5. The domain structure of the ESR1 protein and the positions of the SNPs disclosed herein.

FIG. 6. The distribution and frequency of many of the SNPs of the present invention.

FIG. 7. A graphic representation of the human ESR1 locus.

(a) Complete structure of the human estrogen receptor alpha (ERα). Exons are represented by filled boxes and introns by horizontal lines.

(b) Order and names of contigs used to complete the genomic sequence. GA numbers represent Celera contig numbers. Research Genetics BAC clones are represented by standard plate and well numbering.

FIG. 8. ESR-alpha SNPs Genotyping Results a) in Coriell Samples, b) in Liverpool Samples (T=tumor sample, B=blood sample, LC=Liverpool controls), c) in Liverpool Control sample

FIG. 9. ESR-alpha exons with SNPs. (see FIG. 2 for “N”, “C”, “I”, “A”, “S” representations). Underlined sequences indicate the primer sequences.

### DETAILED DESCRIPTION OF THE INVENTION

General Description

The present invention is based on sequencing genomic DNA from human chromosome 6 and cDNAs to define the genomic structure of estrogen receptor alpha genes and novel polymorphisms and haplotypes in the estrogen receptor gene/protein. Such polymorphisms/haplotypes can lead to a variety of disorders that are mediated/modulated by a variant estrogen receptor, such as a susceptibility to cancer, osteoporosis, cardiovascular disorders, etc. Based on this sequencing approach, the present invention provides genomic nucleotide sequences, cDNA sequences, amino acid sequences and sequence polymorphisms/haplotypes in the ESR-alpha gene, methods of detecting these sequences/polymorphisms/haplotypes in a sample, methods of determining a risk of having or developing a disorder mediated by a variant estrogen receptor and methods of screening for compounds used to treat disorders mediated by a variant estrogen receptor.

Isolated SNP-Containing Nucleic Acid Molecules

The present invention provides isolated nucleic acid molecules that contain one or more SNPs disclosed by the present invention. The present invention further provides isolated nucleic acid molecules that encode the variant protein. Such nucleic acid molecules will consist of, consist essentially of, or comprise one or more SNPs of the present invention. The nucleic acid molecule can have additional nucleic acid residues, such as nucleic acid residues that are naturally associated with it or heterologous nucleotide sequences.

As used herein, an “isolated” SNP-containing nucleic acid molecule is one that contains a SNP of the present invention and is separated from other nucleic acid present in the natural source of the nucleic acid. Generally, the isolated SNP-containing nucleic acid, as used herein, will be comprised of one or more SNP positions disclosed by the present invention with flanking nucleotide sequence on either side of the SNP positions. Preferably the flanking sequence is up to about 300 bases, 100 bases, 50 bases, 30 bases, 15 bases, 10 bases, or 4 bases on either side of a SNP position for detection reagents or as long as the entire protein encoding sequence if it is to be used to produce a protein containing the coding variants disclosed in Figures. The important point is that the nucleic acid is isolated from remote and unimportant flanking sequences and is of appropriate length such that it can be subjected to the specific manipulations or uses described herein such as recombinant expression, preparation of probes and primers for the SNP position, and other uses specific to the SNP-containing nucleic acid sequences.

Moreover, an “isolated” nucleic acid molecule, such as a cDNA molecule containing a SNP of the present invention, can be substantially free of other cellular material, or culture medium when produced by recombinant techniques, or chemical precursors or other chemicals when chemically synthesized. However, the nucleic acid molecule can be fused to other coding or regulatory sequences and still be considered isolated. For example, recombinant DNA molecules contained in a vector are considered isolated. Further examples of isolated DNA molecules include recombinant DNA molecules maintained in heterologous host cells or purified (partially or substantially) DNA molecules in solution. Isolated RNA molecules include in vivo or in vitro RNA transcripts of the isolated SNP-containing DNA molecules of the present invention. Isolated nucleic acid molecules according to the present invention further include such molecules produced synthetically.

Isolated SNP-containing nucleic acid molecules can be in the form of RNA, such as mRNA, or in the form DNA, including cDNA and genomic DNA obtained by cloning or produced by chemical synthetic techniques or by a combination thereof. The nucleic acid, especially DNA, can be double-stranded or single-stranded. Single-stranded nucleic acid can be the coding strand (sense strand) or the non-coding strand (anti-sense strand).

The present invention further provides related nucleic acid molecules that hybridize under stringent conditions to the nucleic acid molecules disclosed herein. As used herein, the term “hybridizes under stringent conditions” is intended to describe conditions for hybridization and washing under which nucleotide sequences encoding a peptide at least 60-70% homologous to each other typically remain hybridized to each other. The conditions can be such that sequences at least about 60%, at least about 70%, or at least about 80%, or at least about 90% or more homologous to each other typically remain hybridized to each other. Such stringent conditions are known to those skilled in the art and can be found in Current Protocols in Molecular Biology, John Wiley & Sons, N.Y. (1989), 6.3.1-6.3.6. One example of stringent hybridization conditions are hybridization in 6×sodium chloride/sodium citrate (SSC) at about 45° C., followed by one or more washes in 0.2×SSC, 0.1% SDS at 50-65° C. Examples of moderate to low stringency hybridization conditions are well known in the art.

Specific Embodiments

Peptide Molecules

The present invention provides nucleic acid sequences that encode variants of the estrogen receptor. These variant molecule/sequences will be referred to herein as the estrogen receptor variants of the present invention, the estrogen receptor proteins of the present invention, or peptides/proteins of the present invention.

The present invention provides isolated estrogen receptor protein molecules that consist of, consist essentially of or are comprised of the amino acid sequences of the estrogen receptor variant proteins disclosed herein.

As used herein, a protein or peptide is said to be “isolated” or “purified” when it is substantially free of cellular material or free of chemical precursors or other chemicals. The peptides of the present invention can be purified to homogeneity or other degrees of purity. The level of purification will be based on the intended use. The critical feature is that the preparation allows for the desired function of the peptide, even if in the presence of considerable amounts of other components.

In some uses, “substantially free of cellular material” includes preparations of the peptide having less than about 30% (by dry weight) other proteins (i.e., contaminating protein), less than about 20% other proteins, less than about 10% other proteins, or less than about 5% other proteins. When the peptide is recombinantly produced, it can also be substantially free of culture medium, i.e., culture medium represents less than about 20% of the volume of the protein preparation.

The language “substantially free of chemical precursors or other chemicals” includes preparations of the peptide in which it is separated from chemical precursors or other chemicals that are involved in its synthesis. In one embodiment, the language “substantially free of chemical precursors or other chemicals” includes preparations of the estrogen receptor protein having less than about 30% (by dry weight) chemical precursors or other chemicals, less than about 20% chemical precursors or other chemicals, less than about 10% chemical precursors or other chemicals, or less than about 5% chemical precursors or other chemicals.

The isolated estrogen receptor proteins can be purified from cells that naturally express it, purified from cells that have been altered to express it (recombinant), or synthesized using known protein synthesis methods. For example, a nucleic acid molecule encoding the estrogen receptor protein is cloned into an expression vector, the expression vector introduced into a host cell and the protein expressed in the host cell. The protein can then be isolated from the cells by an appropriate purification scheme using standard protein purification techniques. Many of these techniques are described in detail below.

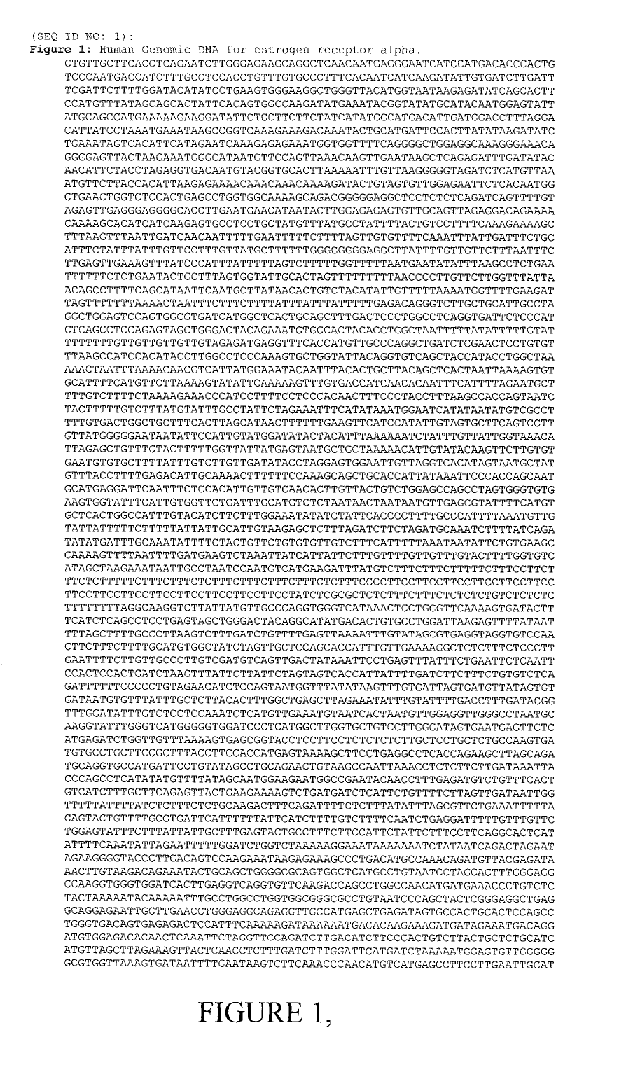

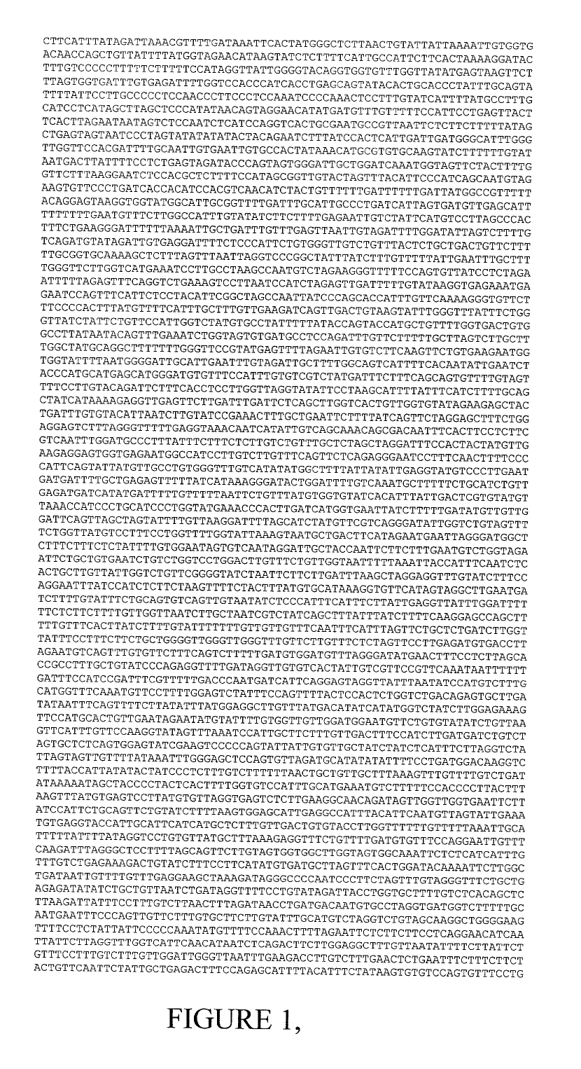

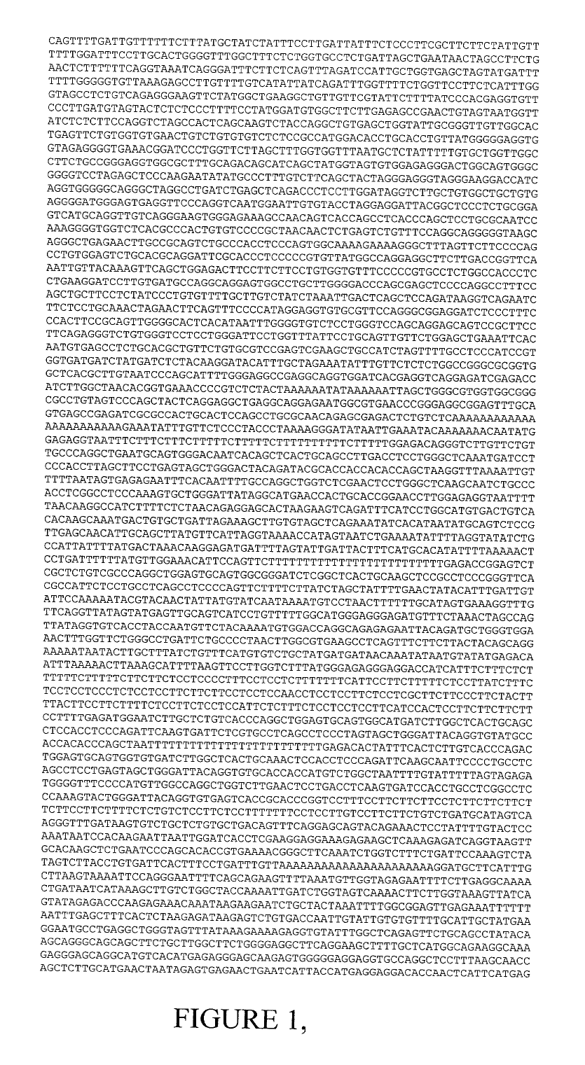

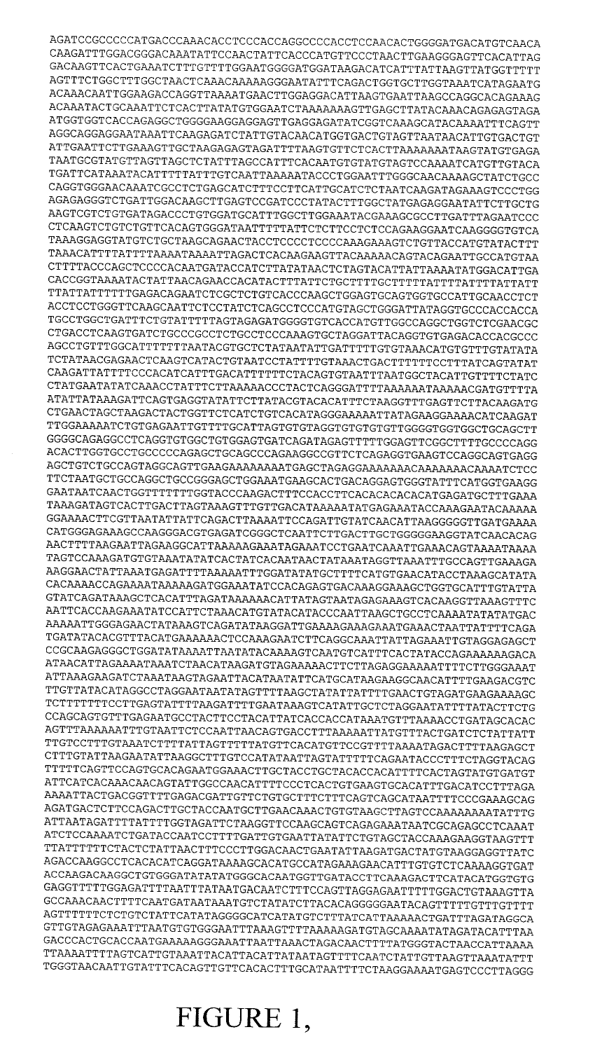

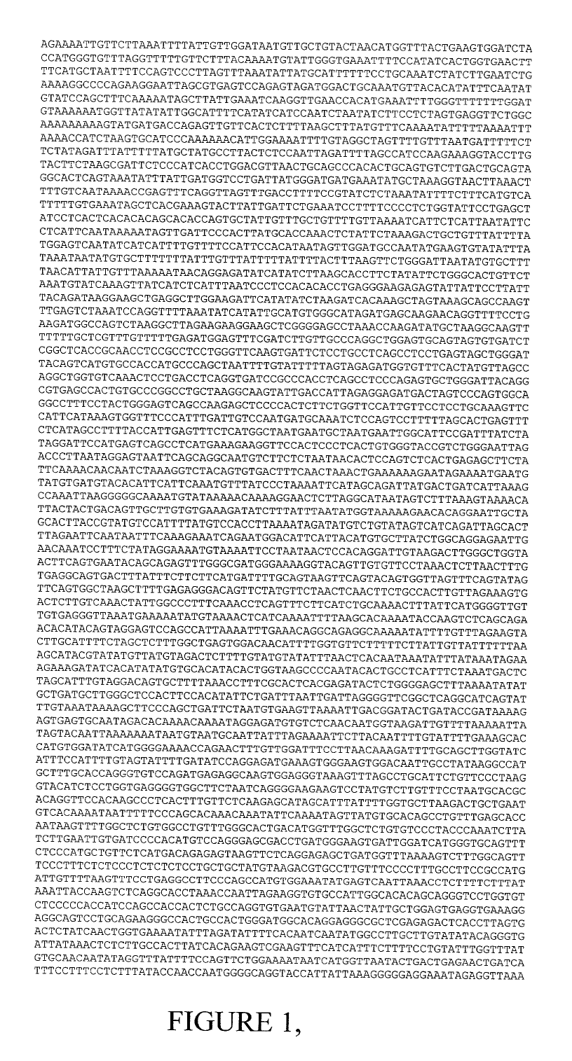

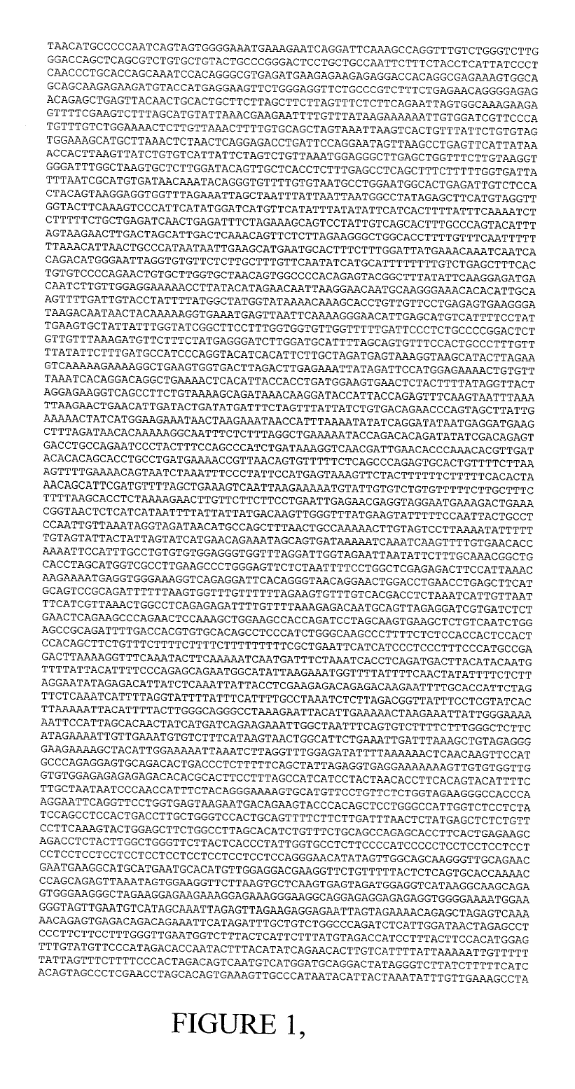

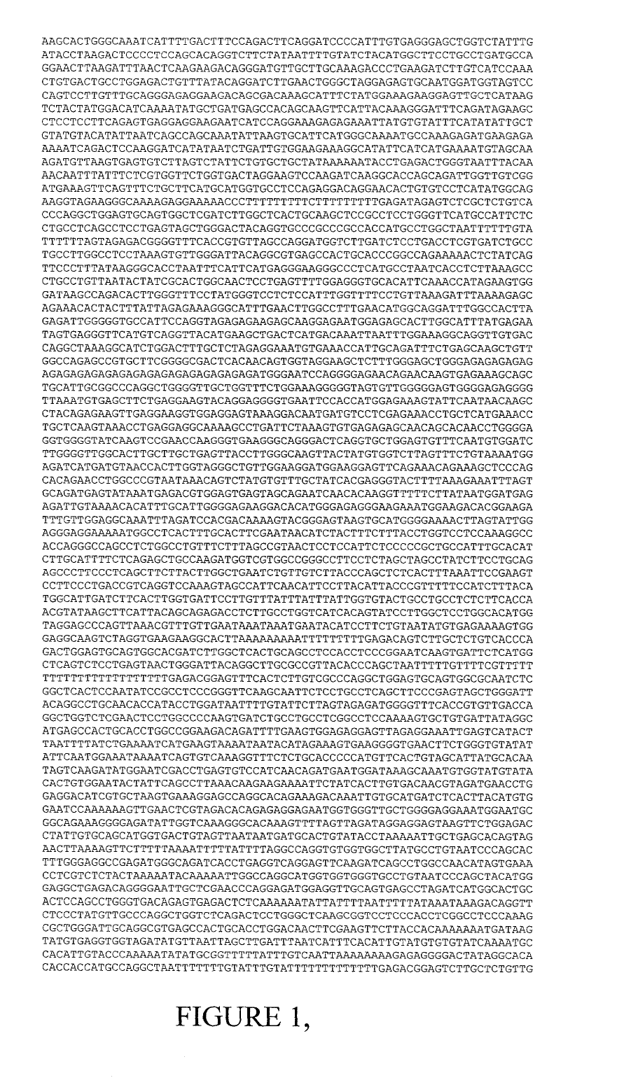

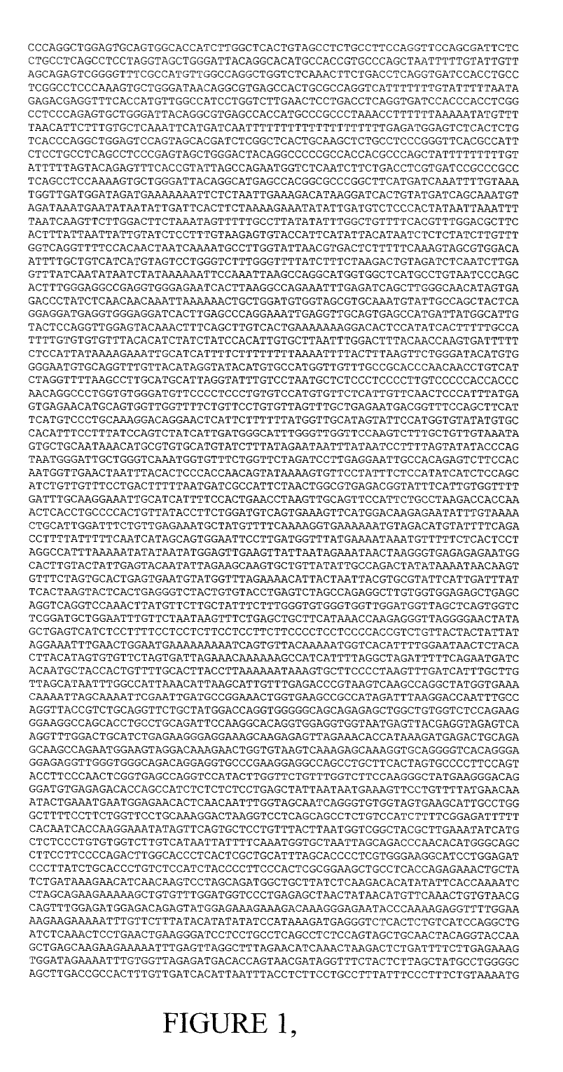

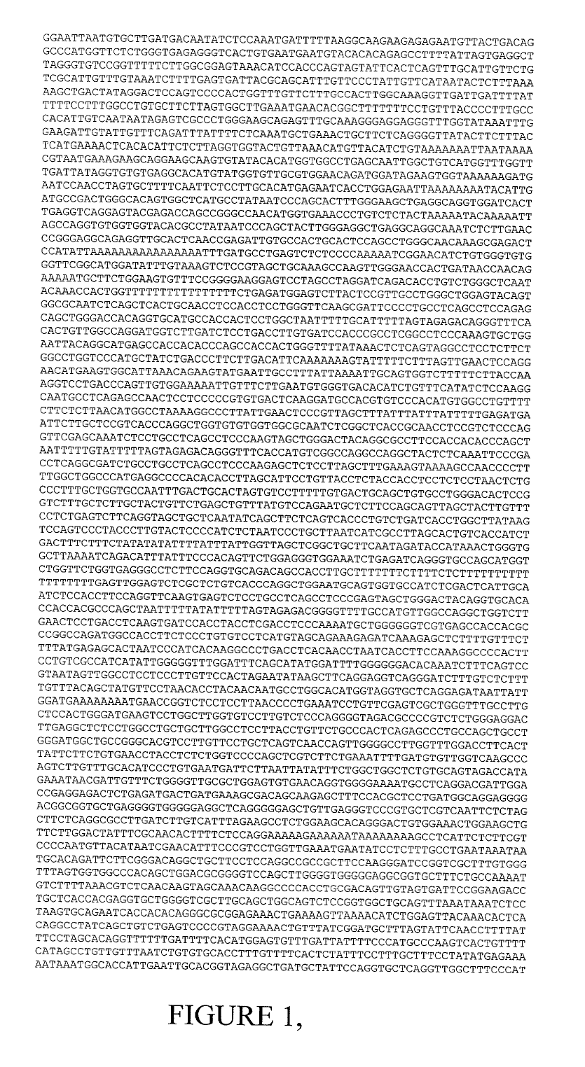

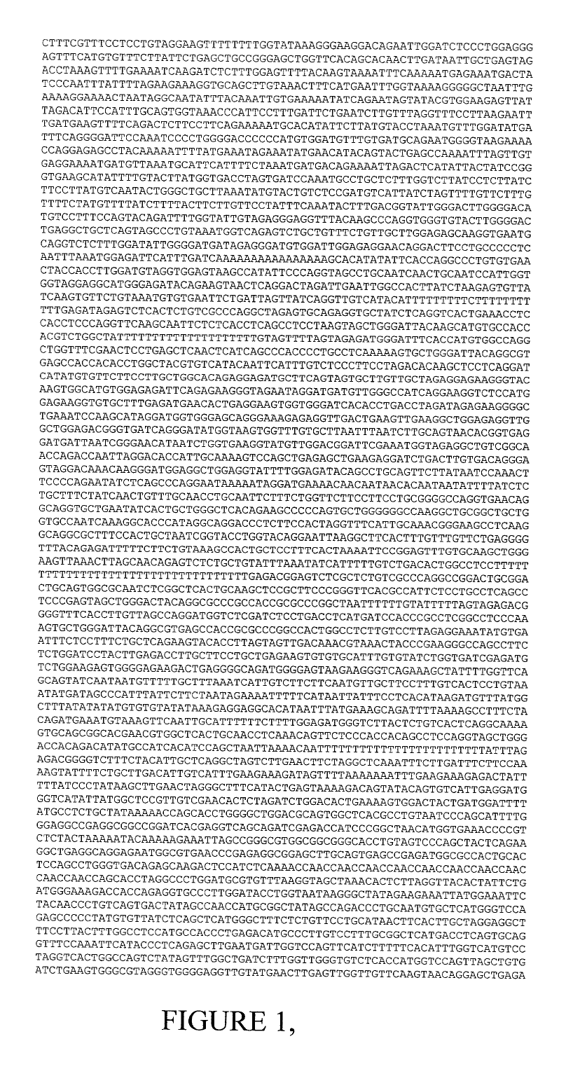

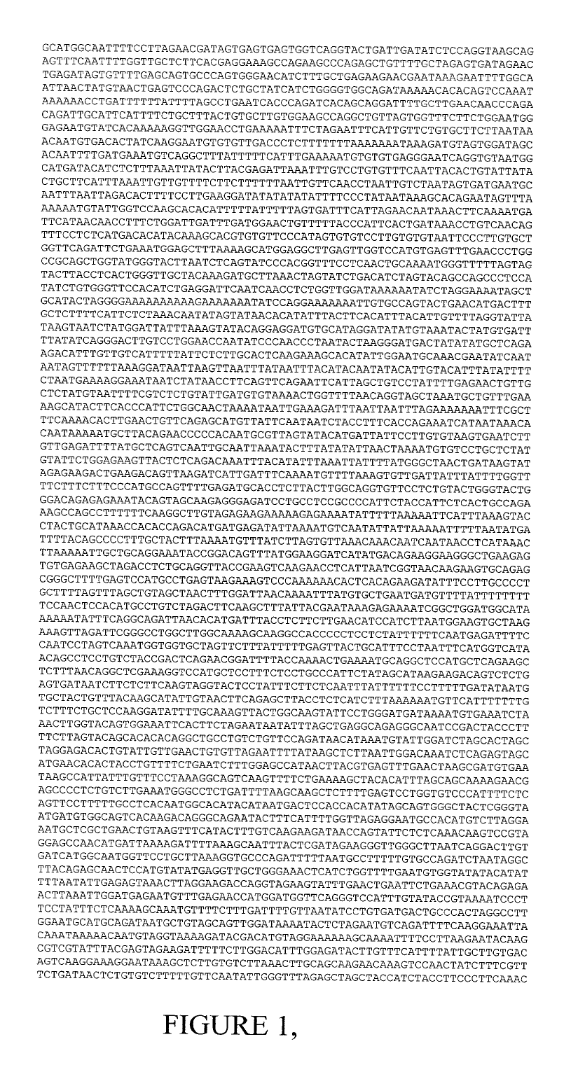

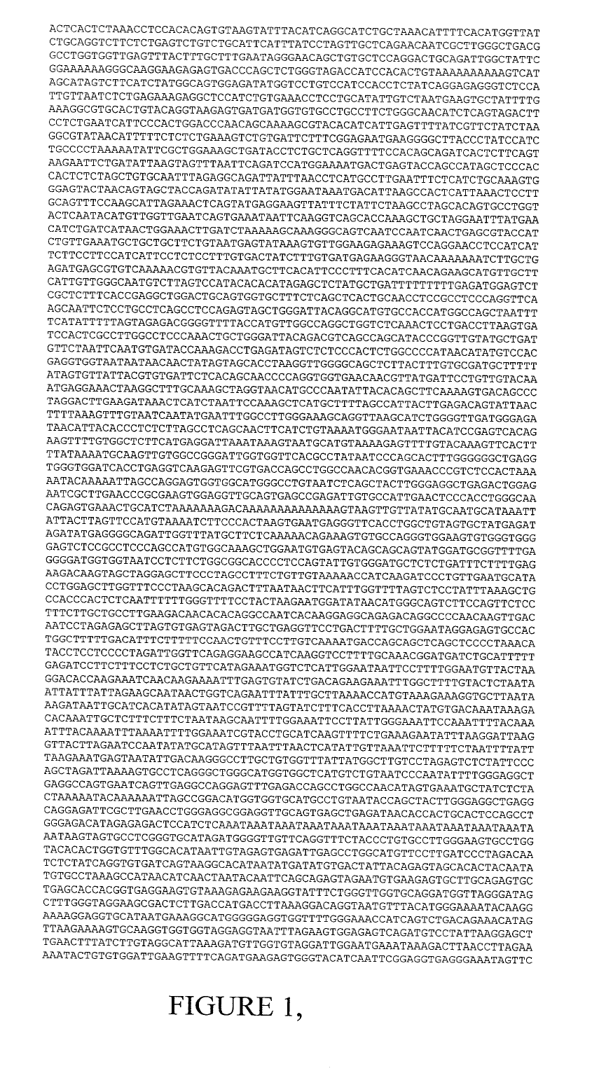

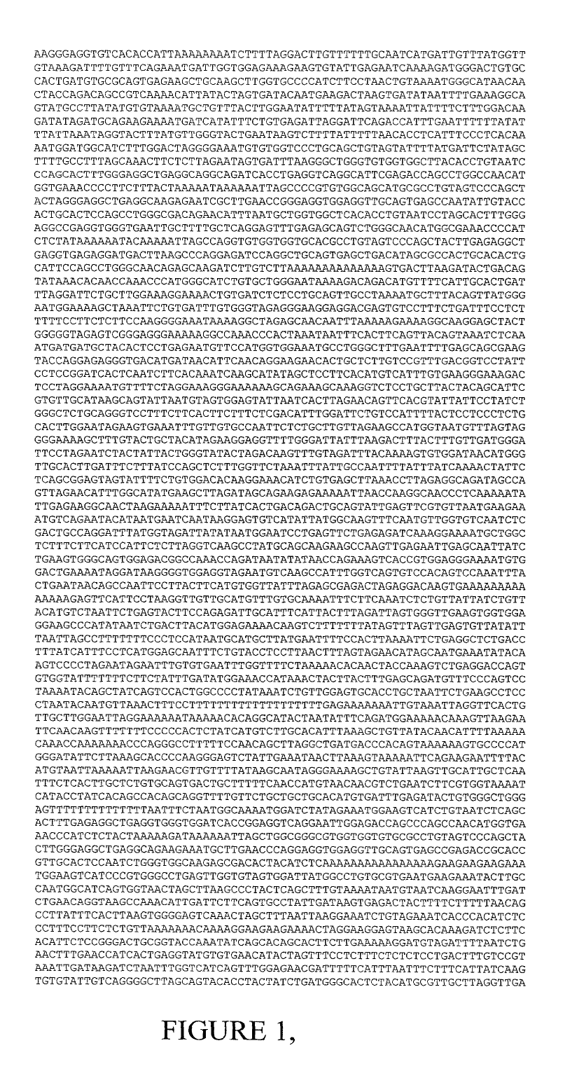

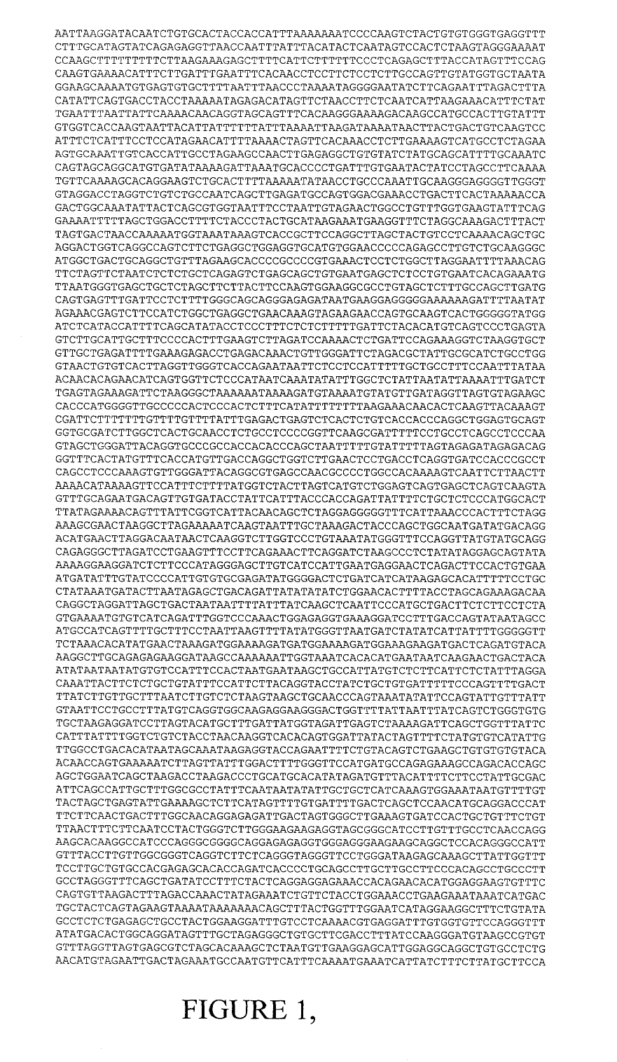

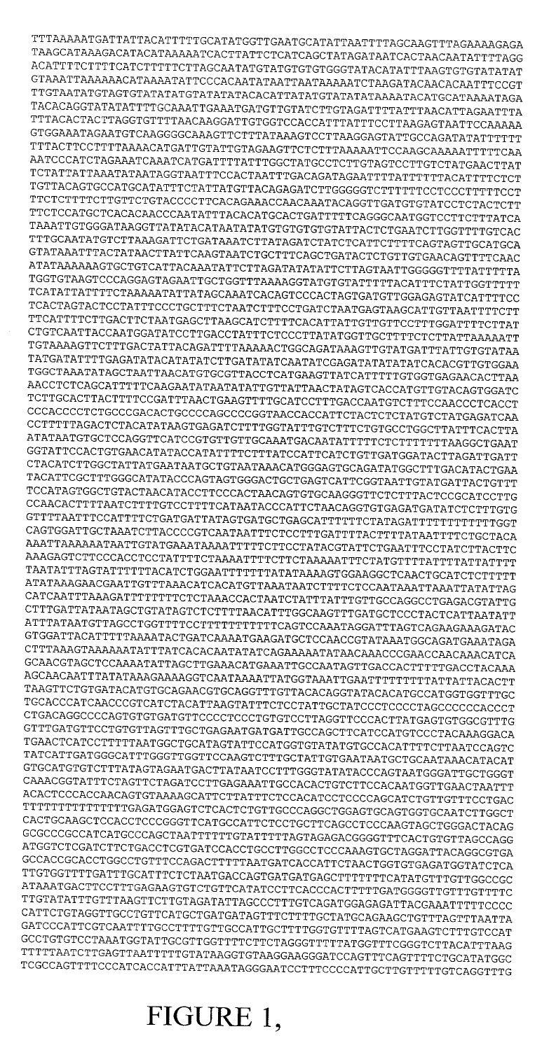

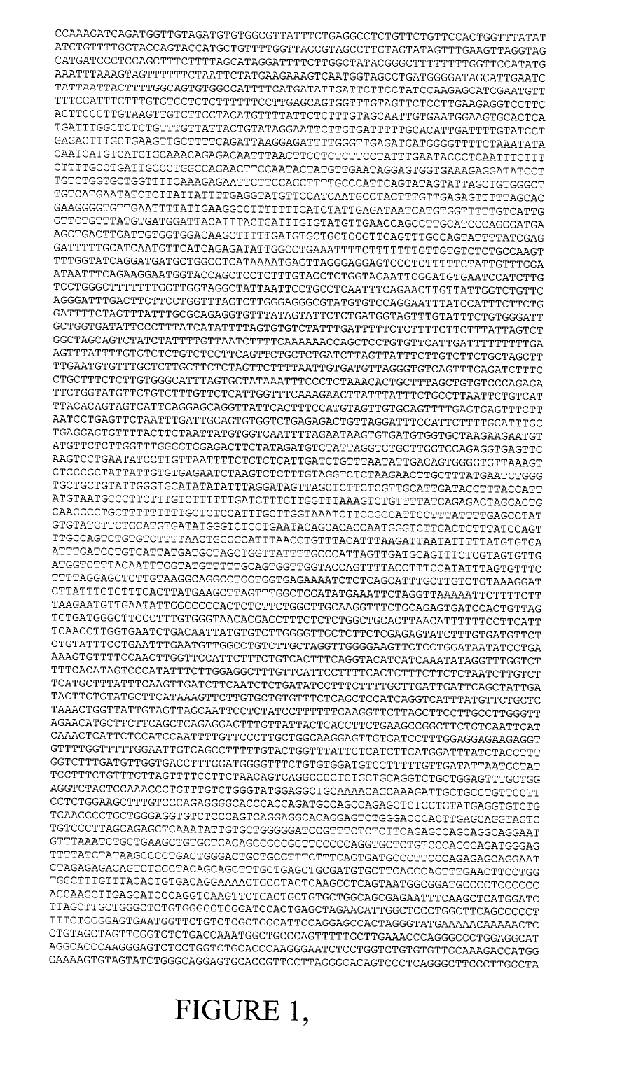

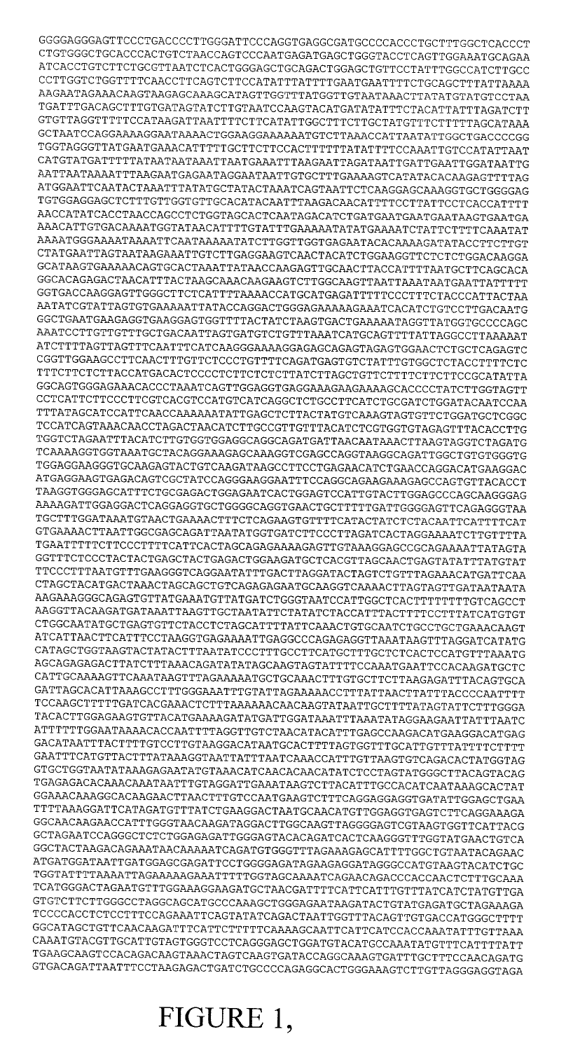

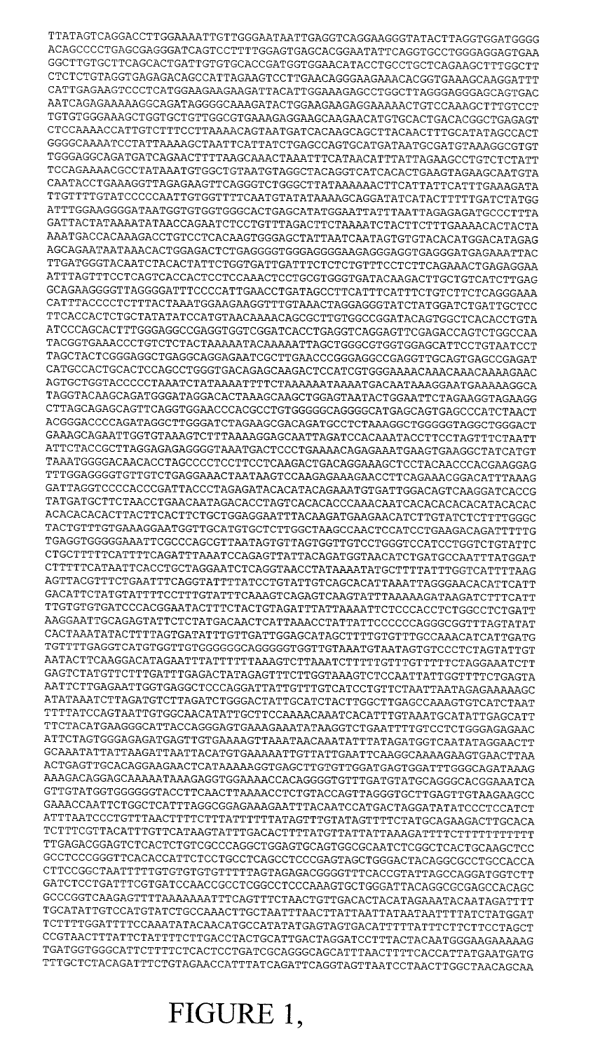

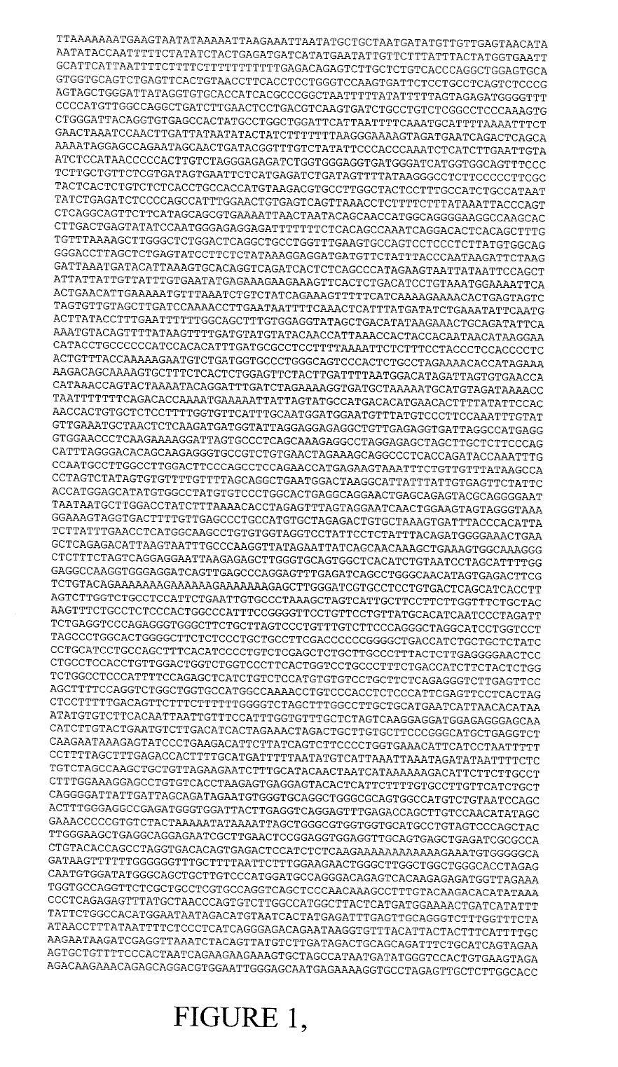

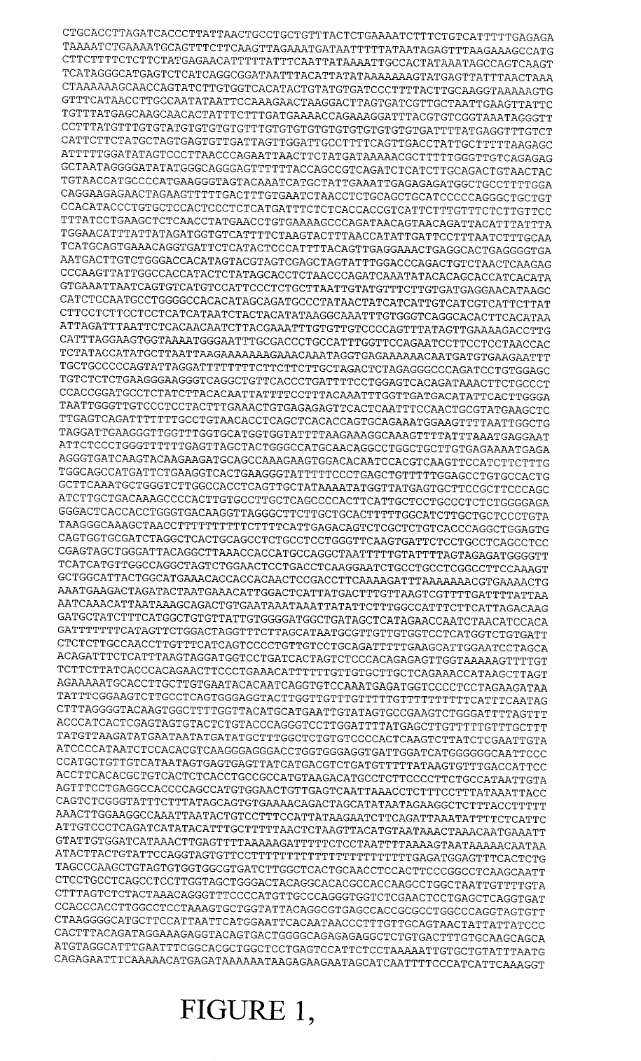

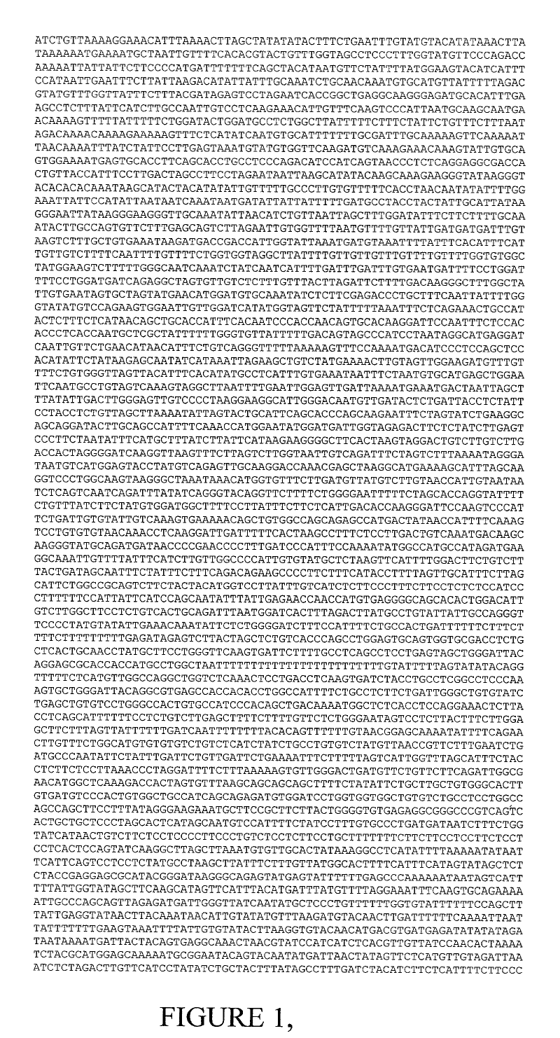

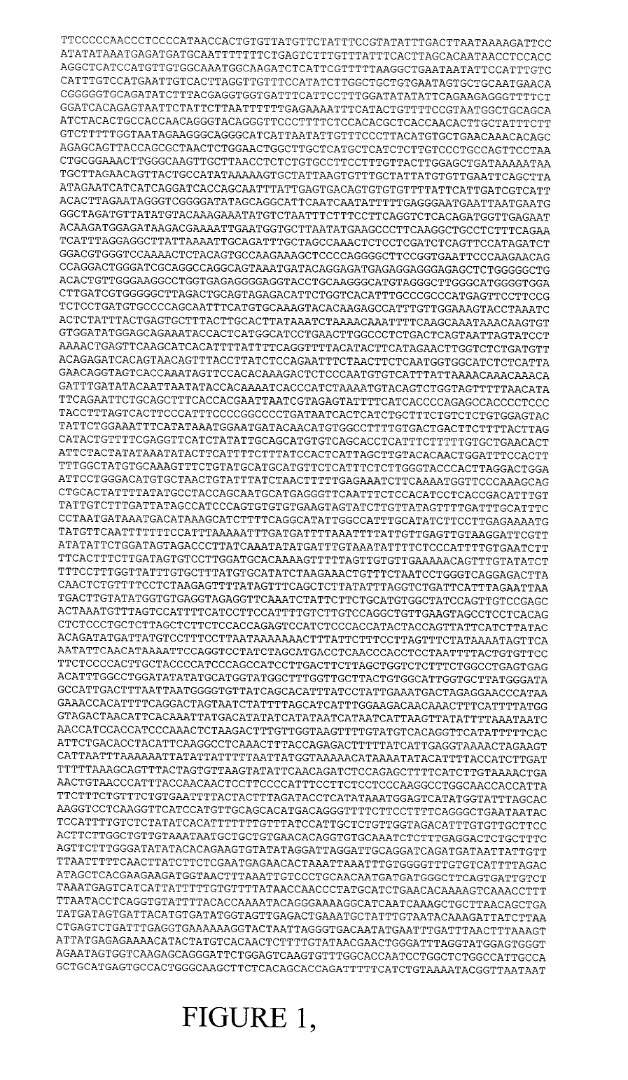

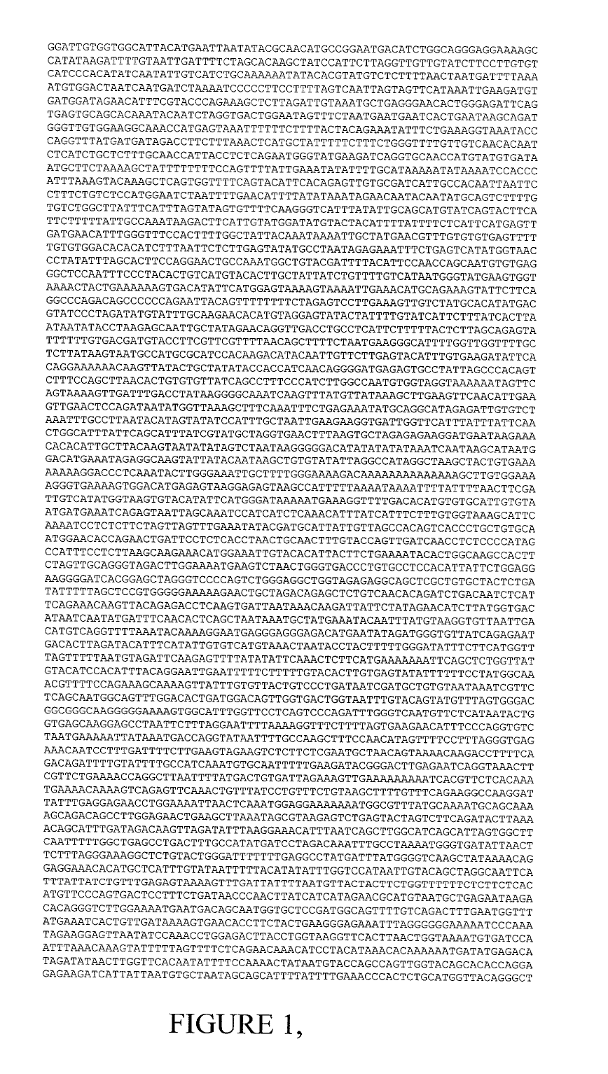

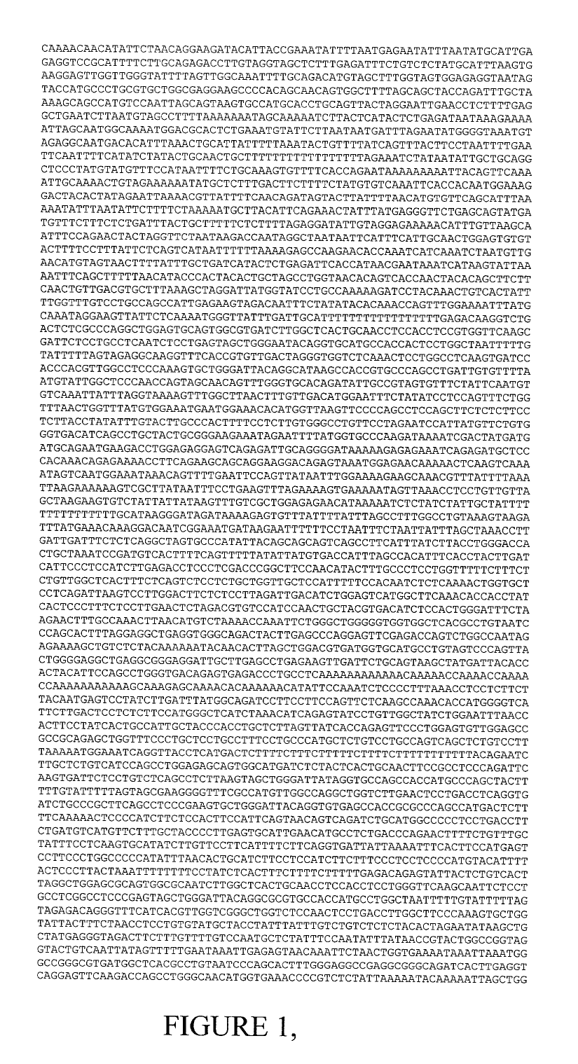

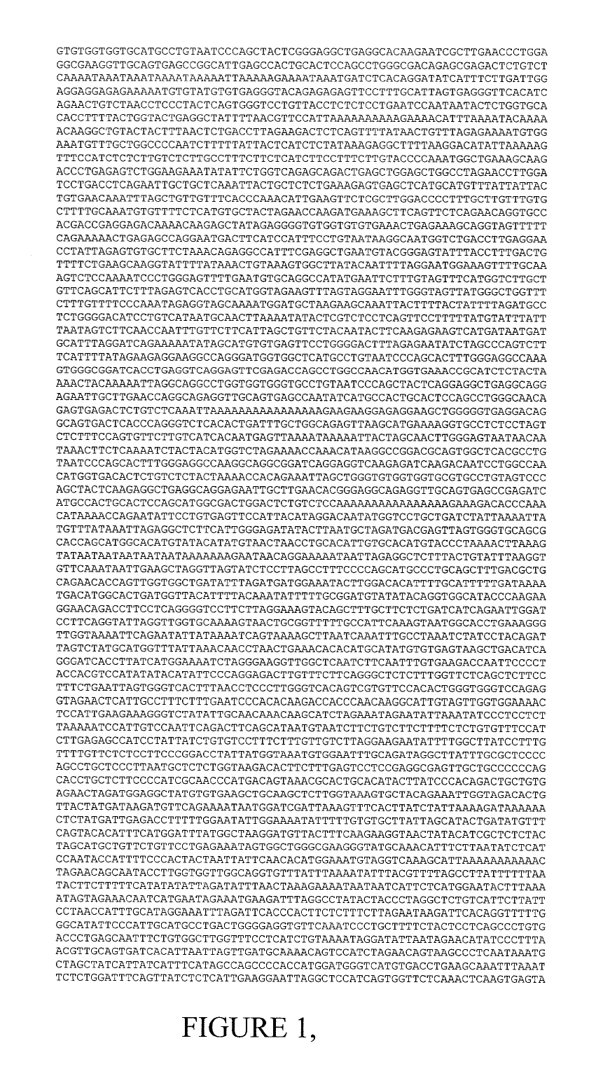

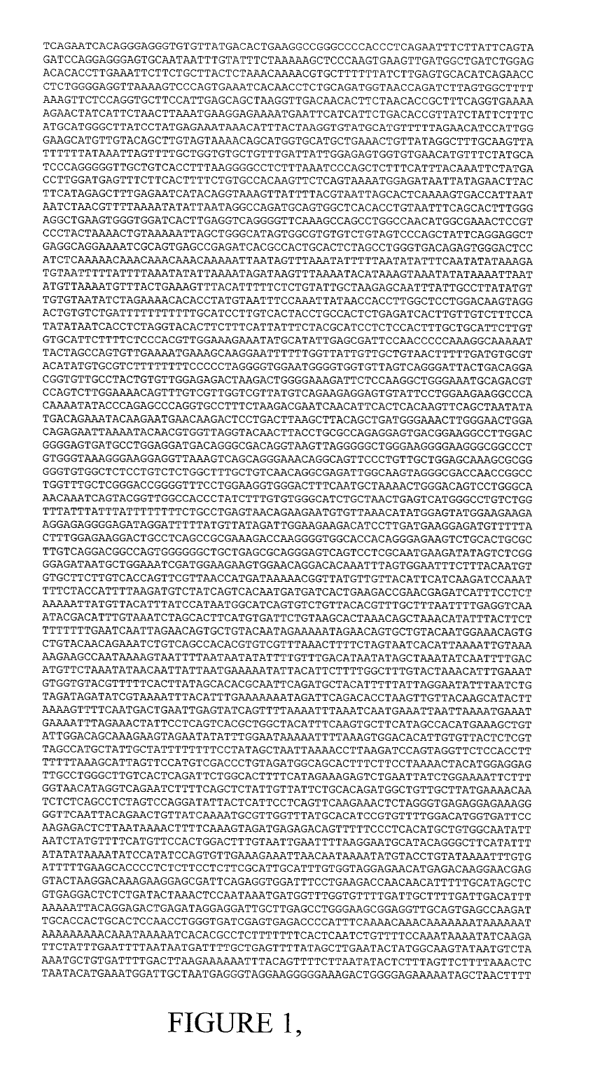

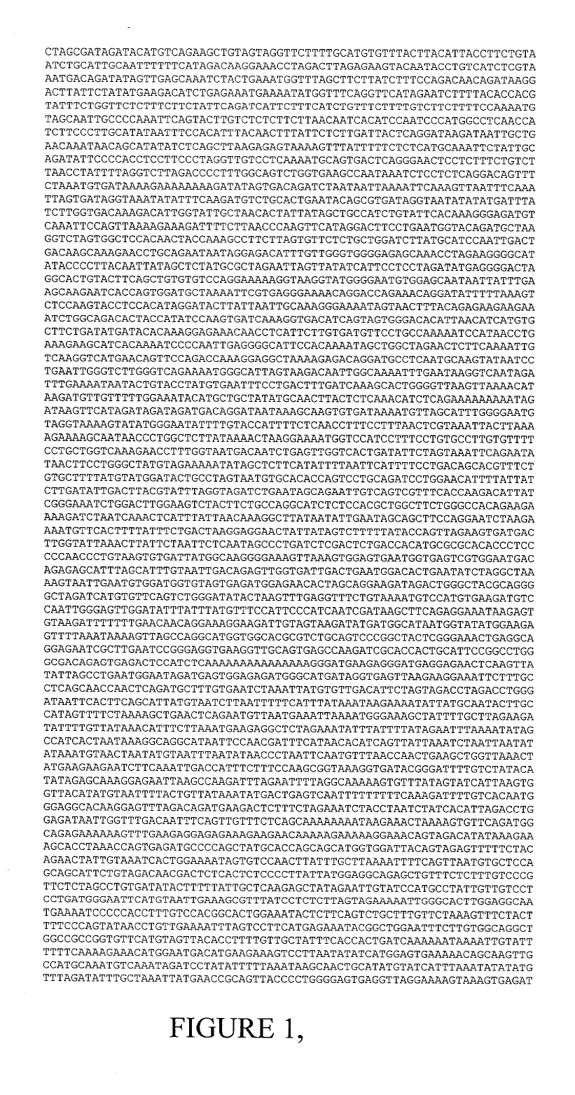

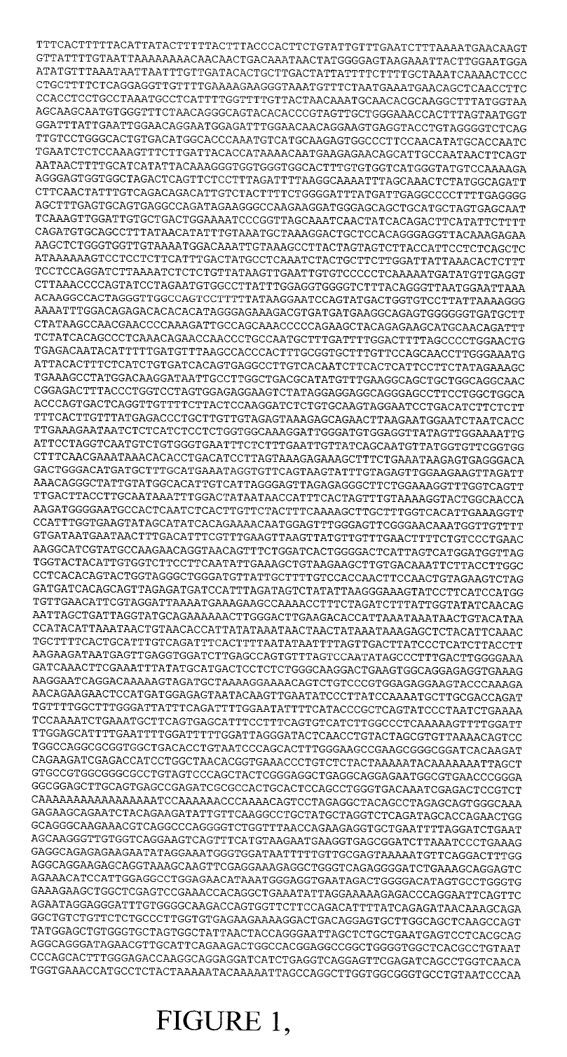

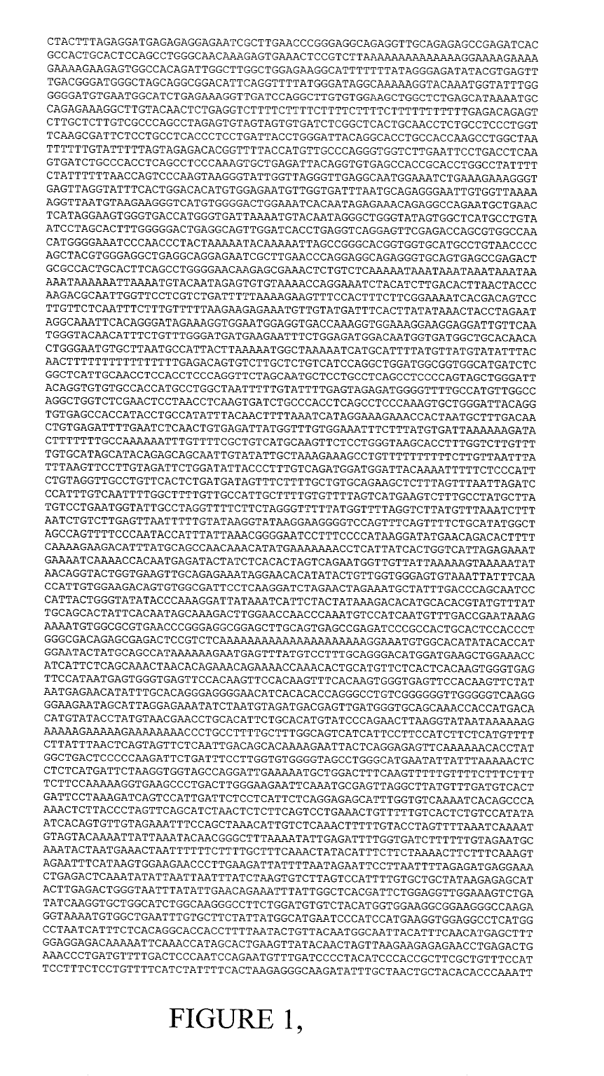

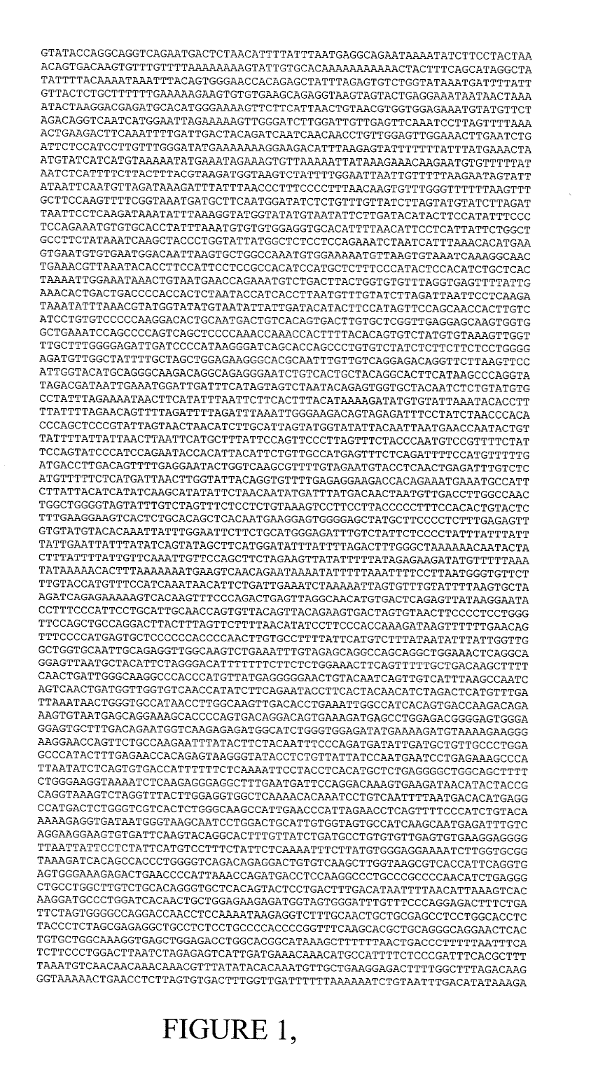

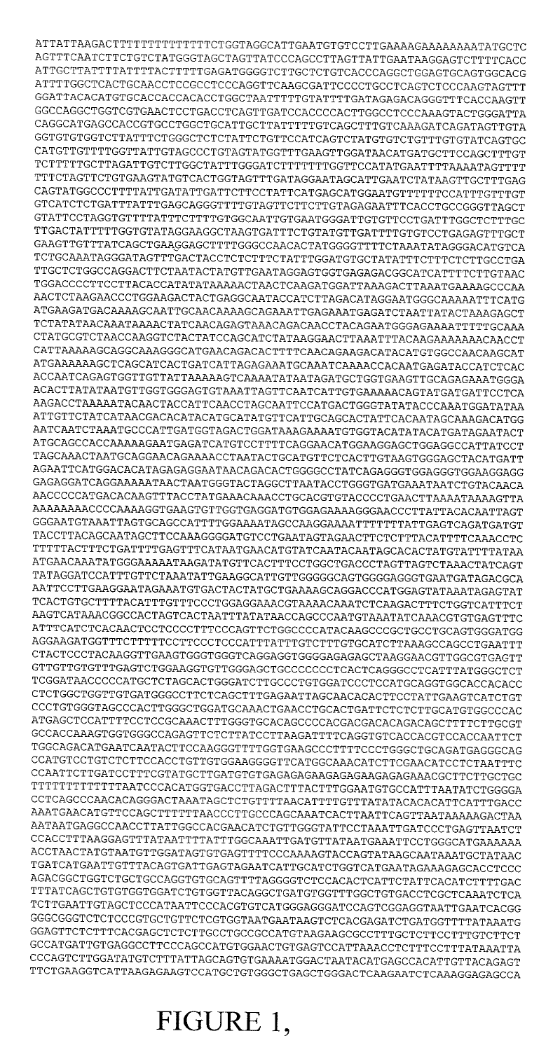

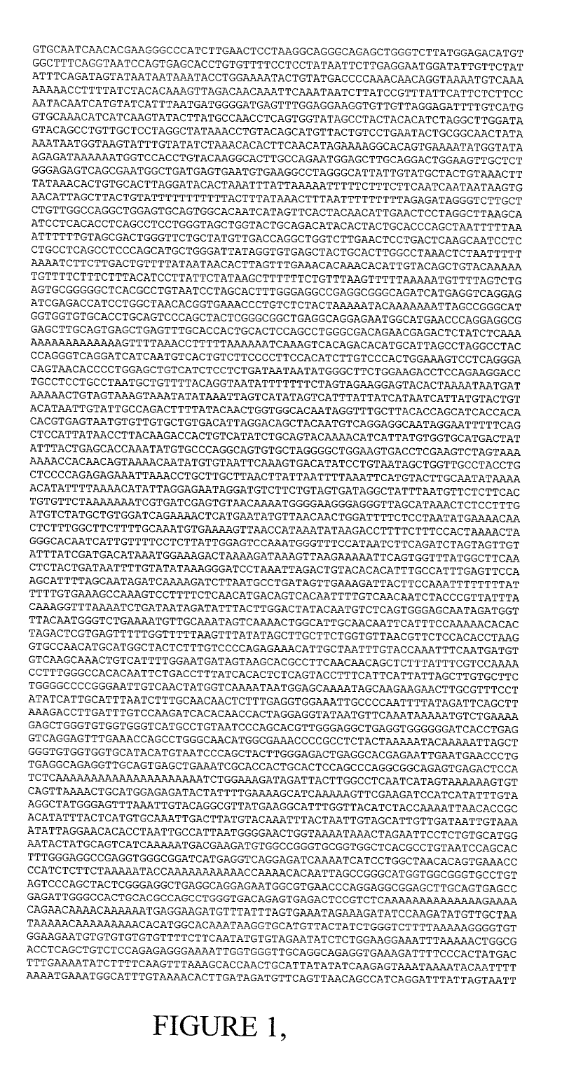

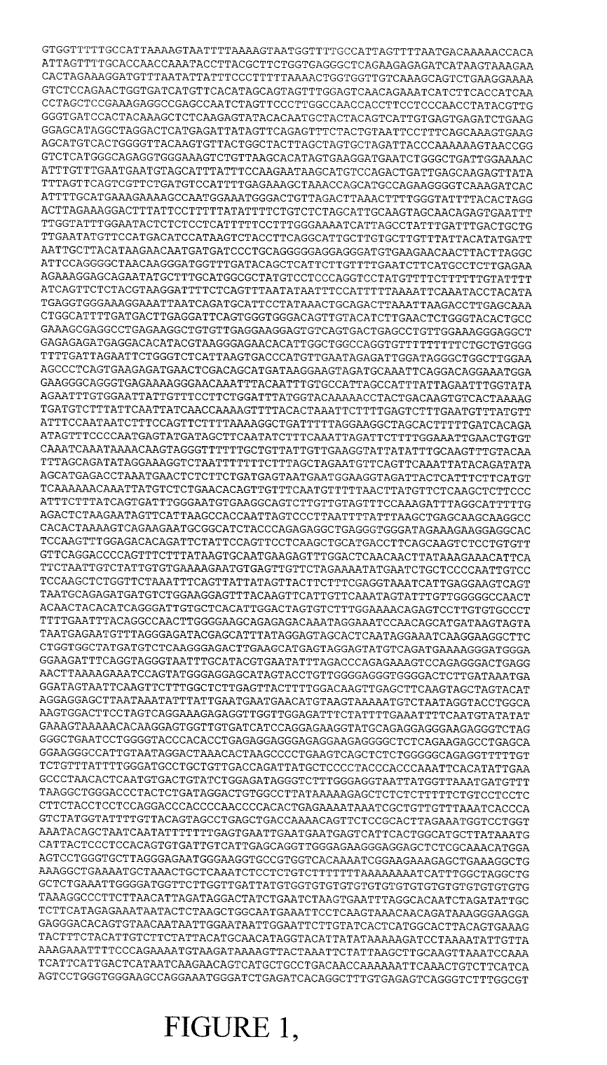

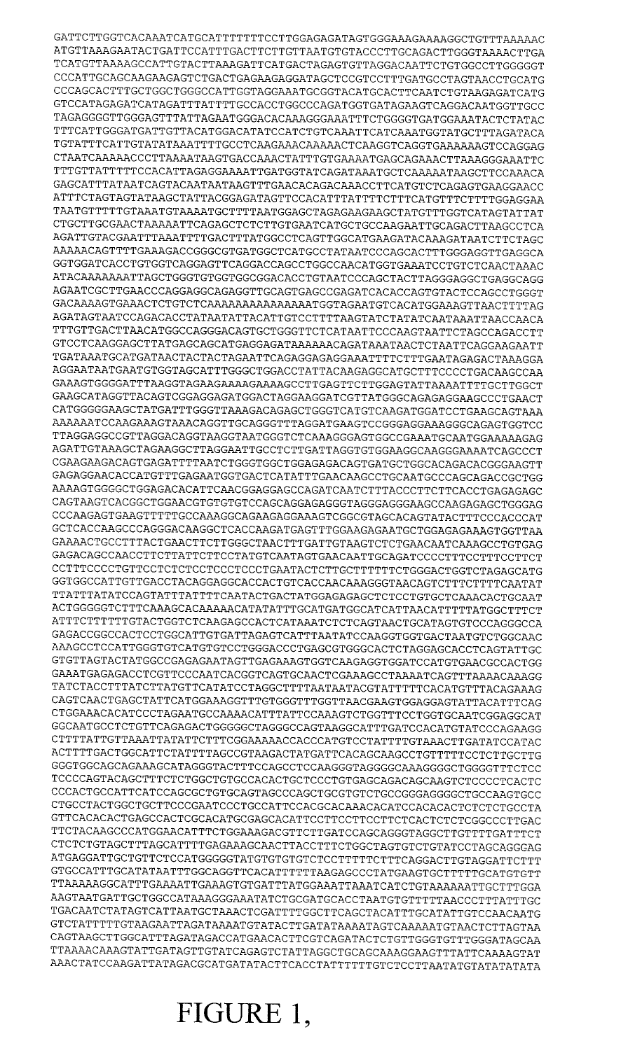

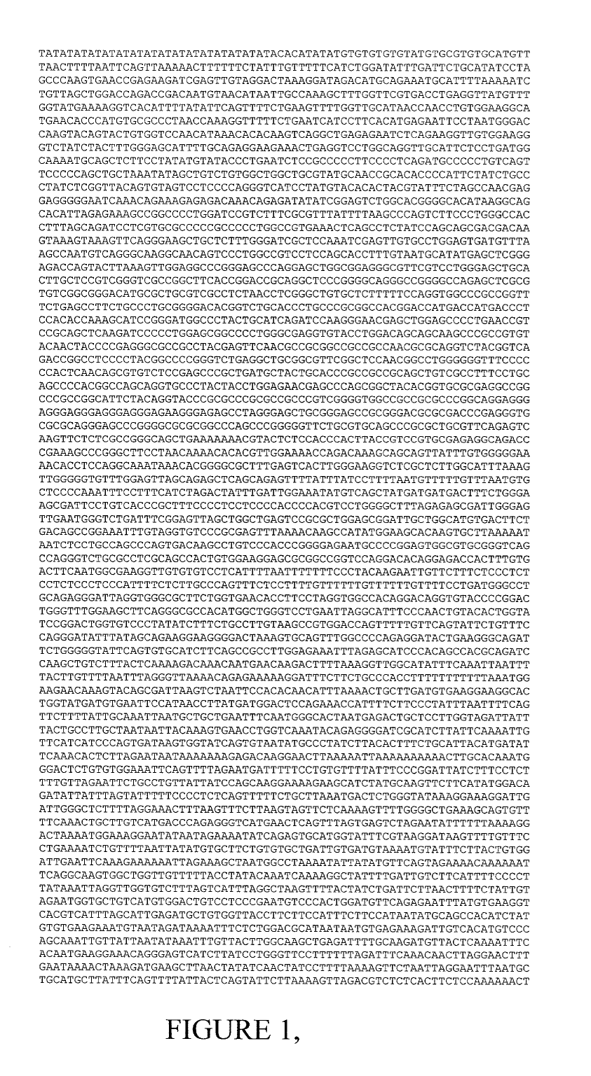

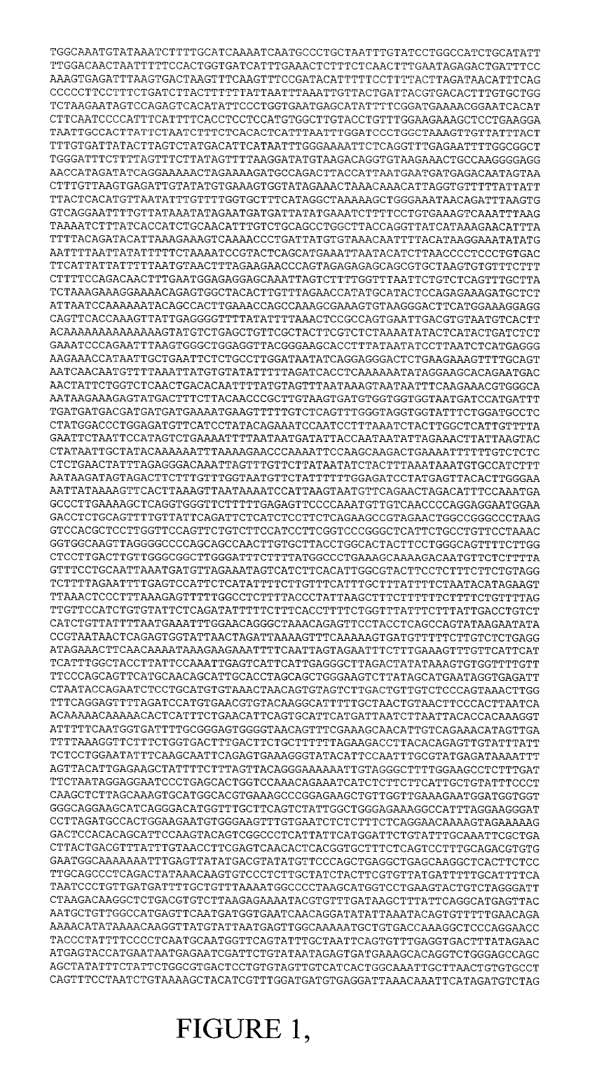

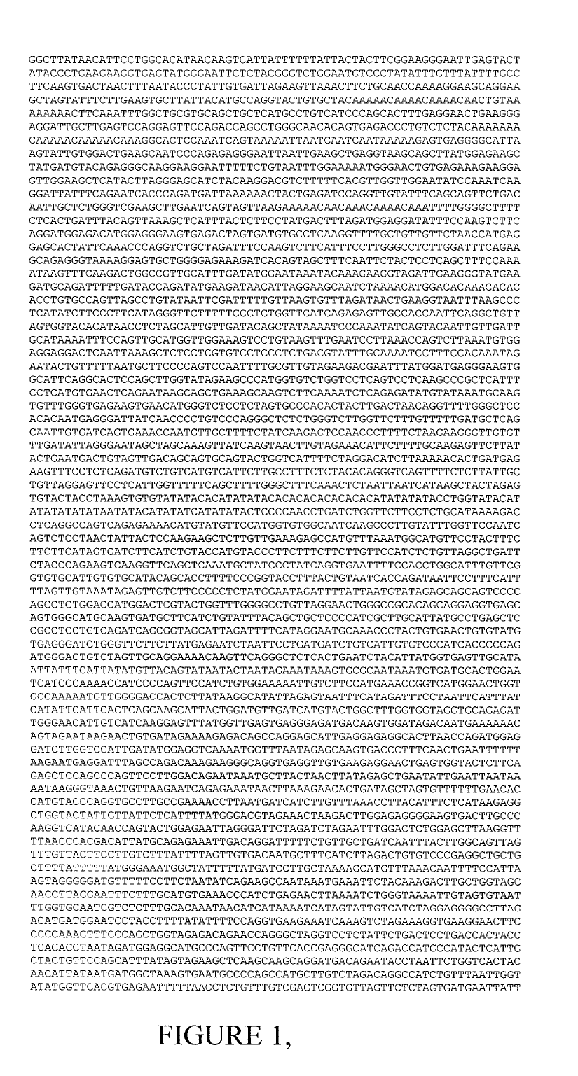

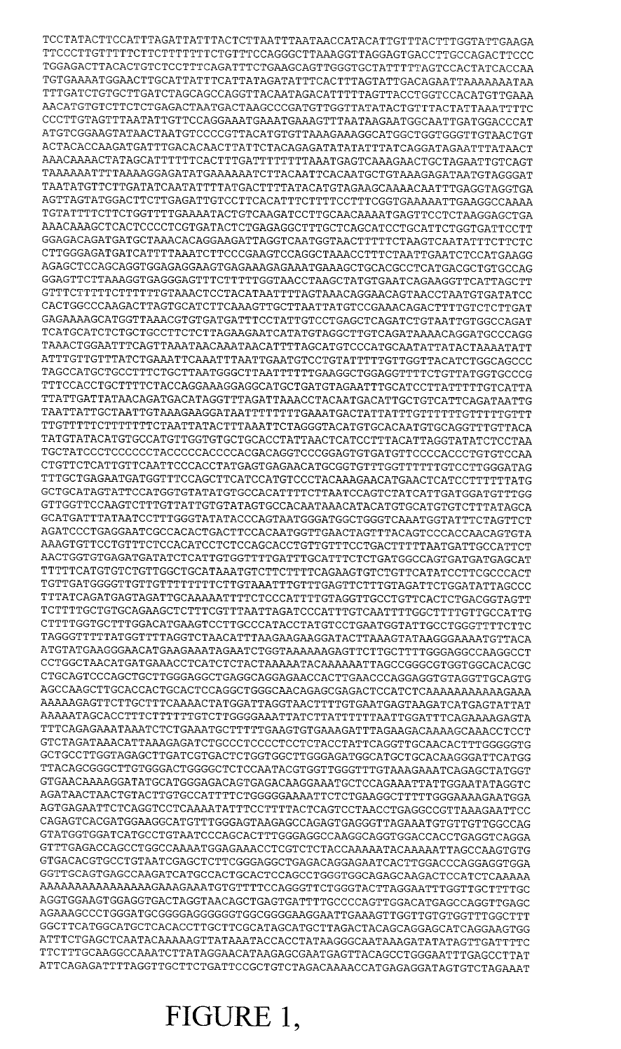

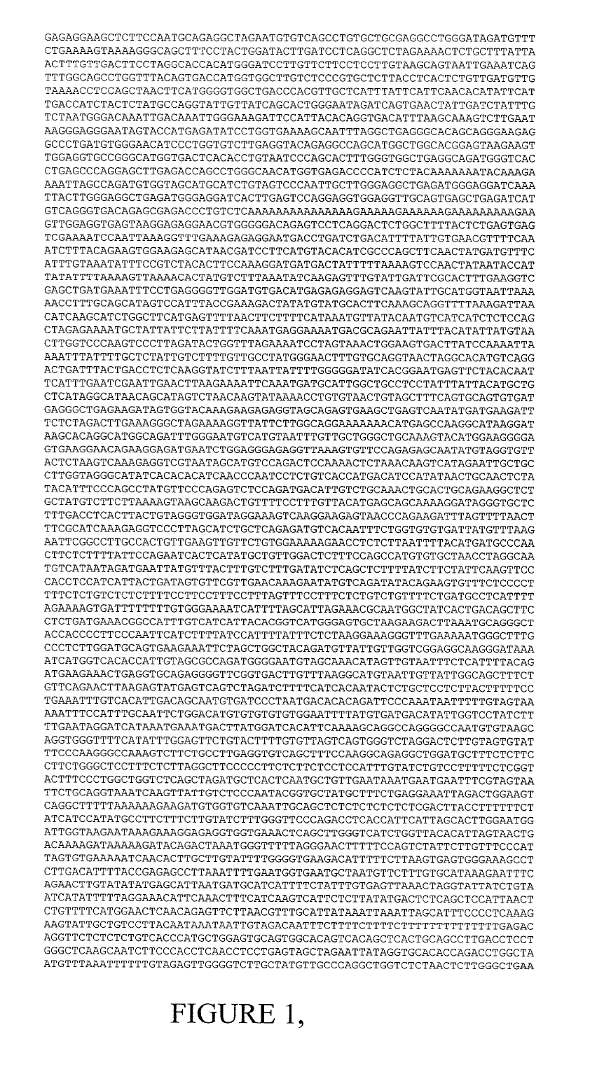

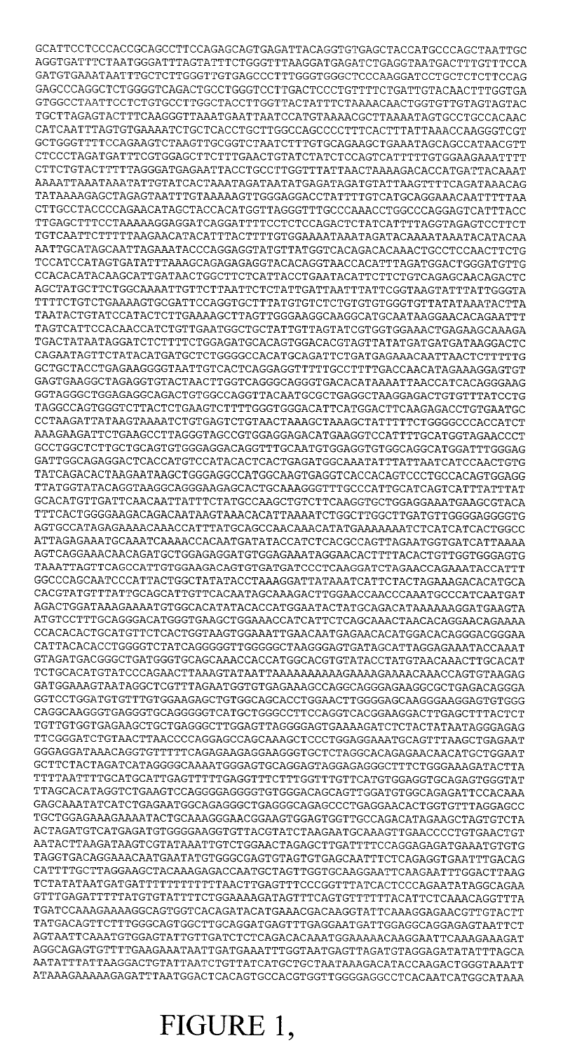

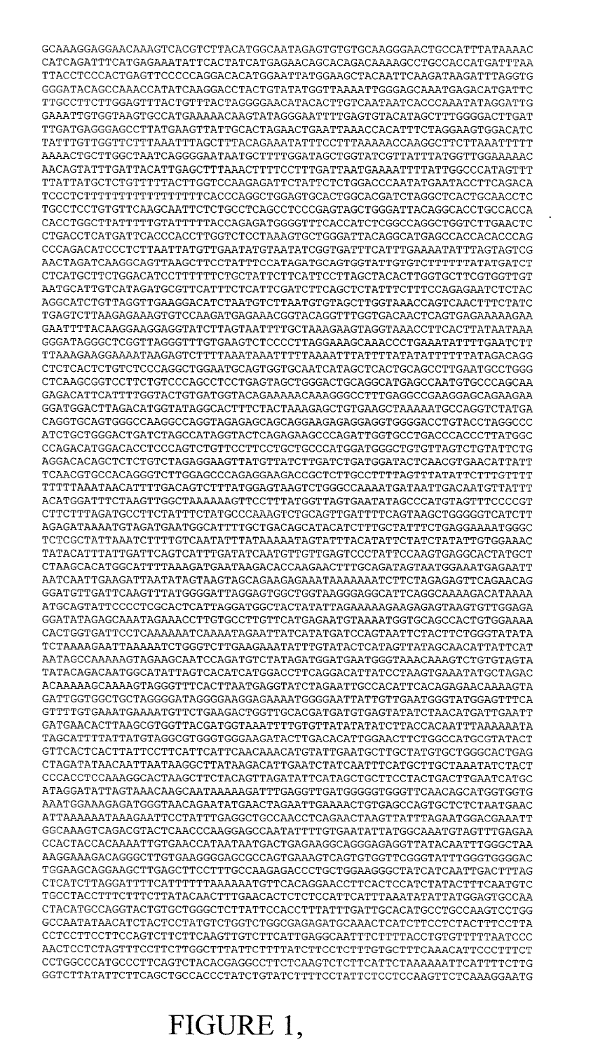

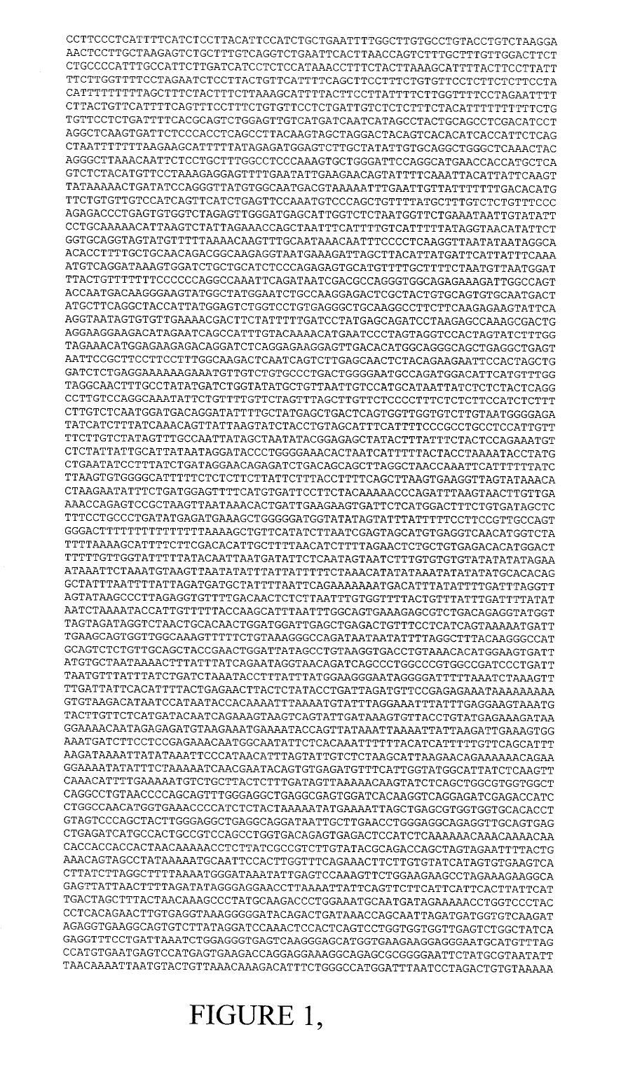

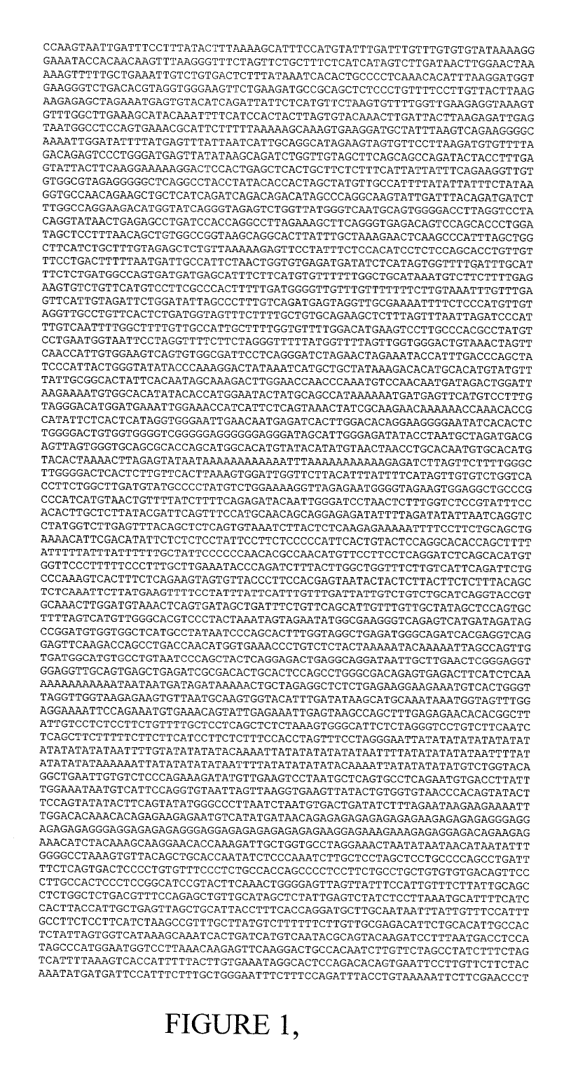

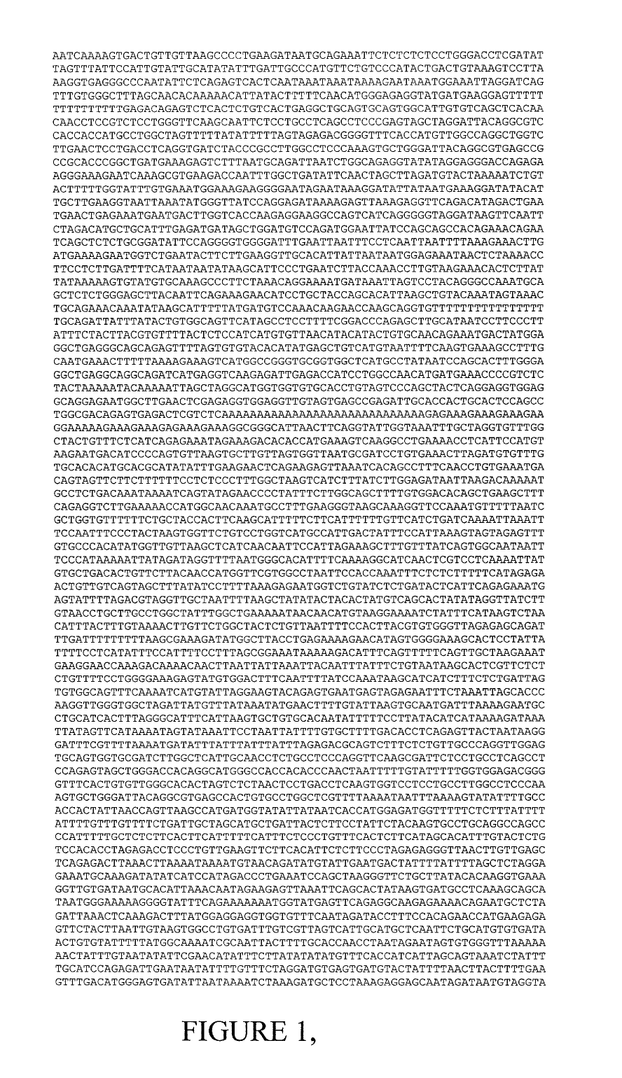

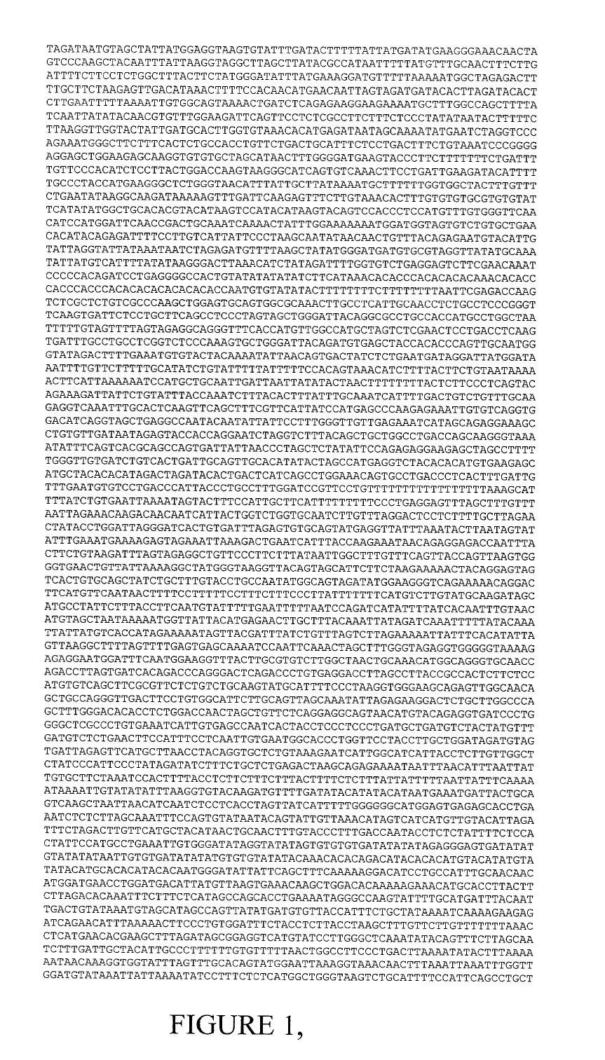

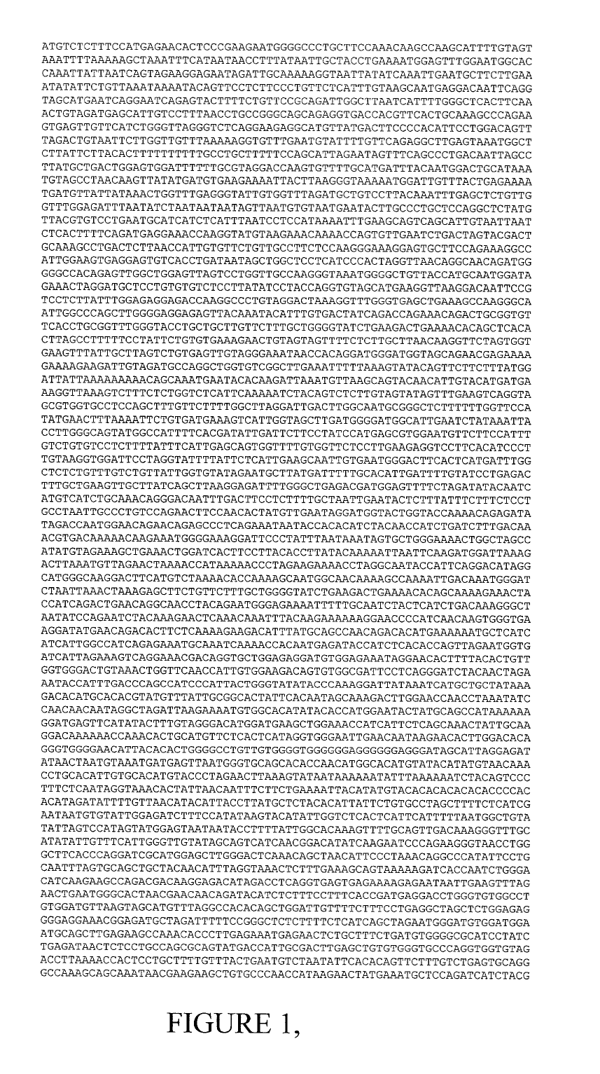

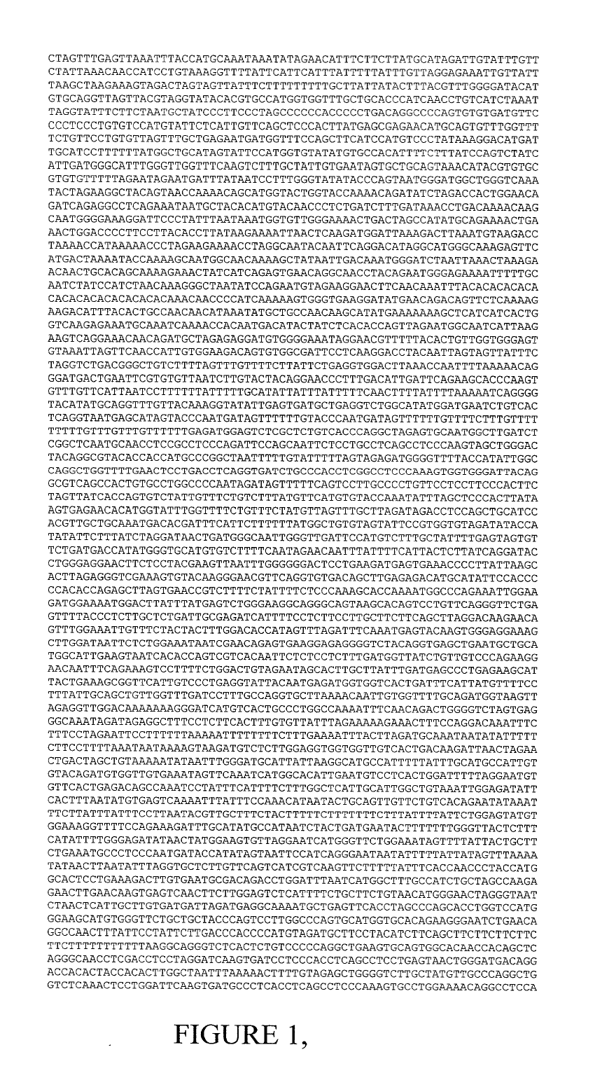

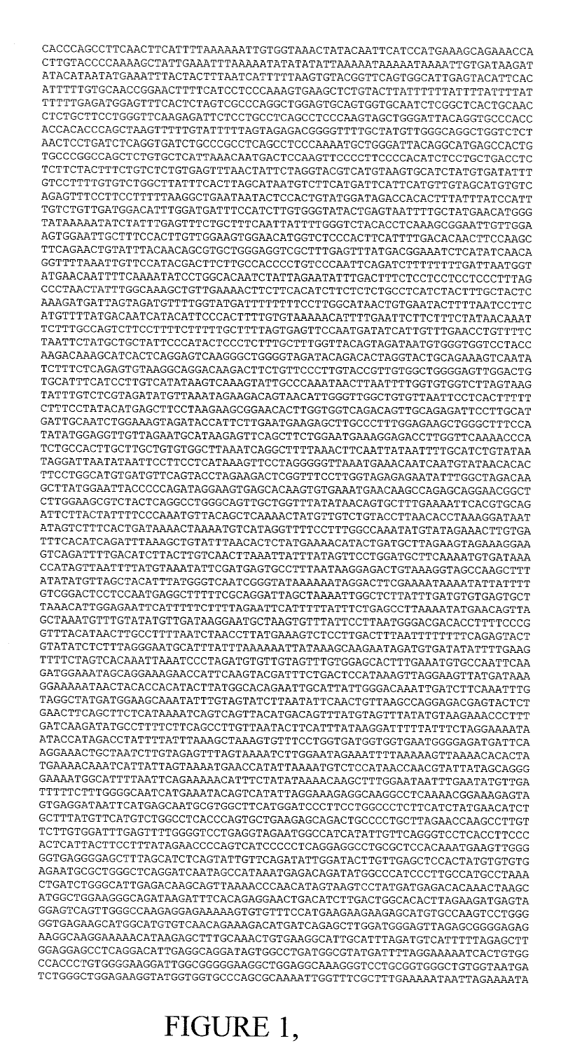

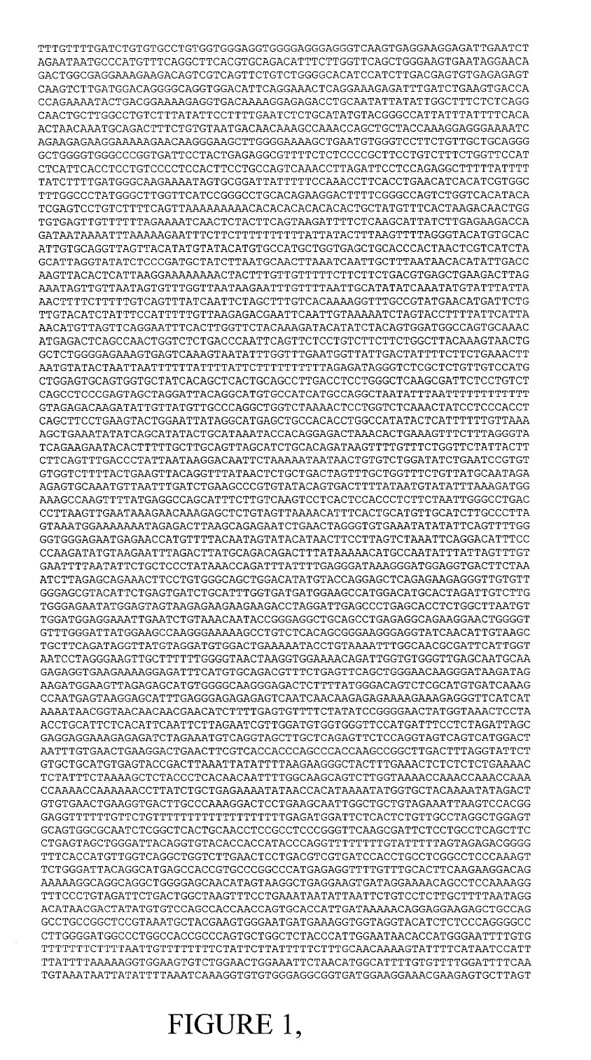

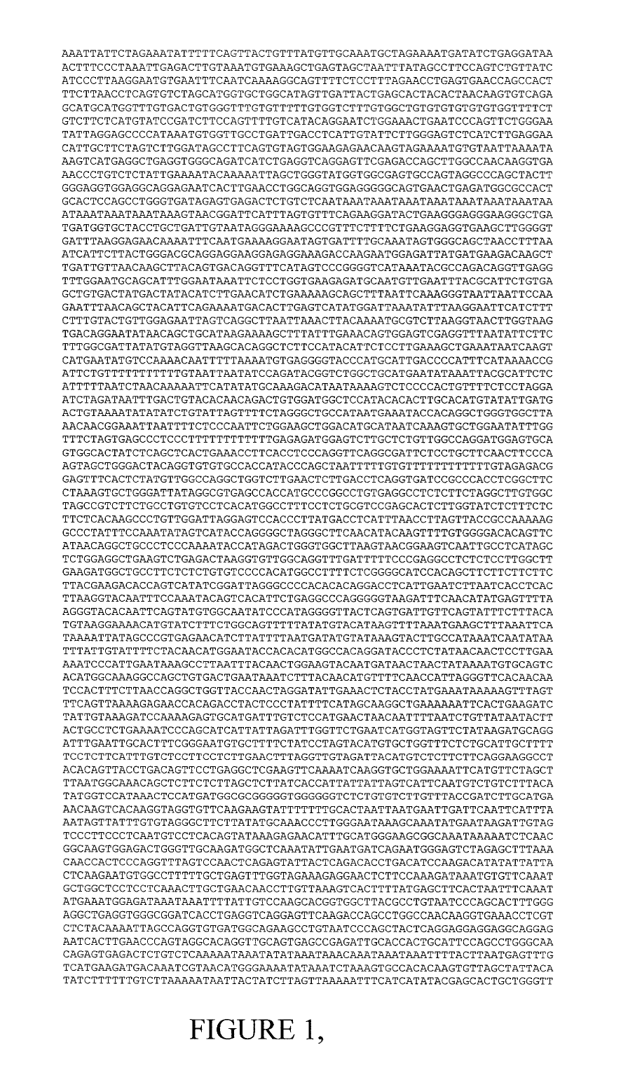

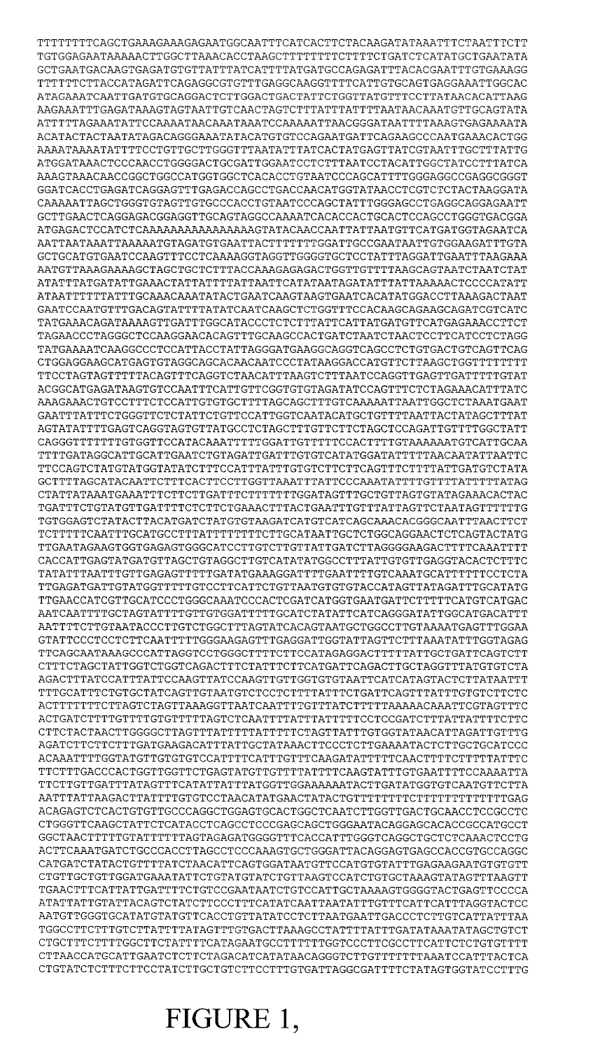

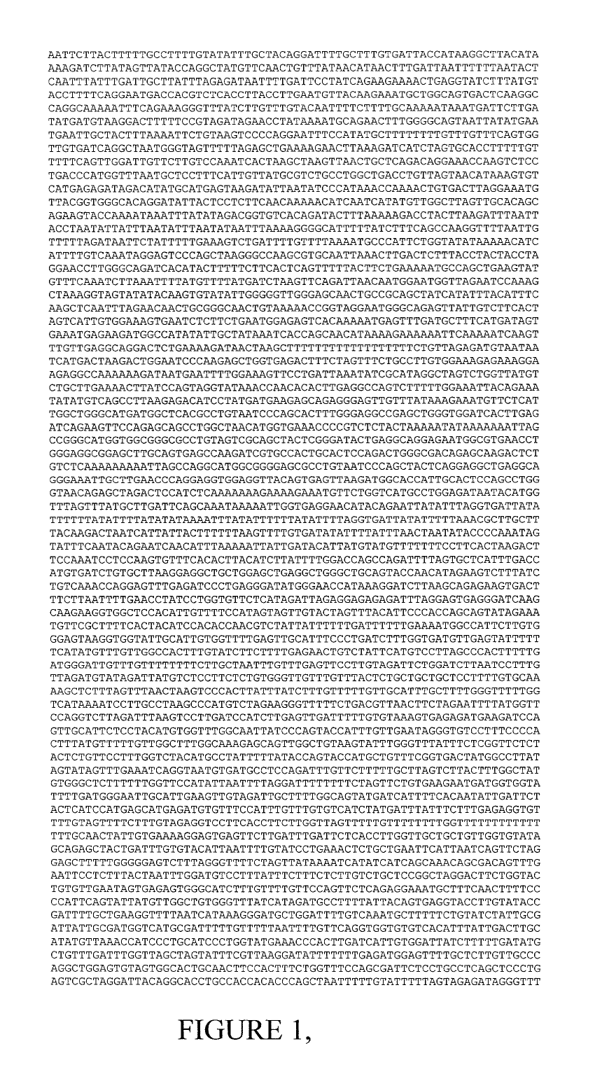

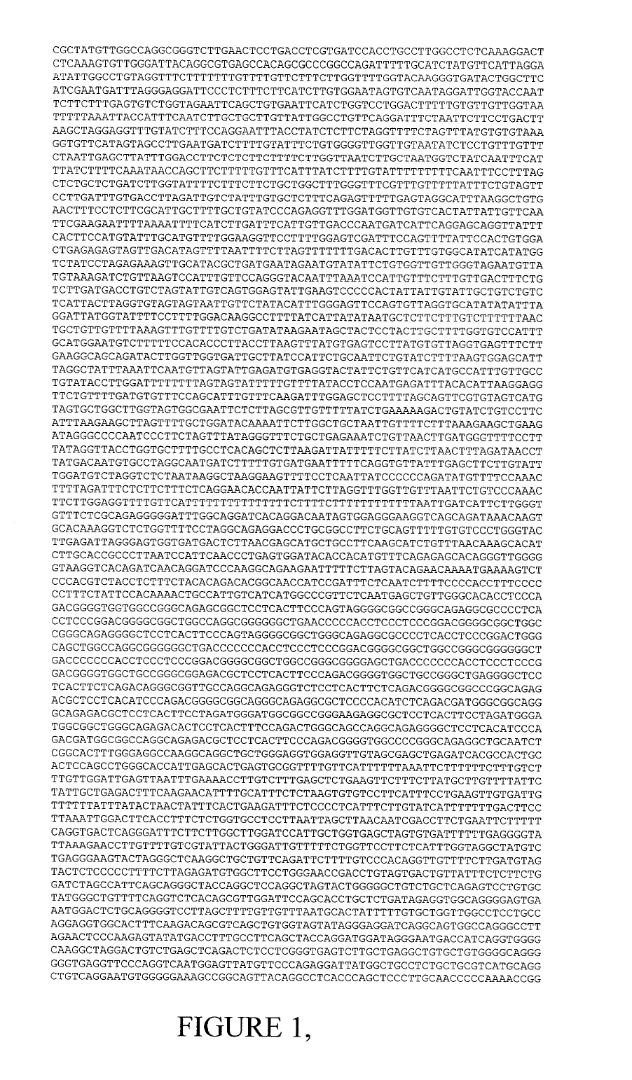

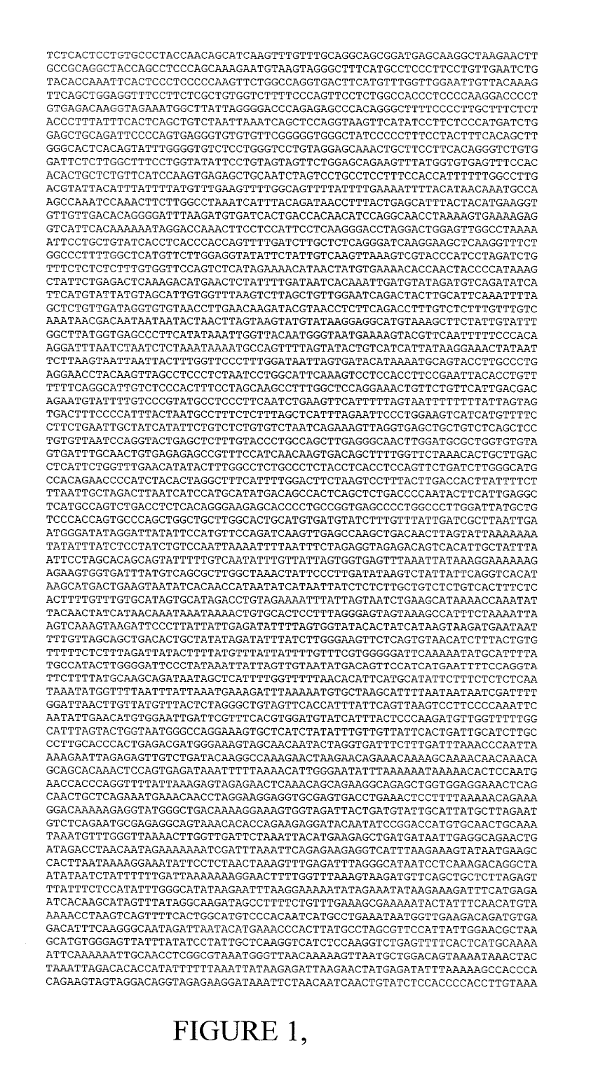

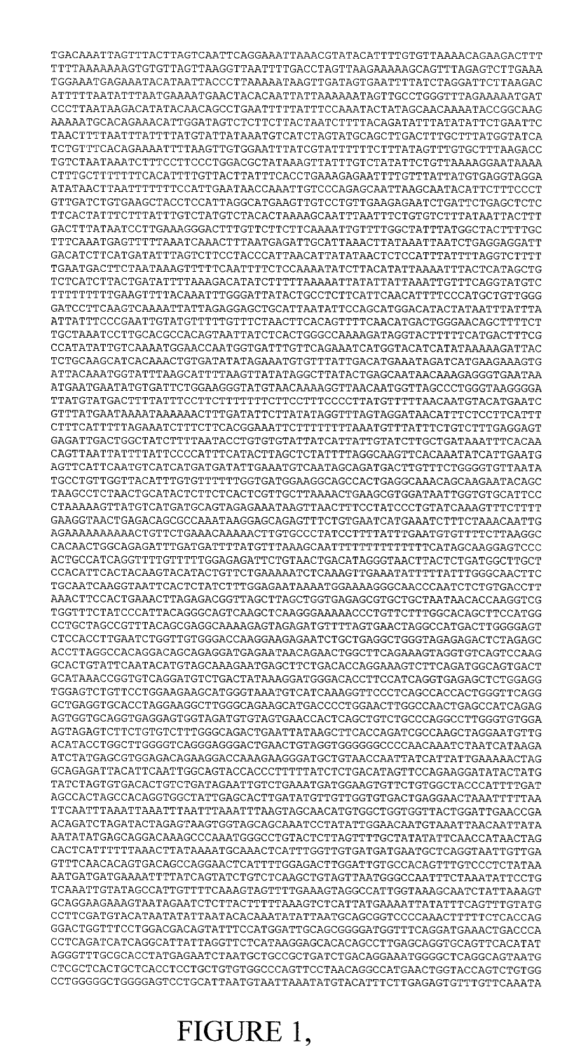

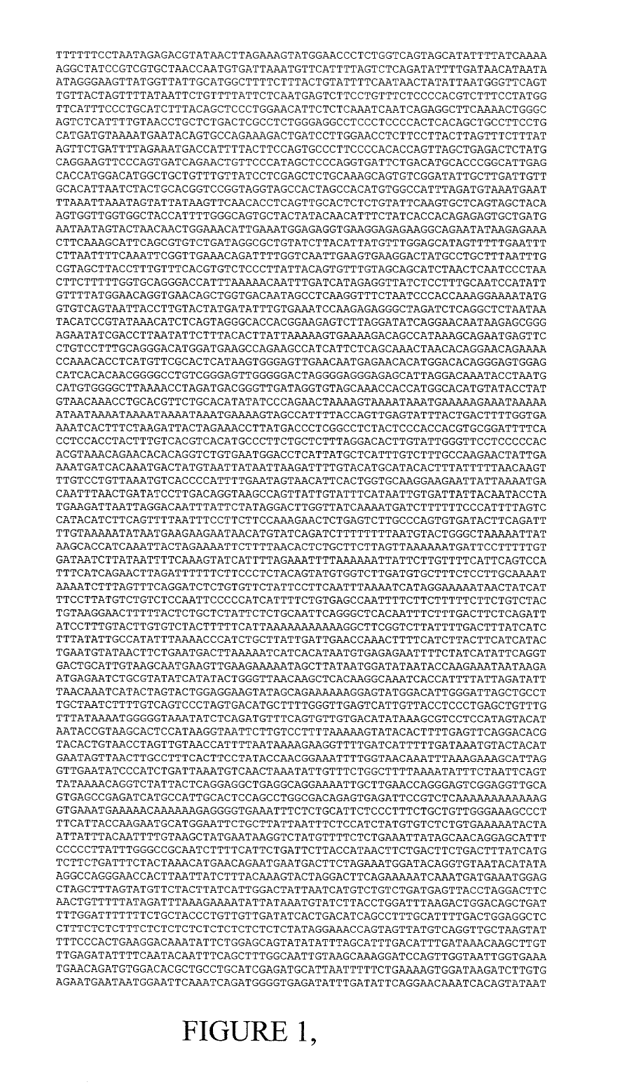

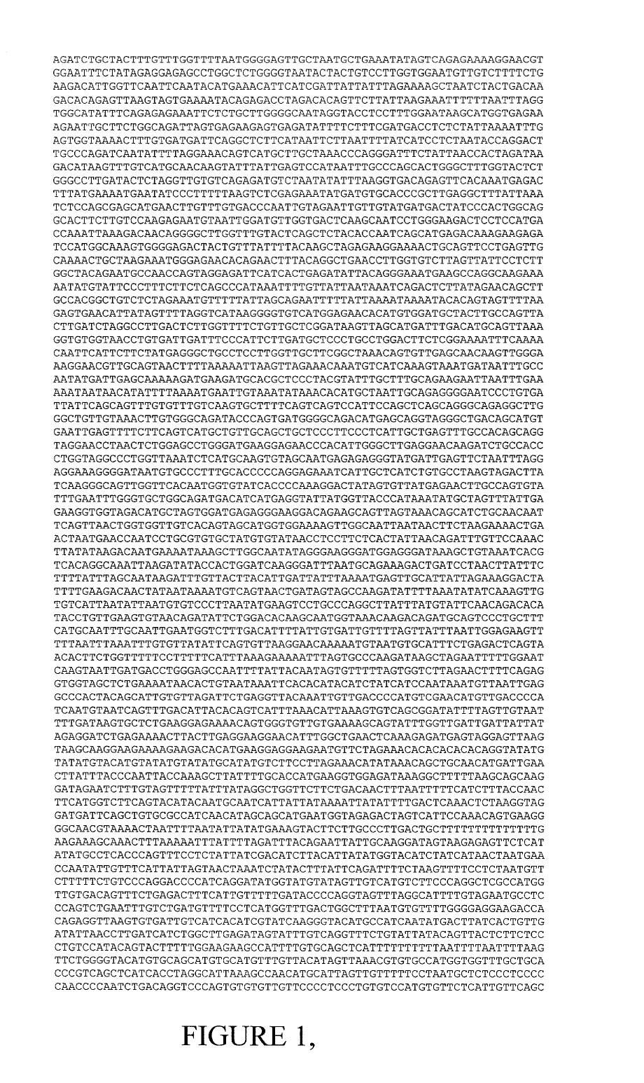

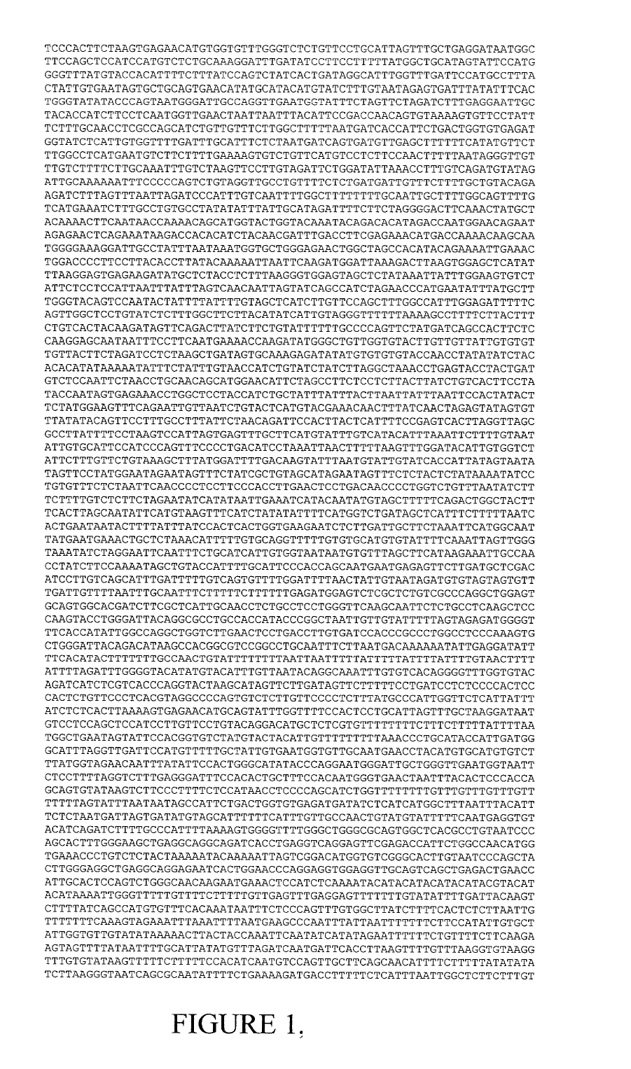

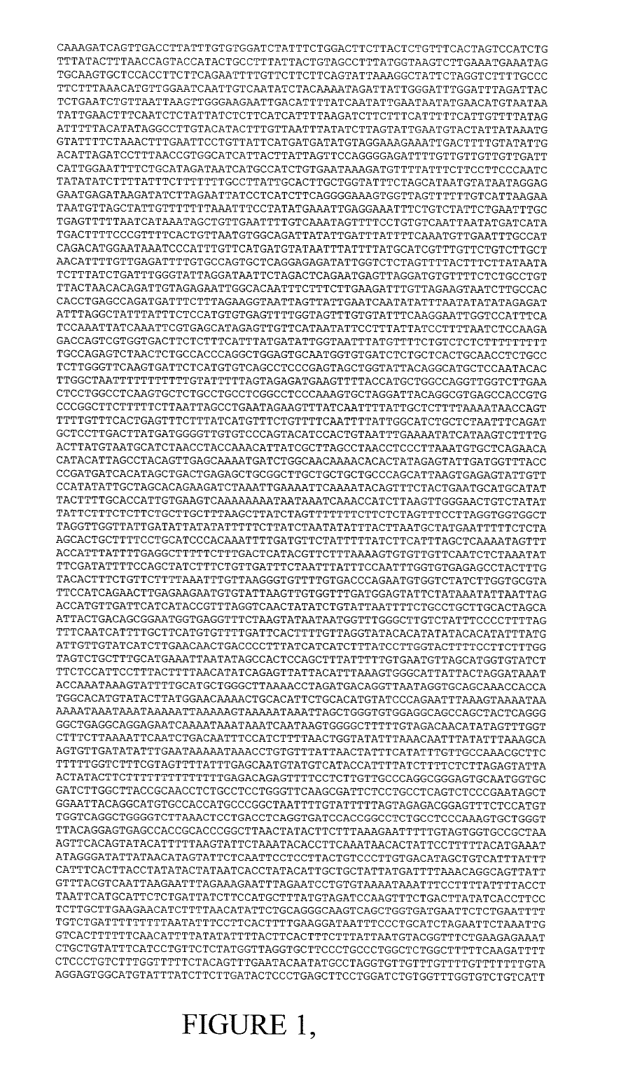

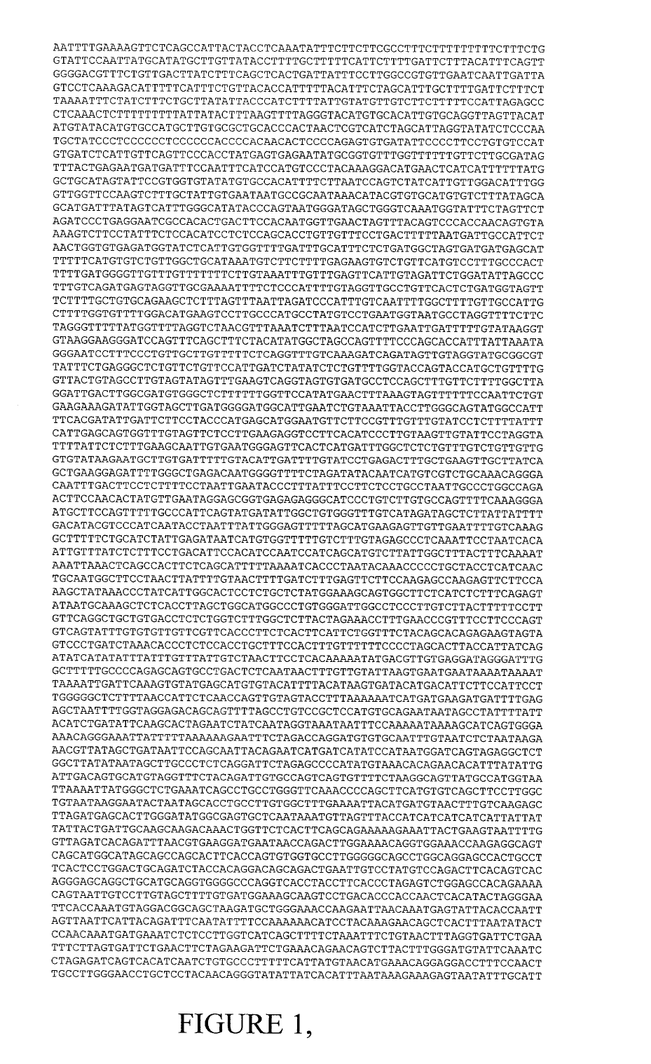

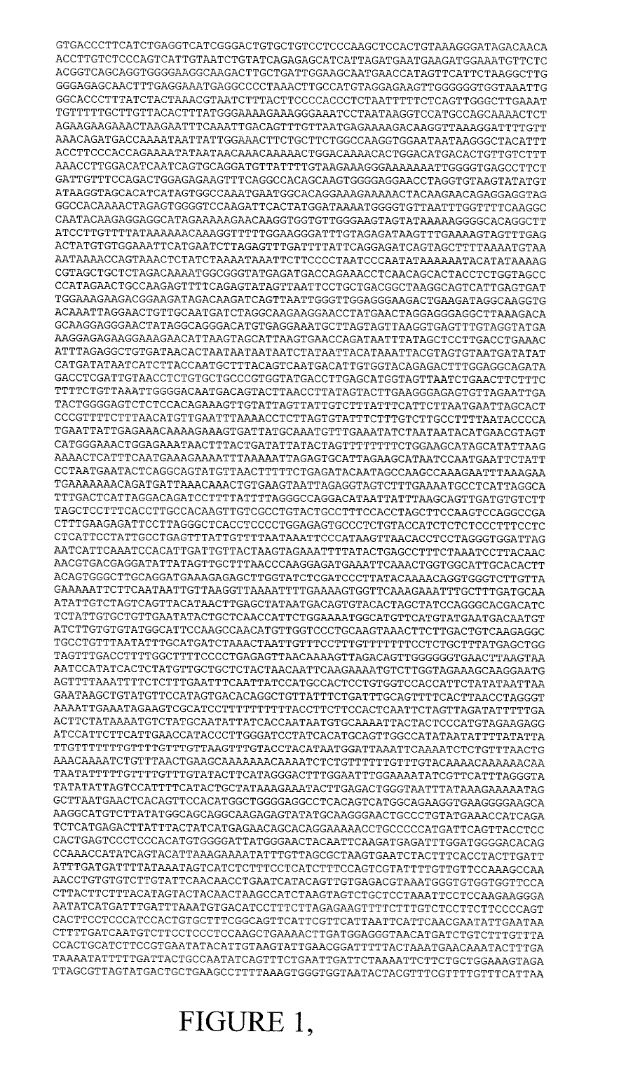

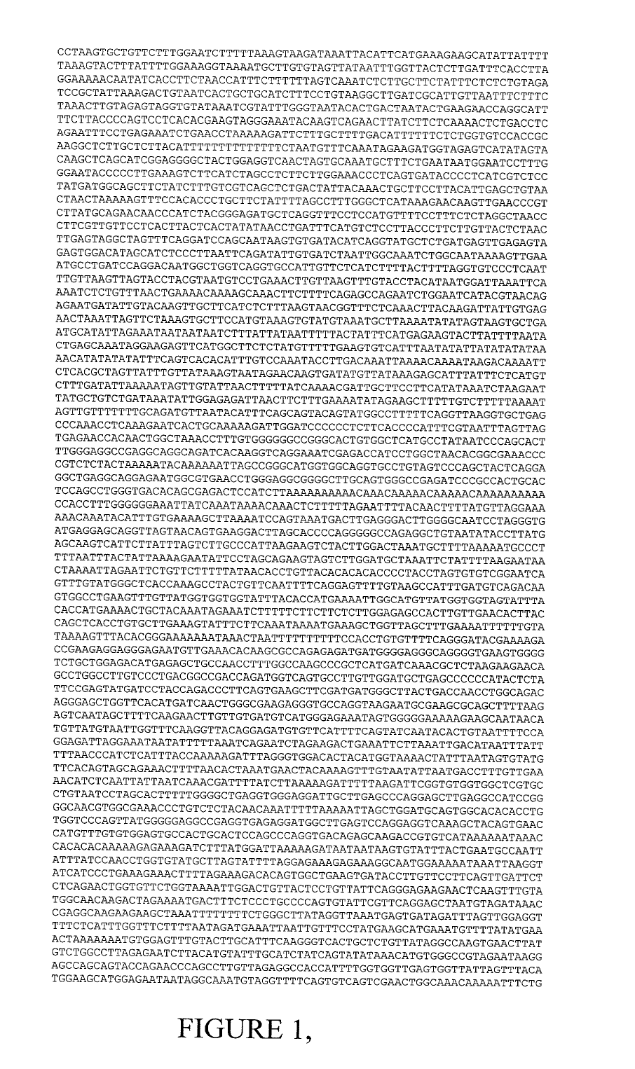

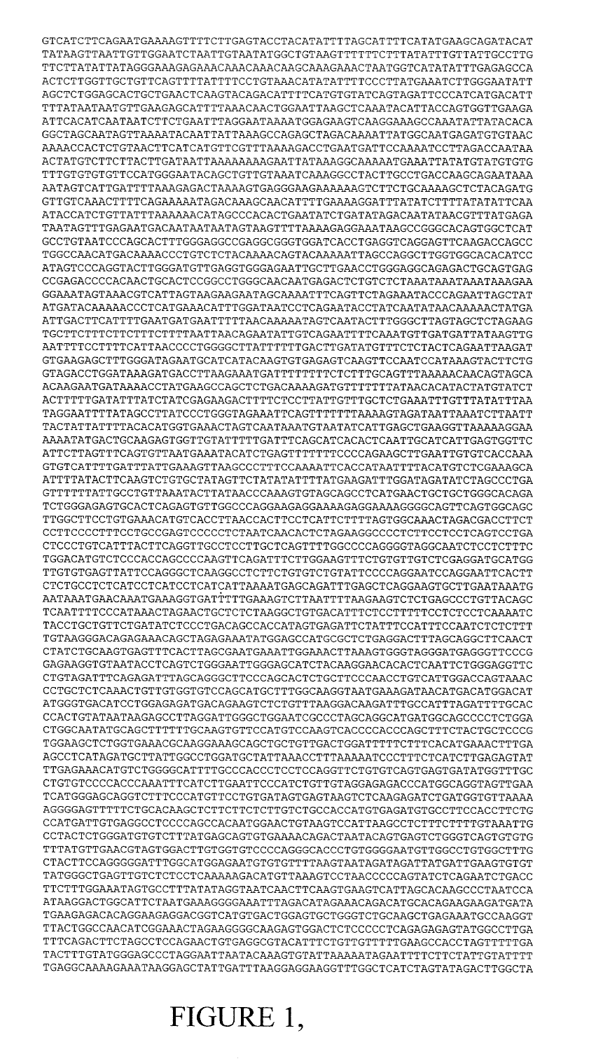

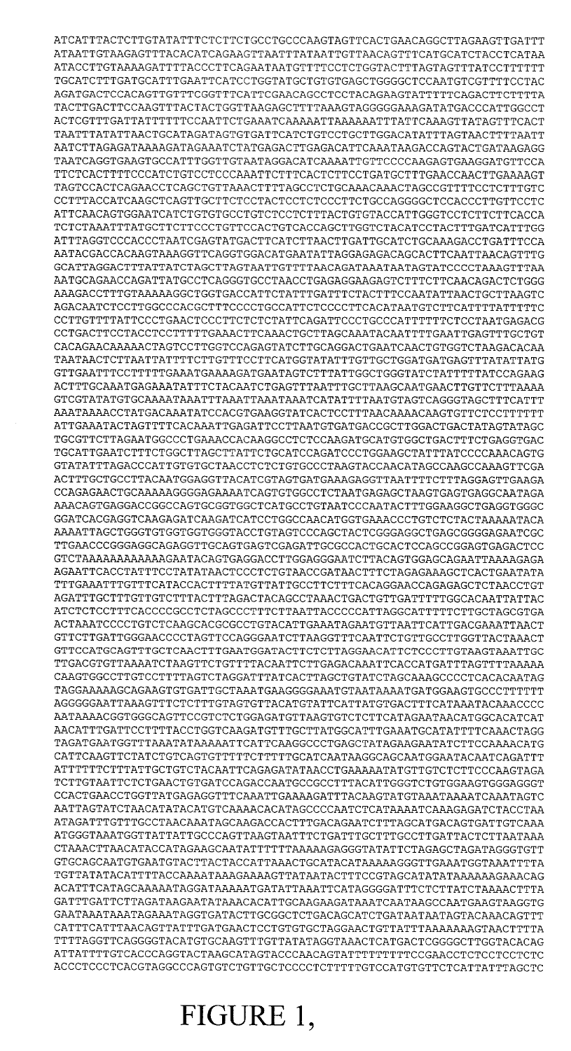

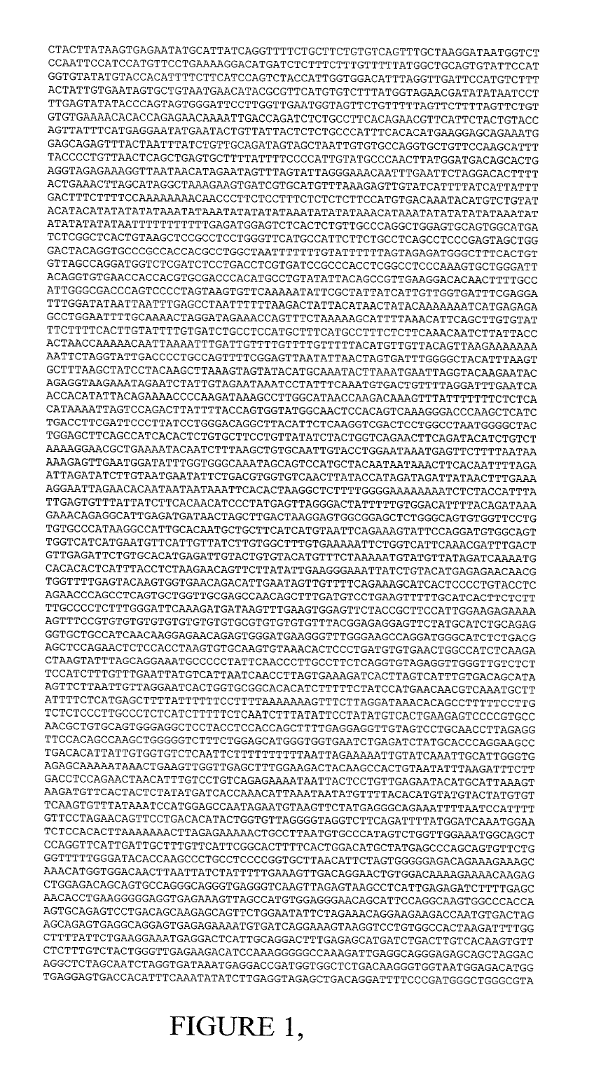

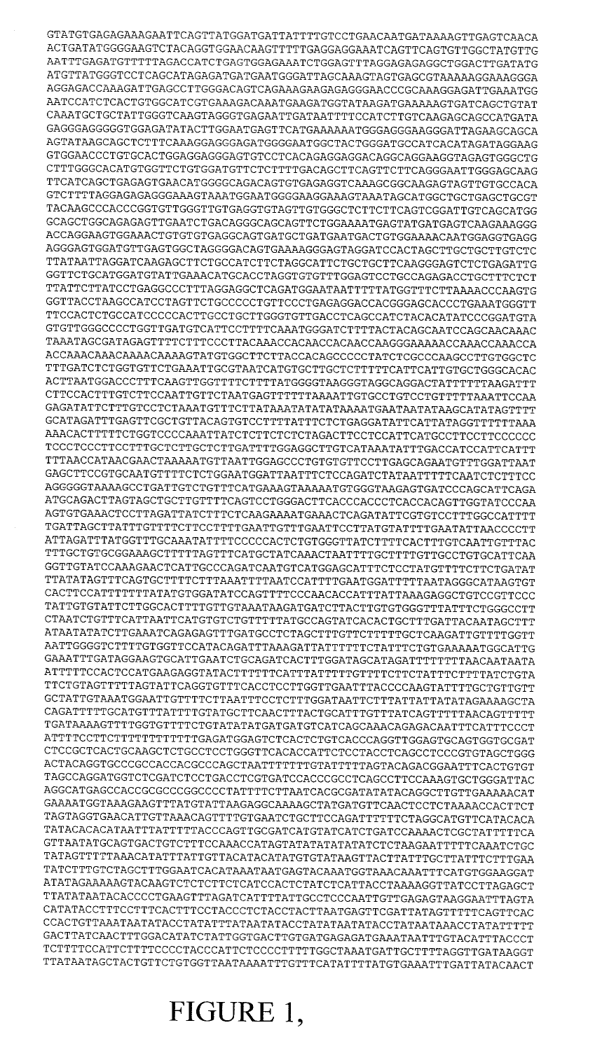

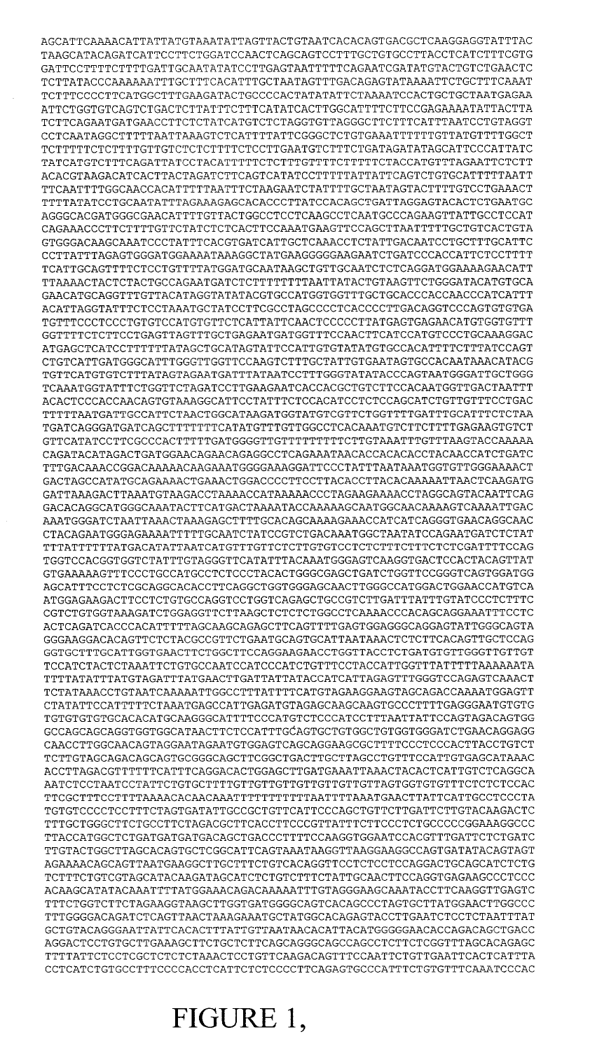

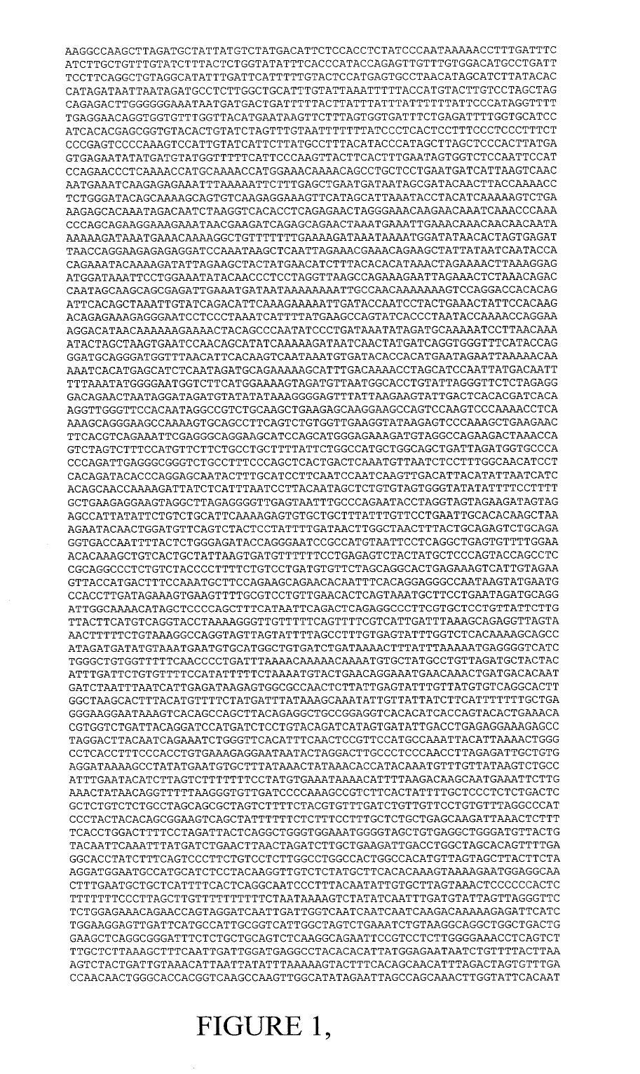

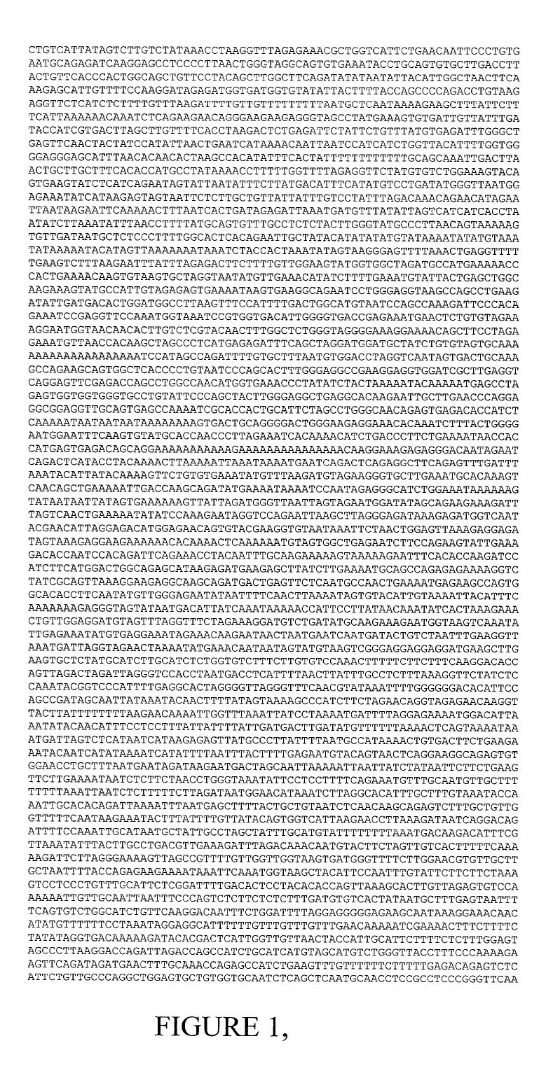

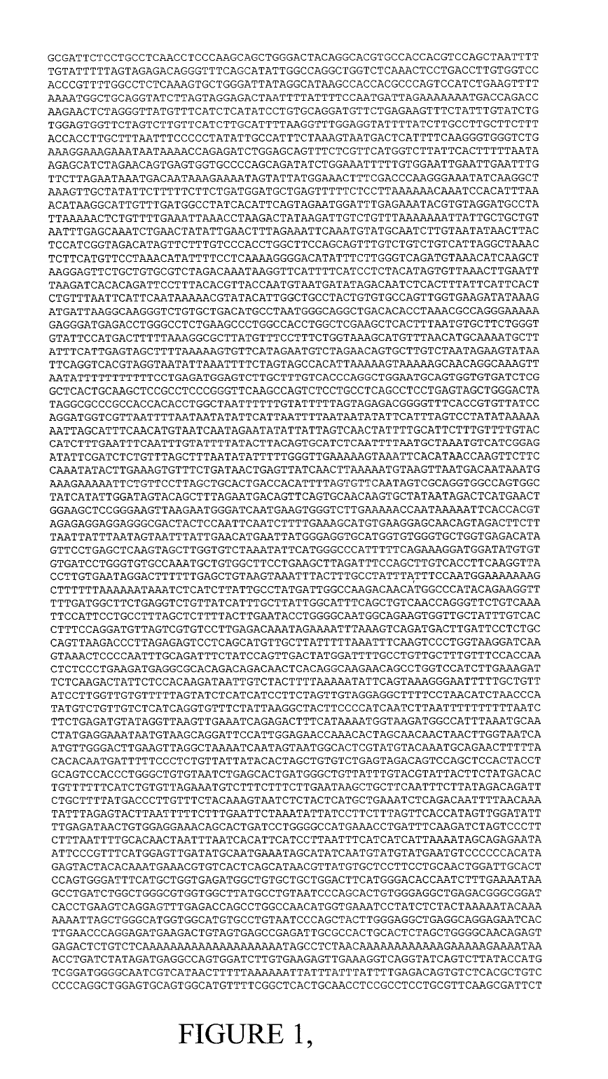

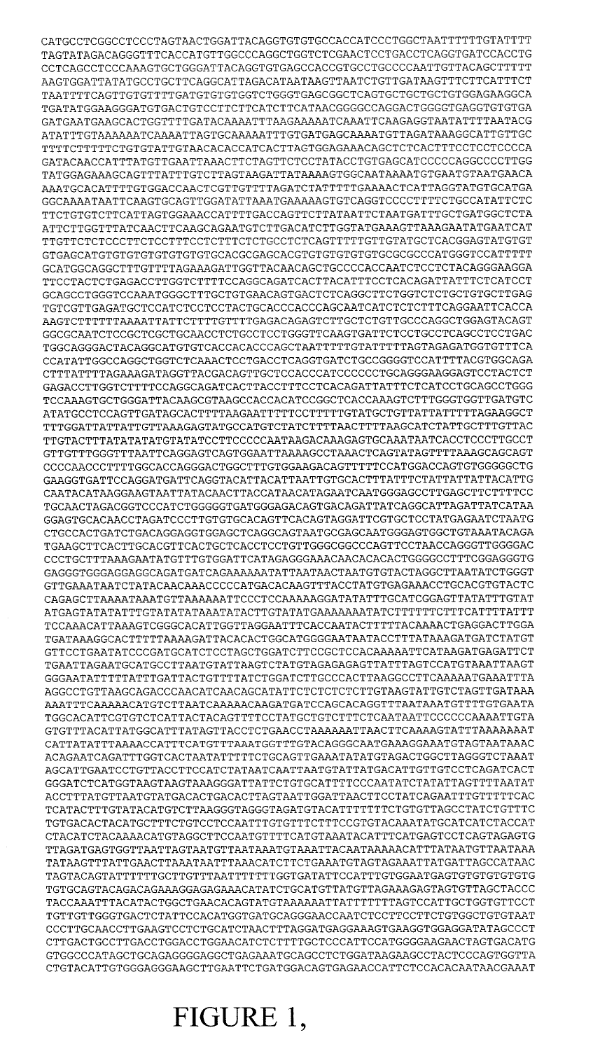

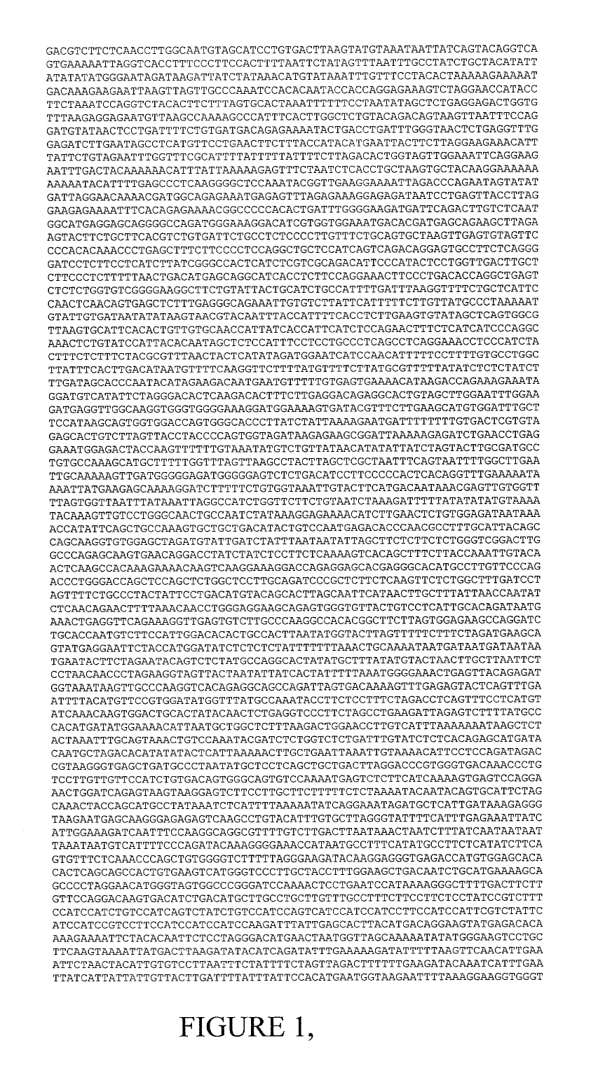

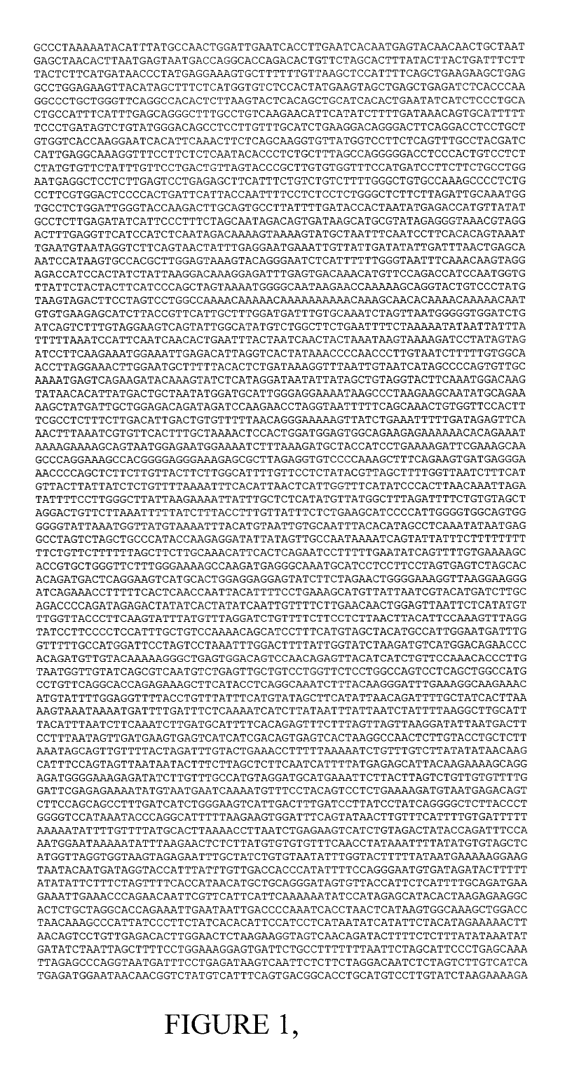

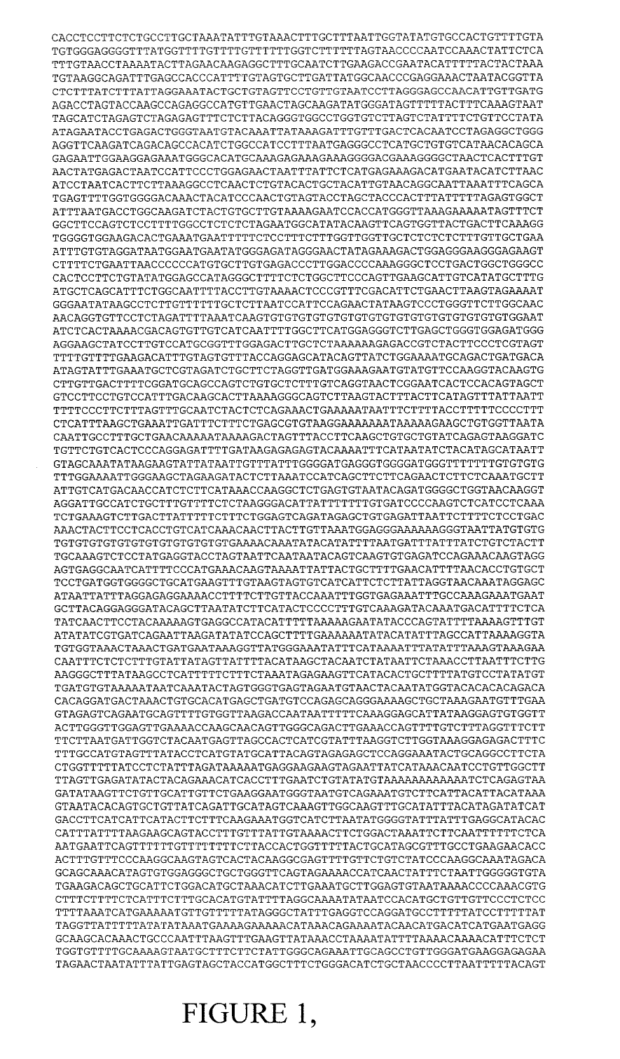

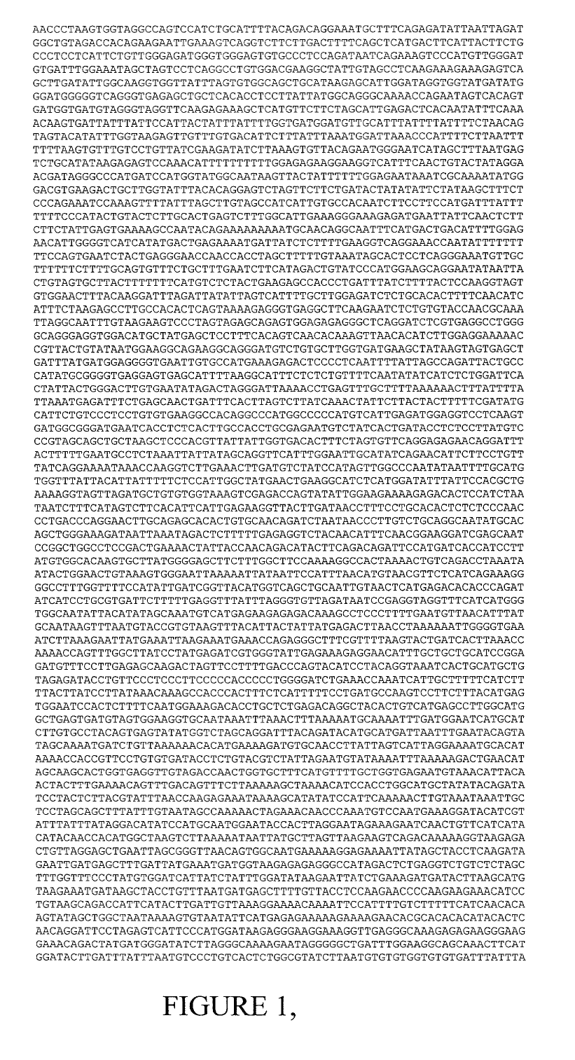

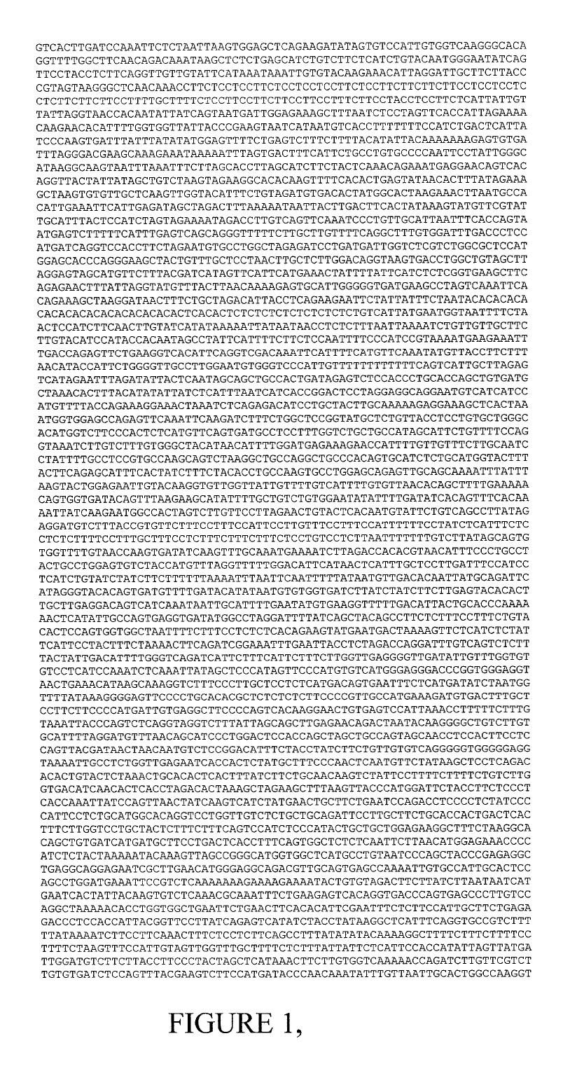

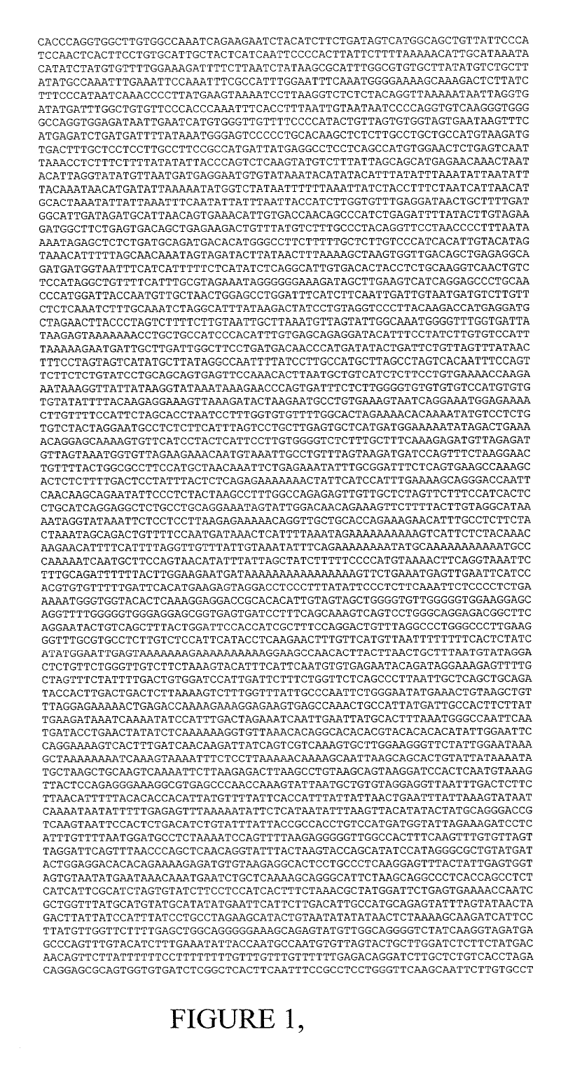

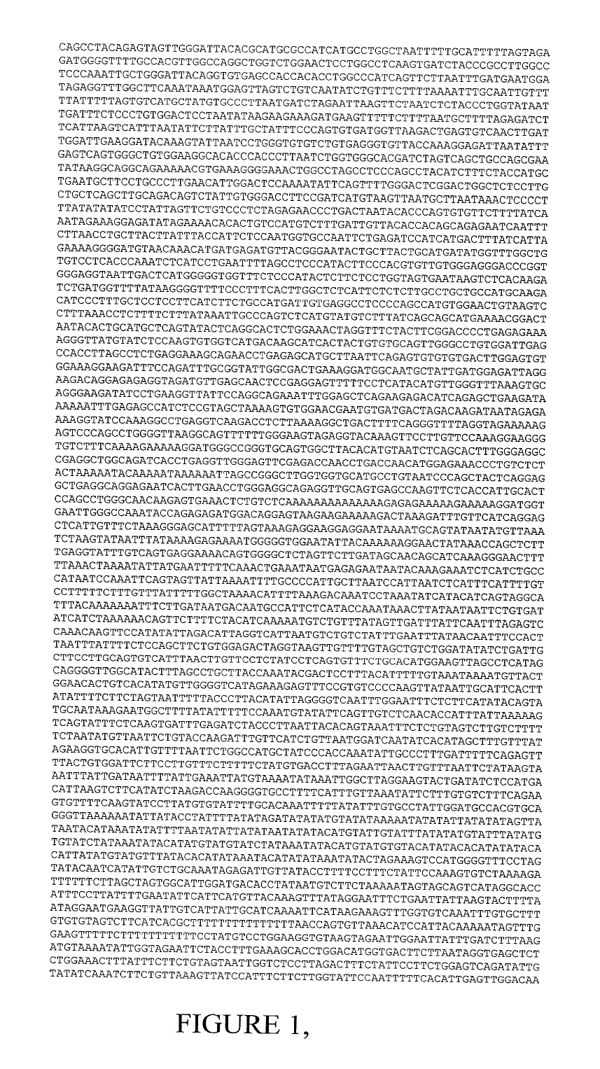

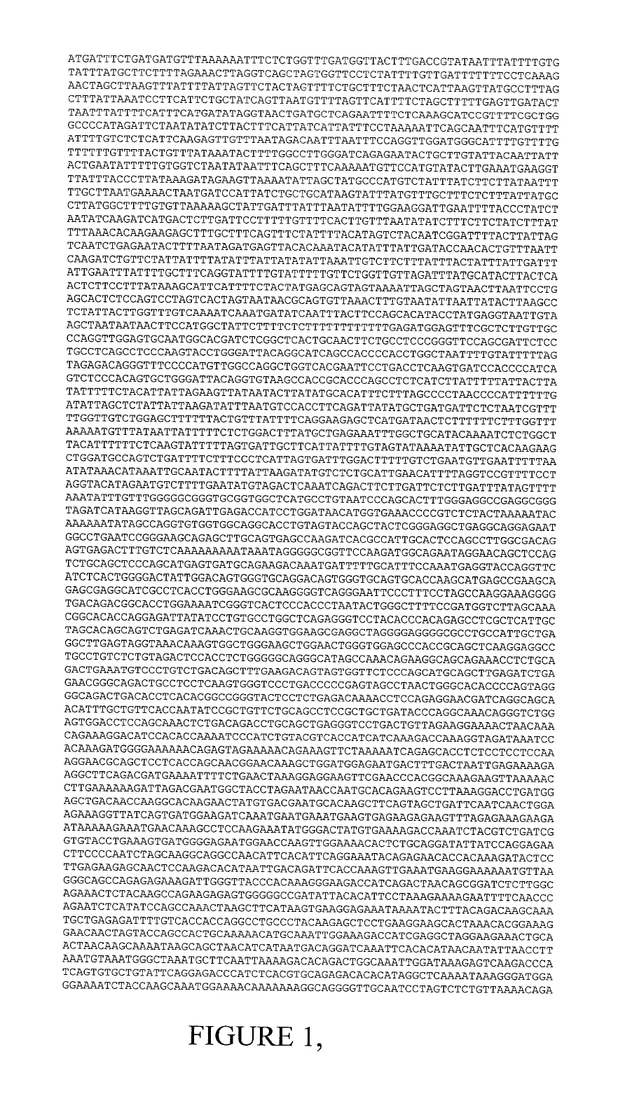

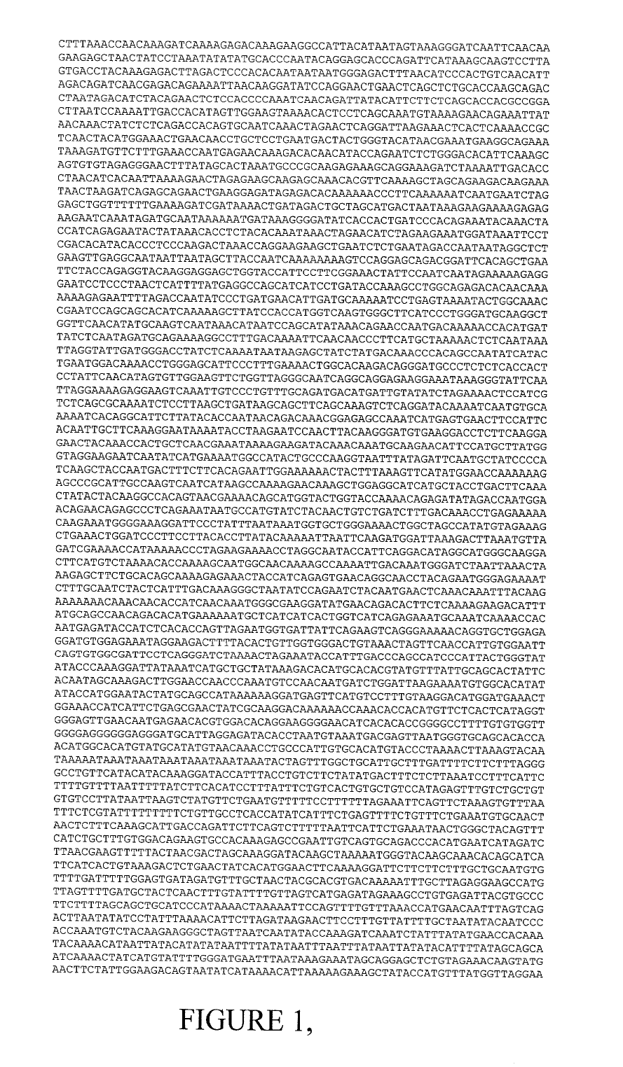

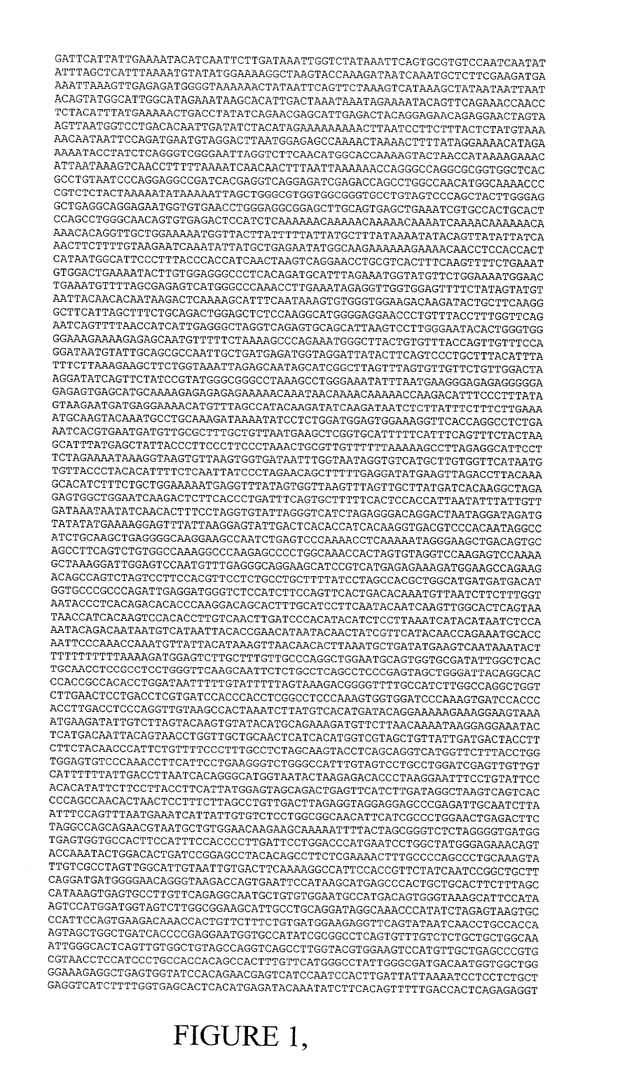

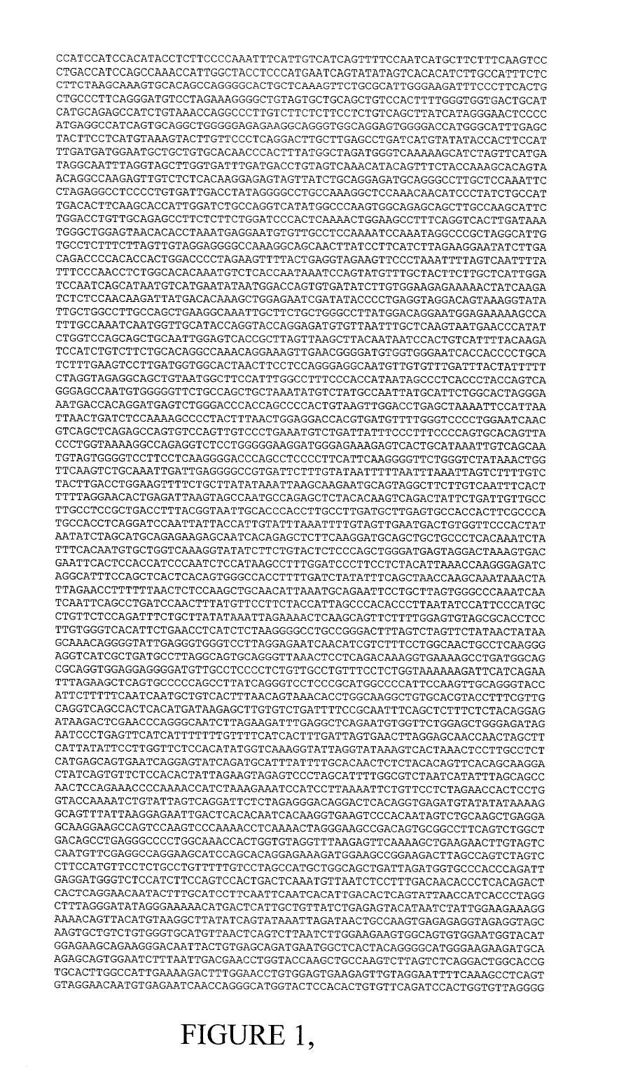

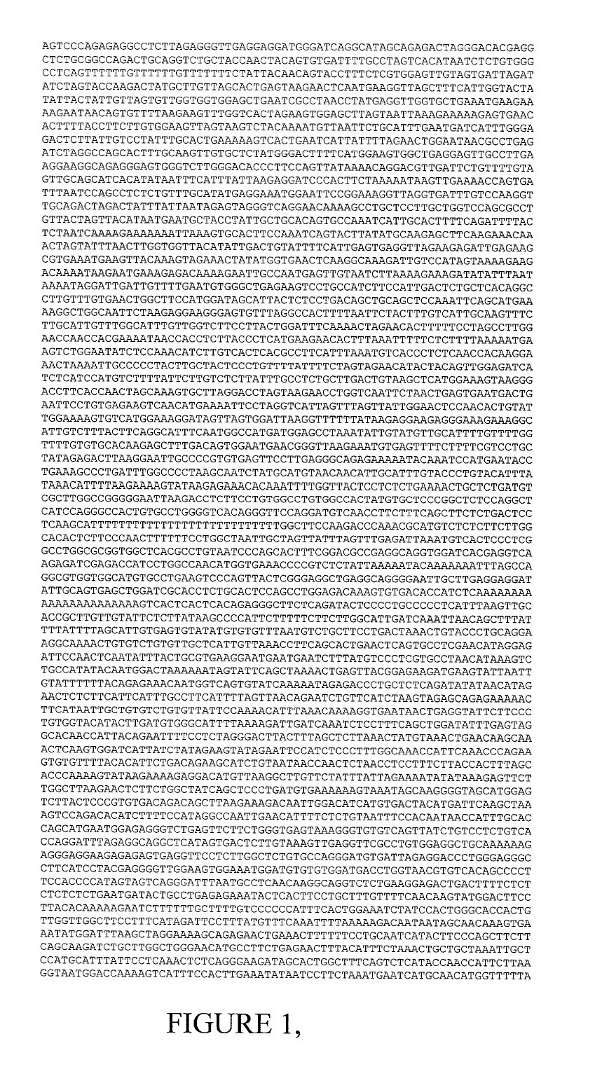

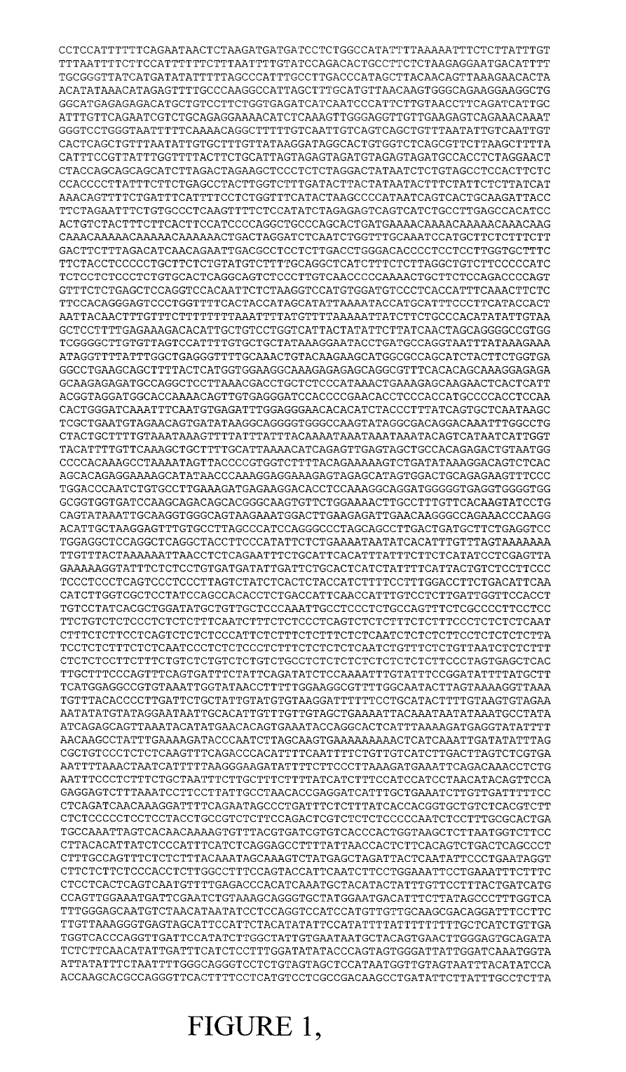

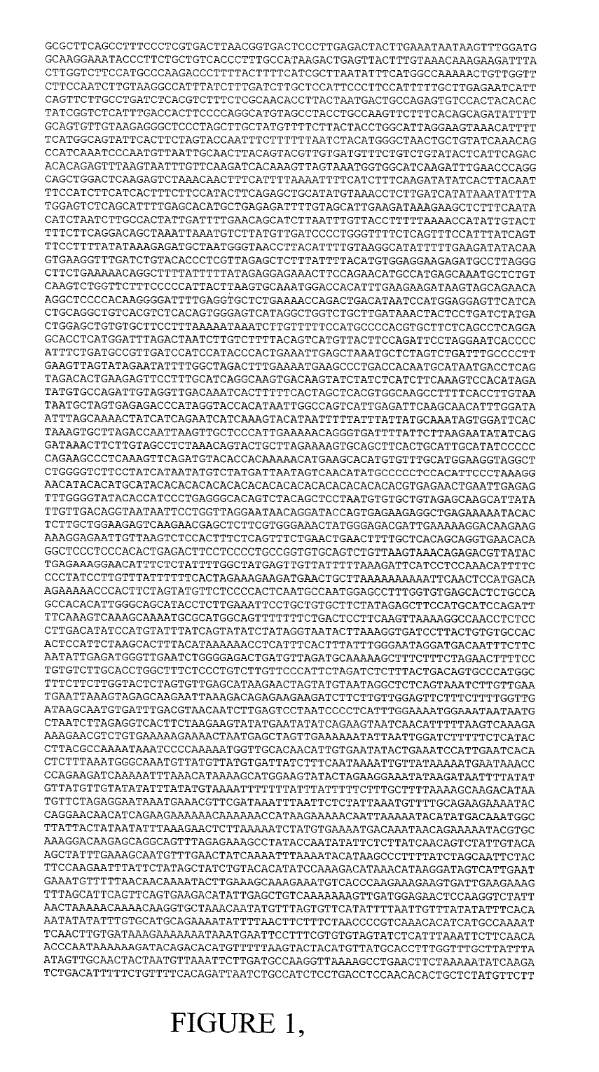

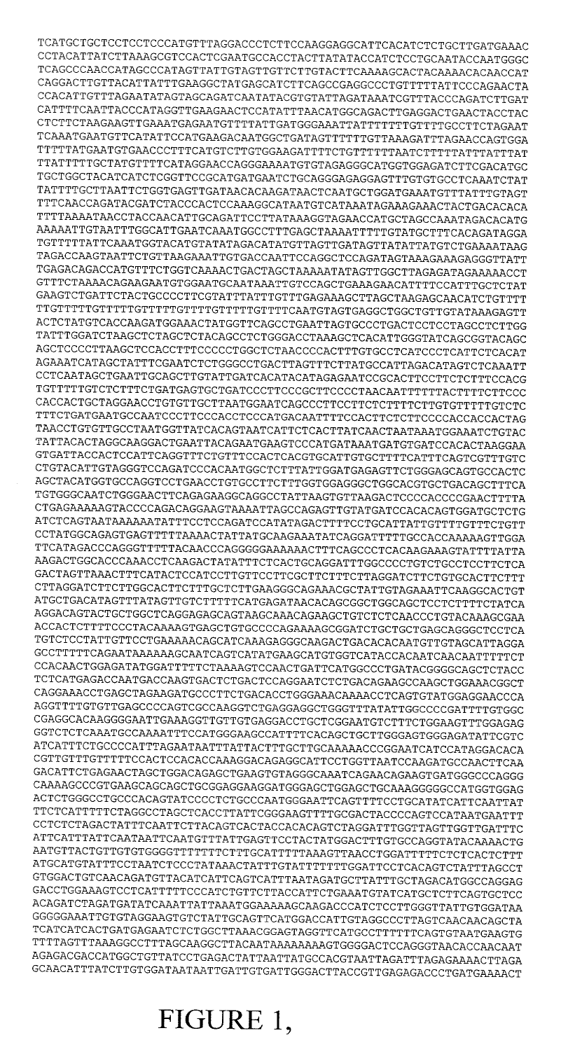

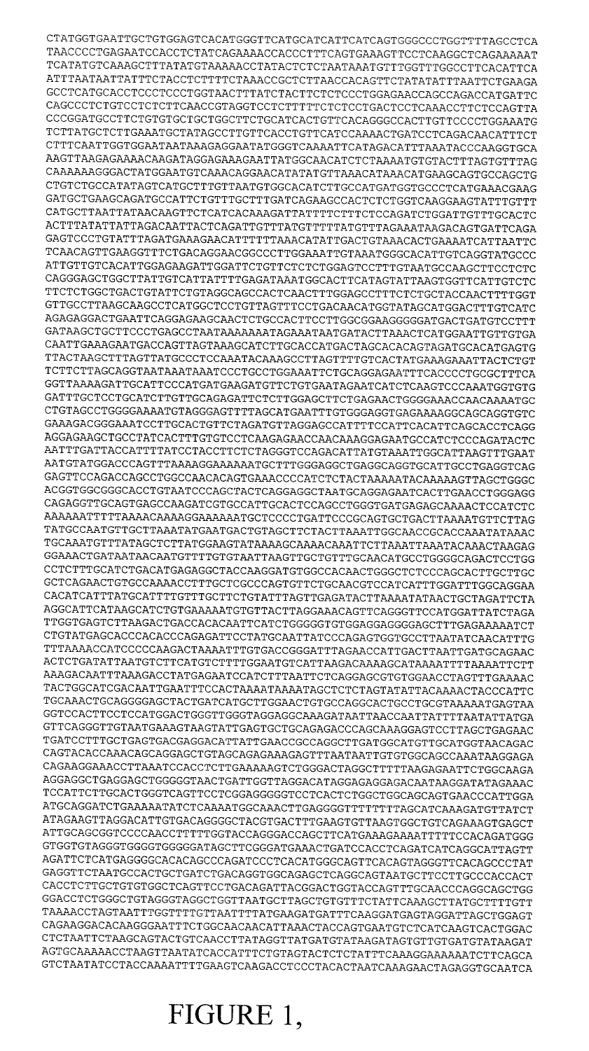

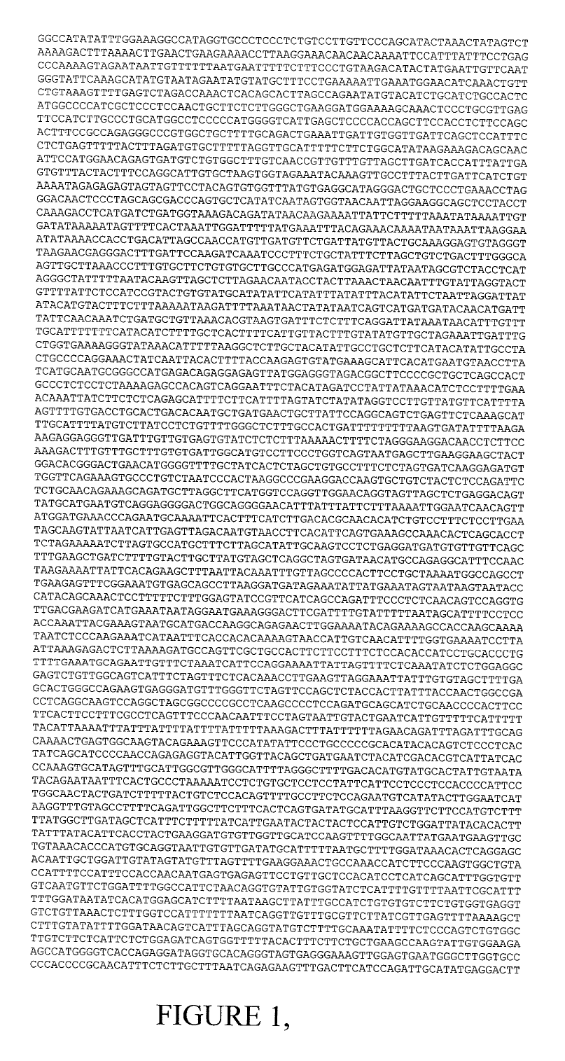

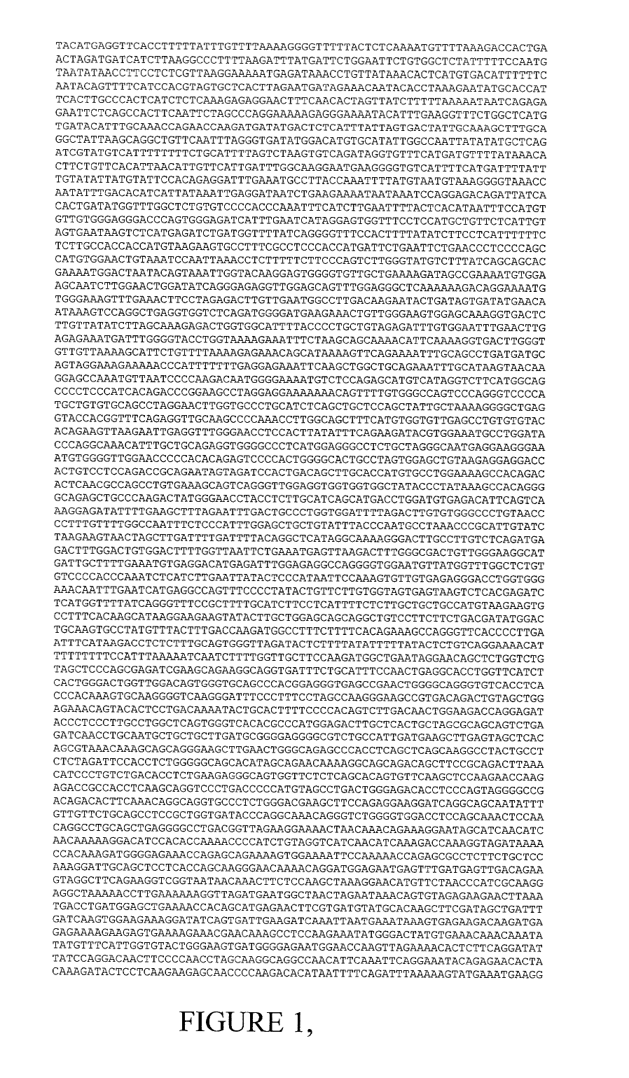

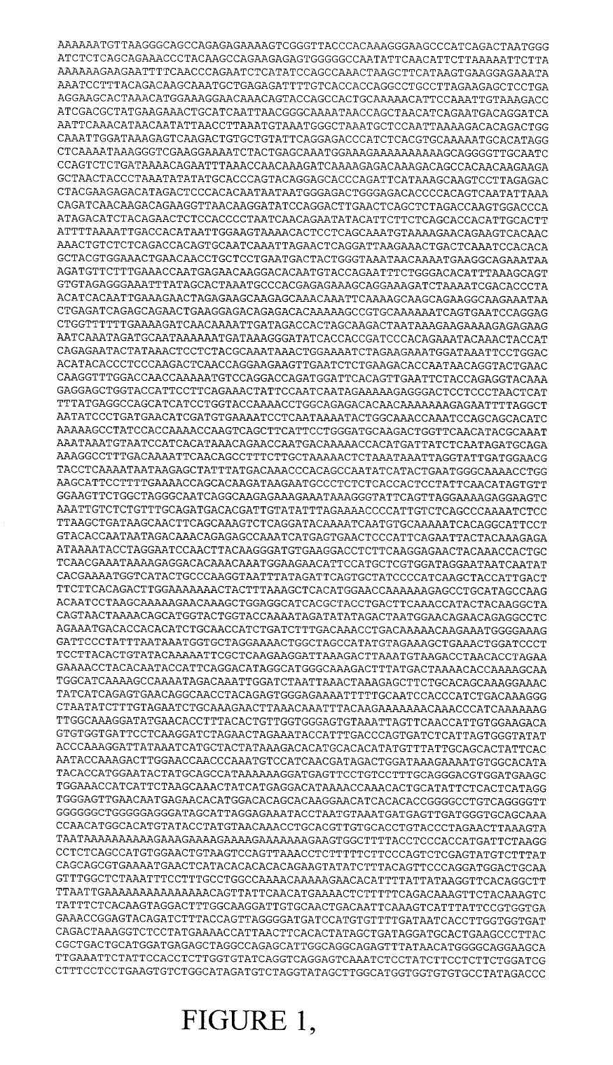

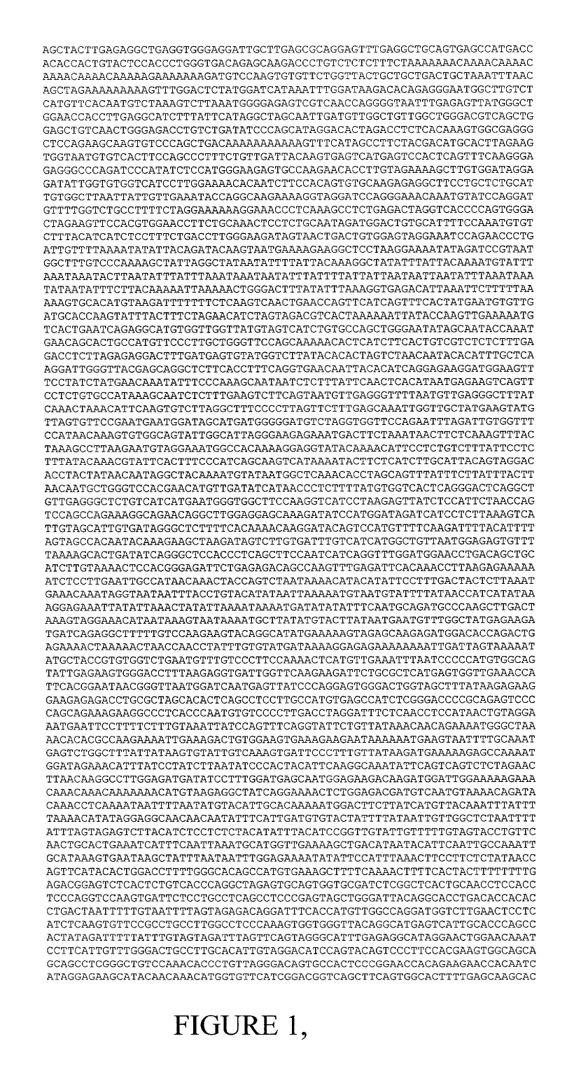

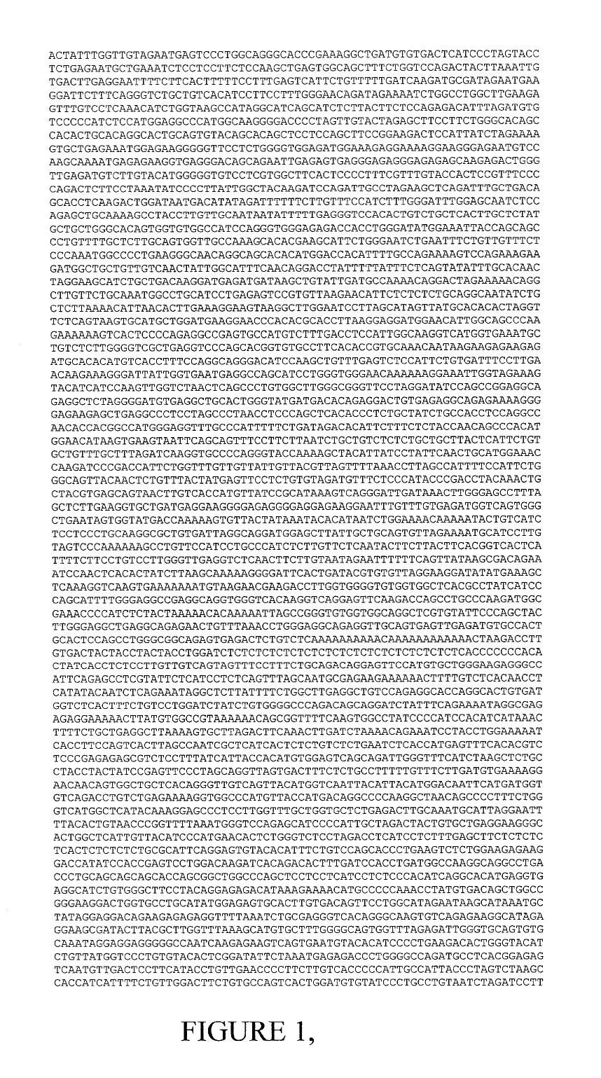

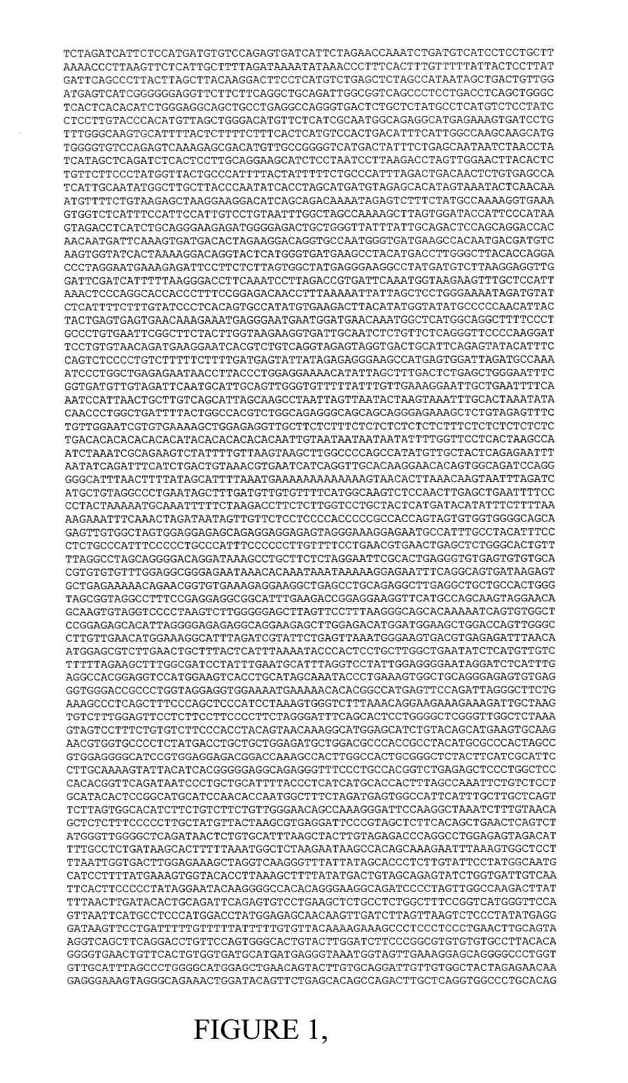

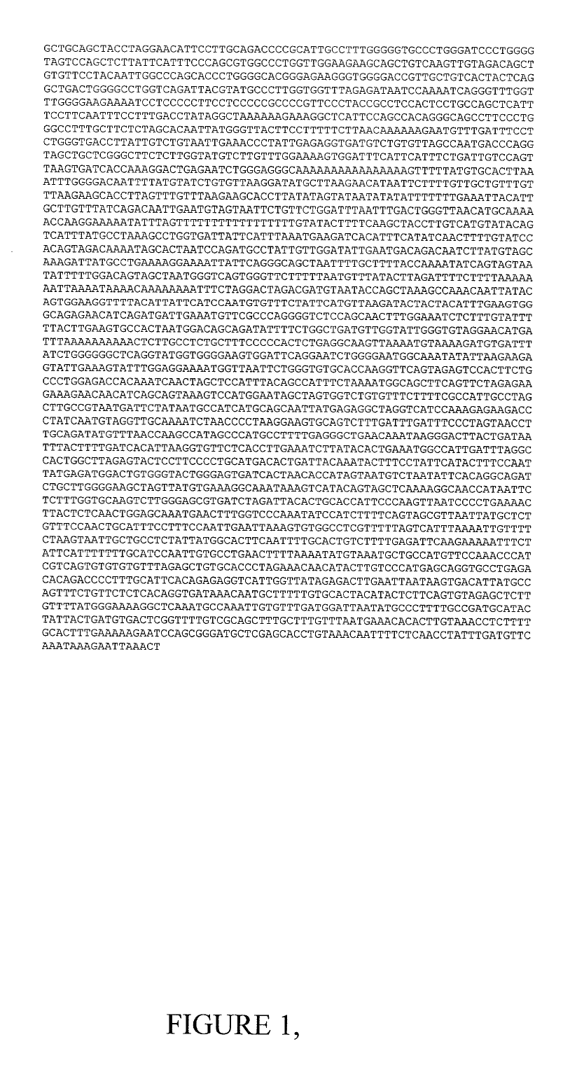

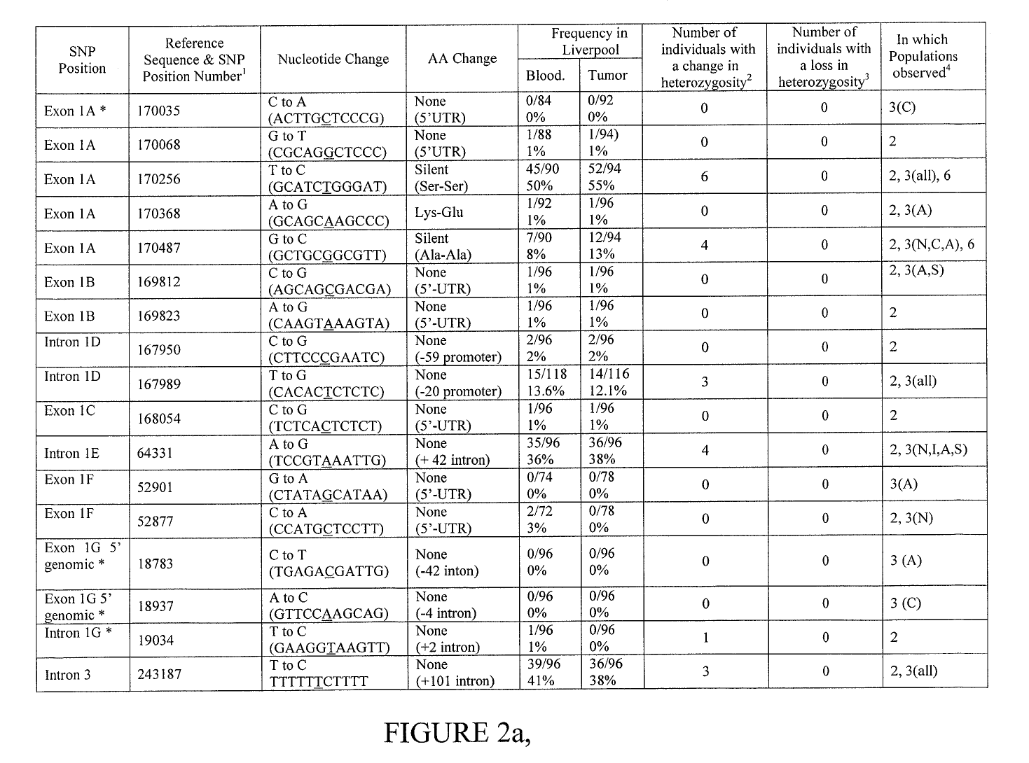

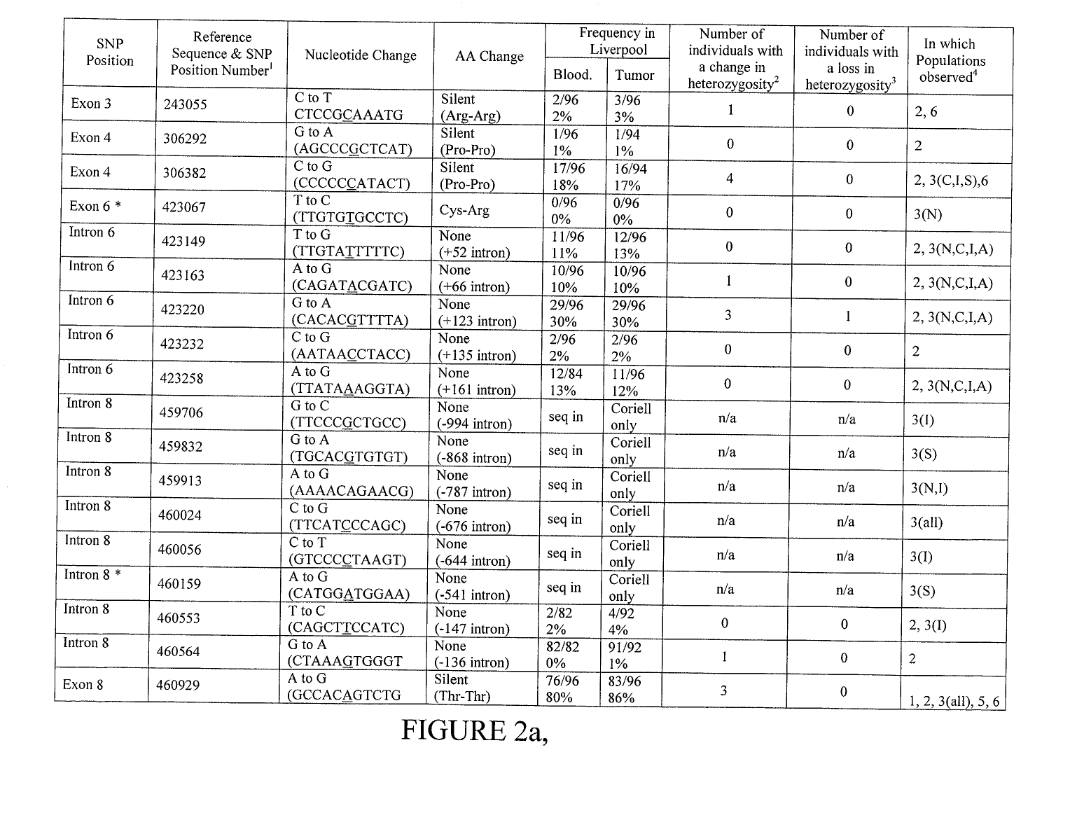

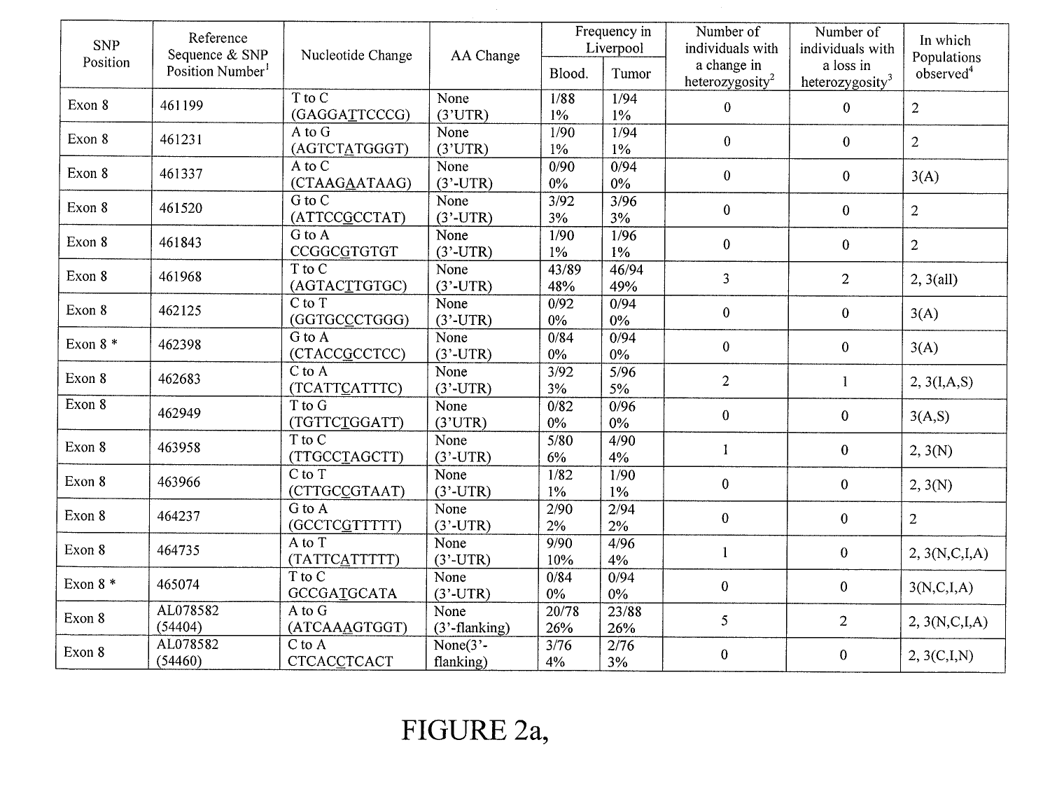

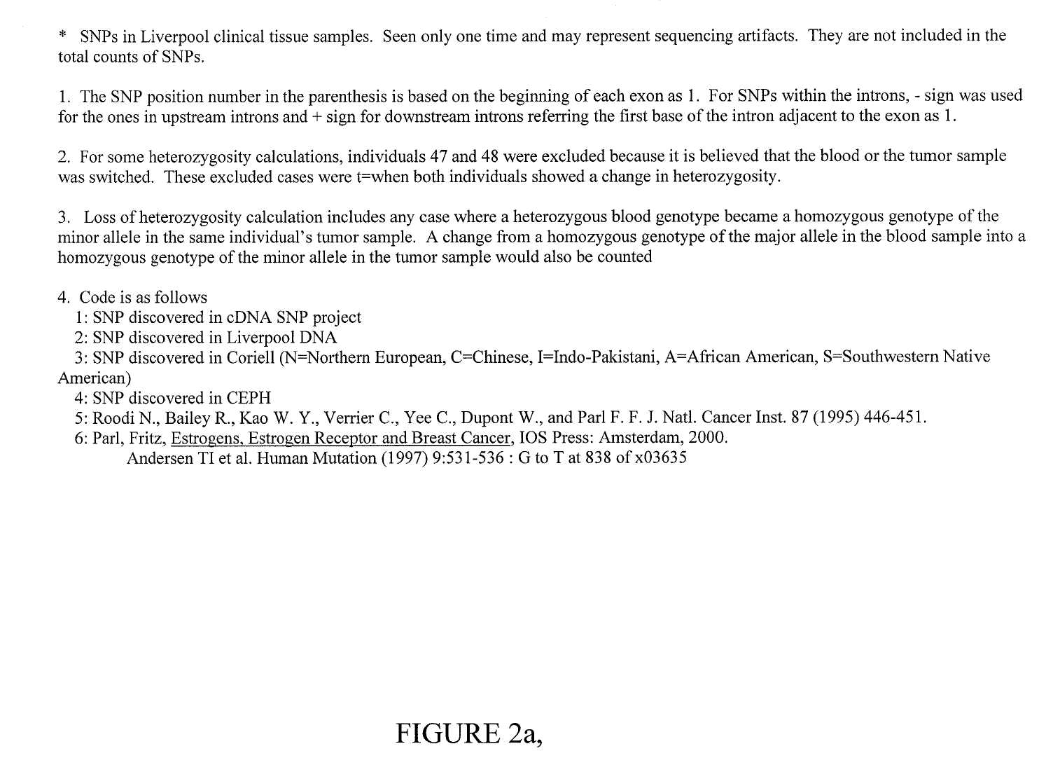

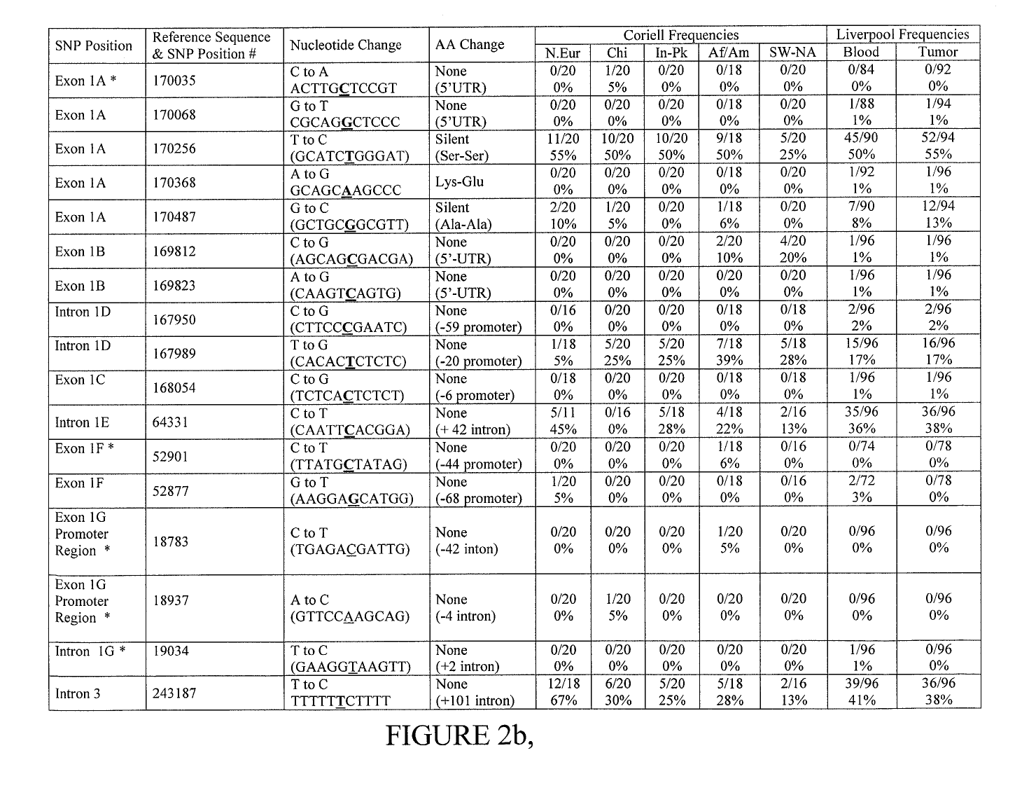

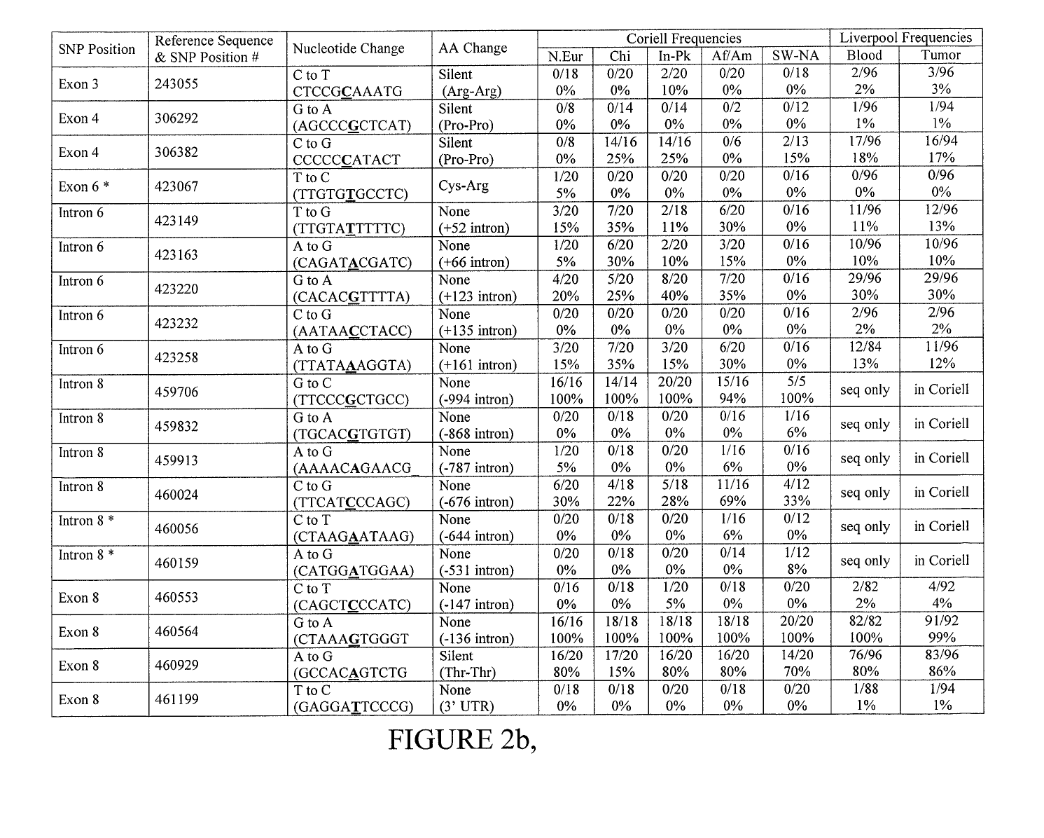

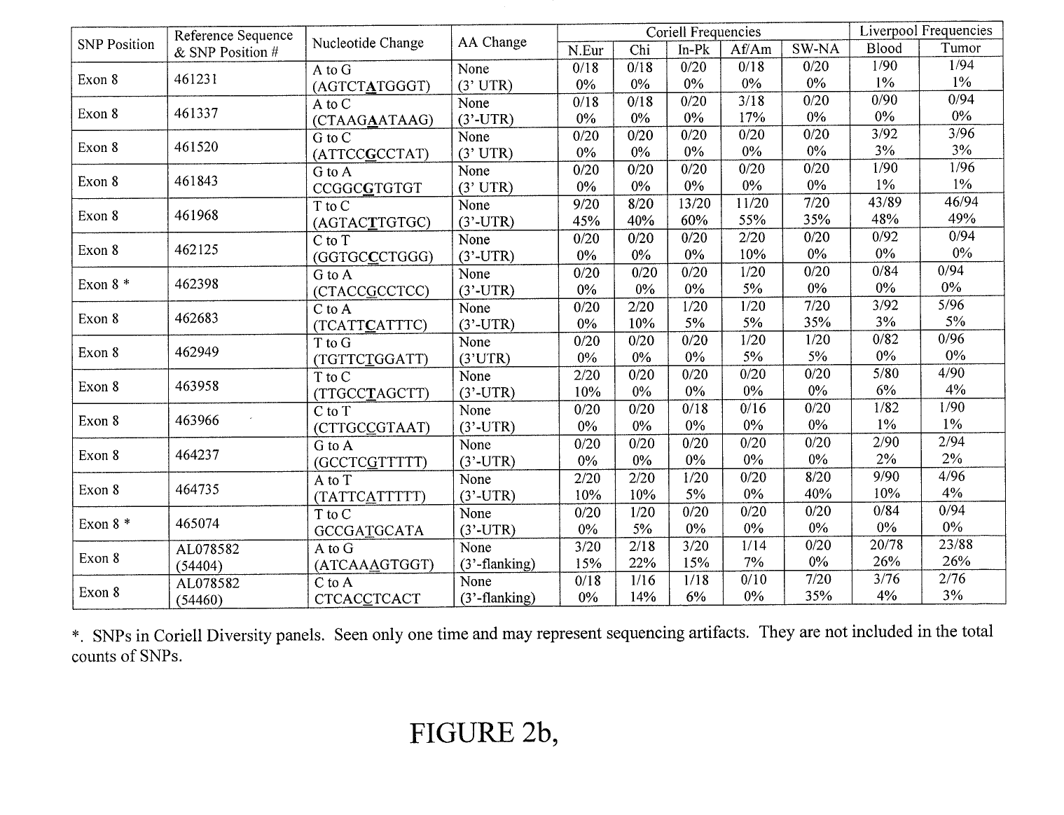

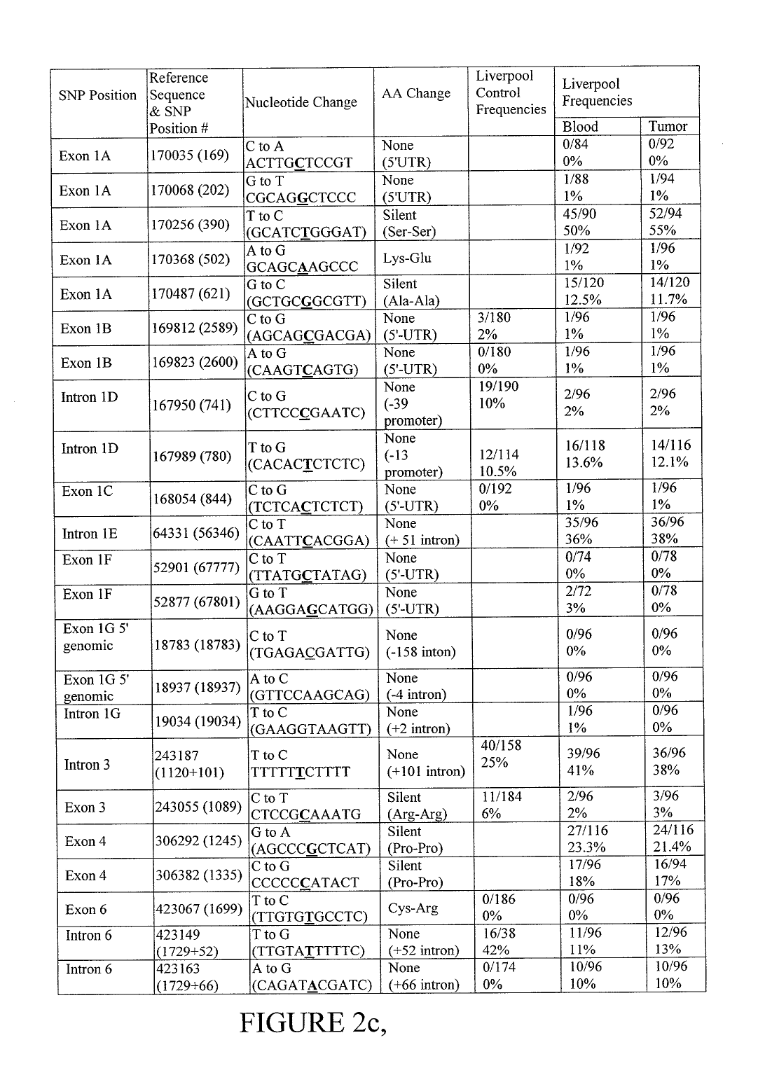

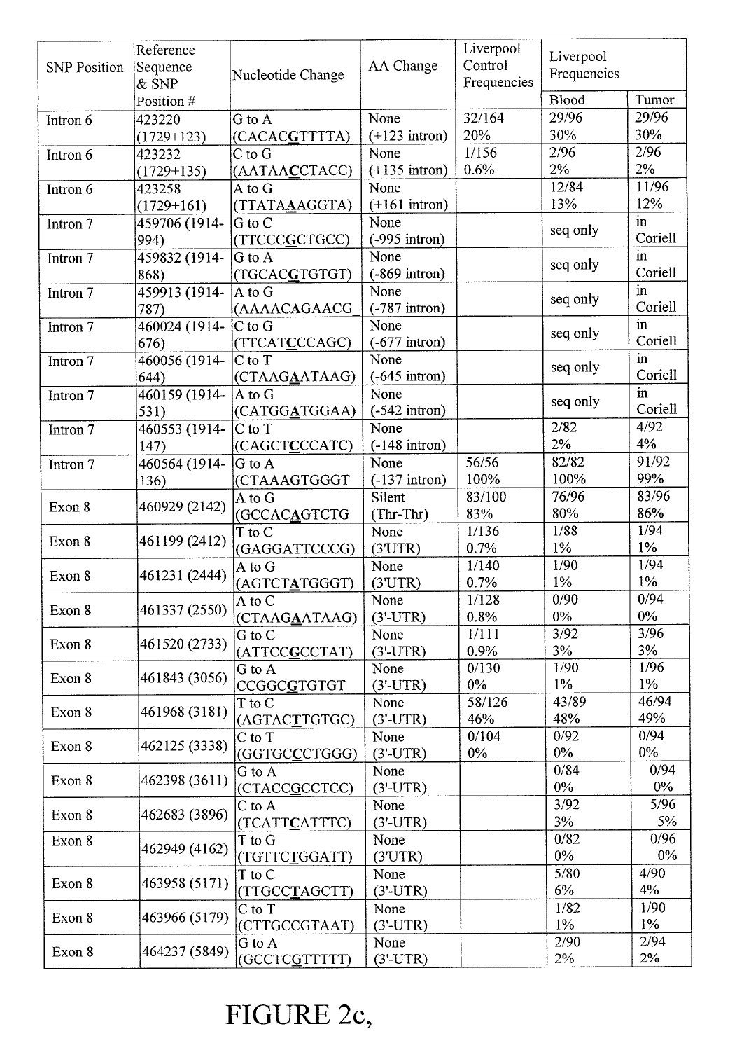

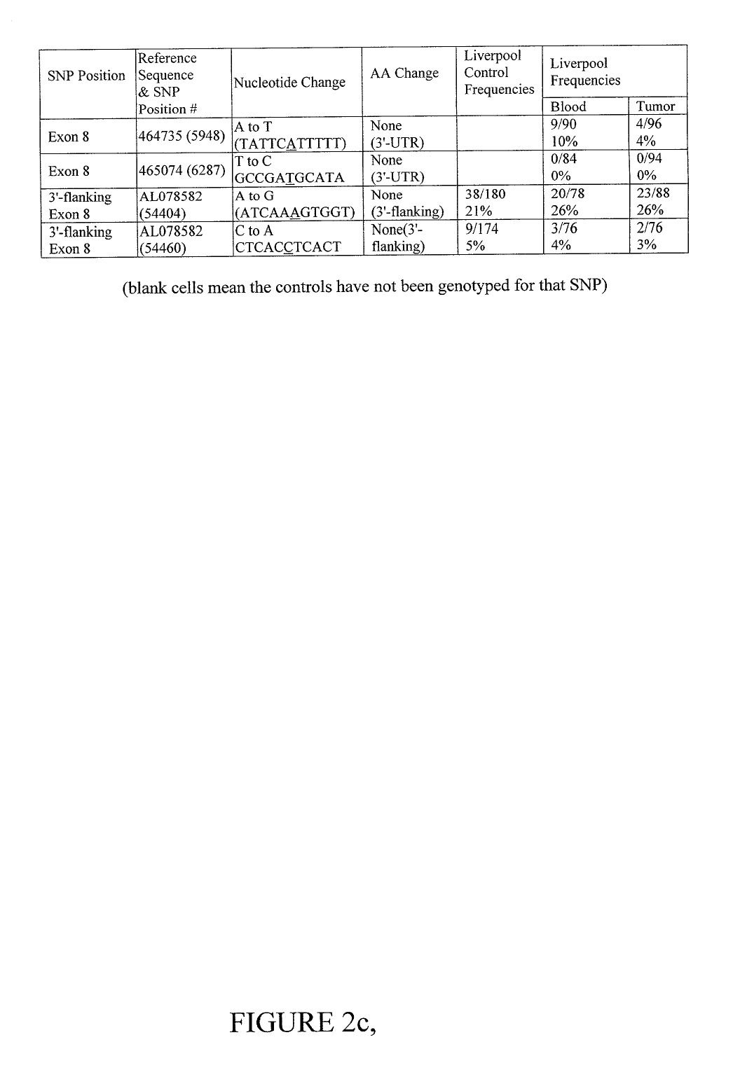

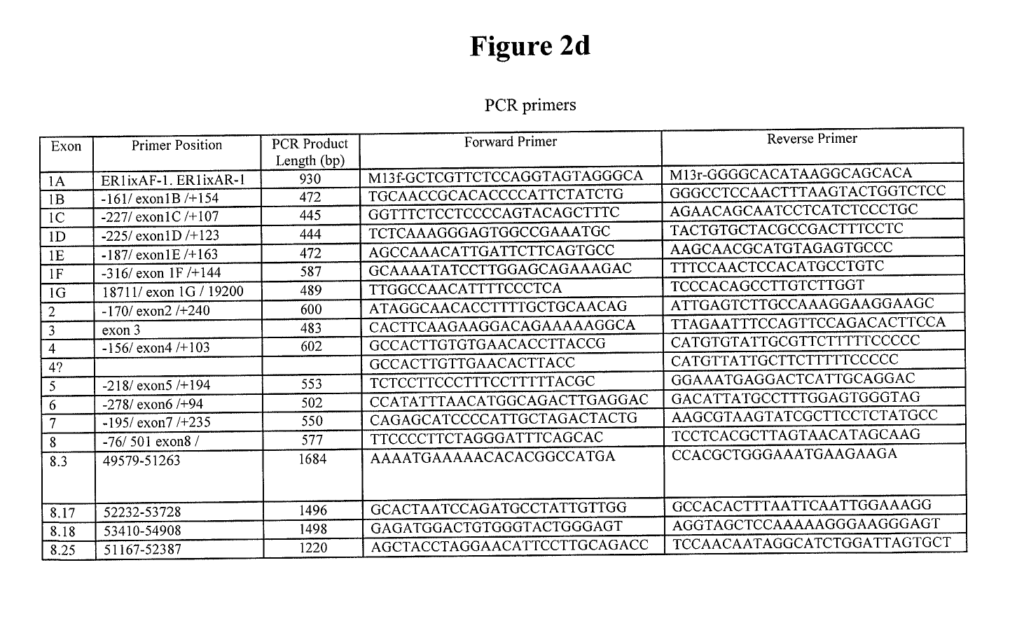

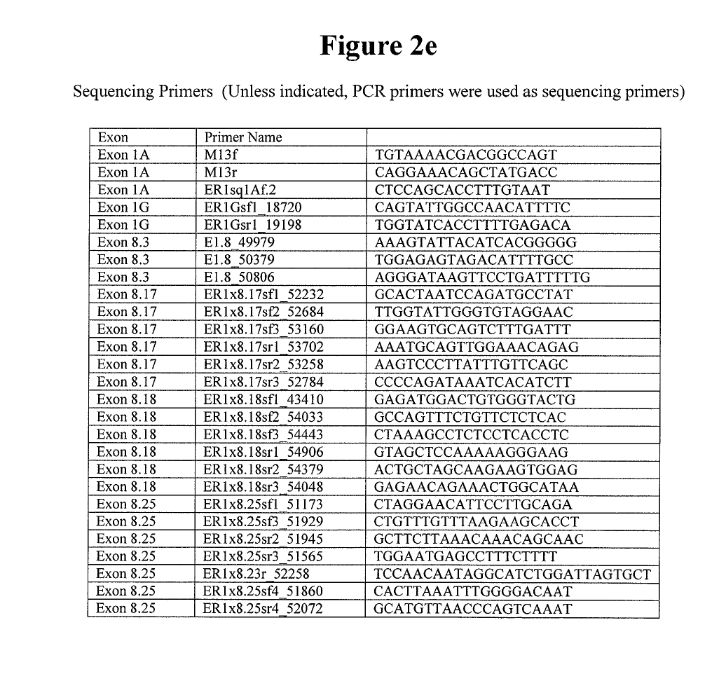

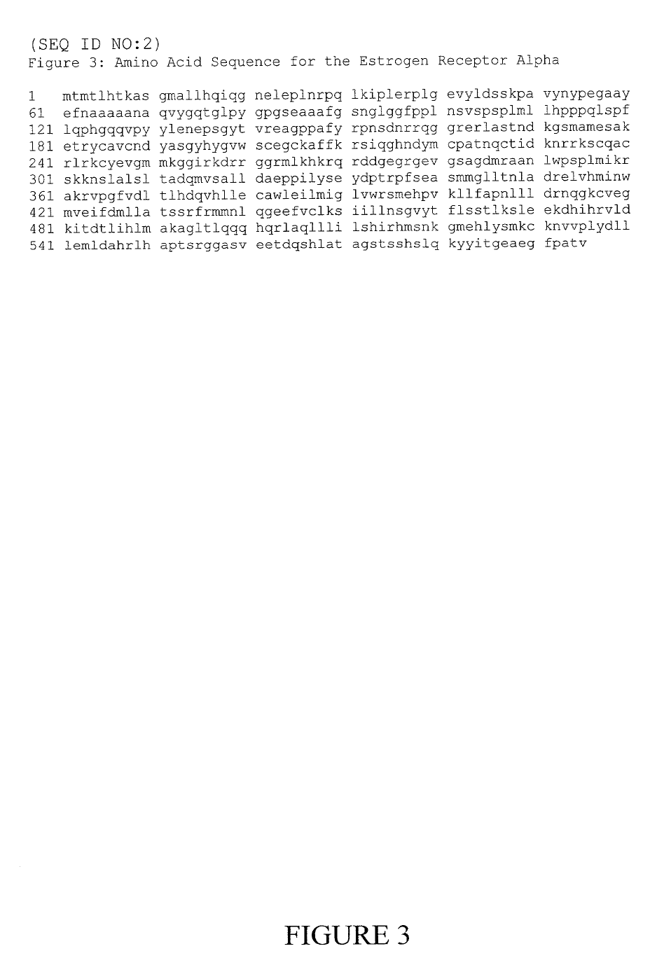

Accordingly, the present invention provides proteins that consist of the amino acid sequences summarized in FIG. 1, including one or more of the sequence polymorphisms provided in FIG. 2. A protein consists of an amino acid sequence when the amino acid sequence is the final amino acid sequence of the protein.

The present invention further provides proteins that consist essentially of the amino acid sequences summarized in FIG. 1, including one or more of the sequence polymorphisms provided in FIG. 2. A protein consists essentially of an amino acid sequence when such an amino acid sequence is present with only a few additional amino acid residues in the final protein.

The present invention further provides a protein that is comprised of the amino acid sequences summarized in FIG. 1, including one or more of the sequence polymorphisms provided in FIG. 2. A protein is comprised of an amino acid sequence when the amino acid sequence is at least part of the final amino acid sequence of the protein. In such a fashion, the protein can be only the peptide or have additional amino acid molecules, such as amino acid residues (contiguous encoded sequence) that are naturally associated with it or heterologous amino acid residues/peptide sequences. Such a protein can have a few additional amino acid residues or can comprise several hundred or more additional amino acids. A brief description of how various types of these proteins can be made/isolated is provided below.

The estrogen receptor protein of the present invention can be attached to heterologous sequences to form chimeric or fusion proteins. Such chimeric and fusion proteins comprise a estrogen receptor protein operatively linked to a heterologous protein having an amino acid sequence not substantially homologous to the estrogen receptor protein. “Operatively linked” indicates that the estrogen receptor protein and the heterologous protein are fused in-frame. The heterologous protein can be fused to the N-terminus or C-terminus of the estrogen receptor protein.

In some uses, the fusion protein does not affect the activity of the estrogen receptor protein per se. For example, the fusion protein can include, but is not limited to, enzymatic fusion proteins, for example beta-galactosidase fusions, yeast two-hybrid GAL fusions, poly-His fusions, MYC-tagged, HI-tagged and Ig fusions. Such fusion proteins, particularly poly-His fusions, can facilitate the purification of recombinant estrogen receptor protein. In certain host cells (e.g., mammalian host cells), expression and/or secretion of a protein can be increased by using a heterologous signal sequence.

A chimeric or fusion protein can be produced by standard recombinant DNA techniques. For example, DNA fragments coding for the different protein sequences are ligated together in-frame in accordance with conventional techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see Ausubel et al., Current Protocols in Molecular Biology, 1992). Moreover, many expression vectors are commercially available that already encode a fusion moiety (e.g., a GST protein). A estrogen receptor protein-encoding nucleic acid can be cloned into such an expression vector such that the fusion moiety is linked in-frame to the estrogen receptor protein.

Polypeptides often contain amino acids other than the 20 amino acids commonly referred to as the 20 naturally-occurring amino acids. Further, many amino acids, including the terminal amino acids, may be modified by natural processes, such as processing and other post-translational modifications, or by chemical modification techniques well known in the art. Common modifications that occur naturally in polypeptides are described in basic texts, detailed monographs, and the research literature, and they are well known to those of skill in the art. Accordingly, the polypeptides also encompass derivatives or analogs in which a substituted amino acid residue is not one encoded by the genetic code, in which a substituent group is included, in which the mature polypeptide is fused with another compound, such as a compound to increase the half-life of the polypeptide (for example, polyethylene glycol), or in which the additional amino acids are fused to the mature polypeptide, such as a leader or secretory sequence or a sequence for purification of the mature polypeptide or a pro-protein sequence.

Known modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Such modifications are well-known to those of skill in the art and have been described in great detail in the scientific literature. Several particularly common modifications, glycosylation, lipid attachment, sulfation, gamma-carboxylation of glutamic acid residues, hydroxylation and ADP-ribosylation, for instance, are described in most basic texts, such as Proteins—Structure and Molecular Properties, 2nd Ed., T. E. Creighton, W. H. Freeman and Company, New York (1993). Many detailed reviews are available on this subject, such as by Wold, F., Posttranslational Covalent Modification of Proteins, B. C. Johnson, Ed., Academic Press, New York 1-12 (1983); Seifter et al. (Meth. Enzymol. 182: 626-646 (1990)) and Rattan et al. (Ann. N. Y. Acad. Sci. 663:48-62 (1992)).

The present invention further provides fragments of the estrogen receptor proteins of the present invention, in addition to proteins and peptides that comprise and consist of such fragments. The fragments to which the invention pertains, however, are not to be construed as encompassing fragments that may be disclosed publicly prior to the present invention.

As used herein, a fragment comprises at least 8 or more contiguous amino acid residues from a estrogen receptor protein. Such fragments can be chosen based on the ability to retain one or more of the biological activities of the estrogen receptor protein or could be chosen for the ability to perform a function, e.g. act as an immunogen. Particularly important fragments are biologically active fragments, peptides which are, for example, about 8 or more amino acids in length, that contain a variant amino acid residue (FIG. 2). Such fragments will typically comprise a domain or motif of the estrogen receptor proteins of the present invention, e.g., active site, ligand binding domain or DNA binding domain. Further, possible fragments include, but are not limited to, domain or motif containing fragments, soluble peptide fragments, and fragments containing immunogenic structures. Predicted domains and functional sites are readily identifiable by computer programs well-known and readily available to those of skill in the art (e.g., PROSITE analysis).

Protein/Peptide Uses

The proteins of the present invention can be used in assays to determine the biological activity of the protein, including in a panel of multiple proteins for high-throughput screening; to raise antibodies or to elicit another immune response; as a reagent (including the labeled reagent) in assays designed to quantitatively determine levels of the protein (or its binding partner or receptor) in biological fluids; and as markers for tissues in which the corresponding protein is preferentially expressed (either constitutively or at a particular stage of tissue differentiation or development or in a disease state). Any or all of these research utilities are capable of being developed into reagent grade or kit format for commercialization as research products. Methods for performing the uses listed above are well known to those skilled in the art. References disclosing such methods include “Molecular Cloning: A Laboratory Manual”, 2d ed., Cold Spring Harbor Laboratory Press, Sambrook, J., E. F. Fritsch and T. Maniatis eds., 1989, and “Methods in Enzymology: Guide to Molecular Cloning Techniques”, Academic Press, Berger, S. L. and A. R. Kimmel eds., 1987.

The estrogen receptor proteins of the present invention are useful for biological assay. Such assays involve any of the known estrogen receptor functions or activities or properties useful for the diagnosis and treatment of estrogen receptor-related conditions.

The estrogen receptor proteins of the present invention are also useful in drug screening assays, in cell-based or cell-free systems. Cell-based systems can be native, i.e., cells that normally express the receptor protein, as a biopsy or expanded in cell culture. In one embodiment, however, cell-based assays involve recombinant host cells expressing the receptor protein.

The estrogen receptor proteins of the present invention can be used to identify compounds that modulate receptor activity. Both the estrogen receptor protein of the present invention and appropriate fragments can be used in high-throughput screens to assay candidate compounds for the ability to bind and/or modulate the activity of the receptor. These compounds can be further screened against a functional receptor to determine the effect of the compound on the receptor activity. Further, these compounds can be tested in animal or invertebrate systems to determine activity/effectiveness. Compounds can be identified that activate (agonist) or inactivate (antagonist) the receptor to a desired degree. Such compounds can be selected for the ability to act on one or more of the variant estrogen receptor proteins of the present invention.

Further, the receptor polypeptides can be used to screen a compound for the ability to stimulate or inhibit interaction between the receptor protein and a target molecule that normally interacts with the receptor protein, e.g. estrogen. The target can be ligand or a binding partner that the receptor protein normally interacts (for example, an estrogen ligand or a DNA sequence). Such assays typically include the steps of combining the receptor protein with a candidate compound under conditions that allow the receptor protein, or fragment, to interact with the target molecule, and to detect the formation of a complex between the protein and the target or to detect the biochemical consequence of the interaction with the receptor protein and the target, such as any of the associated effects of DNA binding or signal transduction.

Candidate compounds include, for example, 1) peptides such as soluble peptides, including Ig-tailed fusion peptides and members of random peptide libraries (see, e.g., Lam et al., Nature 354:82-84 (1991); Houghten et al., Nature 354:84-86 (1991)) and combinatorial chemistry-derived molecular libraries made of D- and/or L- configuration amino acids; 2) phosphopeptides (e.g., members of random and partially degenerate, directed phosphopeptide libraries, see, e.g., Songyang et al., Cell 72:767-778 (1993)); 3) antibodies (e.g., polyclonal, monoclonal, humanized, anti-idiotypic, chimeric, and single chain antibodies as well as Fab, F(ab′)₂, Fab expression library fragments, and epitope-binding fragments of antibodies); and 4) small organic and inorganic molecules (e.g., molecules obtained from combinatorial and natural product libraries).

One candidate compound is a soluble fragment of the receptor that competes for ligand binding. Other candidate compounds include mutant receptors or appropriate fragments containing mutations that affect receptor function and thus compete for ligand. Accordingly, a fragment that competes for ligand, for example with a higher affinity, or a fragment that binds ligand but does not allow release, is encompassed by the invention.

The invention further includes other end point assays to identify compounds that modulate (stimulate or inhibit) receptor activity. The assays typically involve an assay of events in the signal transduction pathway that indicate receptor activity. Thus, the expression of genes that are up- or down-regulated in response to the receptor protein dependent signal cascade can be assayed. In one embodiment, the regulatory region of such genes can be operably linked to a marker that is easily detectable, such as luciferase. Alternatively, phosphorylation of the receptor protein, or a receptor protein target, could also be measured. Any of the biological or biochemical functions mediated by the receptor can be used as an endpoint assay. These include all of the biochemical or biochemical/biological events described herein, in the references cited herein, incorporated by reference for these endpoint assay targets, and other functions known to those of ordinary skill in the art.

The receptor polypeptides are also useful in competition binding assays in methods designed to discover compounds that interact with the receptor. Thus, a compound is exposed to a receptor polypeptide under conditions that allow the compound to bind or to otherwise interact with the polypeptide. Ligands to the receptor is also added to the mixture. If the test compound interacts with the receptor or ligand, it decreases the amount of complex formed or activity from the receptor target. This type of assay is particularly useful in cases in which compounds are sought that interact with specific regions of the receptor.

To perform cell free drug screening assays, it is sometimes desirable to immobilize either the receptor protein, or fragment, or its target molecule to facilitate separation of complexes from uncomplexed forms of one or both of the proteins, as well as to accommodate automation of the assay.

Techniques for immobilizing proteins on matrices can be used in the drug screening assays. In one embodiment, a fusion protein can be provided which adds a domain that allows the protein to be bound to a matrix. For example, glutathione-S-transferase/15625 fusion proteins can be adsorbed onto glutathione sepharose beads (Sigma Chemical, St. Louis, Mo.) or glutathione derivatized microtitre plates, which are then combined with the cell lysates (e.g., ³⁵S-labeled) and the candidate compound, and the mixture incubated under conditions conducive to complex formation (e.g., at physiological conditions for salt and pH). Following incubation, the beads are washed to remove any unbound label, and the matrix immobilized and radiolabel determined directly, or in the supernatant after the complexes are dissociated. Alternatively, the complexes can be dissociated from the matrix, separated by SDS-PAGE, and the level of receptor-binding protein found in the bead fraction quantitated from the gel using standard electrophoretic techniques. For example, either the polypeptide or its target molecule can be immobilized utilizing conjugation of biotin and streptavidin using techniques well known in the art. Alternatively, antibodies reactive with the protein but which do not interfere with binding of the protein to its target molecule can be derivatized to the wells of the plate, and the protein trapped in the wells by antibody conjugation. Preparations of a receptor-binding protein and a candidate compound are incubated in the receptor protein-presenting wells and the amount of complex trapped in the well can be quantitated. Methods for detecting such complexes, in addition to those described above for the GST-immobilized complexes, include immunodetection of complexes using antibodies reactive with the receptor protein target molecule, or which are reactive with receptor protein and compete with the target molecule, as well as enzyme-linked assays which rely on detecting an enzymatic activity associated with the target molecule.

Agents that modulate the protein of the present invention can be identified using one or more of the above assays, alone or in combination. It is generally preferable to use a cell-based or cell free system first and then confirm activity in an animal or other model system. Such model systems are well known in the art and can readily be employed in this context.

Modulators of receptor protein activity identified according to these drug-screening assays can be used to treat a subject with a disorder mediated by the receptor pathway, by treating cells that express the estrogen receptor protein. These methods of treatment include the steps of administering the modulators of protein activity in a pharmaceutical composition as described herein, to a subject in need of such treatment.

This invention further pertains to novel agents identified by the above-described screening assays. Accordingly, it is within the scope of this invention to further use an agent identified as described herein in an appropriate animal model. For example, an agent identified as described herein (e.g., an estrogen receptor modulating agent, an antisense estrogen receptor nucleic acid molecule, an estrogen receptor-specific antibody, or an estrogen receptor-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

The estrogen receptor proteins of the present invention are also useful to provide a target for diagnosing a disease or predisposition to disease mediated by the estrogen receptor. Accordingly, the invention provides methods for detecting the presence, or levels of, the estrogen receptor variants of the present invention (or encoding mRNA) in a cell, tissue, or organism. The method involves contacting a biological sample with a compound capable of interacting with the receptor protein (or gene or mRNA encoding the receptor) such that the interaction can be detected.

One agent for detecting a protein in a sample is an antibody capable of selectively binding to a variant form of the estrogen receptor protein. Such samples include tissues, cells and biological fluids isolated from a subject, as well as tissues, cells and fluids present within a subject.

The estrogen receptor proteins of the present invention also provide targets for diagnosing active disease, or predisposition to disease, in a patient having a variant estrogen receptor, particularly a disease involving the estrogen pathway, such as bone growth, cell differentiation, etc. Thus, the receptor can be isolated from a biological sample and assayed for the presence of a genetic mutation that results in aberrant receptor activity. This includes amino acid substitution, deletion, insertion, rearrangement, (as the result of aberrant splicing events), and inappropriate post-translational modification as provided in FIG. 2. Analytic methods include altered electrophoretic mobility, altered tryptic peptide digest, altered receptor activity in cell-based or cell-free assay, alteration in ligand or antibody-binding pattern, altered isoelectric point, direct amino acid sequencing, and any other of the known assay techniques useful for detecting mutations in a protein. Particularly useful are the variation provided in FIG. 2.

In vitro techniques for detection of peptide include enzyme linked immunosorbent assays (ELISAs), Western blots, immunoprecipitations and immunofluorescence. Alternatively, the peptide can be detected in vivo in a subject by introducing into the subject a labeled anti-peptide antibody. For example, the antibody can be labeled with a radioactive marker whose presence and location in a subject can be detected by standard imaging techniques. Particularly useful are methods that detect the specific allelic variants of the estrogen receptor disclosed herein that are expressed in a subject and methods that detect fragments of a peptide in a sample.

The peptides are also useful in pharmacogenomic analysis. Pharmacogenomics deal with clinically significant hereditary variations in the response to drugs due to altered drug disposition and abnormal action in affected persons. See, e.g., Eichelbaum, M. (Clin. Exp. Pharmacol. Physiol. 23(10-11) :983-985 (1996)), and Linder, M. W. (Clin. Chem. 43(2):254-266 (1997)). The clinical outcomes of these variations result in severe toxicity of therapeutic drugs in certain individuals or therapeutic failure of drugs in certain individuals as a result of individual variation in metabolism. Thus, the genotype of the individual can determine the way a therapeutic compound acts on the body or the way the body metabolizes the compound. Further, the activity of drug metabolizing enzymes effects both the intensity and duration of drug action. Thus, the pharmacogenomics of the individual permit the selection of effective compounds and effective dosages of such compounds for prophylactic or therapeutic treatment based on the individual's genotype. The discovery of genetic polymorphisms in some drug metabolizing enzymes has explained why some patients do not obtain the expected drug effects, show an exaggerated drug effect, or experience serious toxicity from standard drug dosages. Polymorphisms can be expressed in the phenotype of the extensive metabolizer and the phenotype of the poor metabolizer. Accordingly, genetic polymorphism may lead to allelic protein variants of the receptor protein in which one or more of the receptor functions in one population is different from those in another population. The peptides thus allow a target to ascertain a genetic predisposition that can affect treatment modality. Thus, in a ligand-based treatment, polymorphism may give rise to amino terminal extracellular domains and/or other ligand-binding regions that are more or less active in ligand binding, and receptor activation. Accordingly, ligand dosage would necessarily be modified to maximize the therapeutic effect within a given population containing a polymorphism/haplotype. As an alternative to genotyping, specific polymorphic peptides could be identified.

Antibodies

The invention also provides antibodies that selectively bind to the estrogen receptor proteins of the present invention as well as fragments thereof. As used herein, an antibody selectively binds a target protein when it binds the target protein and does not significantly bind to unrelated proteins. An antibody is still considered to selectively bind a protein even if it also binds to other proteins that are not substantially homologous with the target protein so long as such proteins share homology with a fragment or domain of the protein target of the antibody. In this case, it would be understood that antibody binding to the protein is still selective despite some degree of cross-reactivity.

As used herein, an antibody is defined in terms consistent with that recognized within the art: they are multi-subunit proteins produced by a mammalian organism in response to an antigen challenge. The antibodies of the present invention include polyclonal antibodies and monoclonal antibodies, as well as fragments of such antibodies, including, but not limited to, Fab or F(ab′)₂, and Fv fragments.

Many methods are known for generating and/or identifying antibodies to a given target peptide. Several such methods are described by Harlow, Antibodies, Cold Spring Harbor Press, (1989). In general, to generate antibodies, an isolated peptide is used as an immunogen and is administered to a mammalian organism, such as a rat, rabbit or mouse. The full-length protein, an antigenic peptide fragment or a fusion protein can be used.

Antibodies are preferably prepared from regions or discrete fragments of the estrogen receptor protein. Antibodies can be prepared from any region of the peptide as described herein. However, preferred regions will include those involved in function/activity and/or receptor/binding partner interaction. An antigenic fragment will typically comprise at least 10 contiguous amino acid residues. The antigenic peptide can comprise, however, at least 12, 14, 20 or more amino acid residues. Such fragments can be selected on a physical property, such as fragments correspond to regions that are located on the surface of the protein, e.g., hydrophilic regions or can be selected based on sequence uniqueness.

Detection on an antibody of the present invention can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibody Uses

The antibodies can be used to isolate the estrogen receptor protein of the present invention by standard techniques, such as affinity chromatography or immunoprecipitation. The antibodies can facilitate the purification of the natural protein from cells and recombinantly produced protein expressed in host cells. In addition, such antibodies are useful to detect the presence of the estrogen receptor protein of the present invention in cells or tissues to determine the pattern of expression of the protein among various tissues in an organism and over the course of normal development. Further, such antibodies can be used to detect protein in situ, in vitro, or in a cell lysate or supernatant in order to evaluate the abundance and pattern of expression. Also, such antibodies can be used to assess abnormal tissue distribution or abnormal expression during development. Antibody detection of circulating fragments of the full length estrogen receptor protein can be used to identify turnover.

Further, the antibodies can be used to assess expression in disease states such as in active stages of the disease or in an individual with a predisposition toward disease related to the protein's function, particularly diseases involving bone growth/formation/degeneration. When a disorder is caused by an inappropriate tissue distribution, developmental expression, level of expression of the protein, or expressed/processed form, the antibody can be prepared against the normal protein. If a disorder is characterized by a specific mutation in the protein, antibodies specific for this mutant protein can be used to assay for the presence of the specific mutant protein.

The antibodies can also be used to assess normal and aberrant subcellular localization of cells in the various tissues in an organism. The diagnostic uses can be applied, not only in genetic testing, but also in monitoring a treatment modality. Accordingly, where treatment is ultimately aimed at correcting the expression level or the presence of aberrant sequence and aberrant tissue distribution or developmental expression, antibodies directed against the protein or relevant fragments can be used to monitor therapeutic efficacy.

Additionally, antibodies are useful in pharmacogenomic analysis. Thus, antibodies prepared against polymorphic proteins can be used to identify individuals that require modified treatment modalities. The antibodies are also useful as diagnostic tools as an immunological marker for aberrant estrogen receptor protein analyzed by electrophoretic mobility, isoelectric point, tryptic peptide digest, and other physical assays known to those in the art.

The antibodies are also useful for inhibiting protein function, for example, blocking the binding of the estrogen receptor protein to a binding partner such as a ligand. These uses can also be applied in a therapeutic context in which treatment involves inhibiting the protein's function. An antibody can be used, for example, to block binding, thus modulating (agonizing or antagonizing) the peptides activity. Antibodies can be prepared against specific fragments containing sites required for function or against intact protein that is associated with a cell or cell membrane.

The invention also encompasses kits for using antibodies to detect the presence of a protein in a biological sample. The kit can comprise antibodies such as a labeled or labelable antibody and a compound or agent for detecting estrogen receptor protein in a biological sample; means for determining the amount of protein in the sample; means for comparing the amount of estrogen receptor protein in the sample with a standard; and instructions for use.

Nucleic Acid Molecules

The present invention further provides isolated nucleic acid molecules that encode any of the estrogen receptor proteins of the present invention. Such nucleic acid molecules will consist of, consist essentially of, or comprise a nucleotide sequence that encodes one of the estrogen receptor proteins of the present invention.

As used herein, an “isolated” nucleic acid molecule is one that is separated from other nucleic acid present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. However, there can be some flanking nucleotide sequences, for example up to about 5 KB, 4 KB, 3 KB, 2 KB, or 1 KB or less, particularly contiguous peptide encoding sequences and peptide encoding sequences within the same gene but separated by introns in the genomic sequence. The important point is that the nucleic acid is isolated from remote and unimportant flanking sequences such that it can be subjected to the specific manipulations described herein such as recombinant expression, preparation of probes and primers, and other uses specific to the nucleic acid sequences.

Moreover, an “isolated” nucleic acid molecule, such as a cDNA molecule, can be substantially free of other cellular material, or culture medium when produced by recombinant techniques, or chemical precursors or other chemicals when chemically synthesized. However, the nucleic acid molecule can be fused to other coding or regulatory sequences and still be considered isolated.

For example, recombinant DNA molecules contained in a vector are considered isolated. Further examples of isolated DNA molecules include recombinant DNA molecules maintained in heterologous host cells or purified (partially or substantially) DNA molecules in solution. Isolated RNA molecules include in vivo or in vitro RNA transcripts of the isolated DNA molecules of the present invention. Isolated nucleic acid molecules according to the present invention further include such molecules produced synthetically.

Accordingly, the present invention provides nucleic acid molecules that consist of the nucleotide sequences shown in FIG. 1, including one or more of the sequence polymorphisms provided in FIG. 2. A nucleic acid molecule consists of a nucleotide sequence when the nucleotide sequence is the complete nucleotide sequence of the nucleic acid molecule.

The present invention further provides nucleic acid molecules that consist essentially of the nucleotide sequence shown in FIG. 1, including one or more of the sequence polymorphisms provided in FIG. 2. A nucleic acid molecule consists essentially of a nucleotide sequence when such a nucleotide sequence is present with only a few additional nucleic acid residues in the final nucleic acid molecule.

The present invention further provides nucleic acid molecules that are comprised of the nucleotide sequences shown in FIG. 1, including one or more of the sequence polymorphisms provided in FIG. 2. A nucleic acid molecule is comprised of a nucleotide sequence when the nucleotide sequence is at least part of the final nucleotide sequence of the nucleic acid molecule. In such a fashion, the nucleic acid molecule can be only the nucleotide sequence or have additional nucleic acid residues, such as nucleic acid residues that are naturally associated with it or heterologous nucleotide sequences. Such a nucleic acid molecule can have a few additional nucleotides or can comprise several hundred or more additional nucleotides. A brief description of how various types of these nucleic acid molecules can be readily made/isolated is provided below.

The isolated nucleic acid molecules can encode the mature protein plus additional amino or carboxyl-terminal amino acids, or amino acids interior to the mature peptide (when the mature form has more than one peptide chain, for instance). Such sequences may play a role in processing of a protein from precursor to a mature form, facilitate protein trafficking, prolong or shorten protein half-life or facilitate manipulation of a protein for assay or production, among other things. As generally is the case in situ, the additional amino acids may be processed away from the mature protein by cellular enzymes.

As mentioned above, the isolated nucleic acid molecules include, but are not limited to, the sequence encoding the estrogen receptor protein alone, the sequence encoding the mature peptide and additional coding sequences, such as a leader or secretory sequence (e.g., a pre-pro or pro-protein sequence), the sequence encoding the mature peptide, with or without the additional coding sequences, plus additional non-coding sequences, for example introns and non-coding 5′ and 3′ sequences such as transcribed but non-translated sequences that play a role in transcription, mRNA processing (including splicing and polyadenylation signals), ribosome binding and stability of mRNA, as well as genomic regulatory sequences such as promoters. In addition, the nucleic acid molecule may be fused to a marker sequence encoding, for example, a peptide that facilitates purification.

Isolated nucleic acid molecules can be in the form of RNA, such as mRNA, or in the form DNA, including cDNA and genomic DNA obtained by cloning or produced by chemical synthetic techniques or by a combination thereof. The nucleic acid, especially DNA, can be double-stranded or single-stranded. Single-stranded nucleic acid can be the coding strand (sense strand) or the non-coding strand (anti-sense strand).

The invention further provides nucleic acid molecules that encode fragments of the proteins of the present invention. A fragment comprises a contiguous nucleotide sequence greater than 12 or more nucleotides. Further, a fragment could at least 30, 40, 50, 100, 250 or 500 nucleotides in length. The length of the fragment will be based on its intended use. For example, the fragment can encode epitope-bearing regions of the peptide, or can be useful as DNA probes and primers. Such fragments can be isolated using the known nucleotide sequence to synthesize an oligonucleotide probe. A labeled probe can then be used to screen a cDNA library, genomic DNA library, or mRNA to isolate nucleic acid corresponding to the coding region. Further, primers can be used in PCR reactions to clone specific regions of gene.

A probe/primer typically comprises substantially a purified oligonucleotide or oligonucleotide pair. The oligonucleotide typically comprises a region of nucleotide sequence that hybridizes under stringent conditions to at least about 12, 20, 25, 40, 50 or more consecutive nucleotides.

As used herein, the term “hybridizes under stringent conditions” is intended to describe conditions for hybridization and washing under which nucleotide sequences encoding a peptide at least 50-55% homologous to each other typically remain hybridized to each other. The conditions can be such that sequences at least about 65%, at least about 70%, or at least about 75% or more homologous to each other typically remain hybridized to each other. Such stringent conditions are known to those skilled in the art and can be found in Current Protocols in Molecular Biology, John Wiley & Sons, N.Y. (1989), 6.3.1-6.3.6. One example of stringent hybridization conditions are hybridization in 6×sodium chloride/sodium citrate (SSC) at about 45° C., followed by one or more washes in 0.2×SSC, 0.1% SDS at 50-65° C.

Nucleic Acid Molecule Uses

The nucleic acid molecules of the present invention are useful for probes, primers, chemical intermediates, and in biological assays. The probe can correspond to any sequence along the entire length of the nucleic acid molecules provided in FIG. 1, including one or more of the sequence polymorphisms provided in FIG. 2. Accordingly, it could be derived from 5′ noncoding regions, the coding region, and 3′ noncoding regions. However, as discussed, fragments are not to be construed as encompassing fragments disclosed prior to the present invention.

The nucleic acid molecules are also useful as primers for PCR to amplify any given region of a nucleic acid molecule and are useful to synthesize antisense molecules of desired length and sequence.

The nucleic acid molecules are also useful for constructing recombinant vectors. Such vectors include expression vectors that express a portion of, or all of, the peptide sequences. Vectors also include insertion vectors, used to integrate into another nucleic acid molecule sequence, such as into the cellular genome, to alter in situ expression of a gene and/or gene product. For example, an endogenous coding sequence can be replaced via homologous recombination with all or part of the coding region containing one or more specifically introduced mutations.

The nucleic acid molecules are also useful for expressing antigenic portions of the proteins.

The nucleic acid molecules are also useful for designing ribozymes corresponding to all, or a part, of the mRNA produced from the nucleic acid molecules described herein.

The nucleic acid molecules are also useful for constructing host cells expressing a part, or all, of the nucleic acid molecules and peptides.

The nucleic acid molecules are also useful for constructing transgenic animals expressing all, or a part, of the nucleic acid molecules and peptides.

The nucleic acid molecules are also useful for making vectors that express part, or all, of the peptides.

The nucleic acid molecules are also useful as hybridization probes for determining the presence, level, form and distribution of nucleic acid expression. Accordingly, the probes can be used to detect the presence of, or to determine levels of, a specific nucleic acid molecule in cells, tissues, and in organisms. The nucleic acid whose level is determined can be DNA or RNA. Accordingly, probes corresponding to the peptides described herein can be used to assess expression and/or gene copy number in a given cell, tissue, or organism. These uses are relevant for diagnosis of disorders involving an increase or decrease in estrogen receptor protein expression relative to normal results.

In vitro techniques for detection of mRNA include Northern hybridizations and in situ hybridizations. In vitro techniques for detecting DNA include Southern hybridizations and in situ hybridization.

Probes can be used as a part of a diagnostic test kit for identifying cells or tissues that express a estrogen receptor proteins of the present invention, such as by measuring a level of a receptor-encoding nucleic acid in a sample of cells from a subject e.g., mRNA or genomic DNA, or determining if a receptor gene has been mutated.

Nucleic acid expression assays are useful for drug screening to identify compounds that modulate estrogen receptor nucleic acid expression.

The invention thus provides a method for identifying a compound that can be used to treat a disorder associated with nucleic acid expression of the estrogen receptor gene. The method typically includes assaying the ability of the compound to modulate the expression of the estrogen receptor nucleic acid and thus identifying a compound that can be used to treat a disorder characterized by undesired estrogen receptor nucleic acid expression. The assays can be performed in cell-based and cell-free systems. Cell-based assays include cells naturally expressing the estrogen receptor nucleic acid or recombinant cells genetically engineered to express specific nucleic acid sequences.

The assay for estrogen receptor nucleic acid expression can involve direct assay of nucleic acid levels, such as mRNA levels, or on collateral compounds involved in the signal pathway. Further, the expression of genes that are up- or down-regulated in response to the estrogen receptor protein signal pathway can also be assayed. In this embodiment the regulatory regions of these genes can be operably linked to a reporter gene such as luciferase.

Thus, modulators of estrogen receptor gene expression can be identified in a method wherein a cell is contacted with a candidate compound and the expression of mRNA determined. The level of expression of estrogen receptor mRNA in the presence of the candidate compound is compared to the level of expression of estrogen receptor mRNA in the absence of the candidate compound. The candidate compound can then be identified as a modulator of nucleic acid expression based on this comparison and be used, for example to treat a disorder characterized by aberrant nucleic acid expression. When expression of mRNA is statistically significantly greater in the presence of the candidate compound than in its absence, the candidate compound is identified as a stimulator of nucleic acid expression. When nucleic acid expression is statistically significantly less in the presence of the candidate compound than in its absence, the candidate compound is identified as an inhibitor of nucleic acid expression.

The invention further provides methods of treatment, with the nucleic acid as a target, using a compound identified through drug screening as a gene modulator to modulate estrogen receptor nucleic acid expression. Modulation includes both up-regulation (i.e. activation or agonization) or down-regulation (suppression or antagonization) or nucleic acid expression.

Alternatively, a modulator for estrogen receptor nucleic acid expression can be a small molecule or drug identified using the screening assays described herein as long as the drug or small molecule inhibits the estrogen receptor nucleic acid expression.

The nucleic acid molecules are also useful for monitoring the effectiveness of modulating compounds on the expression or activity of the estrogen receptor gene in clinical trials or in a treatment regimen. Thus, the gene expression pattern can serve as a barometer for the continuing effectiveness of treatment with the compound, particularly with compounds to which a patient can develop resistance. The gene expression pattern can also serve as a marker indicative of a physiological response of the affected cells to the compound. Accordingly, such monitoring would allow either increased administration of the compound or the administration of alternative compounds to which the patient has not become resistant. Similarly, if the level of nucleic acid expression falls below a desirable level, administration of the compound could be commensurately decreased.

The nucleic acid molecules are also useful in diagnostic assays for qualitative changes in estrogen receptor nucleic acid, and particularly in qualitative changes that lead to pathology. The nucleic acid molecules can be used to detect mutations in estrogen receptor genes and gene expression products such as mRNA. The nucleic acid molecules can be used as hybridization probes to detect naturally-occurring genetic mutations in the estrogen receptor gene and thereby to determine whether a subject with the mutation is at risk for a disorder caused by the mutation. Mutations include deletion, addition, or substitution of one or more nucleotides in the gene, chromosomal rearrangement, such as inversion or transposition, modification of genomic DNA, such as aberrant methylation patterns or changes in gene copy number, such as amplification. Detection of a mutated form of the estrogen receptor gene associated with a dysfunction provides a diagnostic tool for an active disease or susceptibility to disease when the disease results from overexpression, underexpression, or altered expression of a estrogen receptor protein.

Individuals carrying mutations in the estrogen receptor gene can be detected at the nucleic acid level by a variety of techniques. Genomic DNA can be analyzed directly or can be amplified by using PCR prior to analysis. RNA or cDNA can be used in the same way. In some uses, detection of the mutation involves the use of a probe/primer in a polymerase chain reaction (PCR) (see, e.g. U.S. Pat. Nos. 4,683,195 and 4,683,202), such as anchor PCR or RACE PCR, or, alternatively, in a ligation chain reaction (LCR) (see, e.g., Landegran et al., Science 241:1077-1080 (1988); and Nakazawa et al., PNAS 91:360-364 (1994)), the latter of which can be particularly useful for detecting point mutations in the gene (see Abravaya et al., Nucleic Acids Res. 23:675-682 (1995)). This method can include the steps of collecting a sample of cells from a patient, isolating nucleic acid (e.g., genomic, mRNA or both) from the cells of the sample, contacting the nucleic acid sample with one or more primers which specifically hybridize to a gene under conditions such that hybridization and amplification of the gene (if present) occurs, and detecting the presence or absence of an amplification product, or detecting the size of the amplification product and comparing the length to a control sample. Deletions and insertions can be detected by a change in size of the amplified product compared to the normal genotype. Point mutations can be identified by hybridizing amplified DNA to normal RNA or antisense DNA sequences.

Alternatively, mutations in a estrogen receptor gene can be directly identified, for example, by alterations in restriction enzyme digestion patterns determined by gel electrophoresis.

Further, sequence-specific ribozymes (U.S. Pat. No. 5,498,531) can be used to score for the presence of specific mutations by development or loss of a ribozyme cleavage site. Perfectly matched sequences can be distinguished from mismatched sequences by nuclease cleavage digestion assays or by differences in melting temperature.

Sequence changes at specific locations can also be assessed by nuclease protection assays such as RNase and S1 protection or the chemical cleavage method. Furthermore, sequence differences between a mutant estrogen receptor gene and a wild-type gene can be determined by direct DNA sequencing. A variety of automated sequencing procedures can be utilized when performing the diagnostic assays ((1995) Biotechniques 19:448), including sequencing by mass spectrometry (see, e.g., PCT International Publication No. WO 94/16101; Cohen et al., Adv. Chromatogr. 36:127-162 (1996); and Griffin et al., Appl. Biochem. Biotechnol. 38:147-159 (1993)).

Other methods for detecting mutations in the gene include methods in which protection from cleavage agents is used to detect mismatched bases in RNA/RNA or RNA/DNA duplexes (Myers et al., Science 230:1242 (1985)); Cotton et al., PNAS85:4397 (1988); Saleeba et al., Meth. Enzymol. 217:286-295 (1992)), electrophoretic mobility of mutant and wild type nucleic acid is compared (Orita et al., PNAS 86:2766 (1989); Cotton et al., Mutat. Res. 285:125-144 (1993); and Hayashi et al., Genet. Anal. Tech. Appl. 9:73-79 (1992)), and movement of mutant or wild-type fragments in polyacrylamide gels containing a gradient of denaturant is assayed using denaturing gradient gel electrophoresis (Myers et al., Nature 313:495 (1985)). Examples of other techniques for detecting point mutations include, selective oligonucleotide hybridization, selective amplification, and selective primer extension.

The nucleic acid molecules are also useful for testing an individual for a genotype that while not necessarily causing diseases; nevertheless affects the treatment modality. Thus, the nucleic acid molecules can be used to study the relationship between an individual's genotype and the individual's response to a compound used for treatment (pharmacogenomic relationship). Accordingly, the nucleic acid molecules described herein can be used to assess the mutation content of the estrogen receptor gene in an individual in order to select an appropriate compound or dosage regimen for treatment.

Thus nucleic acid molecules displaying genetic variations that affect treatment provide a diagnostic target that can be used to tailor treatment in an individual. Accordingly, the production of recombinant cells and animals containing these polymorphism/haplotypes allow effective clinical design of treatment compounds and dosage regimens.

The nucleic acid molecules are thus useful as antisense constructs to control estrogen receptor gene expression in cells, tissues, and organisms. A DNA antisense nucleic acid molecule is designed to be complementary to a region of the gene involved in transcription, preventing transcription and hence production of estrogen receptor protein. An antisense RNA or DNA nucleic acid molecule would hybridize to the mRNA and thus block translation of mRNA into estrogen receptor protein.

Alternatively, a class of antisense molecules can be used to inactivate mRNA in order to decrease expression of estrogen receptor nucleic acid. Accordingly, these molecules can treat a disorder characterized by abnormal or undesired estrogen receptor nucleic acid expression. This technique involves cleavage by means of ribozymes containing nucleotide sequences complementary to one or more regions in the mRNA that attenuate the ability of the mRNA to be translated. Possible regions include coding regions and particularly coding regions corresponding to the catalytic and other functional activities of the estrogen receptor proteins of the present invention, such as ligand binding.

The nucleic acid molecules also provide vectors for gene therapy in patients containing cells that are aberrant in estrogen receptor gene expression. Thus, recombinant cells, which include the patient's cells that have been engineered ex vivo and returned to the patient, are introduced into an individual where the cells produce the desired estrogen receptor protein to treat the individual.

The invention also encompasses kits for detecting the presence of a estrogen receptor nucleic acid in a biological sample. For example, the kit can comprise reagents such as a labeled or labelable nucleic acid or agent capable of detecting estrogen receptor nucleic acid in a biological sample; means for determining the amount of estrogen receptor nucleic acid in the sample; and means for comparing the amount of estrogen receptor nucleic acid in the sample with a standard. The compound or agent can be packaged in a suitable container. The kit can further comprise instructions for using the kit to detect estrogen receptor protein mRNA or DNA.

Design of SNP-Containing Nucleic Acids Detection Methods

The SNP-containing nucleic acid molecules of the present invention are useful as probes, primers, chemical intermediates, and in biological assays for SNPs of the present invention. The probes/primers can correspond to one or more of the SNPs provided in FIG. 2 or can correspond to a specific region 5′ and/or 3′ to a SNP position. However, as discussed above, fragments are not to be construed as encompassing fragments that are not associated with SNPs of the present invention or those known in the art for SNP detection. The SNP-containing nucleic acid molecules and information provided herein are also useful for designing primers for PCR to amplify any given SNP of the present invention and to design any formatted SNP detection reagent/kits.

A probe/primer typically comprises substantially a purified oligonucleotide or oligonucleotide pair. The oligonucleotide typically comprises a region of nucleotide sequence that hybridizes under stringent conditions to at least about 12, 20, 25, 40, 50 or more consecutive nucleotides. Depending on the particular application, the consecutive nucleotides can either include the target SNP position, or be a specific region in close enough proximity 5′ and/or 3′ to the SNP position to carry out the desired assay.

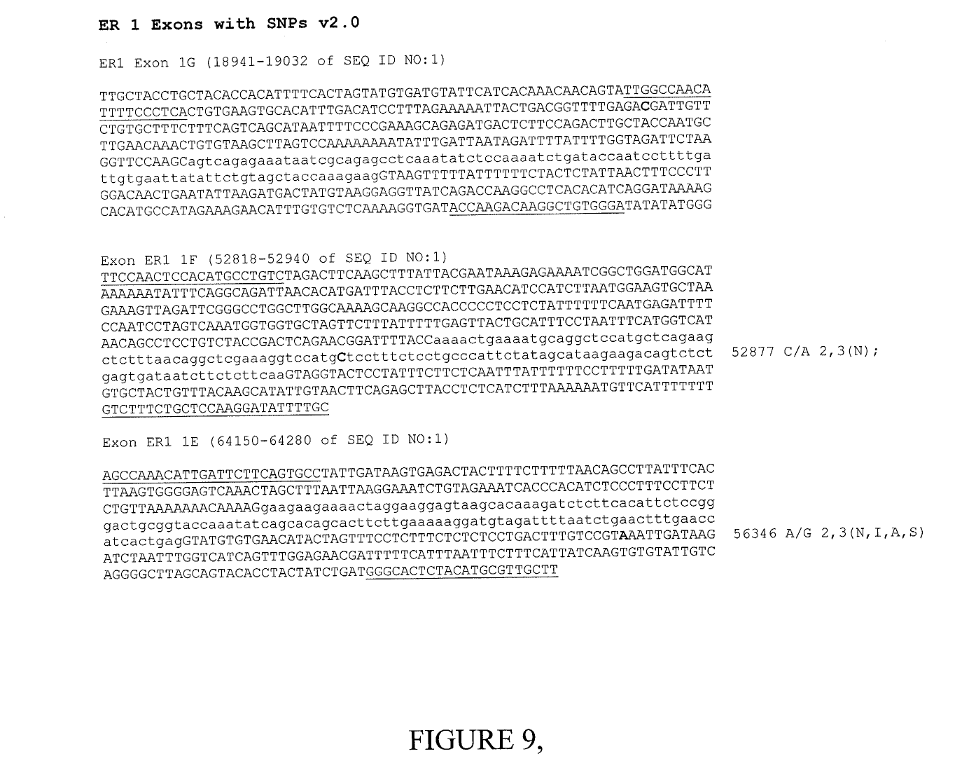

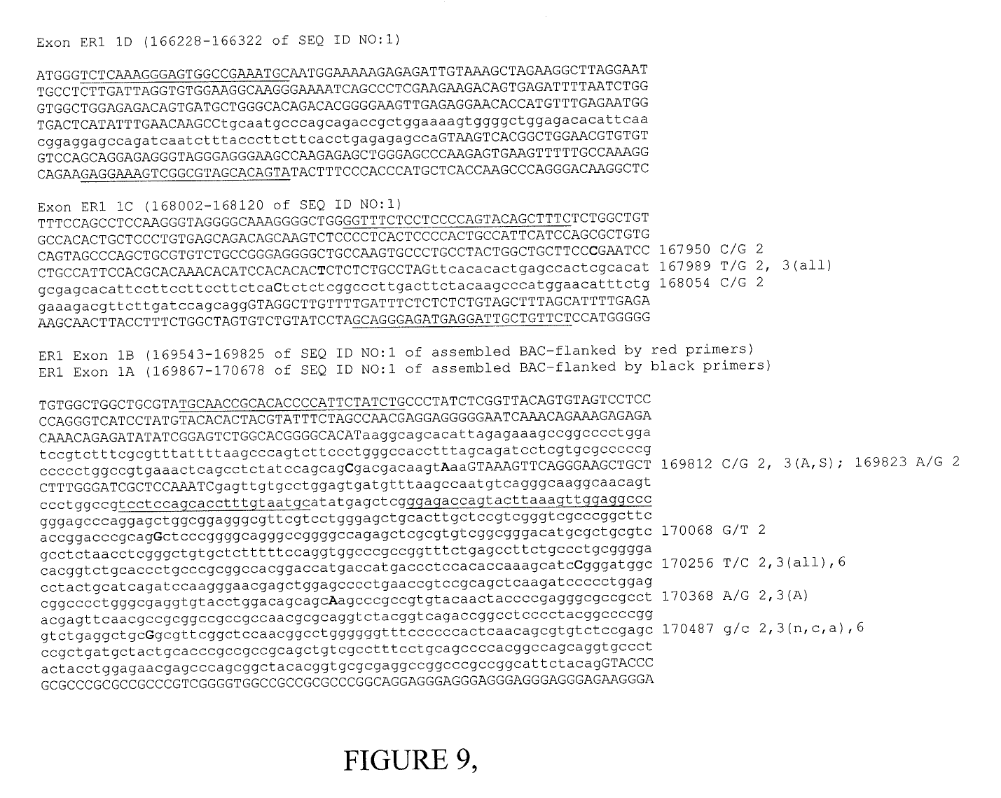

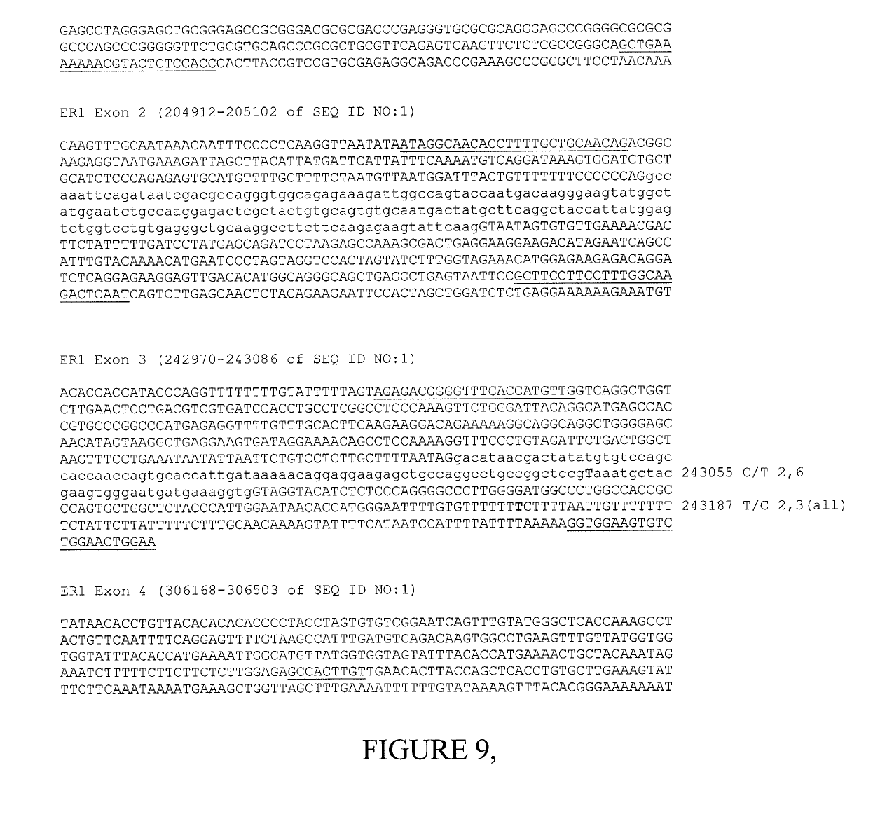

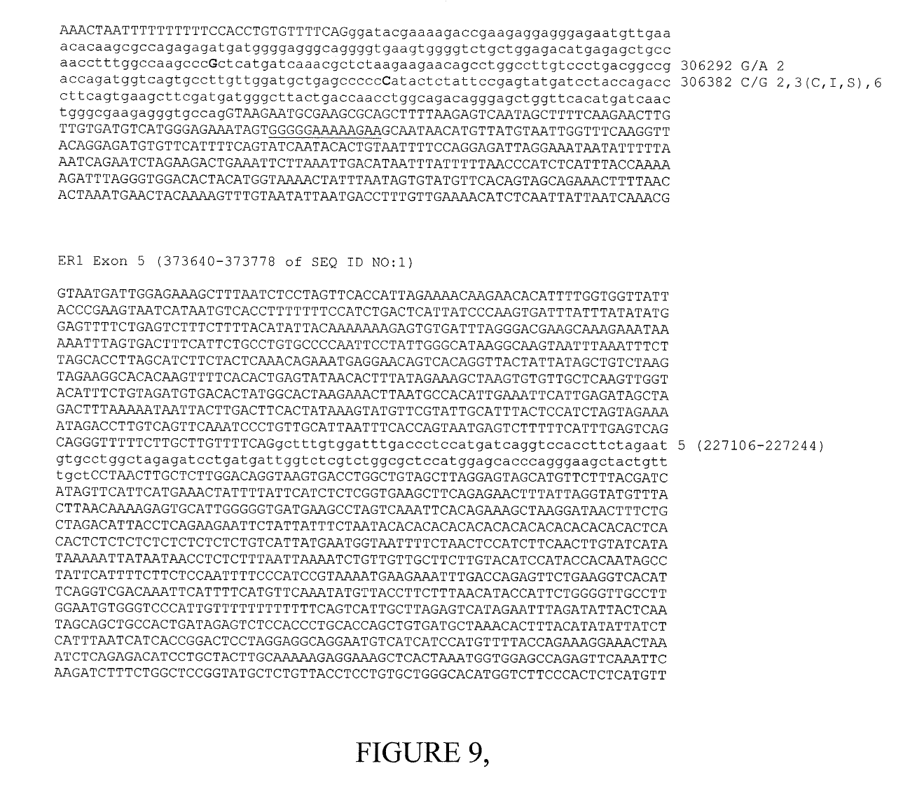

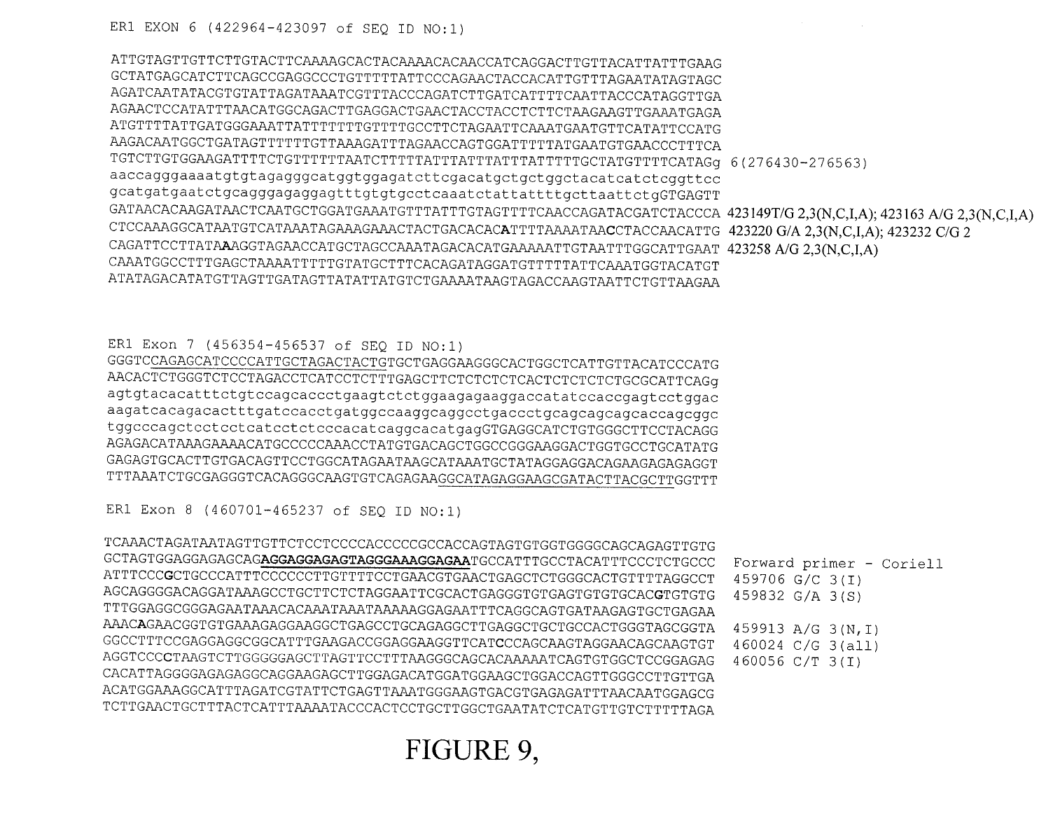

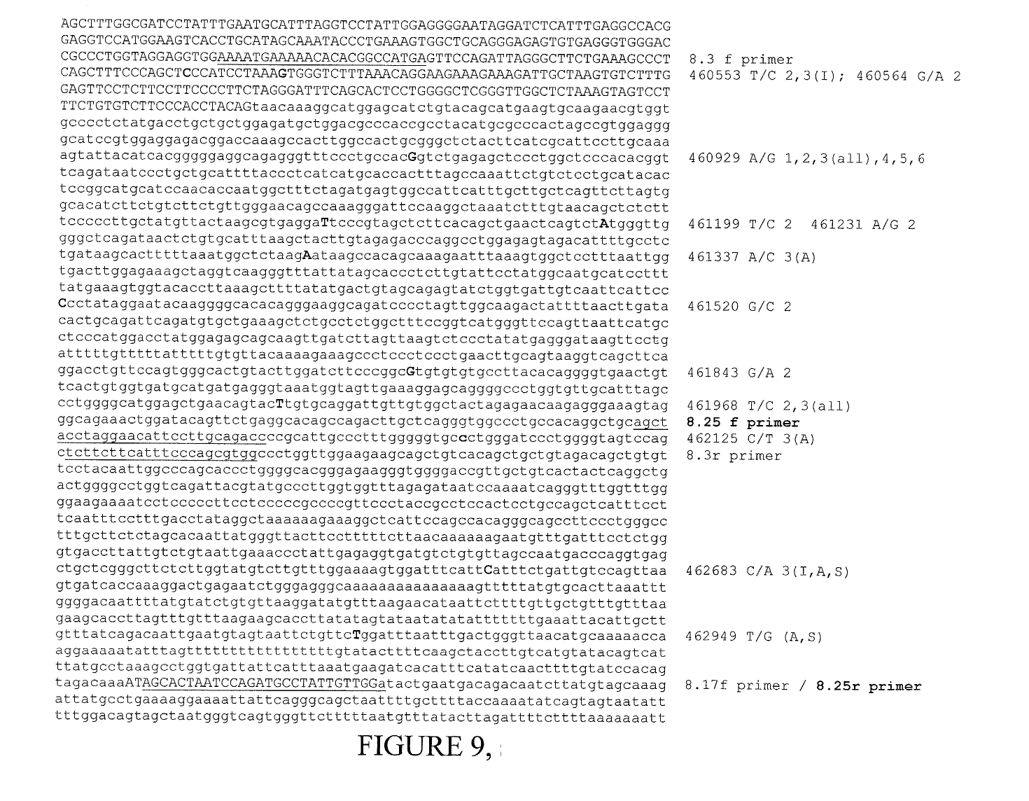

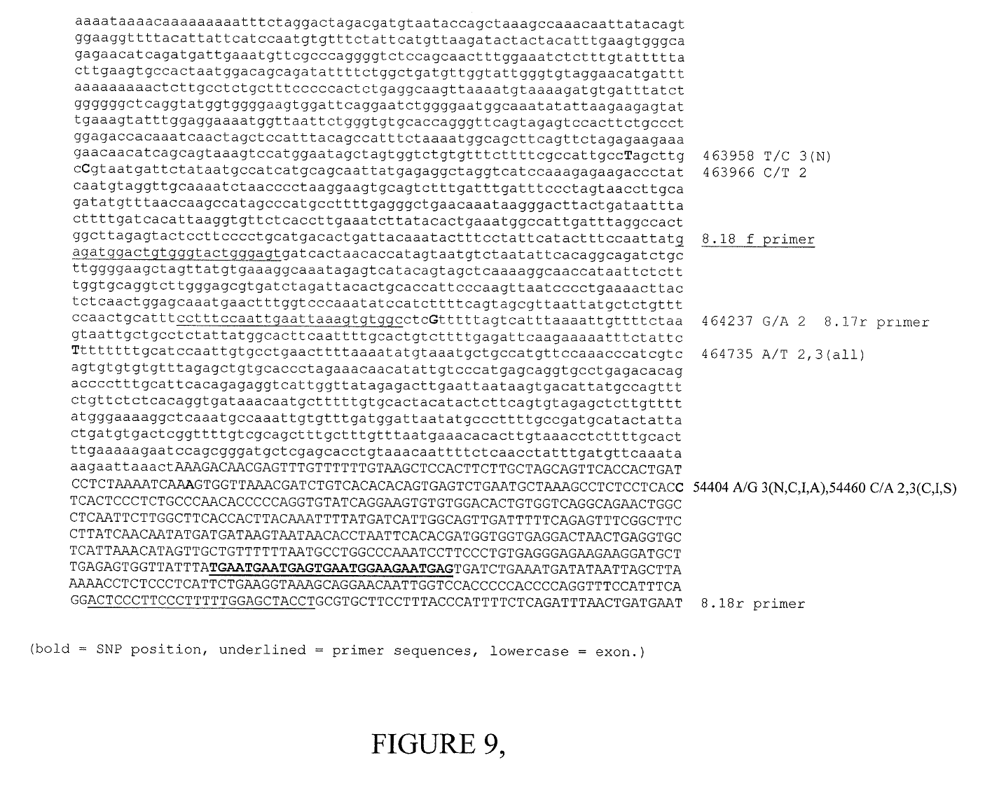

Preferred primer and probe sequences can readily be determined using the sequences provided in FIGS. 1, 2, and 9. It will be apparent to one of skill in the art that such primers and probes are useful as diagnostic probes or amplification primers for genotyping SNPs of the present invention, and can be incorporated into a kit format.

For analyzing SNPs, it may be appropriate to use oligonucleotides specific to alternative SNP alleles (referred to as “allele-specific oligonucleotides”, “allele-specific probes”, or “allele-specific primers”). The design and use of allele-specific probes for analyzing polymorphisms is described by e.g., Saiki et al., Nature 324, 163-166 (1986); Dattagupta, EP 235,726, Saiki, WO 89/11548.

In a hybridization-based assay, allele-specific probes can be designed that hybridize to a segment of target DNA from one individual but do not hybridize to the corresponding segment from another individual due to the presence of different polymorphic forms in the respective segments from the two individuals. Hybridization conditions should be sufficiently stringent that there is a significant difference in hybridization intensity between alleles, and preferably an essentially binary response, whereby a probe hybridizes to only one of the alleles. Some probes are designed to hybridize to a segment of target DNA such that the polymorphic site aligns with a central position (e.g., in a 15-mer at the 7 position; in a 16-mer, at either the 8 or 9 position) of the probe. This design of probe achieves good discrimination in hybridization between different allelic forms.

Allele-specific probes are often used in pairs, the “pairs” may be identical except for a one nucleotide mismatch that represents the allelic variants at the SNP position. One member of a pair perfectly matches a reference form of a target sequence and the other member perfectly matches a variant form. In the case of an array, several pairs of probes can then be immobilized on the same support for simultaneous analysis of multiple polymorphisms within the same target sequence.

In one type of PCR-based assay, an allele-specific primer hybridizes to a site on target DNA overlapping the SNP position and only primes amplification of an allelic form to which the primer exhibits perfect complementarity. See Gibbs, Nucleic Acid Res. 17 2427-2448 (1989). This primer is used in conjunction with a second primer that hybridizes at a distal site. Amplification proceeds from the two-primers, resulting in a detectable product that indicates the particular allelic form is present. A control is usually performed with a second pair of primers, one of which shows a single base mismatch at the polymorphic site and the other of which exhibits perfect complementarity to a distal site. The single-base mismatch prevents amplification and no detectable product is formed. The method works best when the mismatch is included in the 3′-most position of the oligonucleotide aligned with the polymorphism because this position is most destabilizing to elongation from the primer (see, e.g., WO 93/22456). This PCR-based assay can be utilized as part of the TaqMan assay, described below.

SNP Detection Kits, Nucleic Acid Arrays, and Integrated Systems

The present invention further provides SNP detection kits, such as arrays or microarrays of nucleic acid molecules, or probe/primer sets, that are based on the SNPs provided in FIGS. 1, 2, 4, 8, 9

In one embodiment of the present invention, kits are provided which contain the necessary reagents to carry out one or more assays that detect one or more SNPs disclosed herein. The present invention also provides multicomponent integrated systems for analyzing the SNPs provided by the present invention.

SNP detection kits may contain one or more oligonucleotide probes, or pairs of probes, that hybridize at or near each SNP position. Multiple pairs of allele-specific oligonucleotides may be included in the kit to simultaneously assay large numbers of SNPs, at least one of which is one of the SNPs of the present invention. In some kits, such as arrays, the allele-specific oligonucleotides are provided immobilized to a substrate. For example, the same substrate can comprise allele-specific oligonucleotide probes for detecting at least 1; 10; 100; 1000; 10,000; 100,000; 300,000 or substantially all of the polymorphisms shown in FIGS. 1, 2, 4, 8 and 9.

Specifically, the invention provides a compartmentalized kit to receive, in close confinement, one or more containers which comprises: (a) a first container comprising one of the nucleic acid probes, for example an allele-specific oligonucleotide, that can bind to a fragment of the human genome containing a SNP disclosed herein; and (b) one or more other containers comprising one or more of the following: wash reagents or reagents capable of detecting the presence of a bound probe.

In detail, a compartmentalized kit includes any kit in which reagents are contained in separate containers. Such containers include small glass containers, plastic containers, strips of plastic, glass or paper, or arraying material such as silica. Such containers allow one to efficiently transfer reagents from one compartment to another compartment such that the samples and reagents are not cross-contaminated, and the agents or solutions of each container can be added in a quantitative fashion from one compartment to another. Such containers may include a container which will accept the test sample, a container which contains the SNP probe, containers which contain wash reagents (such as phosphate buffered saline, Tris-buffers, etc.), and containers which contain the reagents used to detect the bound probe. The kit can further comprise reagents for PCR or other enzymatic reactions, and instructions for using the kit. One skilled in the art will readily recognize that the previously unidentified SNPs of the present invention can be routinely identified using the sequence information disclosed herein and can be readily incorporated into one of the established kit formats which are well known in the art.

The present invention further provides arrays or microarrays of nucleic acid molecules that are based on the sequence information provided in FIG. 1, including one or more of the variations provided in FIG. 2.

As used herein “Arrays” or “Microarrays” refers to an array of distinct polynucleotides or oligonucleotides synthesized on a substrate, such as paper, nylon or other type of membrane, filter, chip, glass slide, or any other suitable solid support. In one embodiment, the microarray is prepared and used according to the methods described in U.S. Pat. No. 5,837,832, Chee et al., PCT application WO95/11995 (Chee et al.), Lockhart, D. J. et al. (1996; Nat. Biotech. 14: 1675-1680) and Schena, M. et al. (1996; Proc. Natl. Acad. Sci. 93: 10614-10619), all of which are incorporated herein in their entirety by reference. In other embodiments, such arrays are produced by the methods described by Brown et al., U.S. Pat. No. 5,807,522. Arrays or microarrays are commonly referred to as “DNA chips”.

Any number of oligonucleotide probes, such as allele-specific oligonucleotides, may be implemented in an array, wherein each probe or pair of probes corresponds to a different SNP position. The oligonucleotides are synthesized at designated areas on a substrate using a light-directed chemical process. The substrate may be paper, nylon or other type of membrane, filter, chip, glass slide or any other suitable solid support.

Hybridization assays based on oligonucleotide arrays rely on the differences in hybridization stability of short oligonucleotides probes to perfectly matched and mismatched target sequence variants. Efficient access to polymorphism information is obtained through a basic structure comprising high-density arrays of oligonucleotide probes attached to a solid support (e.g., a chip) at selected positions. Each DNA chip can contain thousands to millions of individual synthetic DNA probes arranged in a grid-like pattern and miniaturized to the size of a dime, each corresponding to a particular SNP position or allelic variant. Preferably, probes are attached to a solid support in an ordered, addressable array.

The array/chip technology has already been applied with success in numerous cases. For example, the screening of mutations has been undertaken in the BRCA1 gene, in S. cerevisiae mutant strains, and in the protease gene of HIV-I virus (Hacia et al., 1996; Shoemaker et al., 1996; Kozal et al., 1996). Chips of various formats for use in detecting SNPs can be produced on a customized basis.

An array-based tiling strategy useful for detecting SNPs is described in EP 785280. Briefly, arrays may generally be “tiled” for a large number of specific polymorphisms. “Tiling” refers to the synthesis of a defined set of oligonucleotide probes that are made up of a sequence complementary to the target sequence of interest, as well as preselected variations of that sequence, e.g., substitution of one or more given positions with one or more members of the basis set of monomers, i.e. nucleotides. Tiling strategies are further described in PCT application No. WO 95/11995. In a particular aspect, arrays are tiled for a number of specific SNPs. In particular, the array is tiled to include a number of detection blocks, each detection block being specific for a specific SNP or a set of SNPs. For example, a detection block may be tiled to include a number of probes that span the sequence segment that includes a specific SNP. To ensure probes that are complementary to each allele, the probes are synthesized in pairs differing at the SNP position. In addition to the probes differing at the SNP position, monosubstituted probes are also generally tiled within the detection block. Such methods can readily be applied to the SNP information disclosed herein.

These monosubstituted probes have bases at and up to a certain number of bases in either direction from the polymorphism, substituted with the remaining nucleotides (selected from A, T, G, C and U). Typically the probes in a tiled detection block will include substitutions of the sequence positions up to and including those that are 5 bases away from the SNP. The monosubstituted probes provide internal controls for the tiled array, to distinguish actual hybridization from artefactual cross-hybridization. Upon completion of hybridization with the target sequence and washing of the array, the array is scanned to determine the position on the array to which the target sequence hybridizes. The hybridization data from the scanned array is then analyzed to identify which allele or alleles of the SNP are present in the sample. Hybridization and scanning may be carried out as described in PCT application No. WO 92/10092 and WO 95/11995 and U.S. Pat. No. 5,424,186.

Thus, in some embodiments, the chips may comprise an array of nucleic acid sequences of fragments of about 15 nucleotides in length. In further embodiments, the chip may comprise an array including at least one of the sequences selected from the group consisting of those disclosed in the FIGS. 1, 2, 8, 9, and the sequences complementary thereto, or a fragment thereof, said fragment comprising at least about 8 consecutive nucleotides, preferably 10, 15, 20, more preferably 25, 30, 40, 47, or 50 consecutive nucleotides and containing a polymorphic base. In some embodiments the polymorphic base is within 5, 4, 3, 2, or 1 nucleotides from the center of the polynucleotide, more preferably at the center of said polynucleotide. In other embodiments, the chip may comprise an array containing any number of polynucleotides of the present invention.

An oligonucleotide may be synthesized on the surface of the substrate by using a chemical coupling procedure and an ink jet application apparatus, as described in PCT application WO95/251116 (Baldeschweiler et al.) which is incorporated herein in its entirety by reference. In another aspect, a “gridded” array analogous to a dot (or slot) blot may be used to arrange and link cDNA fragments or oligonucleotides to the surface of a substrate using a vacuum system, thermal, UV, mechanical or chemical bonding procedures. An array, such as those described above, may be produced by hand or by using available devices (slot blot or dot blot apparatus), materials (any suitable solid support), and machines (including robotic instruments), and may contain 8, 24, 96, 384, 1536, 6144 or more oligonucleotides, or any other number which lends itself to the efficient use of commercially available instrumentation.

Using such arrays, the present invention provides methods of identifying the SNPs of the present invention in a sample. Such methods comprise incubating a test sample with an array comprising one or more oligonucleotide probes corresponding to at least one SNP position of the present invention, and assaying for binding of a nucleic acid from the test sample with one or more of the oligonucleotide probes. Such assays will typically involve arrays comprising oligonucleotides probes corresponding to many SNP positions and/or allelic variants of those SNP positions, at least one of which is a SNP of the present invention.

Conditions for incubating a nucleic acid molecule with a test sample vary. Incubation conditions depend on the format employed in the assay, the detection methods employed, and the type and nature of the nucleic acid molecule used in the assay. One skilled in the art will recognize that any one of the commonly available hybridization, amplification or array assay formats can readily be adapted to employ the novel SNPs disclosed herein. Examples of such assays can be found in Chard, T, An Introduction to Radioimmunoassay and Related Techniques, Elsevier Science Publishers, Amsterdam, The Netherlands (1986); Bullock, G. R. et al., Techniques in Immunocytochemistry, Academic Press, Orlando, Fla. Vol. 1 (1982), Vol. 2 (1983), Vol. 3 (1985); Tijssen, P., Practice and Theory of Enzyme Immunoassays: Laboratory Techniques in Biochemistry and Molecular Biology, Elsevier Science Publishers, Amsterdam, The Netherlands (1985).

The test samples of the present invention include, but are not limited to, nucleic acid extracts, cells, and protein or membrane extracts from cells, which may be obtained from any bodily fluids (such as blood, urine, saliva, phlegm, gastric juices, etc.), cultured cells, biopsies, or other tissue preparations. The test sample used in the above-described methods will vary based on the assay format, nature of the detection method and the tissues, cells or extracts used as the sample to be assayed. Methods of preparing nucleic acid, protein, or cell extracts are well known in the art and can be readily be adapted in order to obtain a sample that is compatible with the system utilized.

Multicomponent integrated systems may also be used to analyze SNPs. Such systems miniaturize and compartmentalize processes such as PCR and capillary electrophoresis reactions in a single functional device. An example of such technique is disclosed in U.S. Pat. No. 5,589,136, which describes the integration of PCR amplification and capillary electrophoresis in chips.

Integrated systems can be envisaged mainly when microfluidic systems are used. These systems comprise a pattern of microchannels designed onto a glass, silicon, quartz, or plastic wafer included on a microchip. The movements of the samples are controlled by electric, electroosmotic or hydrostatic forces applied across different areas of the microchip to create functional microscopic valves and pumps with no moving parts. Varying the voltage controls the liquid flow at intersections between the micro-machined channels and changes the liquid flow rate for pumping across different sections of the microchip.

For genotyping SNPs, the microfluidic system may integrate, for example, nucleic acid amplification, minisequencing primer extension, capillary electrophoresis, and a detection method such as laser induced fluorescence detection.

In a first step, the DNA samples are amplified, preferably by PCR. Then, the amplification products are subjected to automated minisequencing reactions using ddNTPs (specific fluorescence for each ddNTP) and the appropriate oligonucleotide minisequencing primers which hybridize just upstream of the targeted polymorphic base. Once the extension at the 3′ end is completed, the primers are separated from the unincorporated fluorescent ddNTPs by capillary electrophoresis. The separation medium used in capillary electrophoresis can be, for example, polyacrylamide, polyethyleneglycol or dextran. The incorporated ddNTPs in the single nucleotide primer extension products are identified by laser-induced fluorescence detection. This microchip can be used to process at least 96 to 384 samples, or more, in parallel.

Vectors/host cells

The invention also provides vectors containing the nucleic acid molecules described herein. The term “vector” refers to a vehicle, preferably a nucleic acid molecule, that can transport the nucleic acid molecules. When the vector is a nucleic acid molecule, the nucleic acid molecules are covalently linked to the vector nucleic acid. With this aspect of the invention, the vector includes a plasmid, single or double stranded phage, a single or double stranded RNA or DNA viral vector, or artificial chromosome, such as a BAC, PAC, YAC, OR MAC.

A vector can be maintained in the host cell as an extrachromosomal element where it replicates and produces additional copies of the nucleic acid molecules. Alternatively, the vector may integrate into the host cell genome and produce additional copies of the nucleic acid molecules when the host cell replicates.

The invention provides vectors for the maintenance (cloning vectors) or vectors for expression (expression vectors) of the nucleic acid molecules. The vectors can function in procaryotic or eukaryotic cells or in both (shuttle vectors).

Expression vectors contain cis-acting regulatory regions that are operably linked in the vector to the nucleic acid molecules such that transcription of the nucleic acid molecules is allowed in a host cell. The nucleic acid molecules can be introduced into the host cell with a separate nucleic acid molecule capable of affecting transcription. Thus, the second nucleic acid molecule may provide a trans-acting factor interacting with the cis-regulatory control region to allow transcription of the nucleic acid molecules from the vector. Alternatively, a trans-acting factor may be supplied by the host cell. Finally, a trans-acting factor can be produced from the vector itself. It is understood, however, that in some embodiments, transcription and/or translation of the nucleic acid molecules can occur in a cell-free system.

The regulatory sequence to which the nucleic acid molecules described herein can be operably linked include promoters for directing mRNA transcription. These include, but are not limited to, the left promoter from bacteriophage λ, the lac, TRP, and TAC promoters from E. coli, the early and late promoters from SV40, the CMV immediate early promoter, the adenovirus early and late promoters, and retrovirus long-terminal repeats.

In addition to control regions that promote transcription, expression vectors may also include regions that modulate transcription, such as repressor binding sites and enhancers. Examples include the SV40 enhancer, the cytomegalovirus immediate early enhancer, polyoma enhancer, adenovirus enhancers, and retrovirus LTR enhancers.

In addition to containing sites for transcription initiation and control, expression vectors can also contain sequences necessary for transcription termination and, in the transcribed region a ribosome binding site for translation. Other regulatory control elements for expression include initiation and termination codons as well as polyadenylation signals. The person of ordinary skill in the art would be aware of the numerous regulatory sequences that are useful in expression vectors. Such regulatory sequences are described, for example, in Sambrook et al., Molecular Cloning: A Laboratory Manual. 2nd. ed., Cold Spring Harbor Laboratory Press, Cold Spring Harbor, N.Y., (1989).

A variety of expression vectors can be used to express a nucleic acid molecule. Such vectors include chromosomal, episomal, and virus-derived vectors, for example vectors derived from bacterial plasmids, from bacteriophage, from yeast episomes, from yeast chromosomal elements, including yeast artificial chromosomes, from viruses such as baculoviruses, papovaviruses such as SV40, Vaccinia viruses, adenoviruses, poxviruses, pseudorabies viruses, and retroviruses. Vectors may also be derived from combinations of these sources such as those derived from plasmid and bacteriophage genetic elements, eg. cosmids and phagemids. Appropriate cloning and expression vectors for prokaryotic and eukaryotic hosts are described in Sambrook et al., Molecular Cloning: A Laboratory Manual. 2nd. ed., Cold Spring Harbor Laboratory Press, Cold Spring Harbor, N.Y., (1989).

The regulatory sequence may provide constitutive expression in one or more host cells (i.e. tissue specific) or may provide for inducible expression in one or more cell types such as by temperature, nutrient additive, or exogenous factor such as a hormone or other ligand. A variety of vectors providing for constitutive and inducible expression in prokaryotic and eukaryotic hosts are well known to those of ordinary skill in the art.

The nucleic acid molecules can be inserted into the vector nucleic acid by well-known methodology. Generally, the DNA sequence that will ultimately be expressed is joined to an expression vector by cleaving the DNA sequence and the expression vector with one or more restriction enzymes and then ligating the fragments together. Procedures for restriction enzyme digestion and ligation are well known to those of ordinary skill in the art.

The vector containing the appropriate nucleic acid molecule can be introduced into an appropriate host cell for propagation or expression using well-known techniques. Bacterial cells include, but are not limited to, E. coli, Streptomyces, and Salmonella typhimurium. Eukaryotic cells include, but are not limited to, yeast, insect cells such as Drosophila, animal cells such as COS and CHO cells, and plant cells.

As described herein, it may be desirable to express the peptide as a fusion protein. Accordingly, the invention provides fusion vectors that allow for the production of the peptides. Fusion vectors can increase the expression of a recombinant protein, increase the solubility of the recombinant protein, and aid in the purification of the protein by acting for example as a ligand for affinity purification. A proteolytic cleavage site may be introduced at the junction of the fusion moiety so that the desired peptide can ultimately be separated from the fusion moiety. Proteolytic enzymes include, but are not limited to, factor Xa, thrombin, and enterokinase. Typical fusion expression vectors include pGEX (Smith et al., Gene 67:31-40 (1988)), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. Examples of suitable inducible non-fusion E. coli expression vectors include pTrc (Amann et al., Gene 69:301-315 (1988)) and pET 11d (Studier et al., Gene Expression Technology: Methods in Enzymology 185:60-89 (1990)).

Recombinant protein expression can be maximized in a host bacteria by providing a genetic background wherein the host cell has an impaired capacity to proteolytically cleave the recombinant protein. (Gottesman, S., Gene Expression Technology: Methods in Enzymology 185, Academic Press, San Diego, Calif. (1990) 119-128). Alternatively, the sequence of the nucleic acid molecule of interest can be altered to provide preferential codon usage for a specific host cell, for example E. coli. (Wada et al., Nucleic Acids Res. 20:2111-2118 (1992)).

The nucleic acid molecules can also be expressed by expression vectors that are operative in yeast. Examples of vectors for expression in yeast e.g., S. cerevisiae include pYepSec1 (Baldari, et al., EMBO J 6:229-234 (1987)), pMFa (Kurjan et al., Cell 30:933-943(1982)), pJRY88 (Schultz et al., Gene 54:113-123 (1987)), and pYES2 (Invitrogen Corporation, San Diego, Calif.).

The nucleic acid molecules can also be expressed in insect cells using, for example, baculovirus expression vectors. Baculovirus vectors available for expression of proteins in cultured insect cells (e.g., Sf 9 cells) include the pAc series (Smith et al., Mol. Cell Biol. 3:2156-2165 (1983)) and the pVL series (Lucklow et al., Virology 170:31-39 (1989)).

In certain embodiments of the invention, the nucleic acid molecules described herein are expressed in mammalian cells using mammalian expression vectors. Examples of mammalian expression vectors include pCDM8 (Seed, B. Nature 329:840(1987)) and pMT2PC (Kaufman et al., EMBO J. 6:187-195 (1987)).

The expression vectors listed herein are provided by way of example only of the well-known vectors available to those of ordinary skill in the art that would be useful to express the nucleic acid molecules. The person of ordinary skill in the art would be aware of other vectors suitable for maintenance propagation or expression of the nucleic acid molecules described herein. These are found for example in Sambrook, J., Fritsh, E. F., and Maniatis, T. Molecular Cloning: A Laboratory Manual 2nd, ed., Cold Spring Harbor Laboratory, Cold Spring Harbor Laboratory Press, Cold Spring Harbor, N.Y., 1989.

The invention also encompasses vectors in which the nucleic acid sequences described herein are cloned into the vector in reverse orientation, but operably linked to a regulatory sequence that permits transcription of antisense RNA. Thus, an antisense transcript can be produced to all, or to a portion, of the nucleic acid molecule sequences described herein, including both coding and non-coding regions. Expression of this antisense RNA is subject to each of the parameters described above in relation to expression of the sense RNA (regulatory sequences, constitutive or inducible expression, tissue-specific expression).

The invention also relates to recombinant host cells containing the vectors described herein. Host cells therefore include prokaryotic cells, lower eukaryotic cells such as yeast, other eukaryotic cells such as insect cells, and higher eukaryotic cells such as mammalian cells.

The recombinant host cells are prepared by introducing the vector constructs described herein into the cells by techniques readily available to the person of ordinary skill in the art. These include, but are not limited to, calcium phosphate transfection, DEAE-dextran-mediated transfection, cationic lipid-mediated transfection, electroporation, transduction, infection, lipofection, and other techniques such as those found in Sambrook, et al. (Molecular Cloning: A Laboratory Manual. 2nd, ed, Cold Spring Harbor Laboratory, Cold Spring Harbor Laboratory Press, Cold Spring Harbor, N.Y., 1989).

Host cells can contain more than one vector. Thus, different nucleotide sequences can be introduced on different vectors of the same cell. Similarly, the nucleic acid molecules can be introduced either alone or with other nucleic acid molecules that are not related to the nucleic acid molecules such as those providing trans-acting factors for expression vectors. When more than one vector is introduced into a cell, the vectors can be introduced independently, co-introduced or joined to the nucleic acid molecule vector.

In the case of bacteriophage and viral vectors, these can be introduced into cells as packaged or encapsulated virus by standard procedures for infection and transduction. Viral vectors can be replication-competent or replication-defective. In the case in which viral replication is defective, replication will occur in host cells providing functions that complement the defects.

Vectors generally include selectable markers that enable the selection of the subpopulation of cells that contain the recombinant vector constructs. The marker can be contained in the same vector that contains the nucleic acid molecules described herein or may be on a separate vector. Markers include tetracycline or ampicillin-resistance genes for prokaryotic host cells and dihydrofolate reductase or neomycin resistance for eukaryotic host cells. However, any marker that provides selection for a phenotypic trait will be effective.

While the mature proteins can be produced in bacteria, yeast, mammalian cells, and other cells under the control of the appropriate regulatory sequences, cell-free transcription and translation systems can also be used to produce these proteins using RNA derived from the DNA constructs described herein.

Where secretion of the peptide is desired, which is difficult to achieve with multi-transmembrane domain containing proteins such as estrogen receptors, appropriate secretion signals are incorporated into the vector. The signal sequence can be endogenous to the peptides or heterologous to these peptides.

Where the peptide is not secreted into the medium, which is typically the case with estrogen receptors, the protein can be isolated from the host cell by standard disruption procedures, including freeze thaw, sonication, mechanical disruption, use of lysing agents and the like. The peptide can then be recovered and purified by well-known purification methods including ammonium sulfate precipitation, acid extraction, anion or cationic exchange chromatography, phosphocellulose chromatography, hydrophobic-interaction chromatography, affinity chromatography, hydroxylapatite chromatography, lectin chromatography, or high performance liquid chromatography.

It is also understood that depending upon the host cell in recombinant production of the peptides described herein, the peptides can have various glycosylation patterns, depending upon the cell, or maybe non-glycosylated as when produced in bacteria. In addition, the peptides may include an initial modified methionine in some cases as a result of a host-mediated process.

Uses of vectors and host cells

The recombinant host cells expressing the peptides described herein have a variety of uses. First, the cells are useful for producing a estrogen receptor protein or peptide that can be further purified to produce desired amounts of estrogen receptor protein or fragments. Thus, host cells containing expression vectors are useful for peptide production.

Host cells are also useful for conducting cell-based assays involving the estrogen receptor protein or estrogen receptor protein fragments, such as those described above as well as other formats known in the art. Thus, a recombinant host cell expressing a native estrogen receptor protein is useful for assaying compounds that stimulate or inhibit estrogen receptor protein function.

Host cells are also useful for identifying estrogen receptor protein mutants in which these functions are affected. If the mutants naturally occur and give rise to a pathology, host cells containing the mutations are useful to assay compounds that have a desired effect on the mutant estrogen receptor protein (for example, stimulating or inhibiting function) which may not be indicated by their effect on the native estrogen receptor protein.

Genetically engineered host cells can be further used to produce non-human transgenic animals. A transgenic animal is preferably a mammal, for example a rodent, such as a rat or mouse, in which one or more of the cells of the animal include a transgene. A transgene is exogenous DNA which is integrated into the genome of a cell from which a transgenic animal develops and which remains in the genome of the mature animal in one or more cell types or tissues of the transgenic animal. These animals are useful for studying the function of a estrogen receptor protein and identifying and evaluating modulators of estrogen receptor protein activity. Other examples of transgenic animals include non-human primates, sheep, dogs, cows, goats, chickens, and amphibians.

A transgenic animal can be produced by introducing nucleic acid into the male pronuclei of a fertilized oocyte, e.g., by microinjection, retroviral infection, and allowing the oocyte to develop in a pseudopregnant female foster animal. Any of the estrogen receptor protein nucleotide sequences can be introduced as a transgene into the genome of a non-human animal, such as a mouse.

Any of the regulatory or other sequences useful in expression vectors can form part of the transgenic sequence. This includes intronic sequences and polyadenylation signals, if not already included. A tissue-specific regulatory sequence(s) can be operably linked to the transgene to direct expression of the estrogen receptor protein to particular cells.

Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the transgene in its genome and/or expression of transgenic mRNA in tissues or cells of the animals. A transgenic founder animal can then be used to breed additional animals carrying the transgene. Moreover, transgenic animals carrying a transgene can further be bred to other transgenic animals carrying other transgenes. A transgenic animal also includes animals in which the entire animal or tissues in the animal have been produced using the homologously recombinant host cells described herein.

In another embodiment, transgenic non-human animals can be produced which contain selected systems that allow for regulated expression of the transgene. One example of such a system is the cre/loxP recombinase system of bacteriophage P1. For a description of the cre/loxP recombinase system, see, e.g., Lakso et al. PNAS 89:6232-6236 (1992). Another example of a recombinase system is the FLP recombinase system of S. cerevisiae (O'Gorman et al. Science 251:1351-1355 (1991). If a cre/loxP recombinase system is used to regulate expression of the transgene, animals containing transgenes encoding both the Cre recombinase and a selected protein is required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. Nature 385:810-813 (1997) and PCT International Publication Nos. WO 97/07668 and WO 97/07669. In brief, a cell, e.g., a somatic cell, from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyst and then transferred to pseudopregnant female foster animal. The offspring born of this female foster animal will be a clone of the animal from which the cell, e.g., the somatic cell, is isolated.

Transgenic animals containing recombinant cells that express the peptides described herein are useful to conduct the assays described herein in an in vivo context. Accordingly, the various physiological factors that are present in vivo and that could effect ligand binding, estrogen receptor protein activation, and signal transduction, may not be evident from in vitro cell-free or cell-based assays. Accordingly, it is useful to provide non-human transgenic animals to assay in vivo estrogen receptor protein function, including ligand interaction, the effect of specific mutant estrogen receptor protein on estrogen receptor protein function and ligand interaction, and the effect of chimeric estrogen receptor protein. It is also possible to assess the effect of null mutations, that is mutations that substantially or completely eliminate one or more estrogen receptor protein functions.

### EXAMPLES

1. SNP Identification and Characterization

Individual exons of estrogen receptor alpha (ESR1) were PCR amplified using primers flanking each adjacent sequence of exon (exon/intron boundaries), and the sequence of amplified fragments was analyzed. As the PCR template, genomic DNA from Coriell Diversity Panels (10 individuals in each of 5 ethnic groups) (see FIG. 2 (b)), and/or Liverpool clinical breast tumor and matching blood samples from 48 patients (from tissue obtained in Liverpool, England, see FIG. 2 (a)) was used. PolyPhred version 2.0 (D. A. Nickerson, S. Taylor, N. Kolker, Univ. of Washington, 1998) was run on the sequences (with default settings) to visualize potential heterozygotes. Tagged sites were examined for quality to verify polymorphisms.

36 SNPs with a frequency greater than 2 and a quality score greater than 20 were found with 13 being unique to the clinical samples. 15 of the SNPs showed at least one instance of a change in heterozygosity in the clinical samples, and 4 showed at least one instance of a loss in heterozygosity. For the analysis, PCR primers were used for SNP identification and detection (see FIG. 2 (e))

Additionally, the primer set in FIG. 2(d) and M13 primers were used for overlapping PCR and clone sequencing.

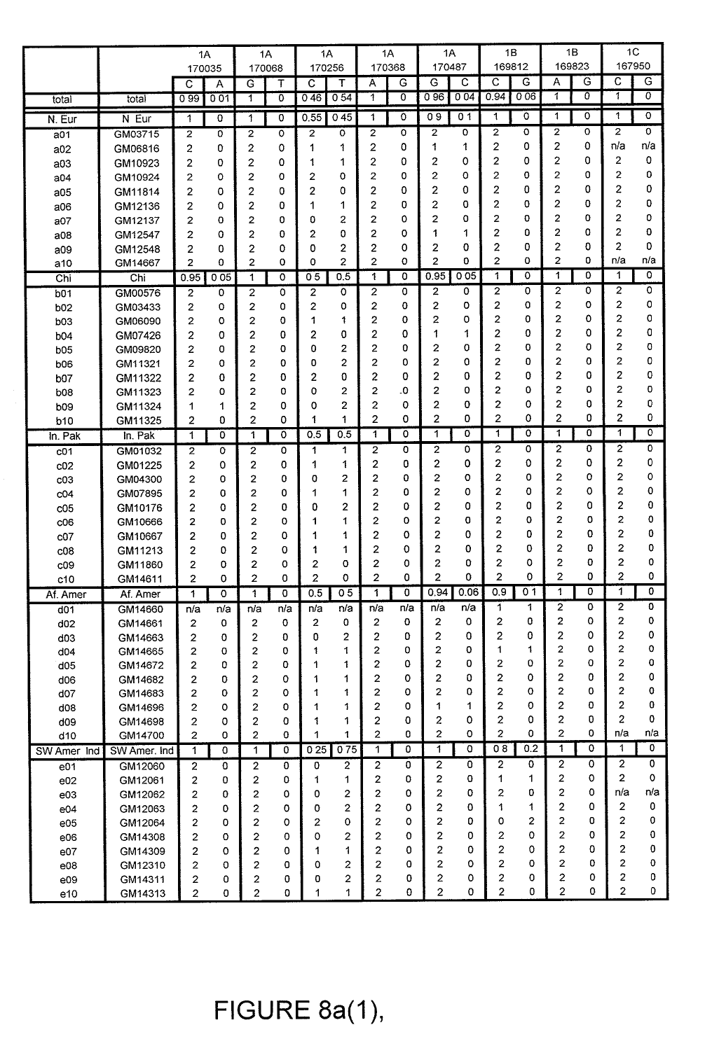

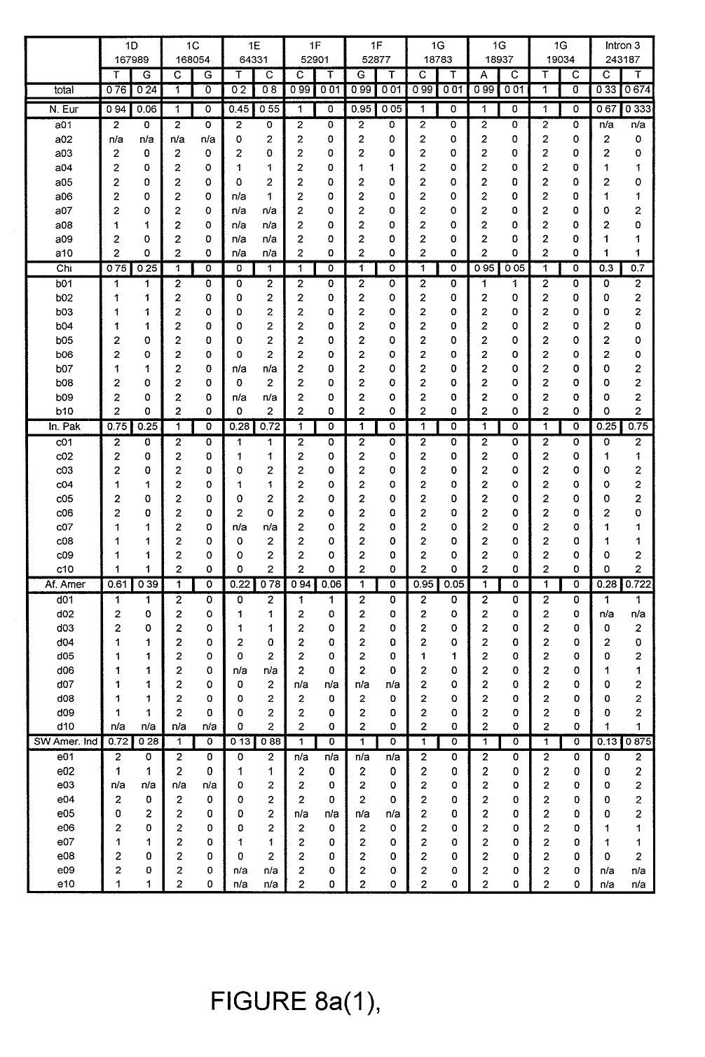

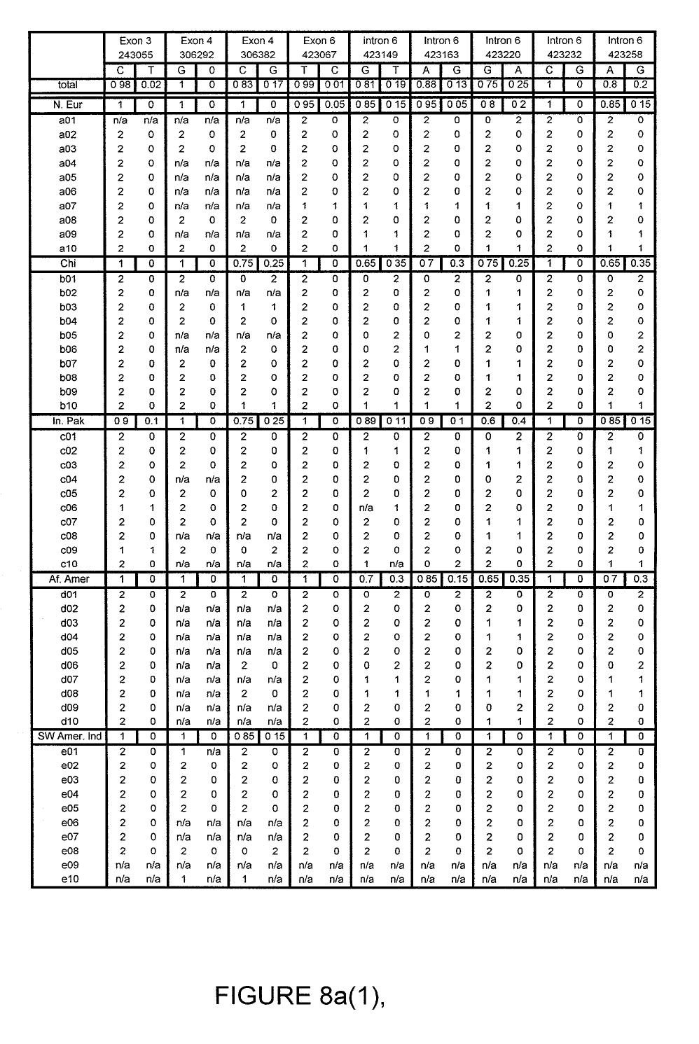

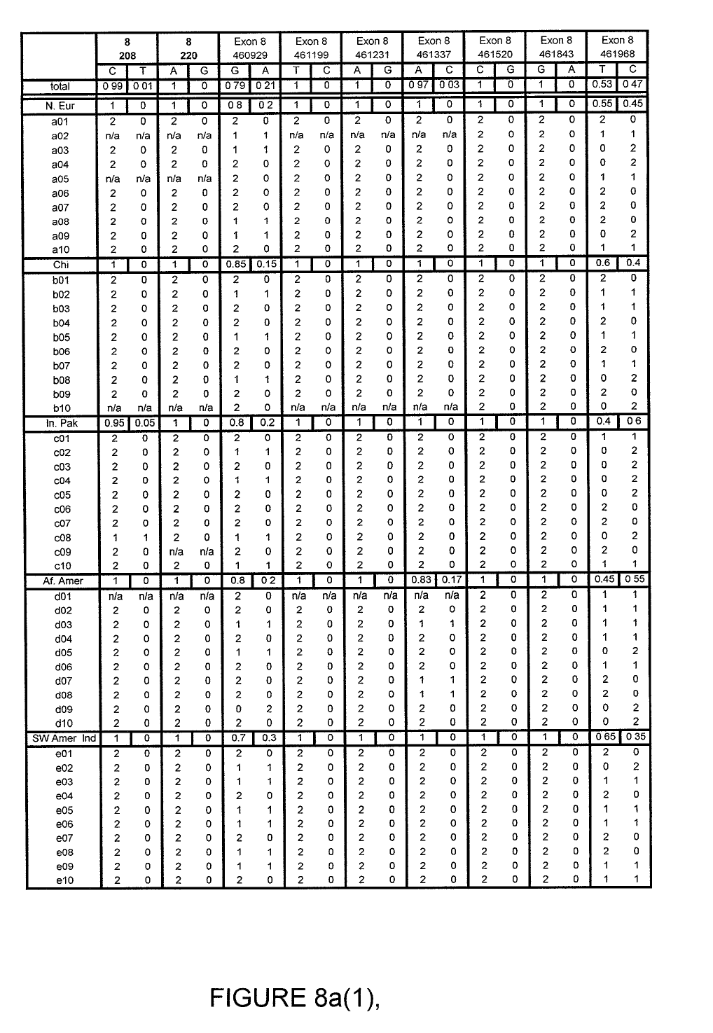

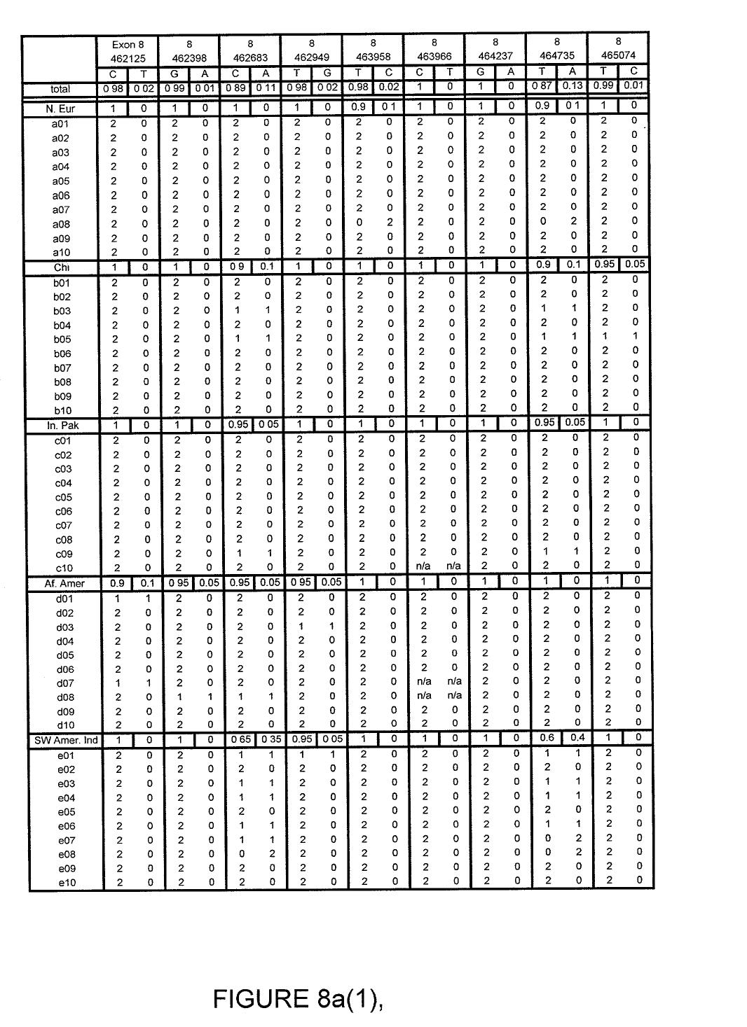

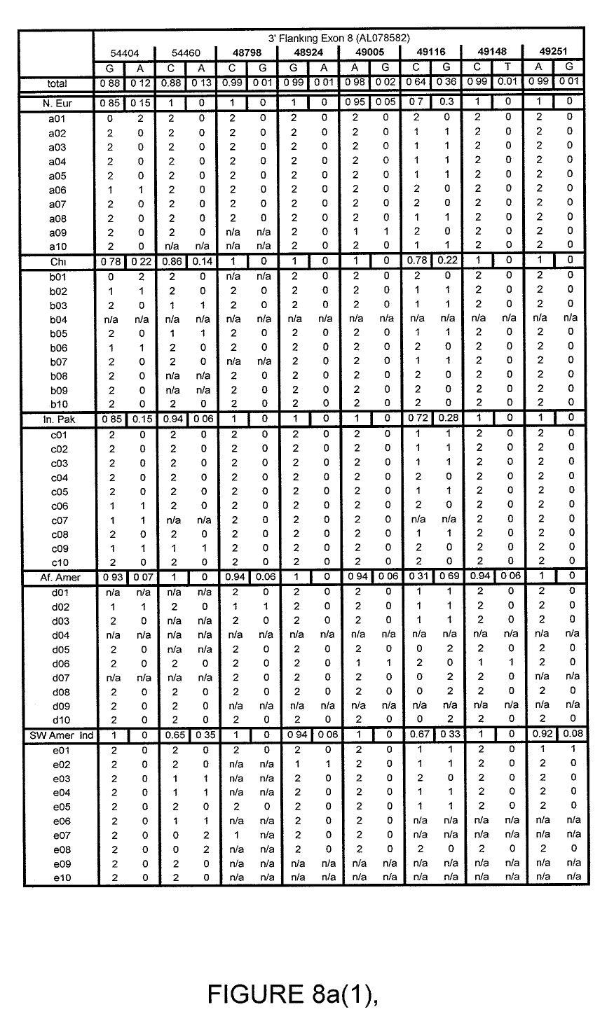

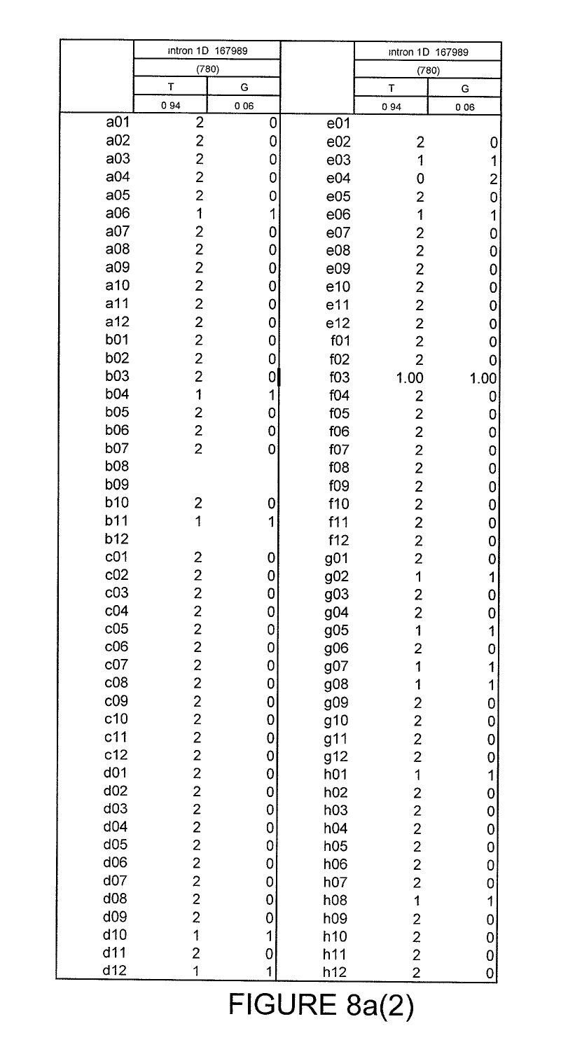

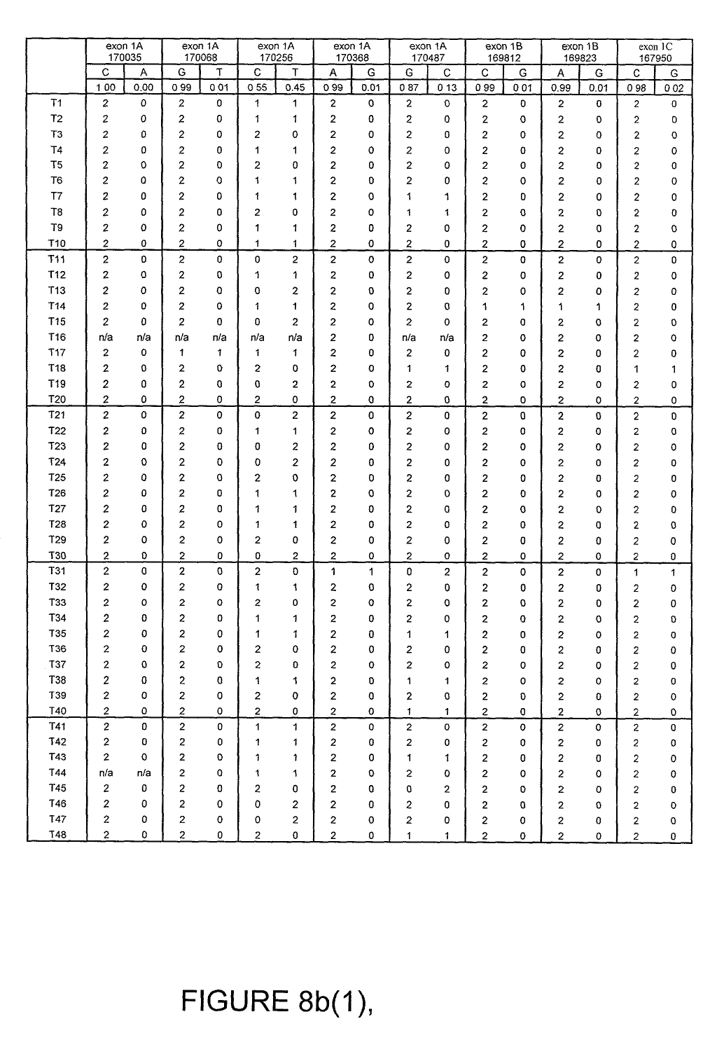

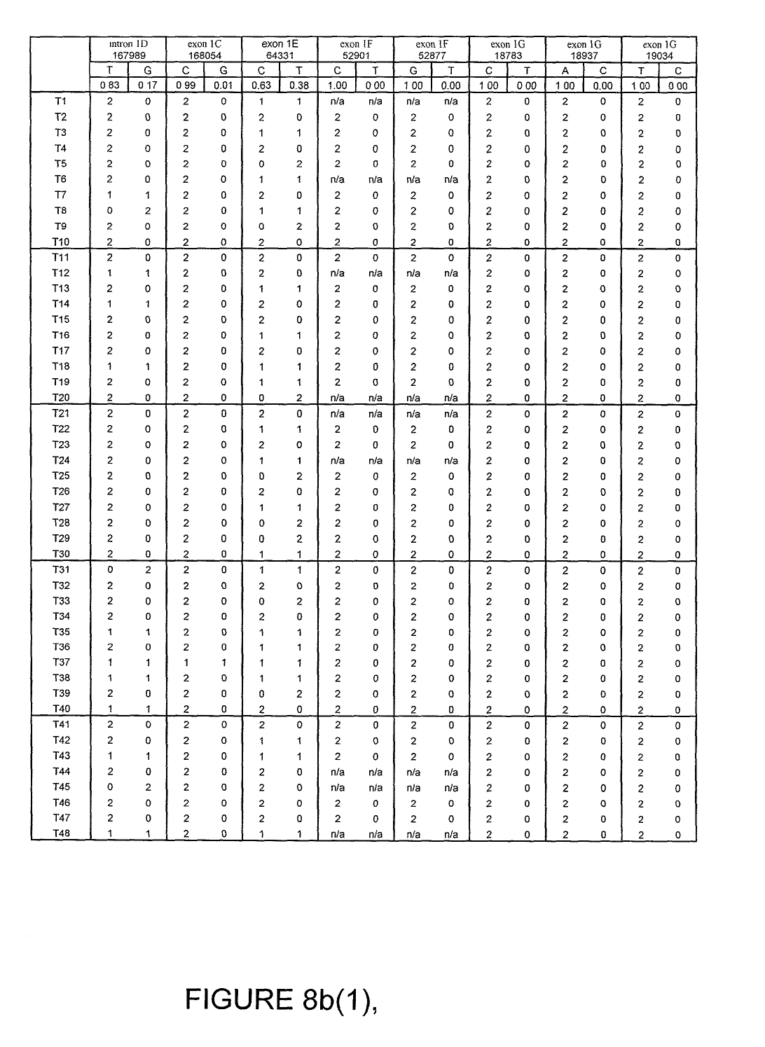

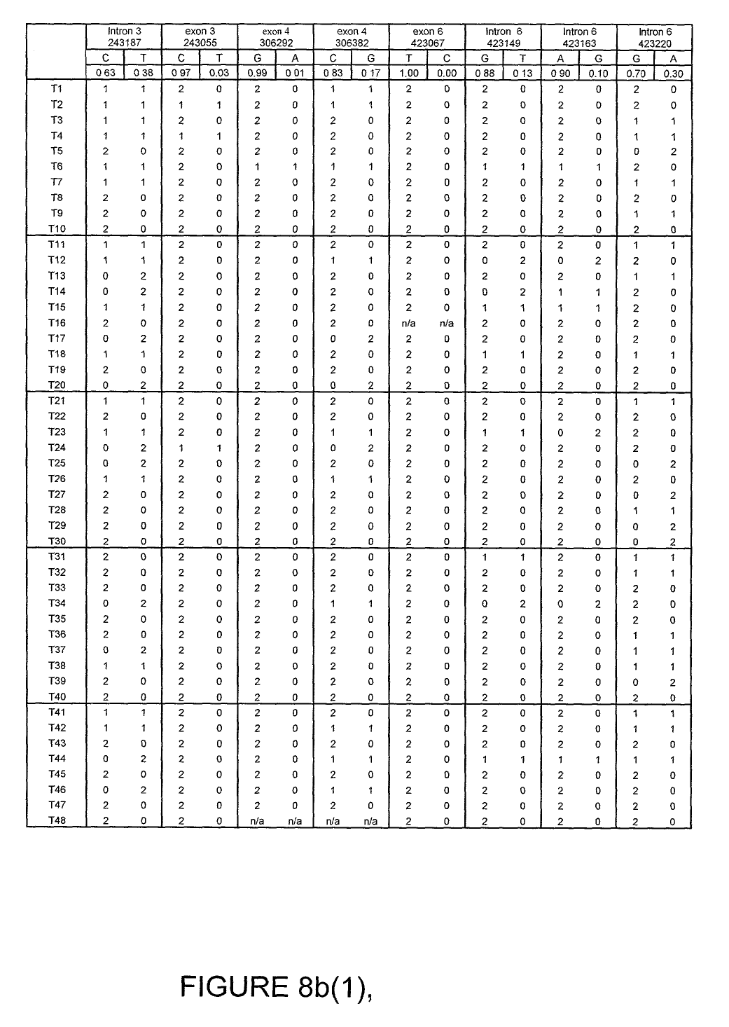

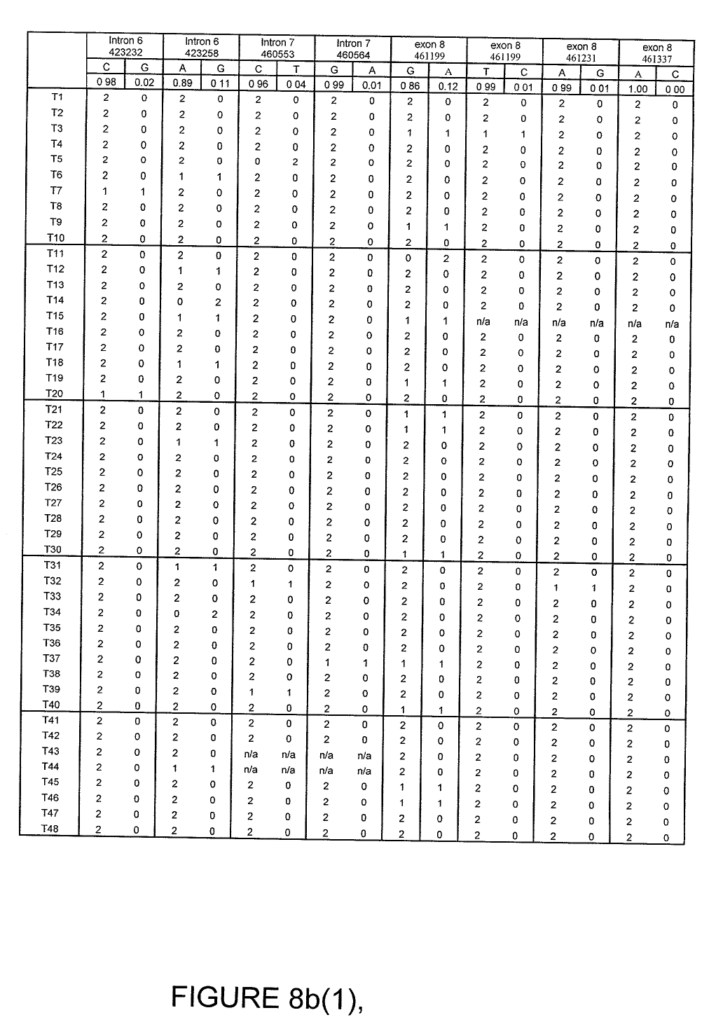

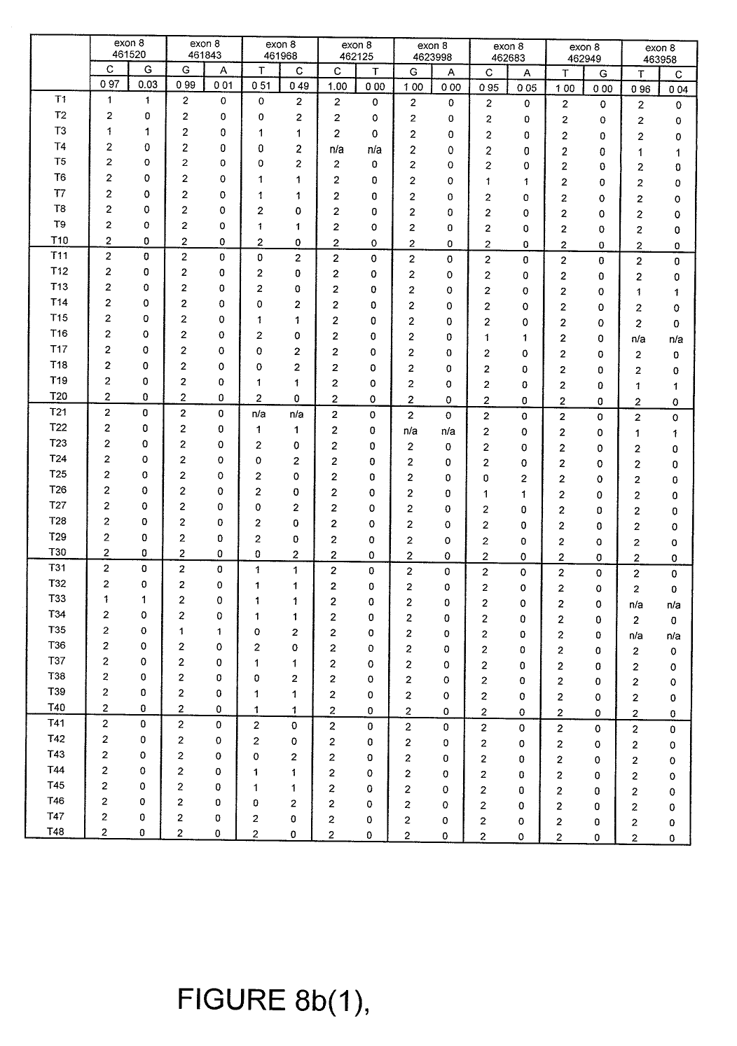

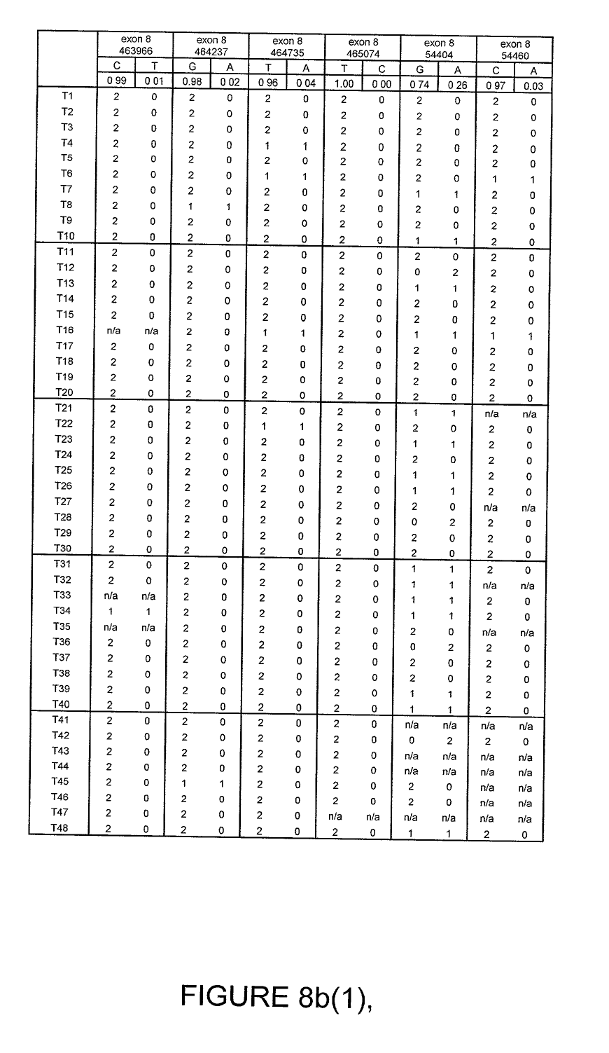

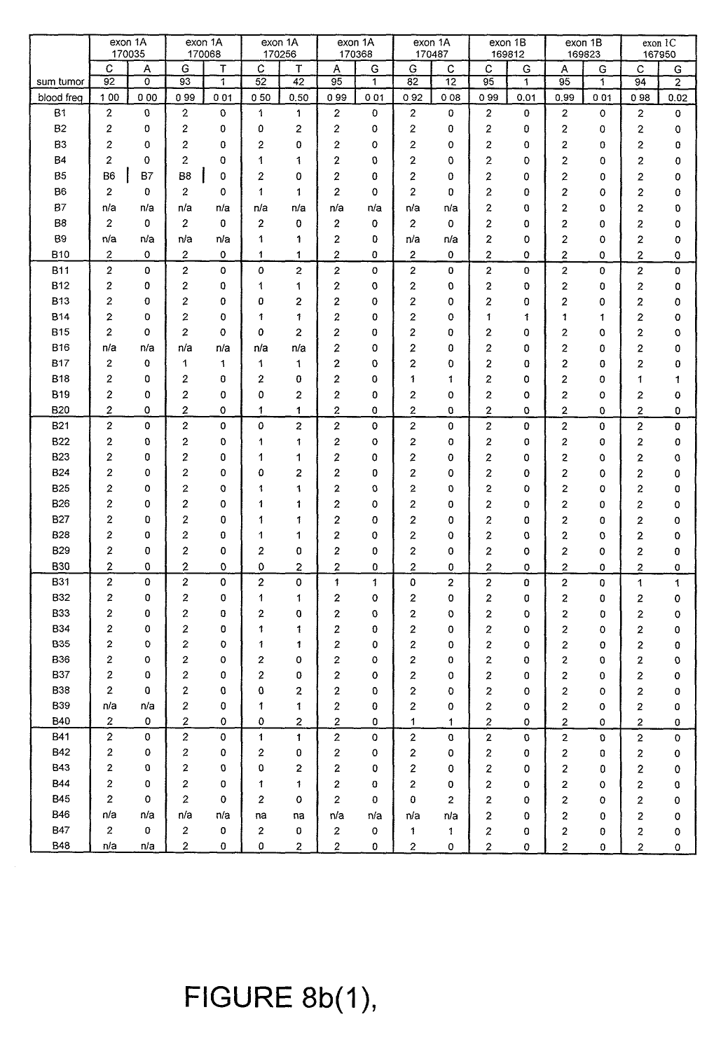

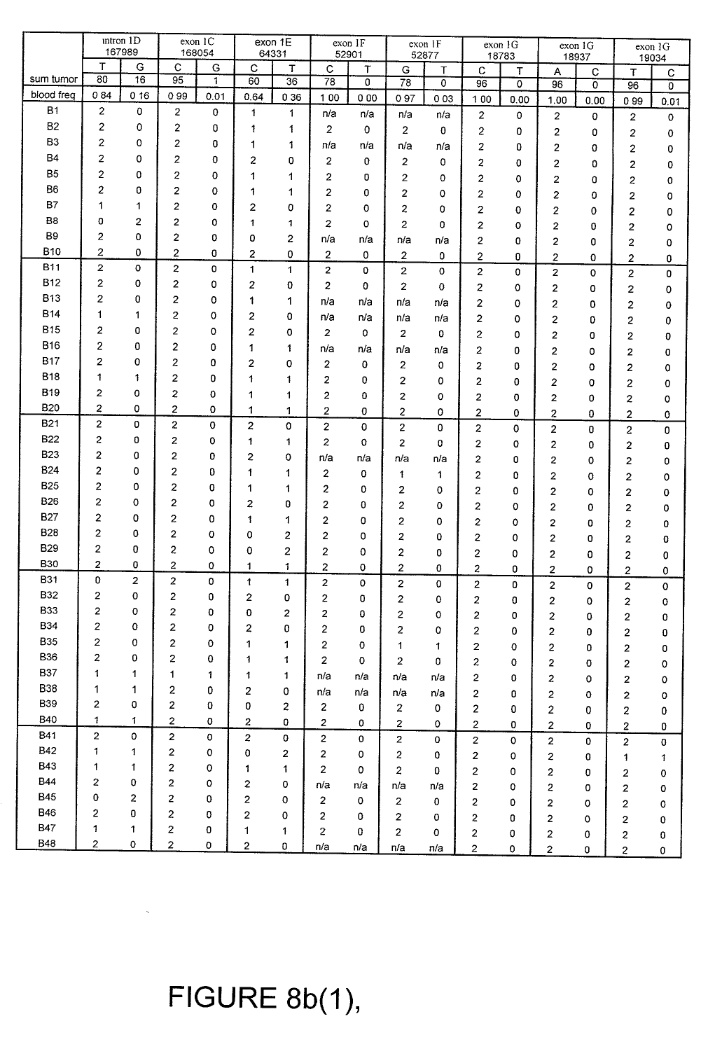

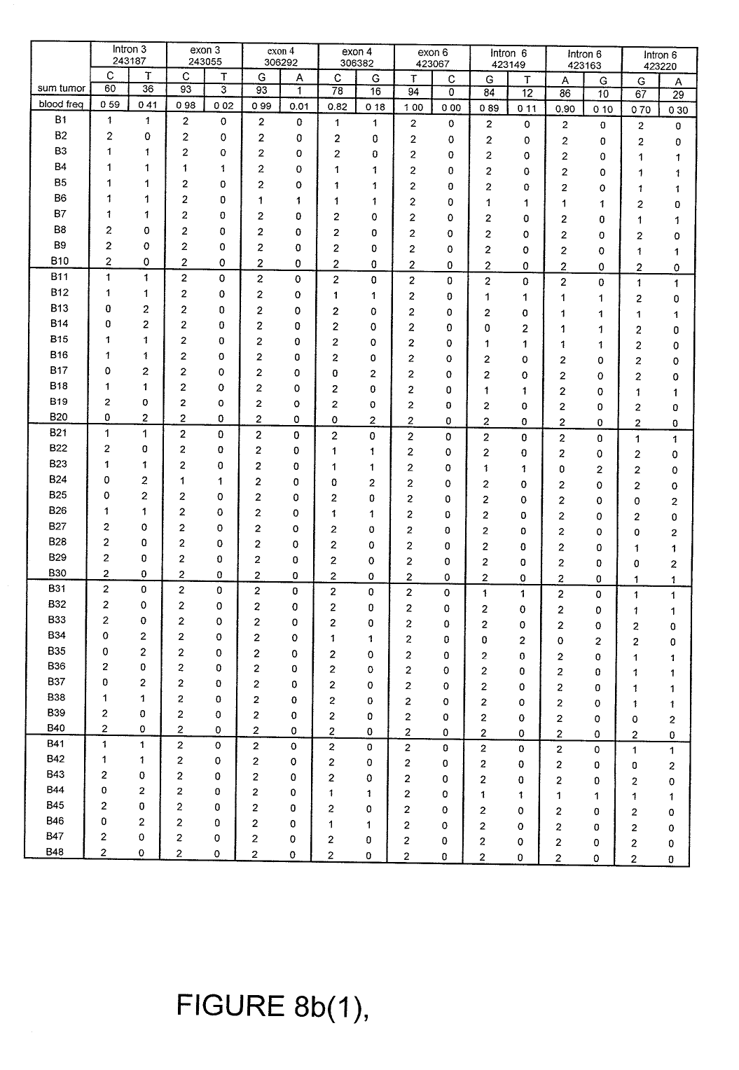

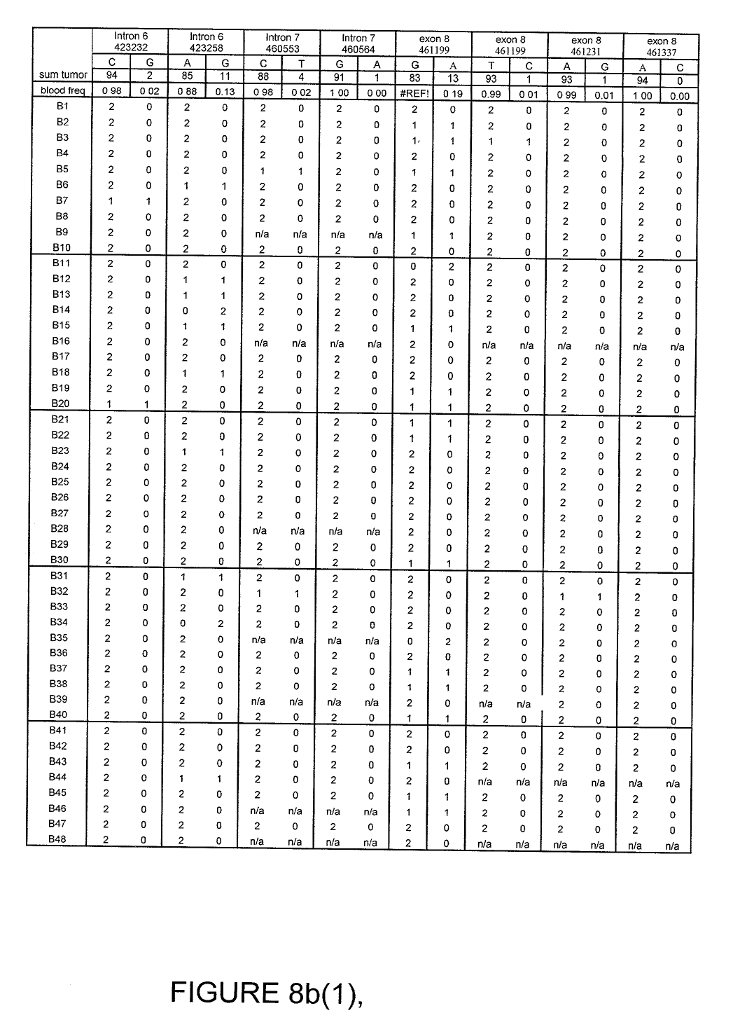

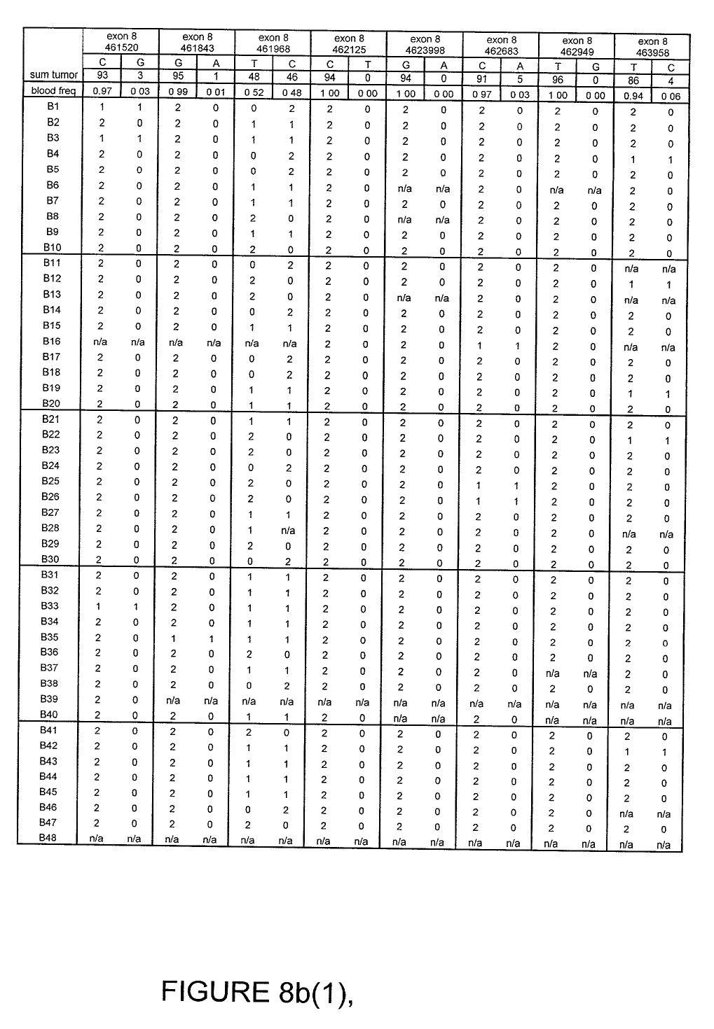

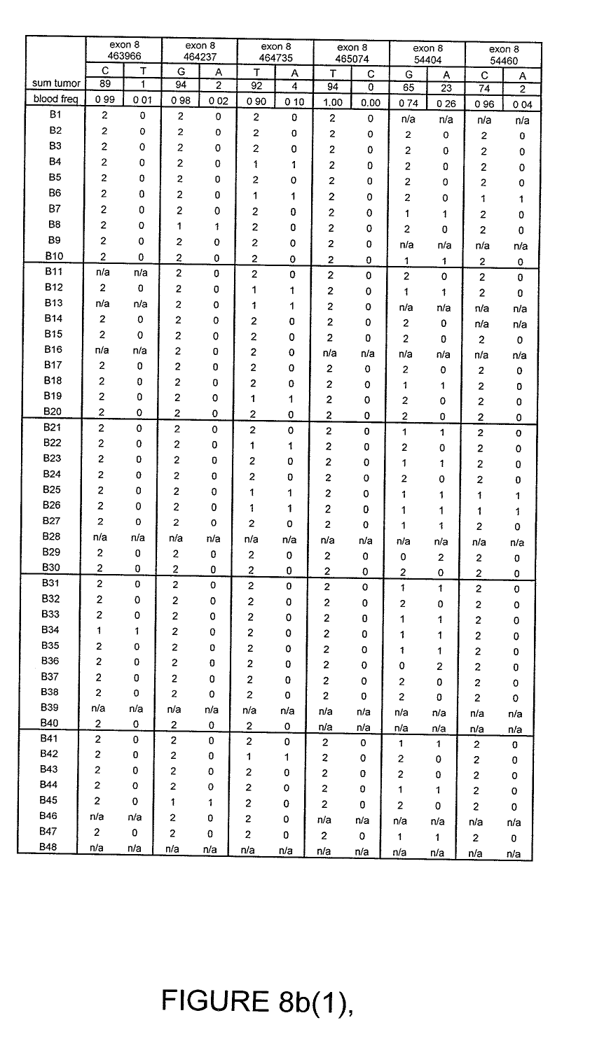

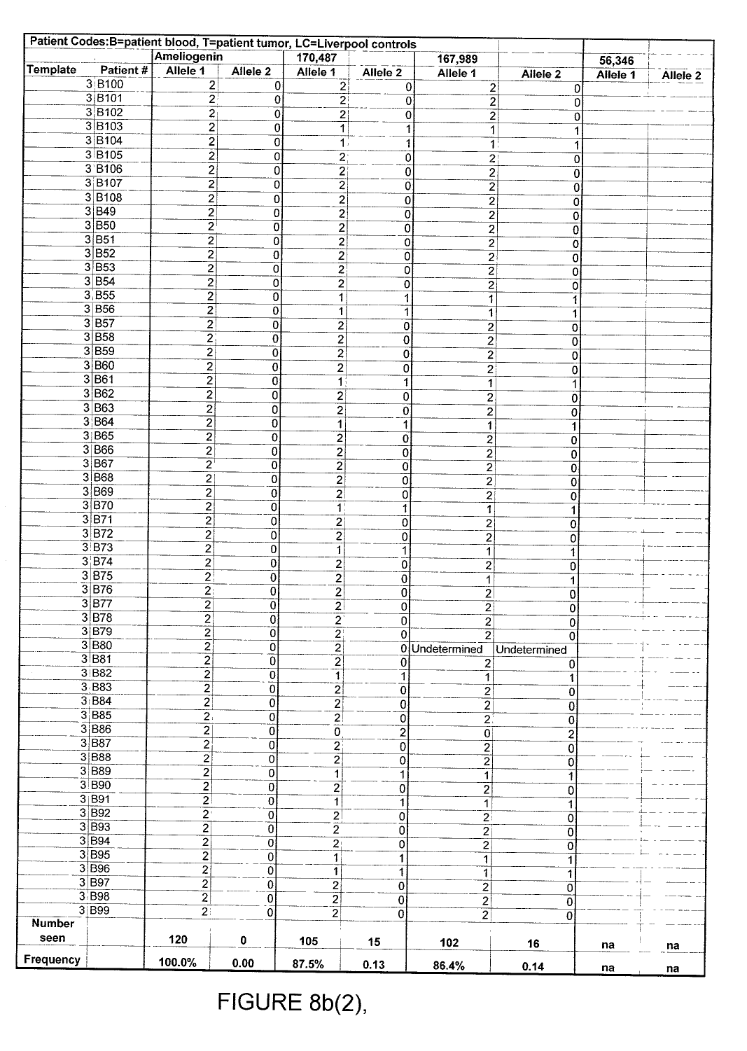

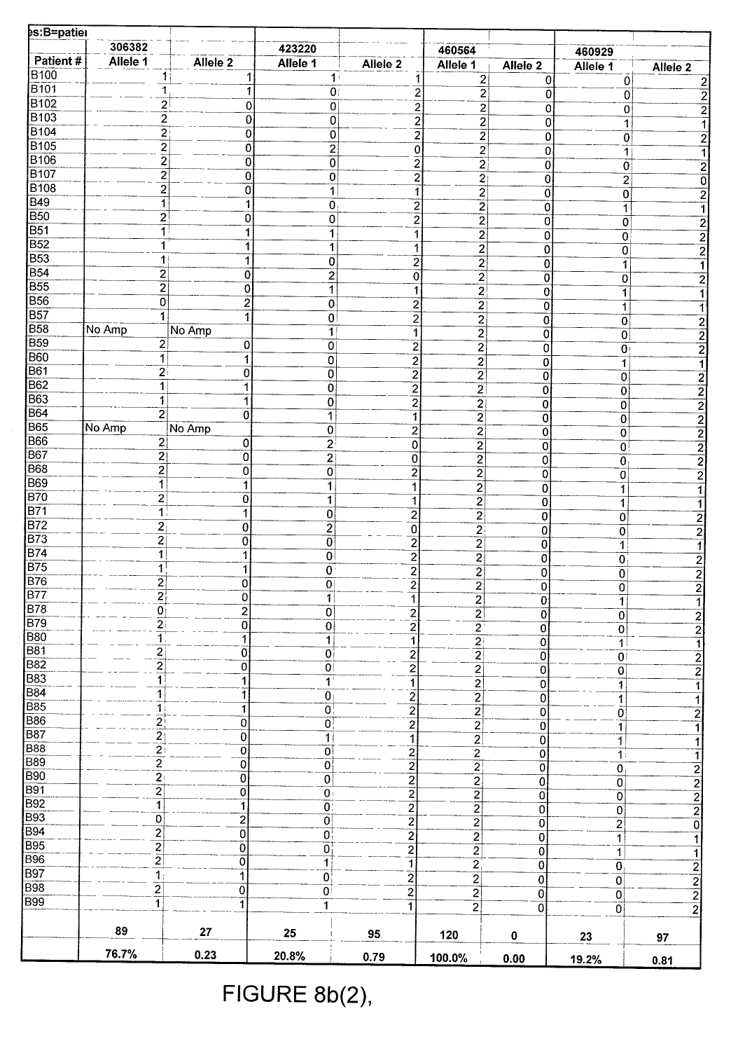

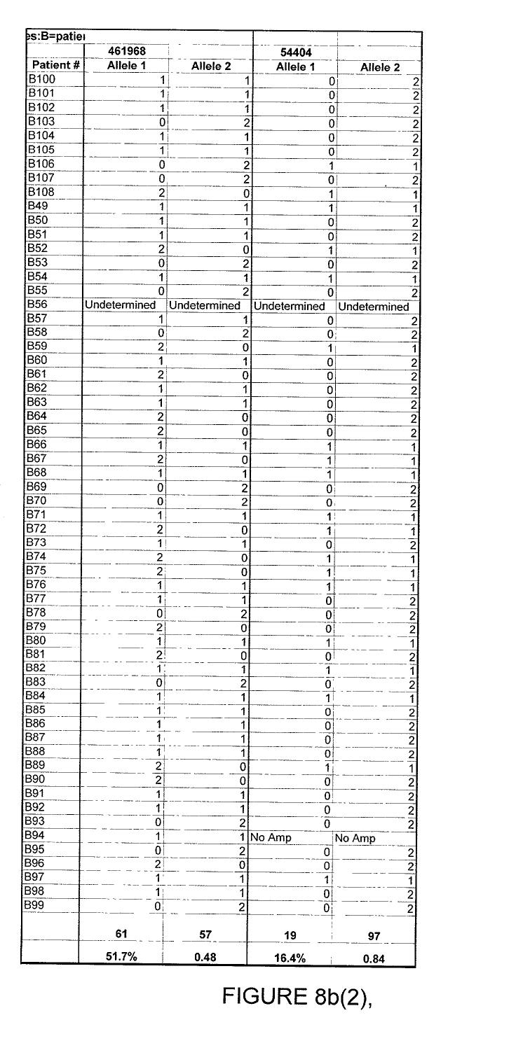

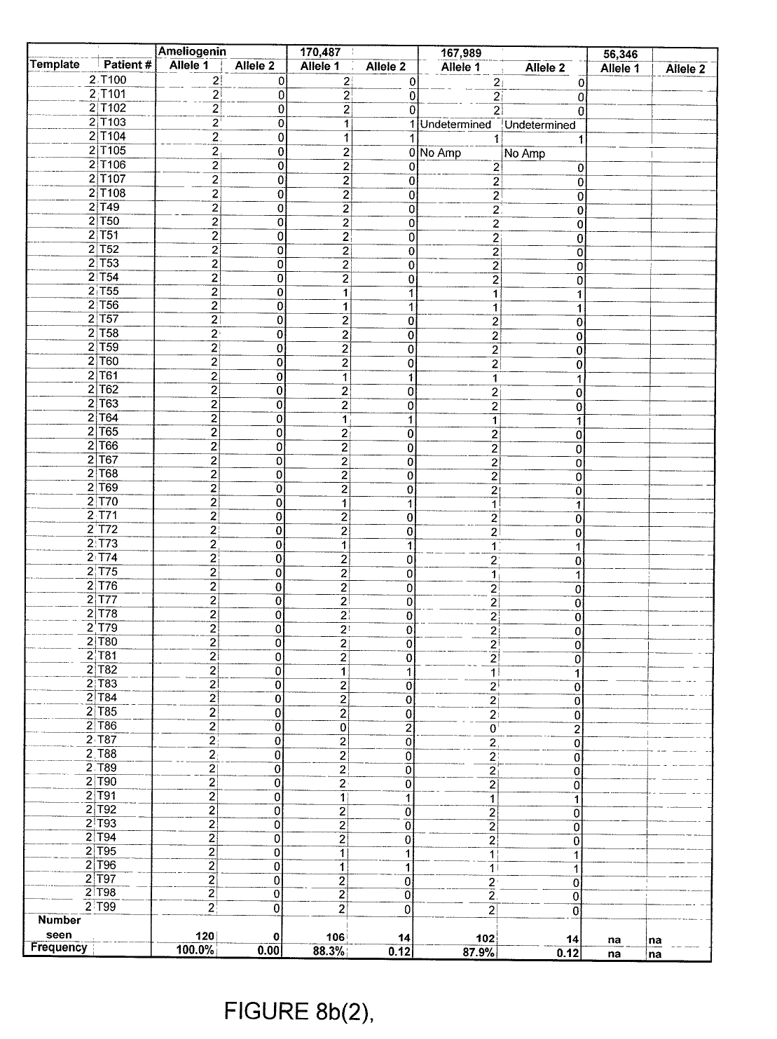

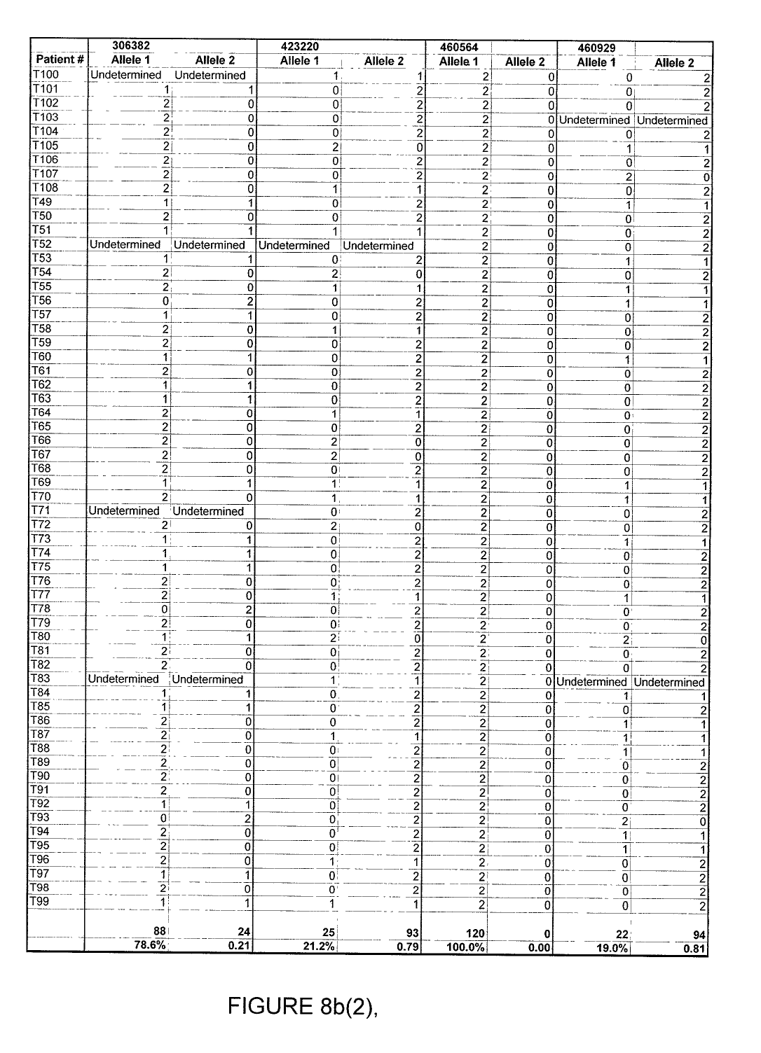

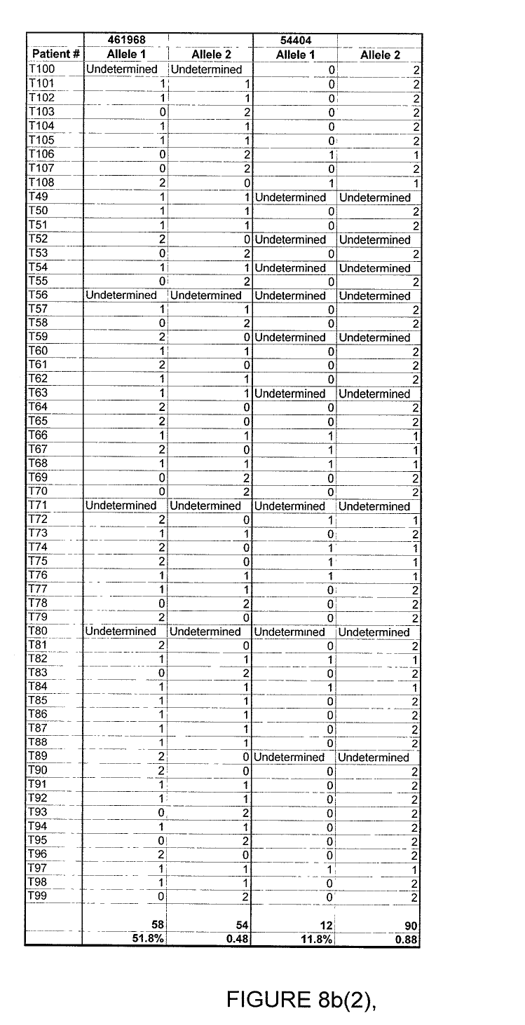

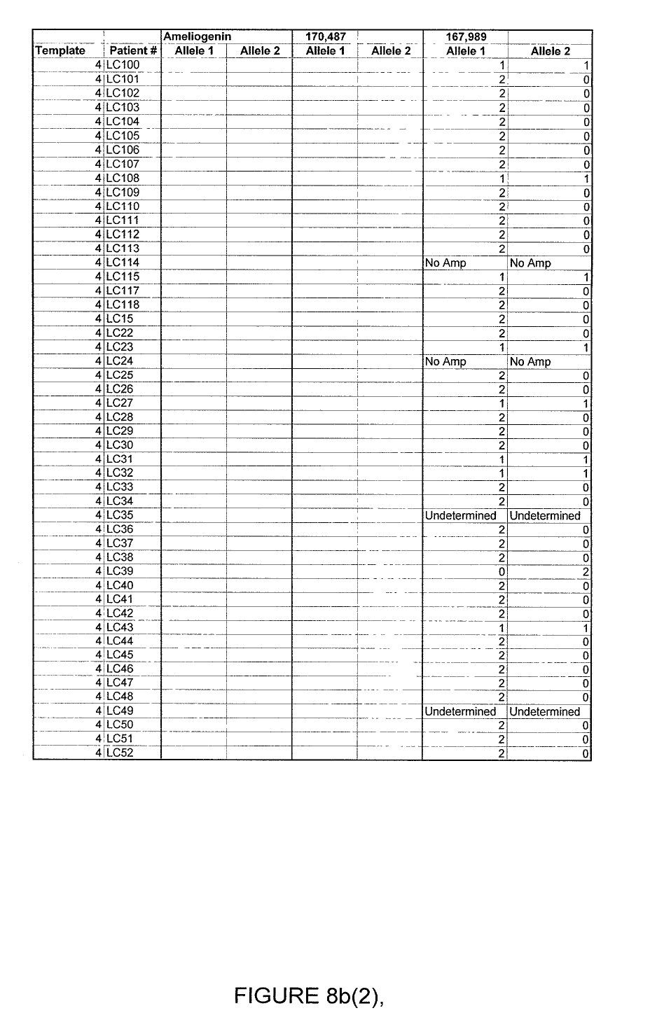

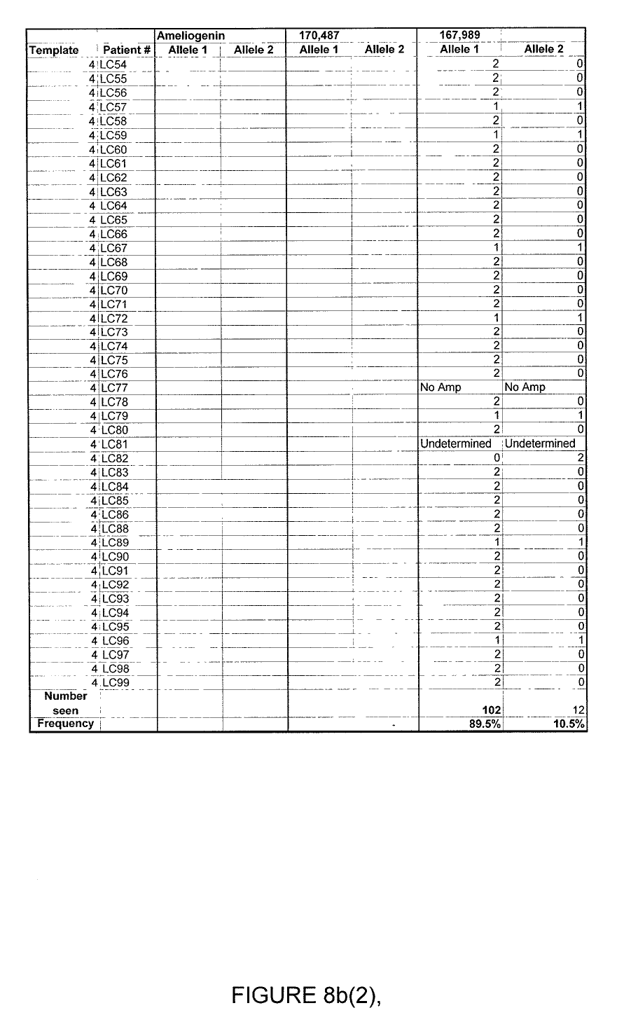

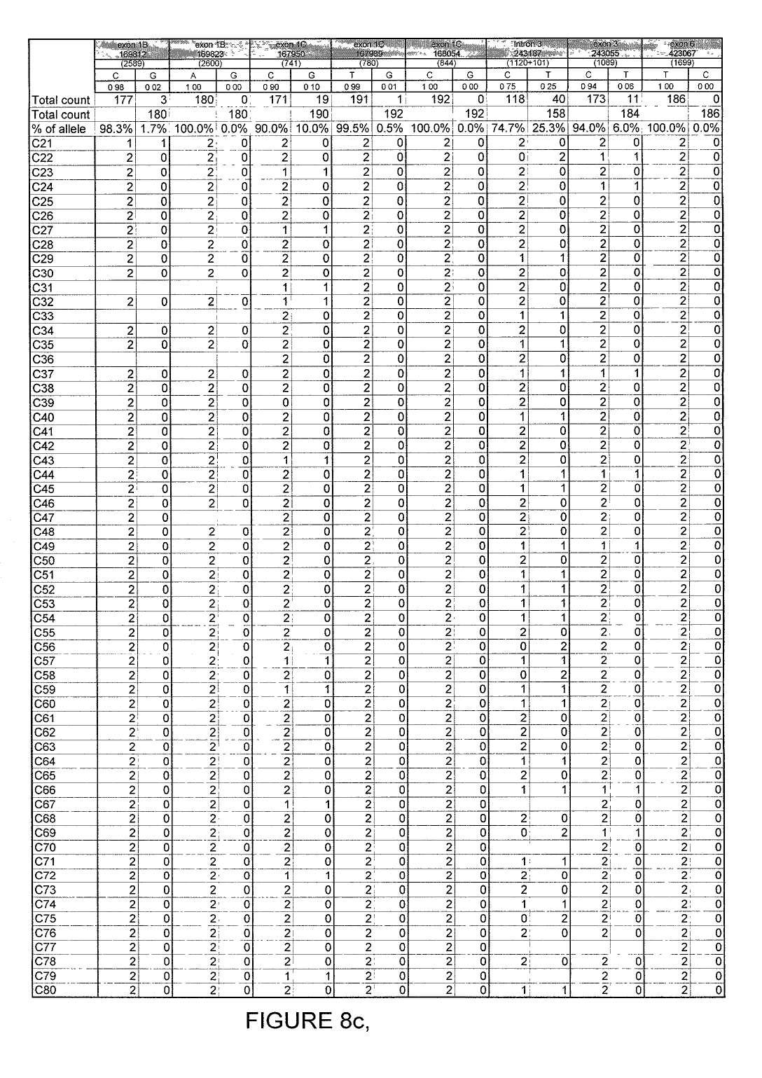

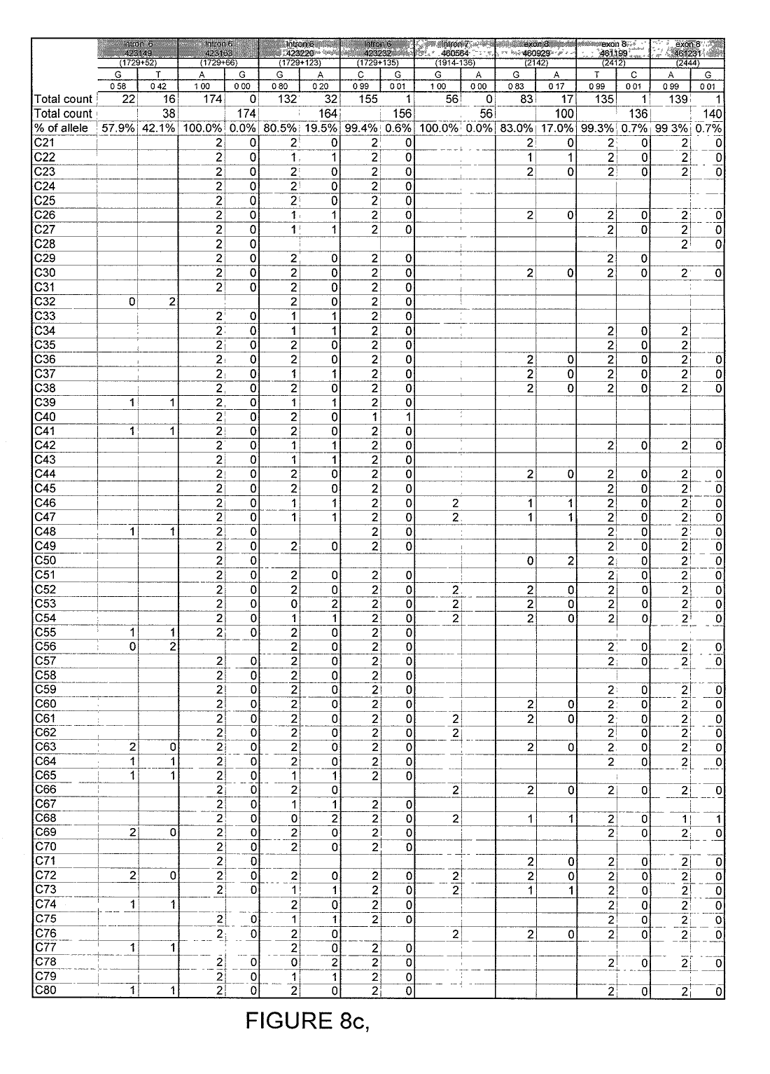

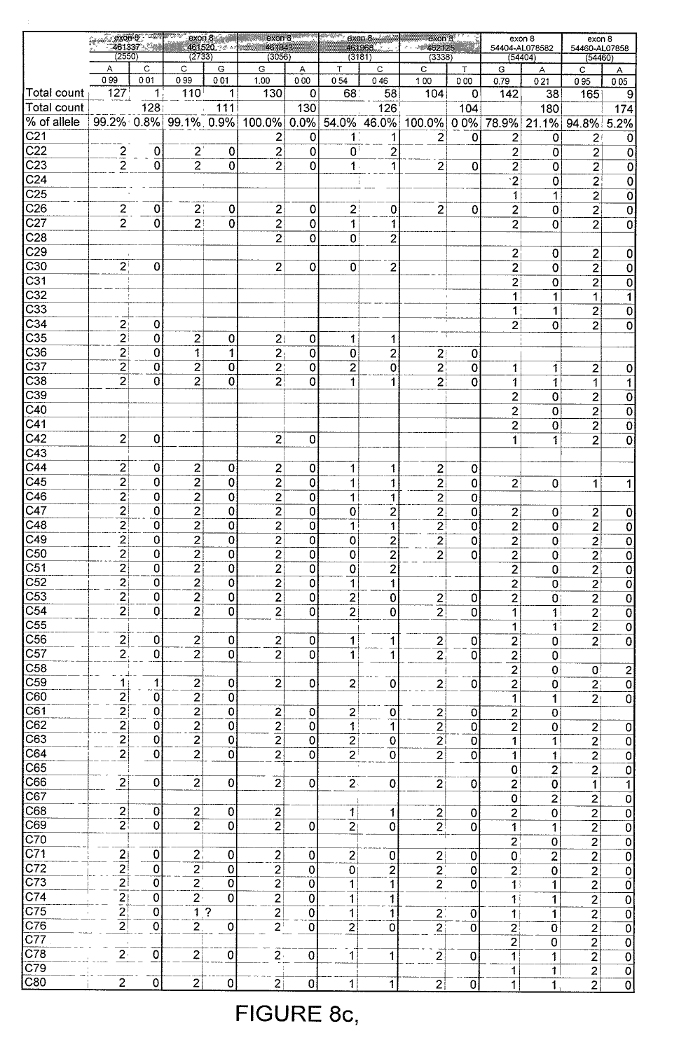

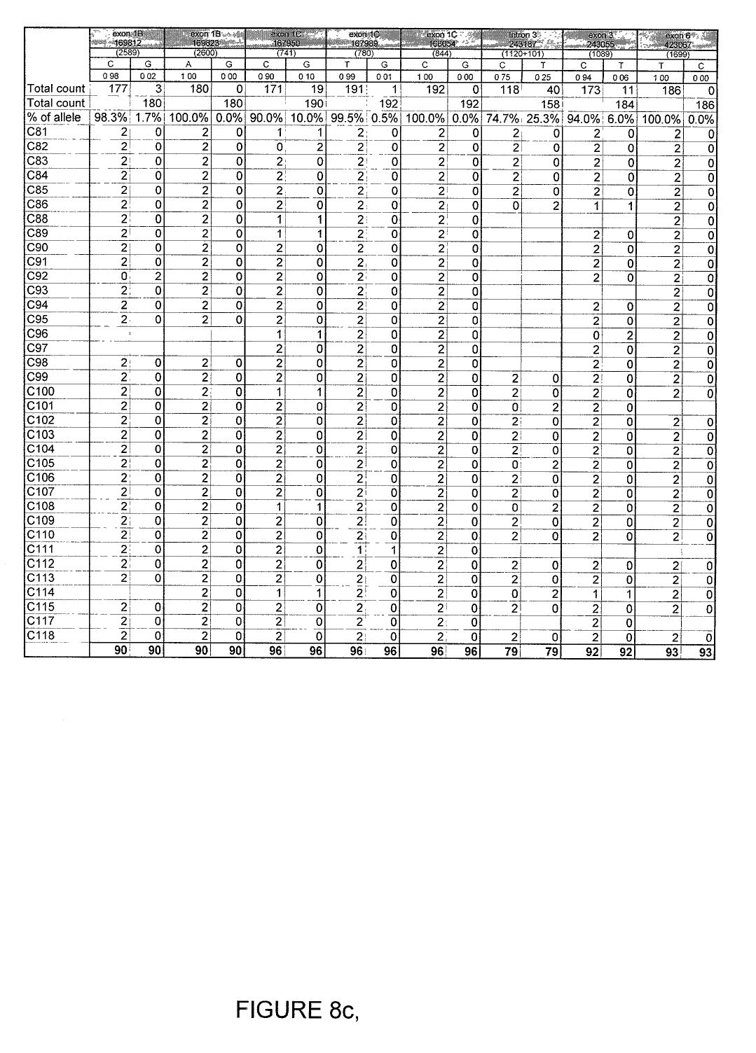

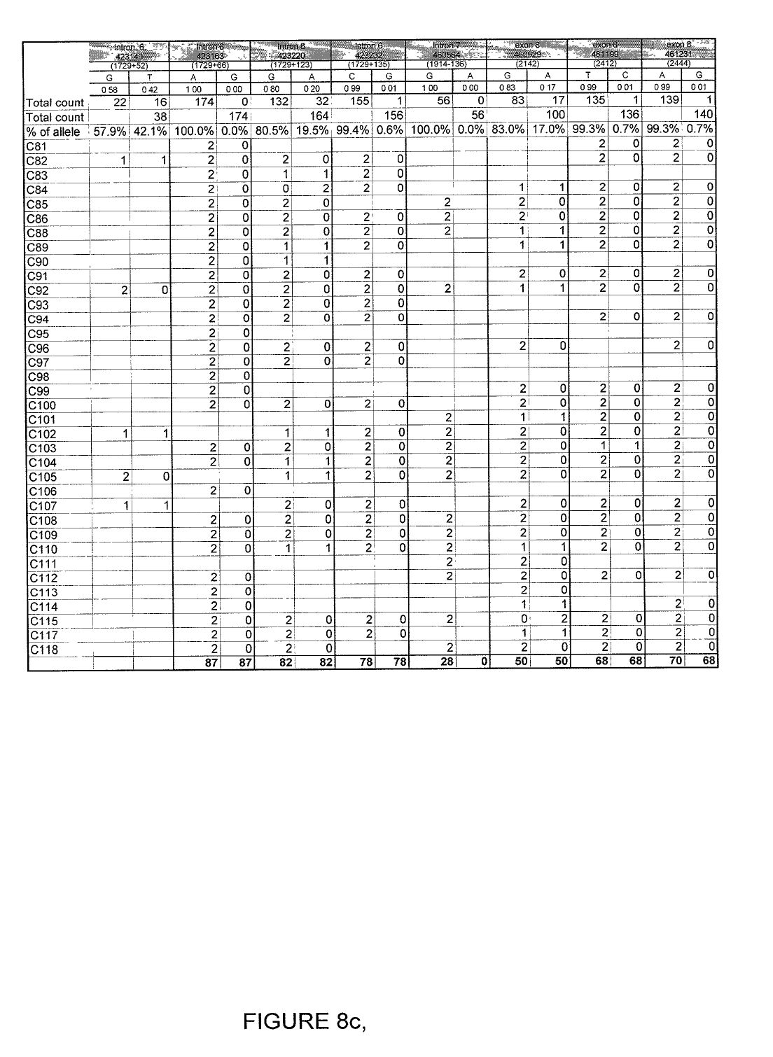

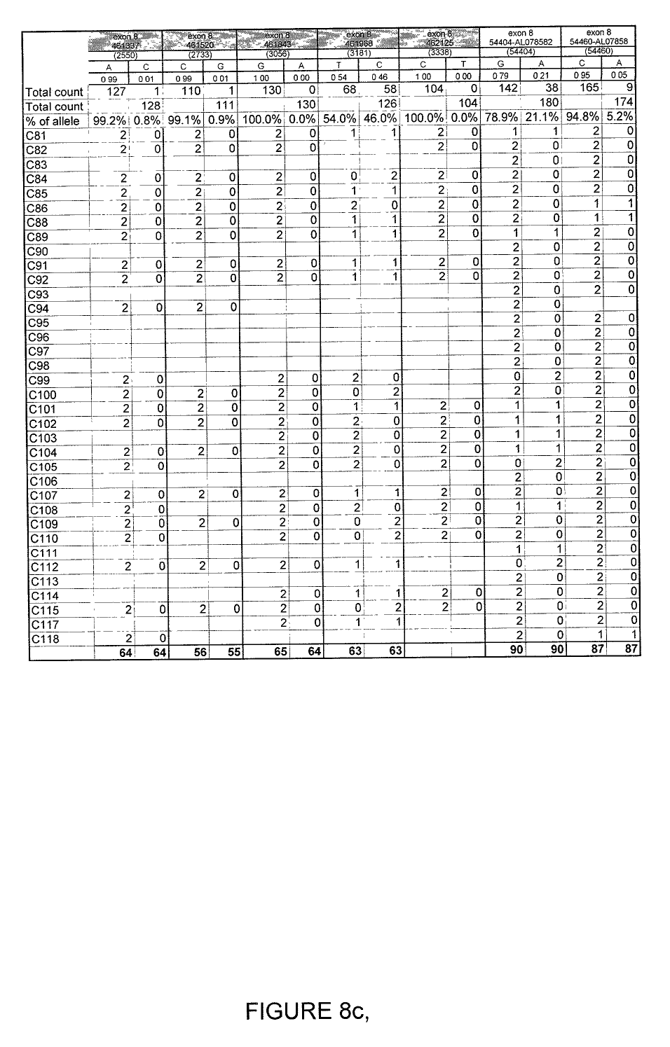

TABLE 1
Summary of SNPs found in the clinical samples.
All SNPs had a frequency greater than 2 and a
quality score greater than 20 (see FIG. 8).

| | |
|---|---|
| Number of SNPs | 39 |
| Number in Liverpool | 34 |
| Number in Coriell | 26 |
| Number unique to Coriell | 5 |
| Number unique in Liverpool | 12 |

TABLE 2
Summary of changes in heterozygosity in clinical samples.
SNPs had a frequency greater than two and a quality score
greater than 20 (See FIG. 8).

| | |
|---|---|
| Number of Liverpool SNPs | 15 |
| With >1 Change in Heterozygosity | |
| Number of Liverpool SNPs | 4 |
| With >1 case of Loss of Heterozygosity | |

FIG. 2 (c) included analysis SNPs in Liverpool control population vs. blood or tumor populations. There were 23 sites that were typed in both cases and controls.

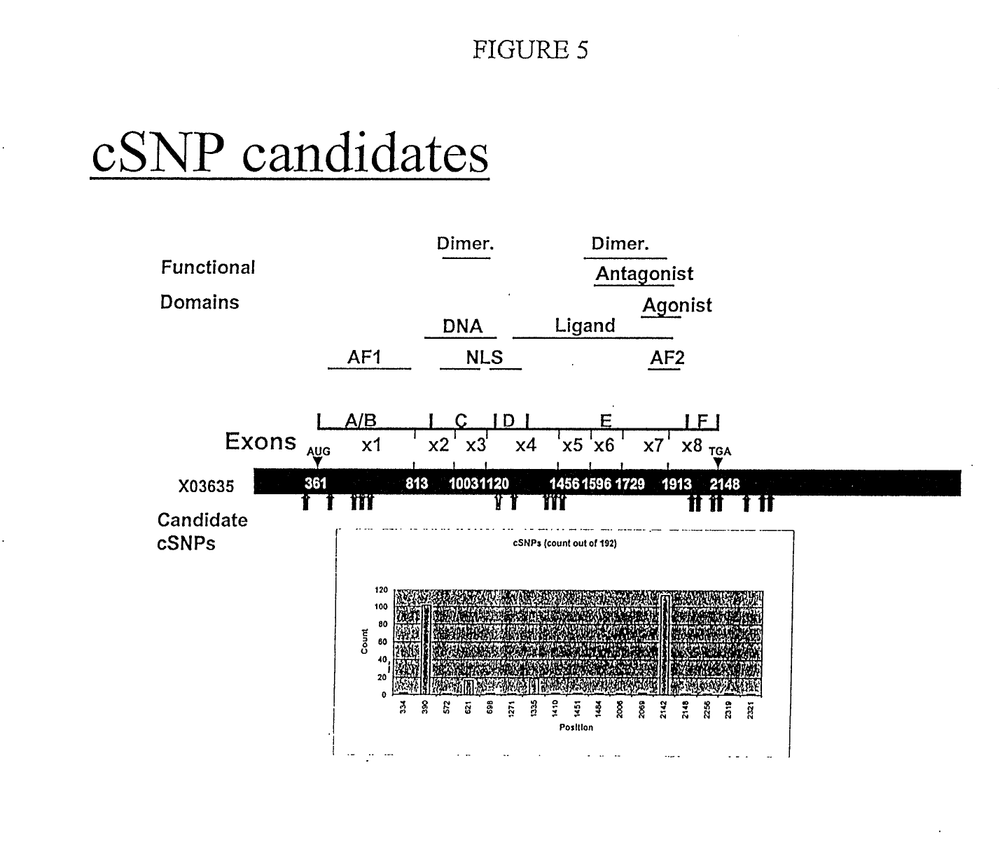

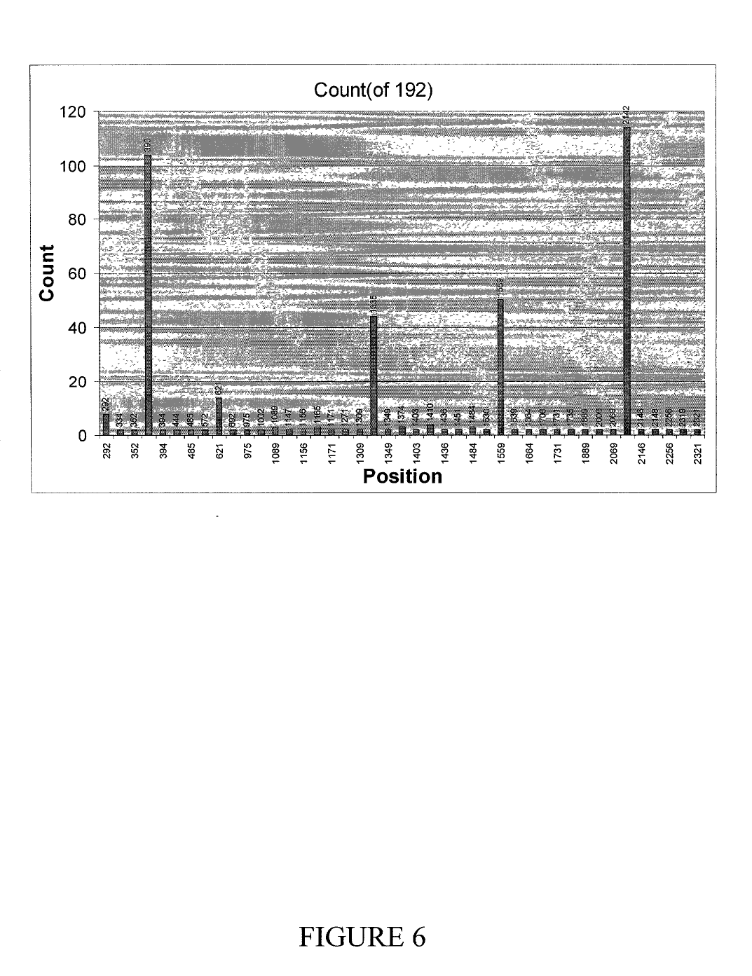

FIG. 5 shows the domain structure of the ESR1 protein and the position of many of the SNPs disclosed herein. FIG. 6 provides a graphical representation of the SNPs and frequency of occurance in the samples tested.

FIG. 8 (a)(1) shows the SNPs and frequency of occurance in Coriell Samples wherein the samples are collected from Northern European, Chinese, Indo-Pakistani, Africa American and Southwestern Native American ethnic groups. In 3′ flanking Exon 8, the positions of the SNPs are based on the sequence of AL078582 that is in Genbank. The result can be used for detection purposes among the specific ethnic groups. FIG. 8 (a)(2) shows the genotyping of SNP on cite 167989 (intron 1D, #9) in the Coriell panel. This panel comprises 100 Caucasians, wherein 51 are males and 49 are females. The result shows that the frequency of the minor allele is 9%.

FIG. 8 (b)(1) shows the SNPs and frequence of occurance in Liverpool samples wherein the samples are selected from the groups of blood samples and breast cancer samples from Norther Europeans.

FIG. 8 (b)(2) shows the Taq Man genotyping results for SNP cite 167, 989 (intron 1D, #9) in additional 60 Liverpool patients. The result shows that the frequency of the minor allele (G) was 14% in blood and 10% in tumors. This is similar to the SNP genotyping in Coriell samples shown in FIG. 8(b)(1), wherein the frequency at the minor allele was 16% in blood and 17% in tumors. The Liverpool control population (95 cases) shows that the frequency at the minor allele was 10.5% (FIG. 2(c)).

2. Haplotype Analysis

The method developed for SNP discovery was designed to recover haplotype data. SNPs could be associated into a specific haplotype. The sample cDNA was from a random population present in unknown proportions. SNPs coming from a specific clone were clustered and built into haplotyes.

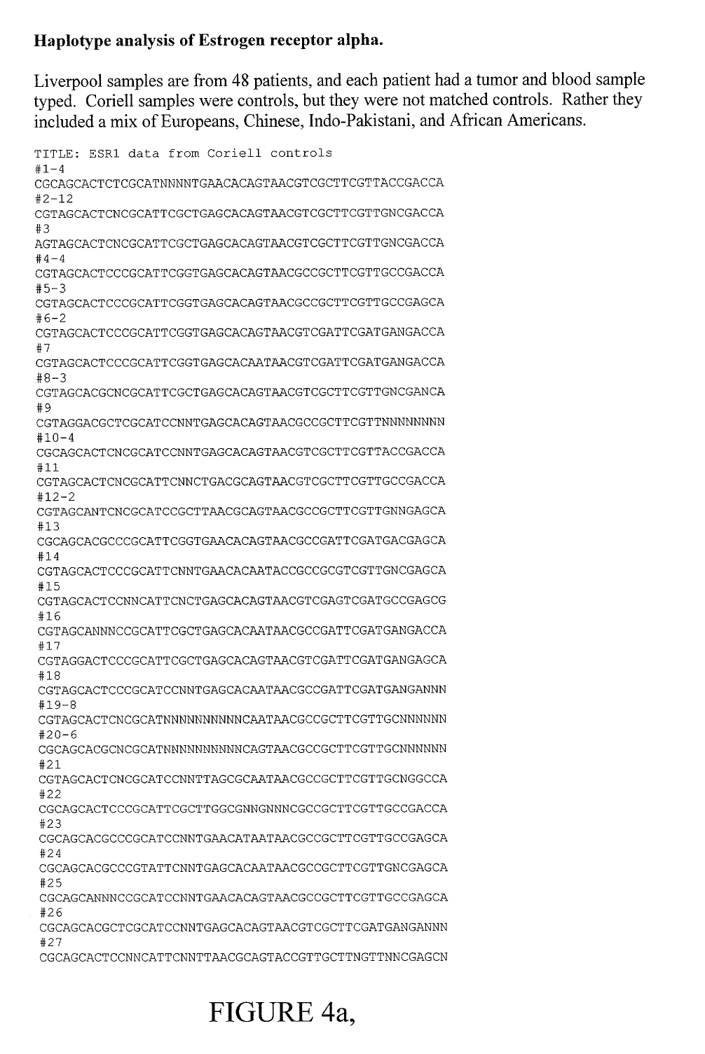

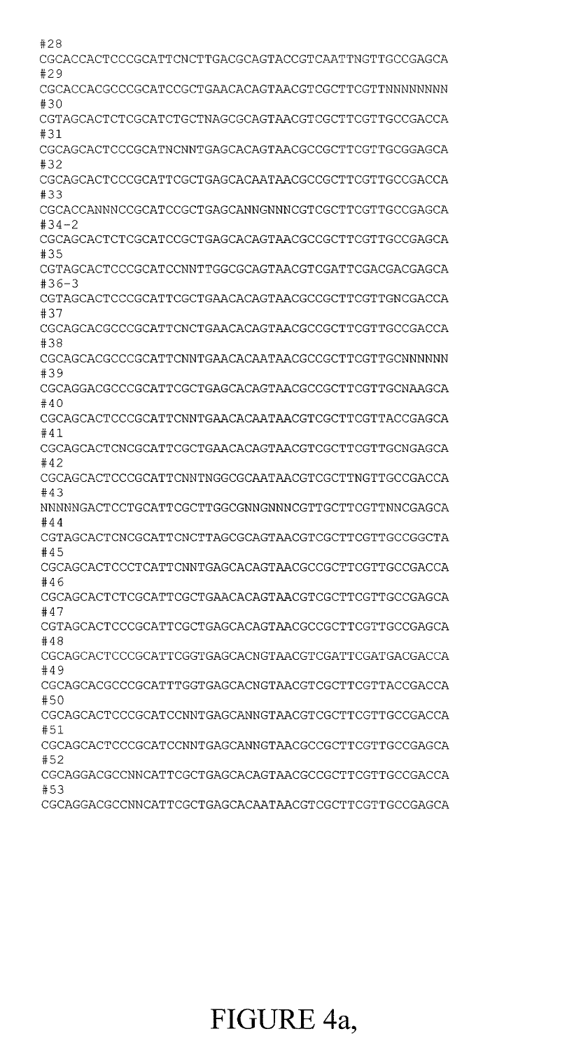

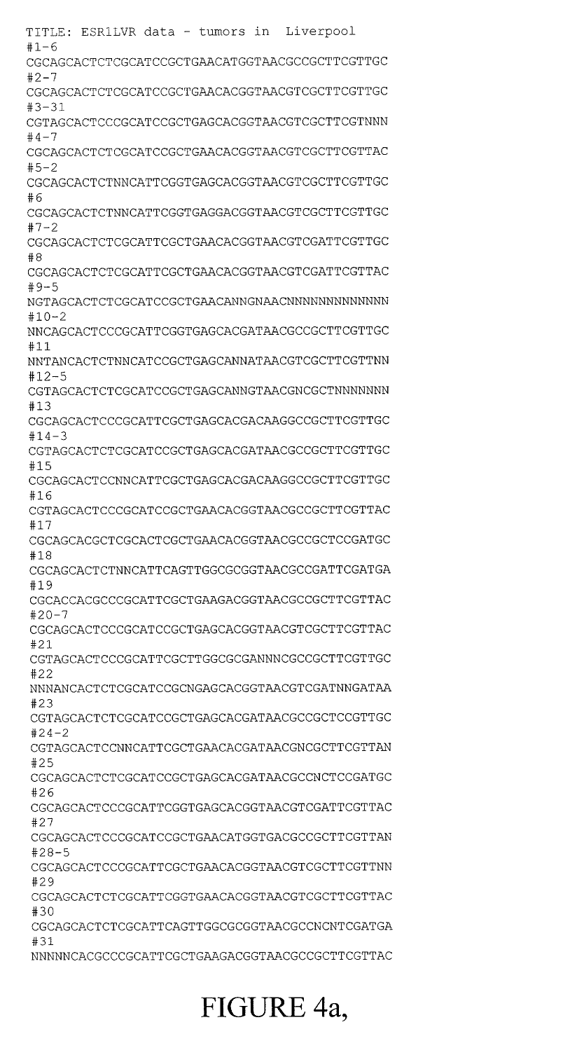

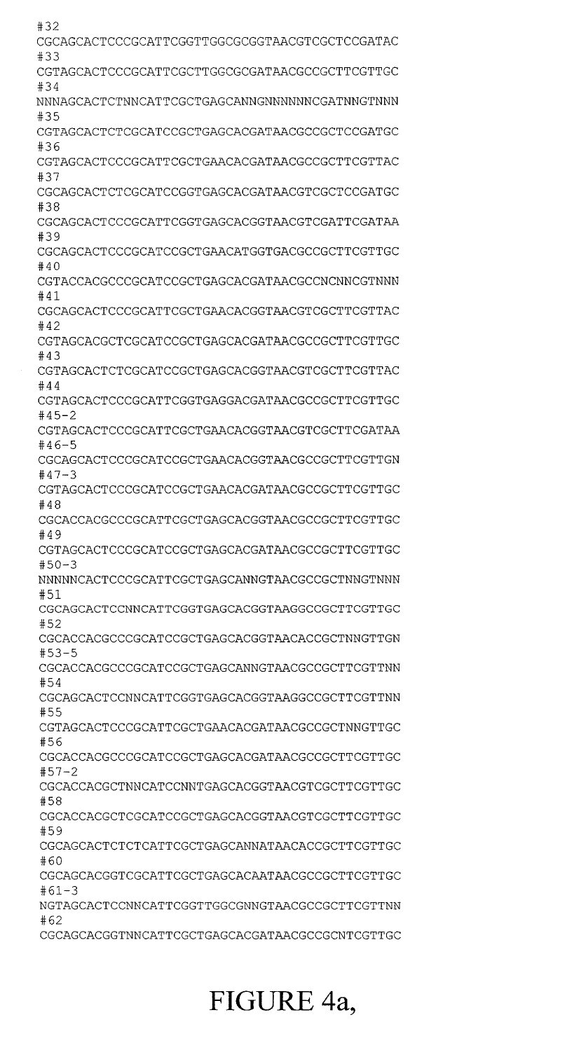

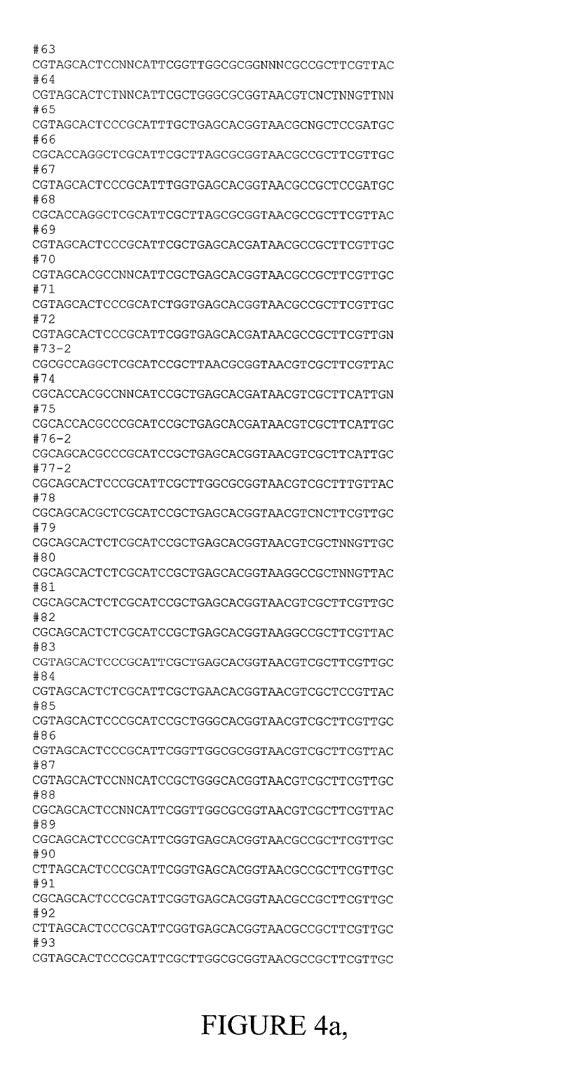

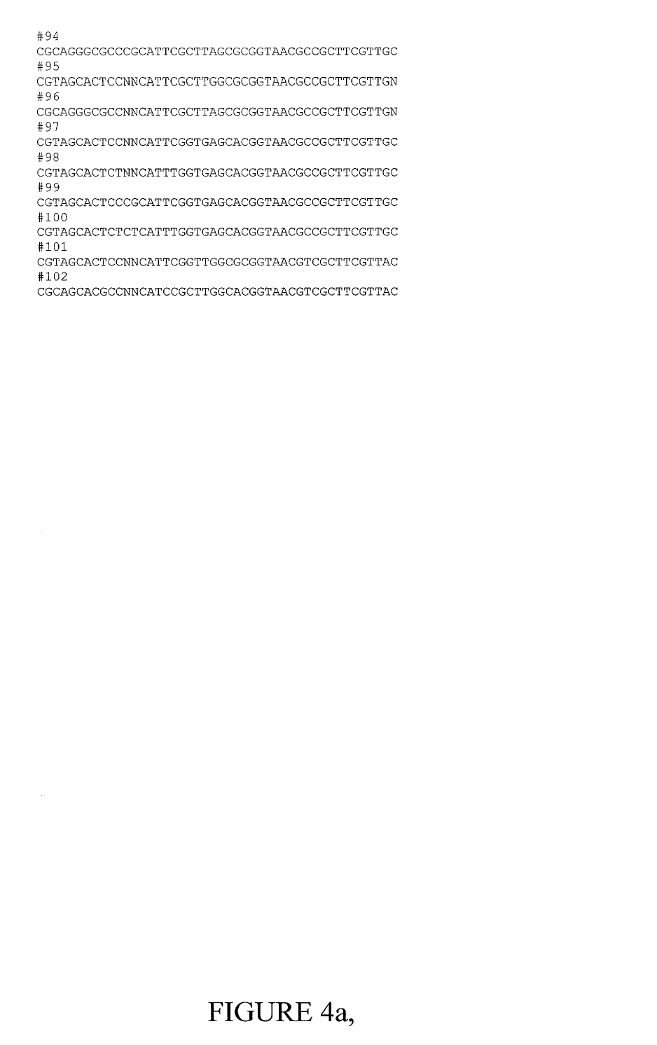

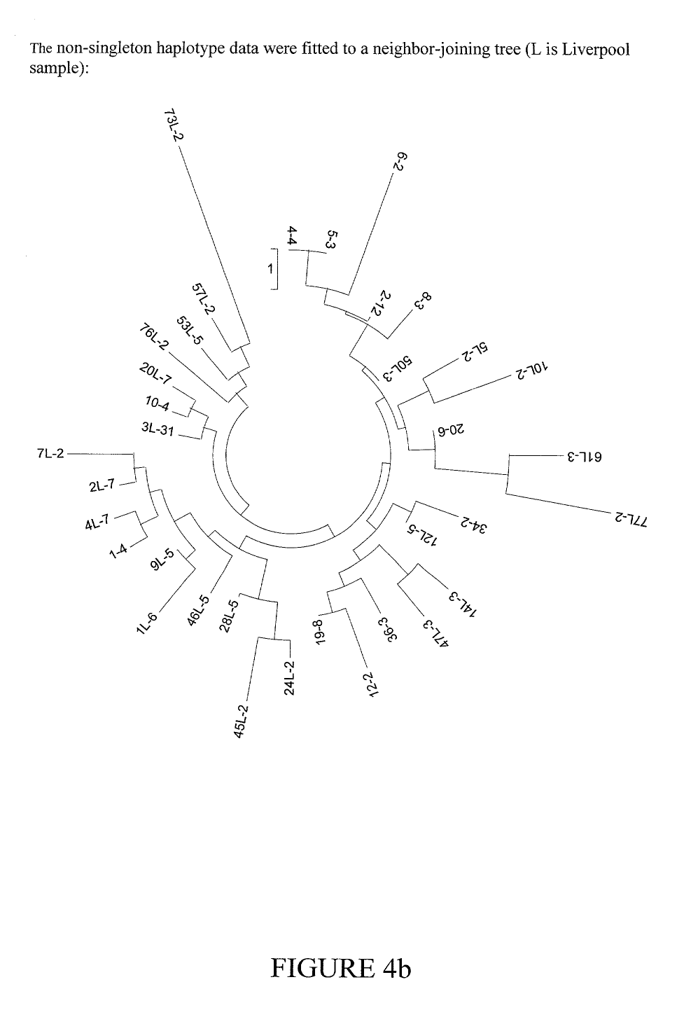

Data consist of two sample types (FIG. 4(a) and (b)). Liverpool samples are from 48 patients, and each patient had a tumor and blood sample typed. Coriell samples were controls, but they were not matched controls. Rather they included a mix of Europeans, Chinese, Indo-Pakistani, and African Americans. 46 SNPs in ESR1 were scored in the Liverpool samples. The same 46 SNPs plus an additional 6 SNPs at 3′ of the RSR1 gene were scored in the Coriell sample.

These data were subjected to an analysis to infer the most likely haplotype phase of the individuals. The results appear in FIG. 4 (a) where each haplotype has a number and the number after the dash is a count (or frequency) of that haplotype.

FIG. 4 (b) is the non-singleton haplotype data that were fitted to a neighbor-joining tree. If a tree were cut at the arrow, the clade including 3L, 10-4, . . . 73L would be partitioned from the rest of the tree, as “Clade X”. The following table will illustrate a difference in the incidence of tumors in haplotypes on Clade X vs. the rest of the tree. The incidence of each haplotype was first counted by adding the numbers after the dashes, wherein L represented the tumorous Liverpool samples and the non-L represented Coriell controls.

| | Clade X | Rest of tree |
|---|---|---|
| Tumor | 49 | 64 |
| Control | 4 | 49 |

A Chi-square was calculated based on the 2×2 table as of 21.29, which, with one degree of freedom that has a probability less than 0.0001. Therefore, the Clade X of the ER1 gene has a much greater chance of being associated with a tumor. This entire lade is so rare elsewhere in the world. Even among Europeans, it was present only once out of 20 haplotypes.

The sites that identified the clade with a large frequency difference between Coriell controls, Liverpool controls, Liverpool tumor samples were:

| | | | | | |
|---|---|---|---|---|---|
| 5 | ESR1-exon 1A | 170487; | 9 | ESR1-intron 1D | 167989; |
| 11 | ESR1-exon 1E | 64331; | 17 | ESR1-exon Intrn 3 | 243187; |
| 20 | ESR1-exon 4 | 306382; | 24 | ESR1-exon Intrn 6 | 423220; |
| 28 | ESR1-exon Intrn 7 | 459832; | 29 | ESR1-intron 7 | 459913; |
| 35 | ESR1-exon 8 | 460929; | 45 | ESR1-exon 8 | 462949 |

3. Promoter region and SNP on Estrogen receptor 1

CAAT and TATA boxes are found in the first 200 b.p. of the sequence and a distance between them lead to believe that they might be functional as a basal promoter.

The distance between CAAT-TATA sites and actual identified start of transcription is about 300 b.p. (usually it is about 20-40 b.p.) and this region contains multiple TF sites such NFAT, NFKB, SP1-family, CEBP and EBOX, majority of them involved in a differential (tissue, cell type) regulation of expression.

Region with T (SNP) has interesting properties. Not only it has conservative (TC)6-repeat, but also (CA)6 on the 5-prime. A conservative motif for is CACAYTCTC at the same region. There is no significant match from known TF sites to this region, and it is likely to be a novel TF site. Very close to T (SNP) is TF site called AHRARNT_{—}02 for aryl hydrocarbon/Arnt heterodimers. It is possible that CACAYTCTC site is either a binding point for the co-factor or help to properly position Arnt complex. It is also known that a single mutation in TF-binding site could decrease affinity of the protein binding several folds and as such may lead into a disease pathway.

In the present invention, the SNP occurs just 13 bp upstream of exon 1C in a short GA repeat (GAGAGAGA). Among SNPs of interest in the clades (FIG. 4), three are silent mutations, one is in the 3′ UTR, and the rest are in intron regions.

The important T to G SNP (#9) is in the site 167989 (Intron ID) of the promoter region (FIG. 2 (a), (b), and (c)). Loss of a gene copy is associated with cancer risk. A promoter mutation that decreases gene expression could cause a similar effect.

G- to T transversion is in an alternate promoter site. There is some effect on estrogen physiology, possible on overall levels of signaling at the nucleus. The predition would be that the “effect” of the relevant clade (and cladistic haplytypes) (FIG. 4) is to increase overall estrogen receptor sensitivity and responsiveness or possible to lead in the direction of alternative growth processes that cycle in the direction of unregulated (non-physiologic) growth signals. Estrogen exposure plays a critical role in breast cancer causation in virtually every epidemiologic study.

In addition, the TF binding sites and SNPs were detected in the ESR1 gene wherein 5 SNPs occured in transcription binding sites as shown below:

| SNP position | Gene structure | Transcription factor |
|---|---|---|
| 167989 | intron 1D | PAX-3 |
| 64331 | exon 1E | CDP |
| 423220 | intron 6 | MEF-2 |
| 423232 | intron 6 | MEF-2 |
| 423258 | intron 6 | SRF, AGL3 |

4. ESR1 Genomic Sequencing—The Complete Genomic Structure of Estrogen Receptor alpha

Estrogen receptor (ER) is a member of the nuclear hormone receptor gene superfamily. This family of genes is characterized by a modular structure with three distinct domains: a variable (N)-terminal domain, a highly conserved DNA binding domain, and a conserved (C)-terminal domain (reviewed in 1, 2). Functionally, the (N)-terminus domain regulates transactivation, the DNA binding domain regulates dimerization and DNA binding, and the (C)-terminus domain regulates transactivation, dimerization, ligand binding, nuclear translocation, silencing, and Heat Shock Protein binding. It was shown that the functions of the individual domains of the nuclear hormone gene superfamily are independent of the receptor in which they are found, and that the domains retain their function even when placed into different heterologous proteins (3, 4, 5). The domain modularity in the nuclear hormone receptor gene superfamily exists because the major subfamilies of these genes evolved through a simple gene duplication early in evolution (6). The nuclear hormone receptor gene family can be separated according to two different classification schemes, one based on hormone binding, the other based on dimerization and how the receptors bind to their respective DNA response elements (for a review, see 2).

The cDNA for ERα was first cloned and sequenced from the MCF-7 breast cancer cell line and was found to have 27% identity and 41% conservation to the v-erb-A gene (7). ERα was mapped to chromosome 6q25.1 using Fluorescence In Situ Hybridization (FISH) and chromosome banding (8). In 1996, a novel estrogen receptor (ERβ) was identified by degenerate PCR (9) and mapped to 14q22-24 by FISH (10). ERα and ERβ were shown to have 96% sequence identity in the DNA binding domain, 58% identity in the ligand-binding domain, and low similarity in the 5′ and 3′ ends as well as in the hinge (domain D). A variety of ERα and ERβ variants have since been described, including single and multiple exon deletions, truncated transcripts, and transcripts containing insertions (11, 12, 13). These variants were isolated from a variety of sources, including normal tissues, tumor tissues and cell lines. The ER status of tumors in breast cancer patients has been used as an indicator of response to endocrine therapy (14, 15), and many studies have examined the role of ER in breast cancer tumor progression, ER-negative status, and hormone antagonist resistance (for a complete review, see 16).

Because of the importance of the ER gene, we set about to clone it in its entirety and determine its complete structure. Initially, we used standard Bacterial Artificial Chromosome (BAC) sequencing to generate sequence information for the coding regions of the genes. As Celera's sequencing of the human genome progressed, the remaining regions of ER were filled in using Celera regional assemblies. A small region of less than 25 kb was filled in on ERα using a public BAC (A1353611.6, positions 1,497-25,941)

Materials and Methods

1) BAC Screening

Appropriate markers were designed for ERα and ERβ exons and used to obtain commercially available BAC clones from Research Genetics (Huntsville, Ala.). A number of positive BACs were selected and individual clones were re-screened for verification.

2) DNA Isolation and Library Preparation

BAC DNA was isolated from verified clones using QIAGEN columns (QIAGEN, Inc., Valencia, Calif.) according to the manufacturer's specifications. Shotgun libraries were prepared following standard protocols (17). Briefly, isolated BAC DNA was sonicated, polished, and size fractionated. Size selected DNA fragments were then subcloned into pUC19 using standard ligation techniques. Ligated DNA was transformed into Electrocompetent cells (Life Technologies, Rockville, Md.) and grown overnight.

3) DNA Sequencing and Annotation

Sequencing reactions were performed using Big Dye Terminator chemistry (Applied Biosystems, Foster City, Calif.) and run on an ABI PRISM 3700 DNA Analyzer (Applied Biosystems). Phred (18), Phrap and Consed (19) were used for base calling, assembly, and finishing, respectively. Exon locations were determined using Cross_Match to compare the published gene sequences to the genomic contig.

Results

1. Estrogen Receptor α

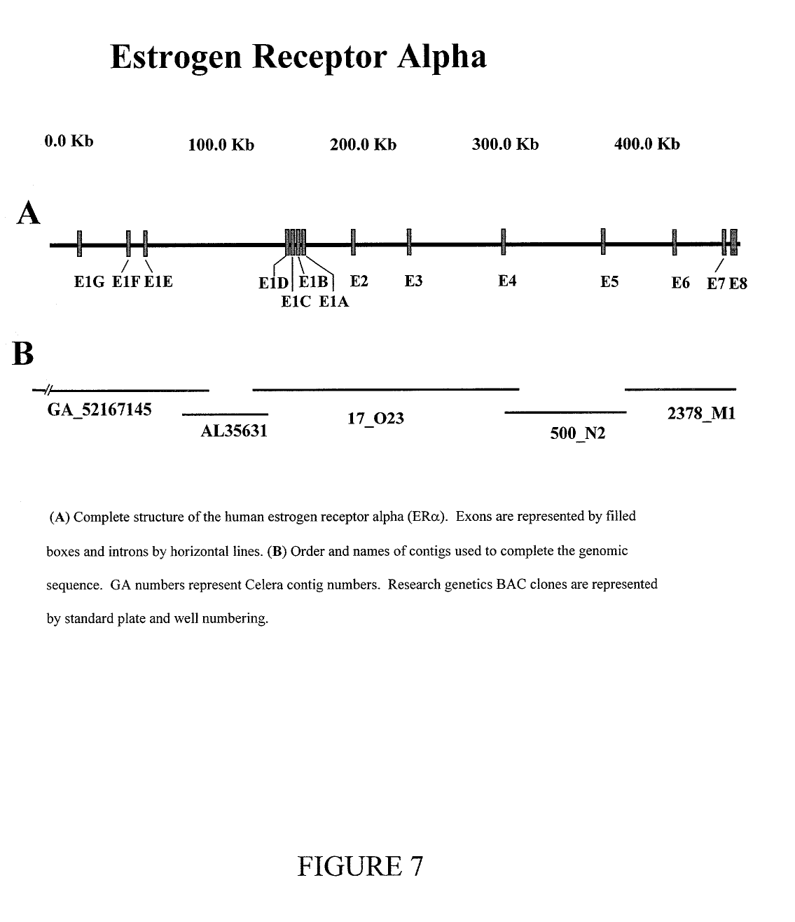

Alignment of the genomic sequence for ERα and published mRNA sequences for ERα show the gene consists of 14 exons and covers 446,296 bp of genomic sequence (FIG. 7, Table 3).

2. Estrogen Receptor β

Alignment of the genomic sequence for ERβ and published mRNA sequences for ERβ show the gene consists of 17 exons and covers 253,748 bp of genomic sequence (FIG. 2, table 1). By analysis with the Celera Genome Browser, we were able to identify a gene, human synaptic nuclei expressed gene 2 (syne-2, accession number NM_{—}015180.1), that is completely contained within intron 9 of ERβ, on the opposite strand. Further analysis of the syne-2 gene showed it consists of 21 exons, and covers 51,471 bp of genomic sequence.

Discussion

Alignment of the complete ERα genomic sequence and various ERβ transcripts shows that the gene covers 446,296 bp of genomic sequence and consists of 14 exons. The alignment of the published sequence for exon 1E (AJ002561) (20) and the ER α genomic sequence revealed that exon 1E actually consists of two separate exons. The newly delineated exon is referred to here as exon 1G to conform to the naming convention previously established. Exon 1G is located approximately 45 kb upstream of exon 1E and conforms to the GT/AG splice site consensus sequence (FIG. 1, table 1).

Alignment of the various ERβ transcripts to the complete ERβ genomic sequence reveals a more complex organization than was previously accepted (13). The 5′ UTR of the ERβcx variant (AB006589) actually consists of seven untranslated exons (referred to here as exons −1 through −7), all of which conform to the GT/AG splice site consensus sequence (FIG. 2, table 1). Sequence alignment of ERβ variants AF061055 and AF061054 (12) showed that these transcripts both contain intron sequence and were probably partially mature transcripts. Both of these partially mature transcripts contain exon 7 and a portion of exon 9, but do not conform to the splice site consensus sequence at the sites where intron sequence is present.

By examining the ER genomic sequences using the Celera Genome Browser, we were able to identify a separate gene contained entirely within intron 9 of ERβ. This gene was identified as human synaptic nuclei expressed gene 2 (syne-2) and was shown to cover over 50 Kb of genomic sequence and consist of 21 exons, all of which conform to the GT/AG splice site consensus sequence (Table 4). The syne-2 gene is located on the antisense strand of ERβ.

Completion of the sequence and structures for ERα and ERβ should contribute to further understanding and characterization of these important receptors.

TABLE 3
Exon-Intron Boundaries and Locations in the Human
Estrogen Receptor: Exon sequences are shown in
upper case and intron sequences are shown in
lower case. Splice sites are shown in bold.

| | | | | | | | | |
|---|---|---|---|---|---|---|---|---|
| | | | | | | | | Intron |
| | Exon | Splice | Contig | Contig | | | Exon Size | size |
| Gen | no. | varient | start | end | 5′ splice donor | 3′ splice acceptor | (bp) | (Kb) |

| | | | | | | | | |
|---|---|---|---|---|---|---|---|---|
| ER1 | 1G | AJ002561 | 18941 | 19032 | - | ACCAAAGAAGgtaagttttt | 91 | 33.79 |
| | 1F | AJ002562 | 52818 | 52940 | - | TTCTCTTCAAgtaggtactc | 122 | 11.21 |
| | 1E | AJ002561 | 64150 | 64280 | aaaacaaaagGAAGAAGAAA | CATCACTGAGgtatgtgtga | 130 | 101.95 |
| | 1D | AJ002560 | 166228 | 166322 | - | GAGAGAGCCAgtaagtcacg | 94 | 1.68 |
| | 1C | X62462 | 168002 | 168120 | - | ATCCAGCAGGgtaggcttgt | 118 | 1 55 |
| | 1B | AJ002559 | 169674 | 169825 | - | GACAAGTAAAgtaaagttca | 151 | 0 04 |
| | 1A | X03635 | 169867 | 170678 | - | CATTCTACAGgtacccgcgc | 811 | 34.23 |
| | 2 | X03635 | 204912 | 205102 | ttccccccagGCCAAATTCA | AGTATTCAAGgtaatagtgt | 190 | 37.87 |
| | 3 | X03635 | 242970 | 243086 | cttttaatagGACATAACGA | ATGAAAGGTGgtaggtacat | 116 | 63.08 |
| | 4 | X03635 | 306168 | 306503 | gtgttttcagGGATACGAAA | AGGGTGCCAGgtaagaatgc | 335 | 67.14 |
| | 5 | X03635 | 373640 | 373778 | ttgttttcagGCTTTGTGGA | TCTTGGACAGgtaagtgacc | 138 | 49 19 |
| | 6 | X03635 | 422964 | 423097 | gttttcatagGAACCAGGGA | CTTAATTCTGgtgagttgat | 133 | 33.26 |
| | 7 | X03635 | 456354 | 456537 | gcgcattcagGAGTGTACAC | GGCACATGAGgtgaggcatc | 183 | 4.16 |
| | 8 | X03635 | 460701 | 465237 | ccacctacagTAACAAAGGC | - | 4536 | - |
| ER2 | −7 | AB006589 | 49552 | 49750 | - | GGTTCTGAAGgtgcgtggtt | 198 | 1.18 |
| | −6 | AB006589 | 50928 | 51235 | tgcctcttagACATCCAAGT | TGTTTGTAAGgtaataaaaa | 307 | 32.62 |
| | −5 | AB006589 | 83858 | 84041 | tatccactagAGGGAGACAT | GAGAACACAGgtgaacttca | 183 | 1.90 |
| | −4 | AB006589 | 85942 | 86154 | ctctccatagAAATCCTGGC | ATTAGCCCTGgtaaggagct | 212 | 2.88 |
| | −3 | AB006589 | 89037 | 89130 | cattcaacagTATCTGGGCT | GTGCAGGTAGgtaggtaaag | 93 | 0.67 |
| | −2 | AB006589 | 89803 | 89988 | ccttttacagGGTTTTGTTT | GTGTTGACAGgtaagatqag | 185 | 3.12 |
| | −1 | AF060555 | 93111 | 93488 | - | TATCTGCAAGgtaagcgccc | 377 | 10.96 |
| | 1 | AF060555 | 104446 | 104897 | ttctttacagCCATTATACT | CTGTAAACAGgtaagtccag | 451 | 2.47 |
| | 2 | AF060555 | 107368 | 107540 | tgctccctagAGAGACACTG | AGCATTCAAGgtacaagaga | 172 | 11.07 |
| | 3 | AF060555 | 118610 | 118726 | tctgctatagGACATAATGA | CTGAAGTGTGgtgagtgctt | 116 | 8.05 |
| | 4 | AF060555 | 126774 | 127073 | tcctcttcagGCTCCCGGAG | AAGATTCCCGgtagggcttt | 299 | 3.09 |
| | 5 | AF060555 | 130158 | 130296 | ctttccccagGCTTTGTGGA | TTCTGGACAGgtgagaaaaa | 138 | 7.56 |
| | 6 | AF060555 | 137853 | 137986 | acttttgtagGGATGAGGGG | CTCAATTCCAgtaaqtaatc | 133 | 14.39 |
| | 7 | AF060555 | 152379 | 152559 | ctttgtccagGTATGTACCC | GGCATGCGAGgtacgcgccc | 180 | 1.65 |
| | 8 | X99101 | 154206 | 154500 | gtccccatagTAACAAGGGC | - | 826 | 5.42 |
| | 9 | AB006589 | 159915 | 160827 | tctacttaagGGCAGAAAAG | - | 912 | 141.65 |
| | 10 | AF060555 | 302474 | 303300 | gtcttgacagCTCTCTCTCA | - | 826 | - |

TABLE 4
Exon-Intron Boundaries and Locations in the Human
Synaptic Nuclei Expressed Gene 2. Exon sequences
are shown in upper case and intron sequences are
shown in lower case. Splice sites are shown in bold.

| | | | | | | | | |
|---|---|---|---|---|---|---|---|---|
| | | | | | | | Exon | Intron |
| | Exon | Splice | Contig | Contig | | | Size | size |
| Gene | no. | varient | start | end | 5′ splice donor | 3′ splice acceptor | (bp) | (Kb) |

| | | | | | | | | |
|---|---|---|---|---|---|---|---|---|
| Syne-2 | 1 | NM_015180 | 212563 | 212391 | - | CACTGTAGAGgtaaactcac | 172 | 2.22 |
| | 2 | NM_015180 | 210175 | 210044 | tttcaaatagACCTGGGACC | GCTGATTAAGgtattgaaat | 131 | 8.94 |
| | 3 | NM_015180 | 201109 | 200946 | ttaaatgcagGAACTAGAAC | CTGCTTAAGGgtaagtcagc | 163 | 1.97 |
| | 4 | NM_015180 | 198981 | 198811 | tcatttgcagGTGGCCATAC | GTTACAGAAGgtaagggagg | 162 | 1.36 |
| | 5 | NM_015180 | 197462 | 197290 | cctttgccagGACTGCATGG | TCGGATCAAGgtaagaaatg | 172 | 12.56 |
| | 6 | NM_015180 | 184732 | 184564 | atatgtgtagGGTGAAGAAG | TGAGCAGCAGgtgggacaat | 168 | 5.79 |
| | 7 | NM_015180 | 178771 | 178584 | gtaatcacagGATCTACAGC | GGCCCATGAAgtaagaacta | 193 | 0.48 |
| | 8 | NM_015180 | 178101 | 177949 | ctcccatcagAATCGAGGAG | GAGGTTTGAGgtaaacacct | 152 | 0.36 |
| | 9 | NM_015180 | 177591 | 177405 | tgtqatgcagGCCTTTCACC | CAGACTCAGGgtgagctcct | 186 | 1.84 |
| | 10 | NM_015180 | 175570 | 175429 | acttttgcagCATTTCACGA | CCAACTGAATgtgagggctg | 141 | 0.71 |
| | 11 | NM_015180 | 174718 | 174522 | ctctcaacagGGCTTGCAAC | CTGCACTCCGgtacggqcac | 196 | 1.19 |
| | 12 | NM_015180 | 173337 | 173051 | tgtggtttagGGCTTGGAAC | GCACTGTCAGgtaacagctg | 286 | 1.76 |
| | 13 | NM_015180 | 171289 | 171140 | ttcgtttcagGTAAATCCAT | ACCACCCTATgtaagtctta | 149 | 2.00 |
| | 14 | NM_015180 | 169139 | 169011 | ctcattctagGGAAAGCTAC | CAGCAGTCAGgtactgcctg | 126 | 0.69 |
| | 15 | NM_015180 | 168327 | 168117 | ttaattccagGTGCCTTCGA | GAGACTGCAGgtgagttaga | 210 | 1 02 |
| | 16 | NM_015180 | 167096 | 166890 | tctctggtagGAGATACTGA | GCAGTGCCAGgtacgctgac | 206 | 0.93 |
| | 17 | NM_015180 | 165957 | 165825 | gttttttaagGACTTCCACC | GGAACTAATGgtaagtttcc | 132 | 1.48 |
| | 18 | NM_015180 | 164342 | 164149 | ctgttttcagCAACTGGAAA | GGCAACCCAGgtgagtctac | 193 | 1.08 |
| | 19 | NM_015180 | 163074 | 162982 | tgaatttcagAACCCAGCCT | CCGAGCAAAGgtaagaagcc | 92 | 0.45 |
| | 20 | NM_015180 | 162537 | 162482 | ctttacccagCAGTTCAGAG | CAGAGAGCAGgtaacggggc | 55 | 0.27 |
| | 21 | NM_015180 | 162214 | 161092 | ctgttggcagGGTCCCCGGC | - | 1122 | - |

References

1. Ribeiro R C, Kushner P J, Baxter J D, Tenbaum S, Baniahmad A. The nuclear hormone receptor gene superfamily. Annu Rev Med 1995;46:443-53.

2. Tenbaum S, Baniahmad A. Nuclear receptors: structure, function and involvement in disease. Int J Biochem Cell Biol. 1997 Dec;29(12):1325-41.

3. Baniahmad C, Baniahmad A, O'Malley B W. A rapid method combining a functional test of fusion proteins in vivo and their purification. Biotechniques. February 1994; 16(2): 194-6.

4. Parker M G, White R. Nuclear receptors spring into action. Nat Struct Biol. February 1996;3(2):113-5.

5. Schwabe J W. Transcriptional control: how nuclear receptors get turned on. Curr Biol. 1996 Apr 1;6(4):372-4.

6. Laudet V, Hanni C, Coll J, Catzeflis F, Stehelin D. Evolution of the nuclear receptor gene superfamily. EMBO J. March 1992; 11(3):1003-13.

7. Green S, Walter P, Kumar V, Krust A, Bornert J M, Argos P, Chambon P. Human oestrogen receptor cDNA: sequence, expression and homology to v-erb-A. Nature. March 1986 13-19;320(6058):134-9.

8. Menasce L P, White G R, Harrison C J, Boyle J M. Localization of the estrogen receptor locus (ESR) to chromosome 6q25.1 by FISH and a simple post-FISH banding technique. Genomics 1993 Jul;17(1):263-5.

9. Mosselman S, Polman J, Dijkema R. ER beta: identification and characterization of a novel human estrogen receptor. FEBS Lett. 1996 Aug 19;392(1):49-53.

10. Enmark E, Pelto-Huikko M, Grandien K, Lagercrantz S, Lagercrantz J, Fried G, Nordenskjold M, Gustafsson J A. Human estrogen receptor beta-gene structure, chromosomal localization, and expression pattern. J Clin Endocrinol Metab. 1997 Dec;82(12):4258-65.

11. Murphy L C, Dotzlaw H, Leygue E, Douglas D, Coutts A, Watson P H. Estrogen receptor variants and mutations. J Steroid Biochem Mol Biol. 1997 Aug;62(5-6):363-72.

12. Moore J T, McKee D D, Slentz-Kesler K, Moore L B, Jones S A, Home E L, Su J L, Kliewer S A, Lehmann J M, Willson T M. Cloning and characterization of human estrogen receptor beta isoforms. Biochem Biophys Res Commun. 1998 Jun 9;247(1):75-8.

13. Ogawa S, Inoue S, Watanabe T, Orimo A, Hosoi T, Ouchi Y, Muramatsu M. Molecular cloning and characterization of human estrogen receptor betacx: a potential inhibitor of estrogen action in human. Nucleic Acids Res. 1998 Aug 1;26(15):3505-12.

14. Osborne C K, Yochmowitz M G, Knight W A 3d, McGuire W L. The value of estrogen and progesterone receptors in the treatment of breast cancer. Cancer 1980 Dec 15;46(12 Suppl):2884-8.

15. DeSombre E R, Carbone P P, Jensen E V, McGuire W L, Wells S A Jr, Wittliff J L, Lipsett M Special report. Steriod receptors in breast cancer. N Engl J Med 1979 Nov 1;301(18):1011-2.

16. Parl, Fritz F., Estrogens, Estrogen receptor, and Breast Cancer. IOS Press, Amsterdam, Netherlands, 2000.

17. Birren B, Green E D, Klapholz S, Myers, R, Riethman H, Roskams J, (eds.) (1997), Genome Analysis: A Laboratory Manual. Cold Spring Harbor Laboratory Press, Cold Spring Harbor, N.Y.

18. Ewing B, Hillier L, Wendl M C, Green P. Base-calling of automated sequencer traces using phred. I. Accuracy assessment. Genome Res March 1998;8(3):175-85.

19. Gordon D, Abajian C, Green P. Consed: a graphical tool for sequence finishing. Genome Res March 1998;8(3):195-202.

20. Flouriot G, Griffin C, Kenealy M, Sonntag-Buck V, Gannon F. Differentially expressed messenger RNA isoforms of the human estrogen receptor-alpha gene are generated by alternative splicing and promoter usage. Mol Endocrinol December 1998;12(12):1939-54.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the above-described modes for carrying out the invention which are obvious to those skilled in the field of molecular biology or related fields are intended to be within the scope of the following claims.

That which is claimed is:

1. An isolated peptide consisting of an amino acid sequence selected from the group consisting of:
(a) the amino acid sequence of a variant estrogen receptor protein provided in FIG. 2;
(b) a fragment of the amino acid sequence of a variant estrogen receptor protein provided in FIG. 2, wherein the fragment comprises at least 10 contiguous amino acids.

2. An isolated peptide comprising an amino acid sequence selected from the group consisting of:
(a) the amino acid sequence of a variant estrogen receptor protein provided in FIG. 2;
(b) a fragment of the amino acid sequence of a variant estrogen receptor protein provided in FIG. 2, wherein the fragment comprises at least 10 contiguous amino acids.

3. An isolated antibody that selectively binds to a peptide of claim 1.

4. An isolated nucleic acid molecule consisting of a nucleotide sequence selected from the group consisting of:
(a) a nucleotide sequence that encodes the amino acid sequence of a variant estrogen receptor protein provided in FIG. 2;
(b) a nucleotide sequence that encodes a fragment of the amino acid sequence of a variant estrogen receptor protein provided in FIG. 2; and
(c) a nucleic acid molecule that is the complement of a nucleic acid molecule of (a)-(b).

5. An isolated nucleic acid molecule comprising a nucleotide sequence selected from the group consisting of:
(a) a nucleotide sequence that encodes the amino acid sequence of a variant estrogen receptor protein provided in FIG. 2;
(b) a nucleotide sequence that encodes a fragment of the amino acid sequence of a variant estrogen receptor protein provided in FIG. 2; and
(c) a nucleic acid molecule that is the complement of a nucleic acid molecule of (a)-(b).

6. A nucleic acid vector comprising the nucleic acid sequences of claim 4.

7. A nucleic acid vector comprising the nucleic acid sequences of claim 5.

8. A host cell containing the vector of claim 6.

9. A host cell containing the vector of claim 7.

10. A method for producing any of the peptides of claim 1 comprising introducing a nucleotide sequence encoding any of the peptide sequences in (a)-(b) into a host cell, and culturing the host cell under conditions in which the proteins are expressed from the nucleic acid.

11. A method for producing any of the peptides of claim 2 comprising introducing a nucleotide sequence encoding any of the peptide sequences in (a)-(b) into a host cell, and culturing the host cell under conditions in which the proteins are expressed from the nucleic acid.

12. A method for detecting the presence of any of the peptides of claim 1 in a sample, said method comprising contacting said sample with an agent that specifically allows detection of the presence of the peptide in the sample and then detecting the presence of the peptide.

13. A kit comprising reagents used for the method of claim 12, wherein the reagents comprise an agent that specifically binds to said peptide.

14. A method for detecting the presence of a nucleic acid sequence of claim 4 in a sample, the method comprising contacting the sample with an oligonucleotide that hybridizes to the nucleic acid sequences under stringent conditions and determining whether the oligonucleotide binds to the nucleic acid sequence in the sample.

15. A kit comprising reagents used for the method of claim 14, wherein the reagents comprise a compound that hybridizes under stringent conditions to any of the nucleic acid molecules.

16. A method for identifying an agent that binds to any of the peptides of claim 1, said method comprising contacting the peptide with an agent and assaying the contacted mixture to determine whether a complex is formed with the agent bound to the peptide.

17. A method of identifying an individual having or at risk of developing a disorder mediated by a variant estrogen receptor comprising the step of analyzing nucleic acid molecules isolated from said individual for alterations in the estrogen receptor gene sequence, wherein an alteration in said estrogen receptor gene selected from the group consisting of the variants provided in FIG. 2 and FIG. 4 identifies an individual as having or at risk of developing said bone disorder.
